(19) United States
(12) Patent Application Publication
(10) Pub. No.: US 2020/0308129 A1
(43) Pub. Date: Oct. 1, 2020

(54) SUBSTITUTED AROMATIC AMINES FOR USE IN ORGANIC ELECTROLUMINESCENT DEVICES

(71) Applicant: Merck Patent GmbH, Darmstadt (DE)

(72) Inventors: Elvira MONTENEGRO, Weinheim (DE); Teresa MUJICA-FERNAUD, Darmstadt (DE); Florian MAIER-FLAIG, Weinheim (DE); Frank VOGES, Bad Duerkheim (DE); Alexander COMELY, Barcelona (ES); Rosa MORENO FLORES, Barcelona (ES)

(21) Appl. No.: 16/772,768

(22) PCT Filed: Dec. 12, 2018
(86) PCT No.: PCT/EP2018/084461

§ 371 Date: Jun. 13, 2020

(30) Foreign Application Priority Data
Dec. 15, 2017 (EP) 17207840.4

(51) Int. Cl. C07D 307/91; C07C 211/61; C07D 209/82

(52) U.S. Cl. CPC C07D 307/91; H01L 51/5056; C07D 209/82; C07C 211/61

(57) ABSTRACT

The present application relates to a specific fluorene derivative, to its use in an electronic device and to an electronic device comprising said fluorene derivatives. Further, the present application relates to a process for the preparation of such fluorene compounds and to oligomers, polymers or dendrimers as well as formulations or compositions comprising one or more of said fluorene compounds.

The present application relates to a fluorene compound of a formula (I) defined hereinafter, to its use in electronic devices, in particular organic electroluminescent devices such as organic light emitting devices (OLEDs), and to an electronic device comprising a compound of formula (I). Further, the present application relates to a process for the preparation of said compound and to oligomers, polymers or dendrimers as well as formulations or compositions comprising one or more of said compound.

Electronic devices in the context of this application are understood to mean what are called “organic electronic devices”, which contain organic semiconductor materials as functional materials. More particularly, these devices are understood to mean organic electroluminescent (EL) devices, especially organic light emitting diodes (OLEDs).

The general structure and mode of operation of organic electroluminescent devices is known to the skilled person and is described, for example, in U.S. Pat. Nos. 4,539,507, 5,151,629, EP 0676461 and WO 98/27136. In general, organic electroluminescent devices contain spaced electrodes separated by one or more layers comprising organic compounds, which form the so-called organic light emitting structure and emit electromagnetic radiation, typically light, in response to the application of an electrical potential difference across the electrodes.

In electronic devices, especially EL devices such as OLEDs, there is great interest in improving the performance data, especially lifetime, efficiency and operating voltage. In these aspects, it has not yet been possible to find any entirely satisfactory solution.

A great influence on the performance data of electronic devices is possessed by layers having a hole-transporting function, for example hole-injecting layers, hole transport layers, electron blocking layers and also emitting layers. For use in these layers, there is a continuous search for new materials having hole-transporting properties.

In the course of the present invention, it has been found that fluorene compounds or derivatives, which have an amine or bridged amine group in the 2-position and a further substituent, which is selected from particular chemical groups, in one or more of the 5-, 6-, and 8-position, preferably in the 5-position, of the fluorene basic structure, are very well suited for use as materials with hole transporting function, in particular for use as materials of the hole transporting layer, the electron blocking layer and the emitting layer, more particularly for use in the electron blocking layer. An electron blocking layer is understood in this context to be a layer which is directly adjacent to the emitting layer on the anode side, and which serves to block electrons which are present in the emitting layer from entering the hole transporting layers of the EL device.

When used in electronic devices, in particular in EL devices such as OLEDs, they lead to excellent results in terms of lifetime, operating voltage and quantum efficiency of the devices. The compounds are also characterized by very good hole-conducting properties, very good electron-blocking properties, high glass transition temperature, high oxidation stability, good solubility, high thermal stability, and low sublimation temperature.

The present application therefore relates to a compound of the formula (I)

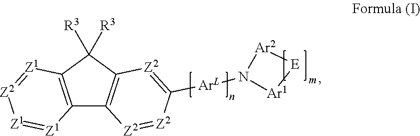

in which the variables are defined as follows:
Z¹ is, identically or differently on each occurrence, selected from CR¹, CR² and N;
Z² is, identically or differently on each occurrence, selected from CR² and N;
Ar^{L} is selected from aromatic ring systems having 6 to 40 aromatic ring atoms, which may be substituted by one or more radicals R⁴, and heteroaromatic ring systems having 5 to 40 aromatic ring atoms, which may be substituted by one or more radicals R⁴;
Ar¹, Ar² are, identically or differently, selected from aromatic ring systems having 6 to 40 aromatic ring atoms, which may be substituted by one or more radicals R⁴, and heteroaromatic ring systems having 5 to 40 aromatic ring atoms, which may be substituted by one or more radicals R⁴;
E is a single bond or is a divalent group selected from —C(R⁴)₂—, —N(R⁴)—, —O—, and —S—;
R¹ is selected, identically or differently on each occurrence, from Si(R⁵)₃, straight-chain alkyl, alkoxy or thioalkyl groups having 1 to 20 C atoms, branched or cyclic alkyl, alkoxy or thioalkyl groups having 3 to 20 C atoms, aromatic ring systems having 6 to 40 aromatic ring atoms, and heteroaromatic ring systems having 5 to 40 aromatic ring atoms, where the said alkyl, alkoxy and thioalkyl groups and the said aromatic and heteroaromatic ring systems may in each case be substituted by one or more radicals R⁵;
R² is selected, identically or differently on each occurrence, from H, D, F, CI, Br, I, C(═O)R⁵, CN, Si(R⁵)₃, N(R⁵)₂, P(═O)(R⁵)₂, OR⁵, S(═O)R⁵, S(═O)₂R⁵, SCN, SF₅, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R² may be connected to each other to form a ring; where the said alkyl, alkoxy, alkenyl and alkynyl groups and the said aromatic and heteroaromatic ring systems may in each case be substituted by one or more radicals R⁵, and where one or more CH₂ groups in the said alkyl, alkoxy, alkenyl and alkynyl groups may in each case be replaced by —R⁵C═CR⁵—, —C≡O—, Si(R⁵)₂, C═O, C═S, C═NR⁵, —C(═O)O—, —C(═O)NR⁵—, NR⁵, P(═O)(R⁵), —O—, —S—, SO or SO₂;
R³ is selected, identically or differently on each occurrence, from H, D, F, CI, Br, I, C(═O)R⁴, CN, Si(R⁴)₃, NO₂, P(═O)(R⁴)₂, S(═O)R⁴, S(═O)₂R⁴, straight-chain alkyl, alkoxy or thioalkyl groups having 1 to 20 C atoms, or branched or cyclic alkyl, alkoxy or thioalkyl groups having 3 to 20 C atoms, alkenyl or alkynyl groups having 2 to 20 C atoms, where the said alkyl, alkoxy, thioalkyl, alkenyl and alkynyl groups may in each case be substituted by one or more radicals R⁵ and where one or more CH₂ groups in the said alkyl, alkoxy, thioalkyl, alkenyl and alkynyl groups may in each case be replaced by —R⁵C═CR⁵—, —C≡O—, Si(R⁵)₂, C═O, C═S, C═NR⁵, —C(═O)O—, —C(═O)NR⁵—, NR⁵, P(═O)(R⁵), —O—, —S—, SO or SO₂ and where in the said alkyl, alkoxy, thioalkyl, alkenyl and alkynyl groups one or more H atoms may be replaced by D, F, Cl, Br, I, CN or NO₂, or aromatic or heteroaromatic ring systems having 5 to 30 aromatic ring atoms, where the said aromatic and heteroaromatic ring systems may in each case be substituted by one or more radicals R⁵, or aryloxy groups having 5 to 60 aromatic ring atoms, or arylalkyl groups having 5 to 60 aromatic ring atoms, where the said aryloxy and arylalkyl groups may in each case be substituted by one or more radicals R⁵, where the two radicals R³ may be connected to each other to form a ring, so that a spiro compound is built at position 9 of the fluorene group, where spirobifluorenes are excluded;
R⁴ is, identically or differently on each occurrence, selected from H, D, F, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R⁴ may be connected to each other to form a ring; where the said alkyl, alkoxy, alkenyl and alkynyl groups and the said aromatic and heteroaromatic ring systems may in each case be substituted by one or more radicals R⁵, and where one or more CH₂ groups in the said alkyl, alkoxy, alkenyl and alkynyl groups may in each case be replaced by —R⁵C═CR⁵—, —C≡O—, Si(R⁵)₂, C═O, C═S, C═NR⁵, —C(═O)O—, —C(═O)NR⁵—, NR⁵, P(═O)(R⁵), —O—, —S—, SO or SO₂;
R⁵ is, identically or differently at each occurrence, selected from H, D, F, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R⁵ may be connected to each other to form a ring; where the said alkyl, alkoxy, alkenyl and alkynyl groups and the said aromatic and heteroaromatic ring systems may in each case be substituted by one or more radicals R⁶, and where one or more CH₂ groups in the said alkyl, alkoxy, alkenyl and alkynyl groups may in each case be replaced by —R⁶C═CR⁶—, —C≡O—, Si(R⁶)₂, C═O, C═S, C═NR⁶, —C(═O)O—, —C(═O)NR⁶—, NR⁶, P(═O)(R⁶), —O—, —S—, SO or SO₂;
R⁶ is selected, identically or differently at each occurrence, from H, D, F, CN, alkyl groups having 1 to 20 C atoms, aromatic ring systems having 6 to 40 C atoms, and heteroaromatic ring systems having 5 to 40 aromatic ring atoms; where two or more radicals R⁶ may be connected to each other to form a ring; and where the said alkyl groups, aromatic ring systems and heteroaromatic ring systems may be substituted by F and CN;
m is 0 or 1, where in the case of m=0, the group E is not present and the groups Ar¹ and Ar² are not connected;
n is 0 or 1; where in the case of n=0, the group Ar^{L} is not present and the nitrogen atom and the fluorene group are directly connected;
   characterized in that at least one of groups Z¹ is CR¹.

In formula (I) only one resonance form of the fluorene basic structure is illustrated, and it is clear to a person skilled in the art that other resonance structures with alternating single and double bonds between the atoms forming the aromatic rings exist to describe electron delocalization within the fluorene basic structure, all of which resonance structures are equivalent and thus comprised within the scope of the present invention.

The following definitions apply to the chemical groups used as general definitions. They only apply insofar as no more specific definitions are given.

An aryl group in the sense of this invention contains 6 to 40 aromatic ring atoms, of which none is a heteroatom. An aryl group here is taken to mean either a simple aromatic ring, for example benzene, or a condensed aromatic polycycle, for example naphthalene, phenanthrene, or anthracene. A condensed aromatic polycycle in the sense of the present application consists of two or more simple aromatic rings condensed with one another.

A heteroaryl group in the sense of this invention contains 5 to 40 aromatic ring atoms, at least one of which is a heteroatom. The heteroatoms are preferably selected from N, O and S. A heteroaryl group here is taken to mean either a simple heteroaromatic ring, such as pyridine, pyrimidine or thiophene, or a condensed heteroaromatic polycycle, such as quinoline or carbazole. A condensed heteroaromatic polycycle in the sense of the present application consists of two or more simple heteroaromatic rings condensed with one another.

An aryl or heteroaryl group, which may in each case be substituted by the above-mentioned radicals and which may be linked to the aromatic or heteroaromatic ring system via any desired positions, is taken to mean, in particular, groups derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzo-thiadiazole.

An aryloxy group in the sense of this invention is understood to mean an aryl group as defined above, which is bonded via an oxygen atom.

An arylalkyl group in the sense of this invention is understood to mean an aryl group as defined above, to which an alkyl group as defined below is bonded.

An aromatic ring system in the sense of this invention contains 6 to 40 C atoms in the ring system and does not comprise any heteroatoms as aromatic ring atoms. An aromatic ring system in the sense of this application therefore does not comprise any heteroaryl groups. An aromatic ring system in the sense of this invention is intended to be taken to mean a system which does not necessarily contain only aryl groups, but instead in which, in addition, a plurality of aryl groups may be connected by a non-aromatic unit such as one or more optionally substituted C, Si, N, O or S atoms. The non-aromatic unit in such case comprises preferably less than 10% of the atoms other than H, relative to the total number of atoms other than H of the whole aromatic ring system. Thus, for example, systems such as 9,9′-spirobifluorene, 9,9′-diarylfluorene, triarylamine, diaryl ether, and stilbene are also intended to be taken to be aromatic ring systems in the sense of this invention, as are systems in which two or more aryl groups are connected, for example, by a linear or cyclic alkyl, alkenyl or alkynyl group or by a silyl group. Furthermore, systems in which two or more aryl groups are linked to one another via single bonds are also taken to be aromatic ring systems in the sense of this invention, such as, for example, systems such as biphenyl and terphenyl.

Preferably, an aromatic ring system is understood to be a chemical group, in which the aryl groups which constitute the chemical group are conjugated with each other. This means that the aryl groups are connected with each other via single bonds or via connecting units which have a free pi electron pair which can take part in the conjugation. The connecting units are preferably selected from nitrogen atoms, single C═C units, single C≡O units, multiple C═C units and/or C≡O units which are conjugated with each other, —O—, and —S—.

A heteroaromatic ring system in the sense of this invention contains 5 to 40 aromatic ring atoms, at least one of which is a heteroatom. The heteroatoms are preferably selected from N, O or S. A heteroaromatic ring system is defined as an aromatic ring system above, with the difference that it must obtain at least one heteroatom as one of the aromatic ring atoms. It thereby differs from an aromatic ring system according to the definition of the present application, which cannot comprise any heteroatom as aromatic ring atom.

An aromatic ring system having 6 to 40 aromatic ring atoms or a heteroaromatic ring system having 5 to 40 aromatic ring atoms is in particular a group which is derived from the above mentioned aryl or heteroaryl groups, or from biphenyl, terphenyl, quarterphenyl, fluorene, spirobifluorene, dihydrophenanthrene, dihydropyrene, tetrahydropyrene, indenofluorene, truxene, isotruxene, spiro-truxene, spiroisotruxene, and indenocarbazole.

For the purposes of the present invention, a straight-chain alkyl group having 1 to 20 C atoms or a branched or cyclic alkyl group having 3 to 20 C atoms or an alkenyl or alkynyl group having 2 to 20 C atoms, in which, in addition, individual H atoms or CH₂ groups may be substituted by the groups mentioned above under the definition of the radicals, is preferably taken to mean the radicals methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl.

An alkoxy or thioalkyl group having 1 to 20 C atoms is preferably taken to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclo-pentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

Preferably, in compounds of formula (I) Z¹ is selected from CR¹ and CR².

Furthermore, preferably, Z² is CR².

Furthermore, it is preferred that a maximum of two groups Z¹ and a maximum of three groups Z² per aromatic ring of the compound of formula (I) is N. More preferably, in the compound of formula (I) a maximum of two groups Z¹ and a maximum of three groups Z² is N.

In a preferred embodiment of the present invention, group Ar^{L} is selected from aromatic ring systems having 6 to 30 aromatic ring atoms, which may be substituted by one or more radicals R⁴. More preferably, Ar^{L} is selected from divalent groups derived from benzene, biphenyl, terphenyl, naphthyl, fluorenyl, indenofluorenyl, spirobifluorenyl, dibenzofuranyl, dibenzothiophenyl, and carbazolyl, which may each be substituted by one or more radicals R⁴. Most preferably, Ar^{L} is a divalent group derived from benzene, which may be substituted by one or more radicals R⁴.

Preferred groups Ar^{L} conform to the following formulae Ar^{L}-1 to Ar^{L}-82

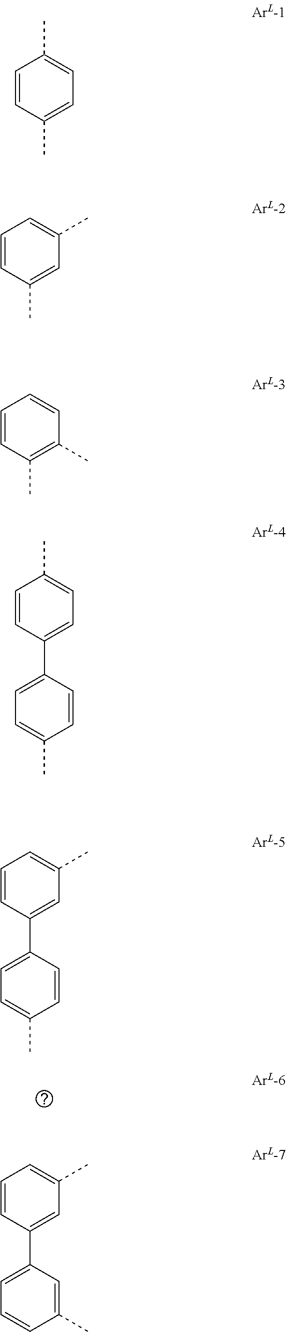

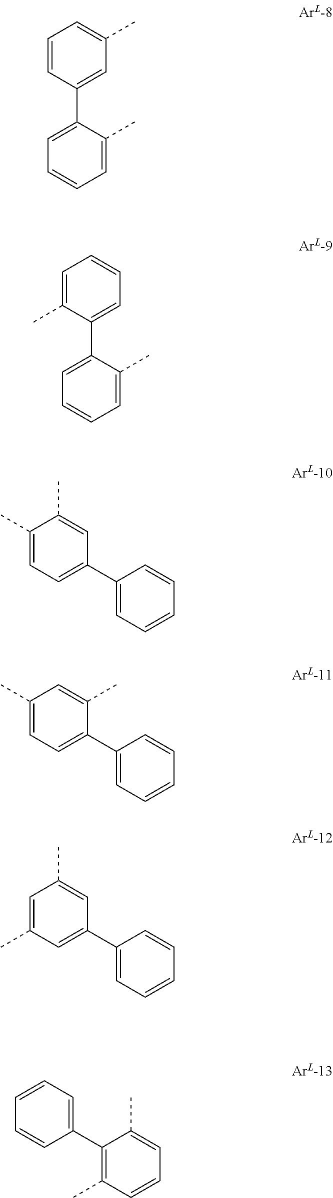

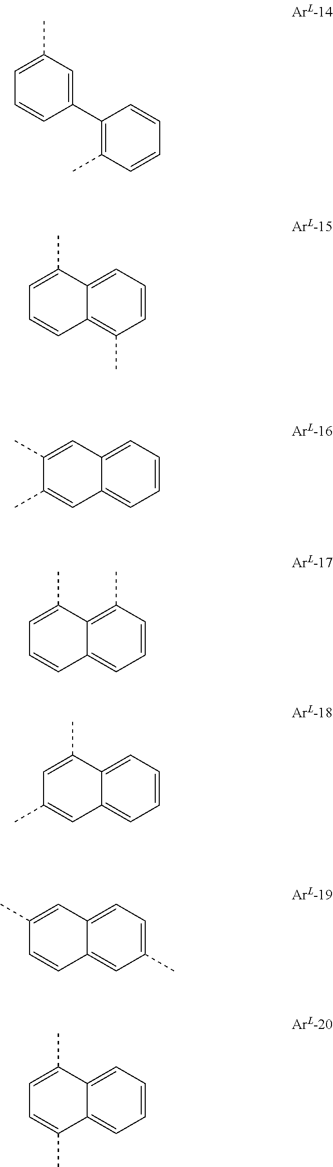

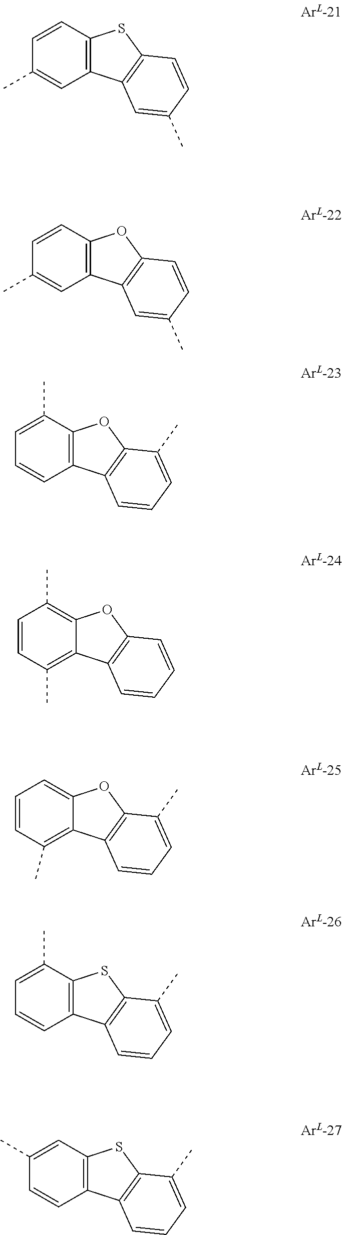

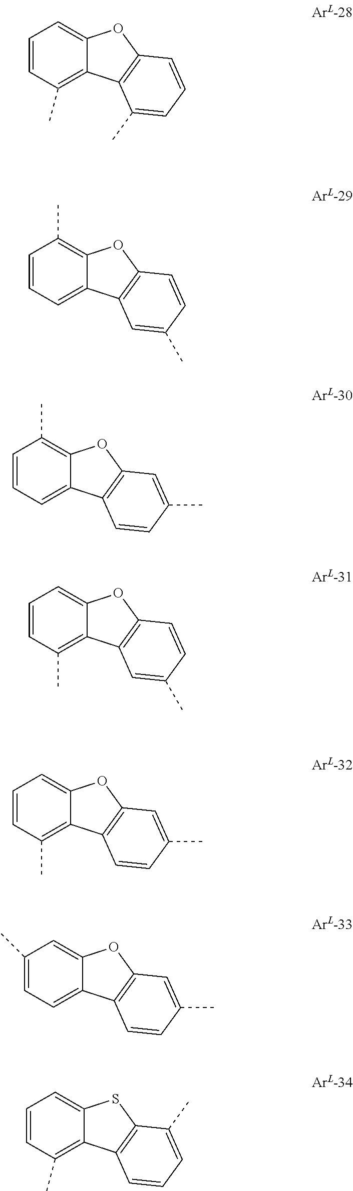

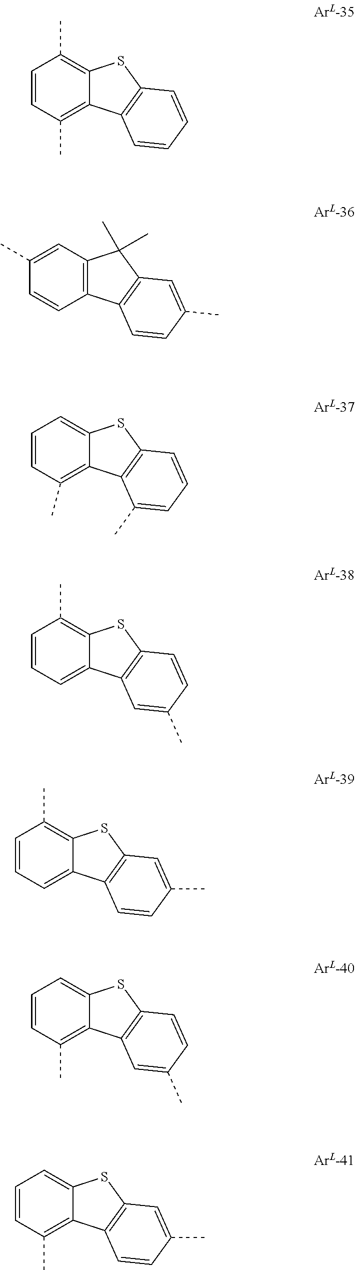

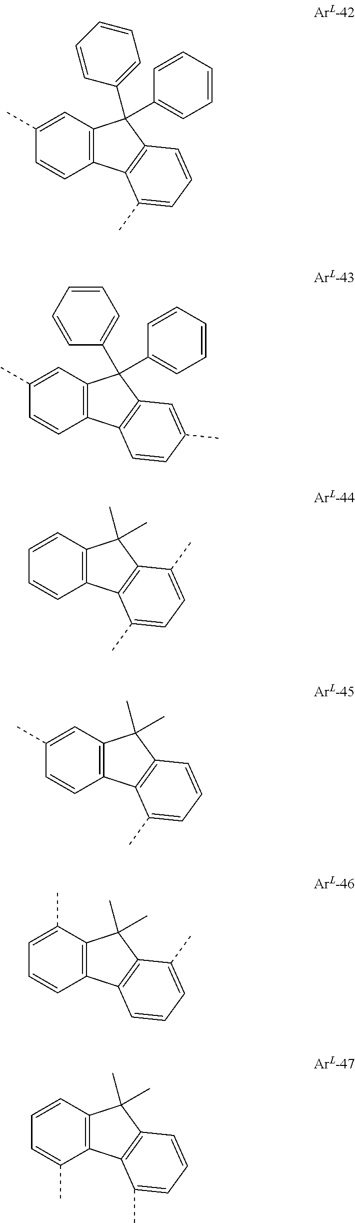

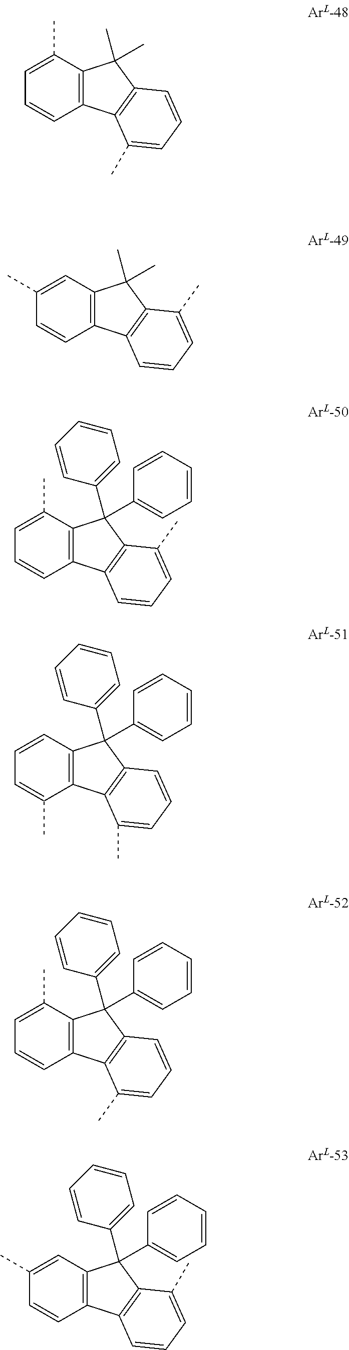

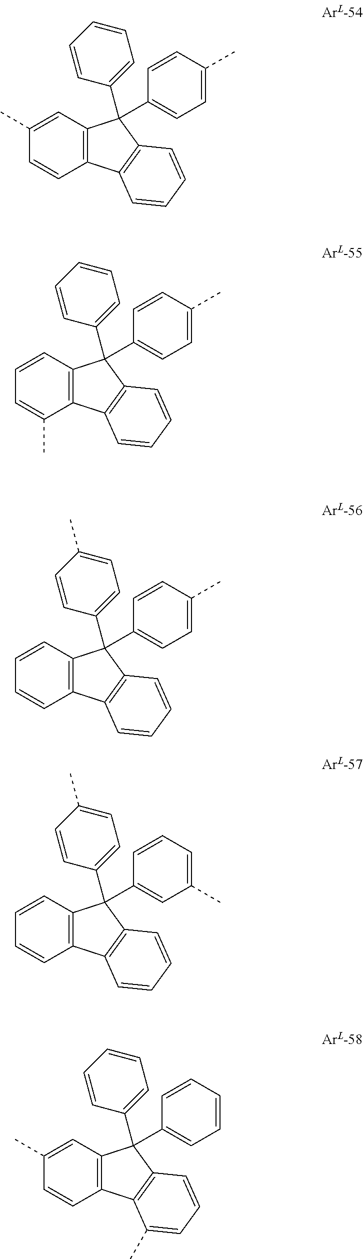

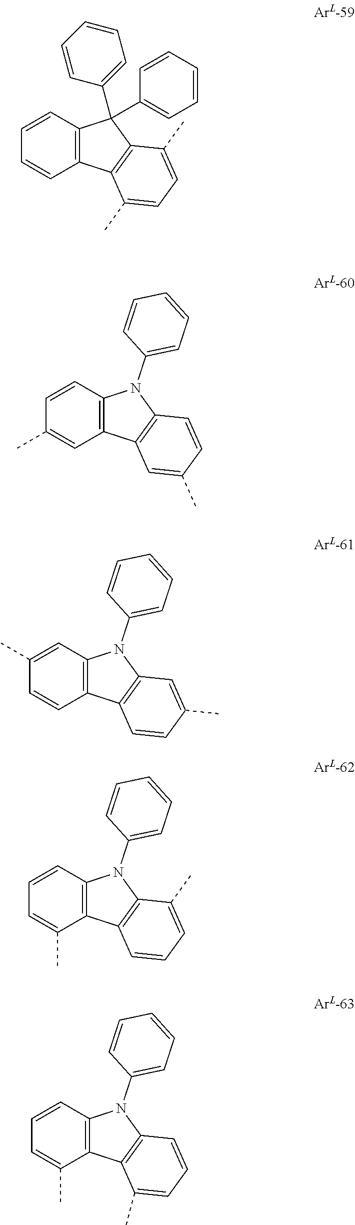

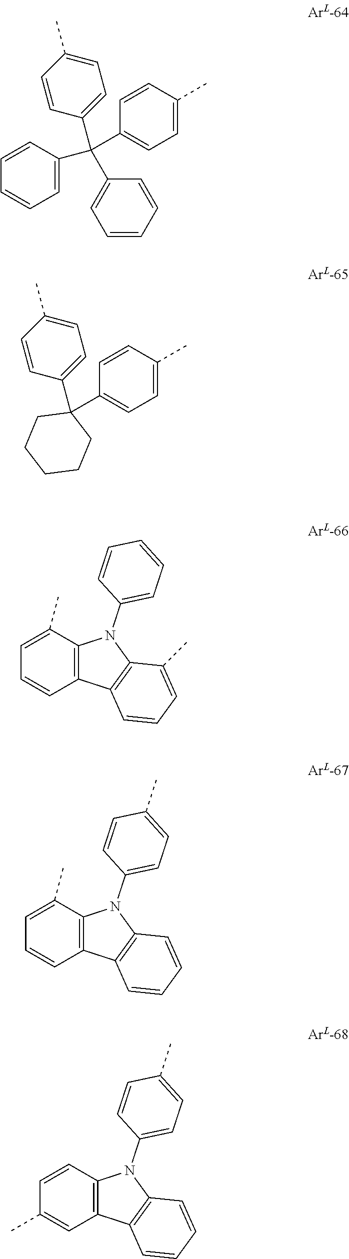

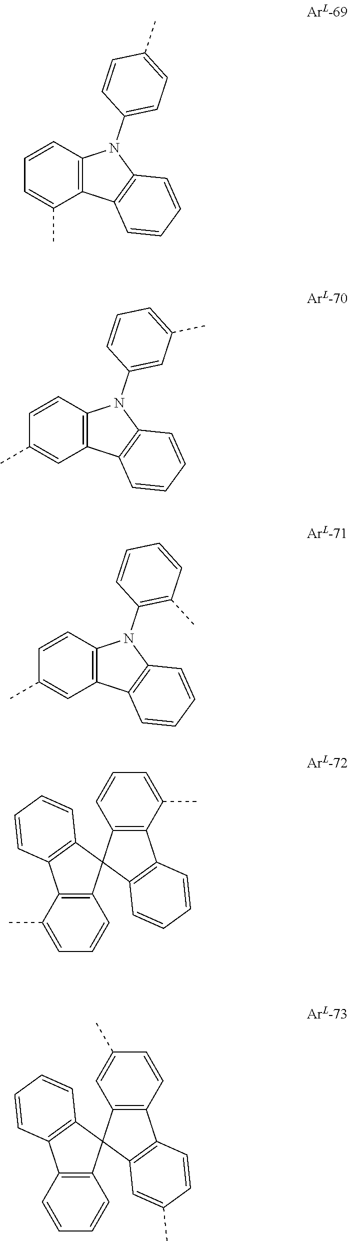

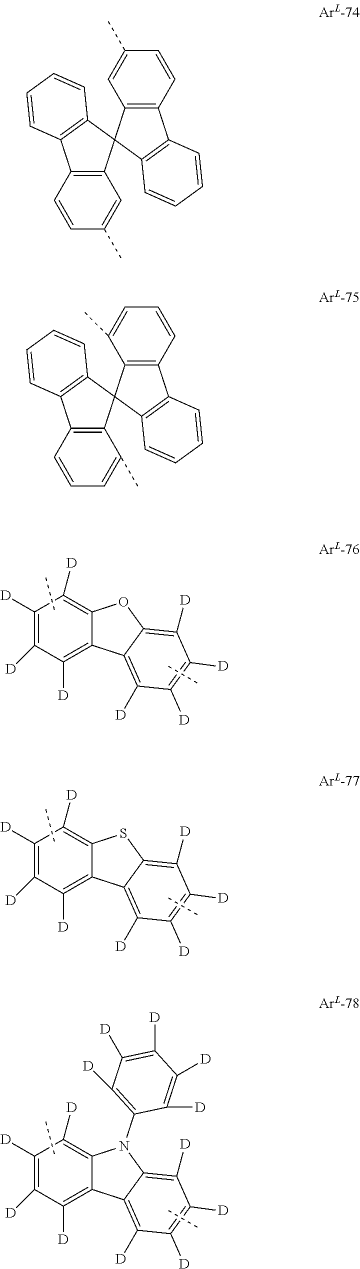

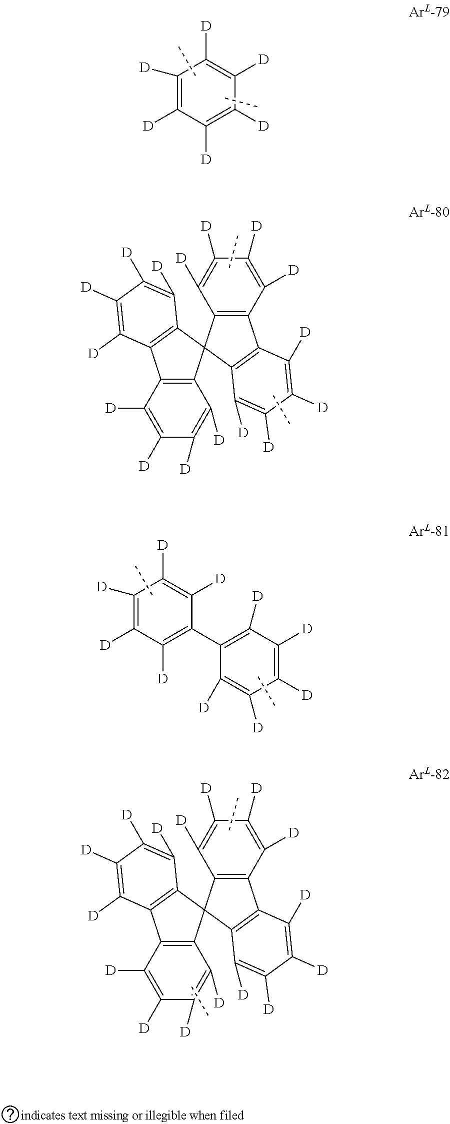

where the dotted lines represent the bonds of the divalent group to the rest of the formula (I).

Particularly preferred among the groups above are the groups according to one of formulae Ar^{L}-1, Ar^{L}-2, Ar^{L}-3, Ar^{L}-4, Ar^{L}-15, Ar^{L}-20, Ar^{L}-25, and Ar^{L}-36.

Particularly preferred among the groups above are the groups according to one of formulae Ar^{L}-76, Ar^{L}-77, Ar^{L}-78, Ar^{L}-79, Ar^{L}-80, Ar^{L}-81 and Ar^{L}-82.

It is preferred that index n is 0, meaning that the group Ar^{L} is not present, so that the fluorene and the nitrogen atom of the amine are directly connected with each other.

Preferably, at least one of groups Ar¹ and Ar² is selected from a radical comprising at least two rings selected from aromatic and heteroaromatic rings, which radical may optionally be substituted by one or more radicals R⁴. That is, at least one of groups Ar¹ and Ar² is an aromatic ring system that comprises two or more simple aromatic rings as aryl groups, or a heteroaromatic ring system that comprises two or more simple aromatic rings, at least one which contains a heteroatom as one of the aromatic ring atoms to form a simple heteroaromatic ring as heteroaryl group. According to the invention, within said at least one radical of group Ar¹ or Ar² two aromatic or heteroaromatic rings may be condensed or may be connected to each other via a divalent group selected from —C(R⁴)₂—, —N(R⁴)—, —O—, and —S—.

More preferably, said at least one radical of group Ar¹ or Ar² comprises at least two aromatic rings. That is, at least one of groups Ar¹ and Ar² is an aromatic ring system that comprises two or more simple aromatic rings as aryl groups, which aromatic rings may be condensed or may be connected to each other via a divalent group selected from —C(R⁴)₂—, —N(R⁴)—, —O—, and —S—.

Even more preferably, groups Ar¹ and Ar² are, identically or differently, selected from radicals comprising at least two rings selected from aromatic and heteroaromatic rings, which radicals may each optionally be substituted by one or more radicals R⁴. That is, each of groups Ar¹ and Ar² is either an aromatic ring system that comprises two or more simple aromatic rings as aryl groups, or a heteroaromatic ring system that comprises two or more simple aromatic rings, at least one which contains a heteroatom as one of the aromatic ring atoms to form a simple heteroaromatic ring as heteroaryl group. According to the invention, within at least one of said radicals or within both of said radicals of groups Ar¹ and Ar² two aromatic or heteroaromatic rings may be condensed or may be connected to each other via a divalent group selected from —C(R⁴)₂—, —N(R⁴)—, —O—, and —S—.

It is particularly preferred that said radicals of groups Ar¹ and Ar² each comprises at least two aromatic rings. That is, groups Ar¹ and Ar² are, identically or differently, selected from aromatic ring systems that comprise two or more simple aromatic rings as aryl groups, wherein within one or within both of said groups Ar¹ and Ar² the aromatic rings may be condensed or may be connected to each other via a divalent group selected from —C(R⁴)₂—, —N(R⁴)—, —O—, and —S—.

According to another embodiment, it is preferred that said aromatic or heteroaromatic rings are neither condensed nor connected.

Preferably, groups Ar¹ and Ar² are, identically or differently, selected from radicals derived from the following groups, which are each optionally substituted by one or more radicals R⁴, or from combinations of 2 or 3 radicals derived from the following groups, which are each optionally substituted by one or more radicals R⁴: phenyl, biphenyl, terphenyl, quarterphenyl, naphthyl, fluorenyl, especially 9,9′-dimethylfluorenyl and 9,9′-diphenylfluorenyl, benzofluorenyl, spirobifluorenyl, indenofluorenyl, dibenzofuranyl, dibenzothiophenyl, carbazolyl, benzofuranyl, benzothiophenyl, indolyl, quinolinyl, pyridyl, pyrimidyl, pyrazinyl, pyridazinyl and triazinyl.

Particularly preferred groups Ar¹ and Ar² are, identically or differently, selected from phenyl, biphenyl, terphenyl, quarterphenyl, naphthyl, fluorenyl, especially 9,9′-dimethylfluorenyl and 9,9′-diphenylfluorenyl, benzofluorenyl, spirobifluorenyl, indenofluorenyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, each of which may optionally be substituted by one or more radicals R⁴.

Preferred groups Ar¹ and Ar² are, identically or differently, selected from groups of the following formulae

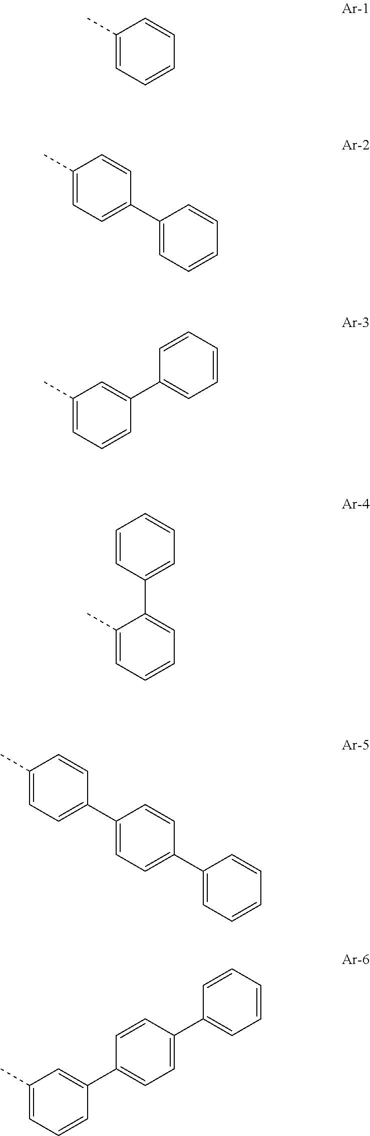

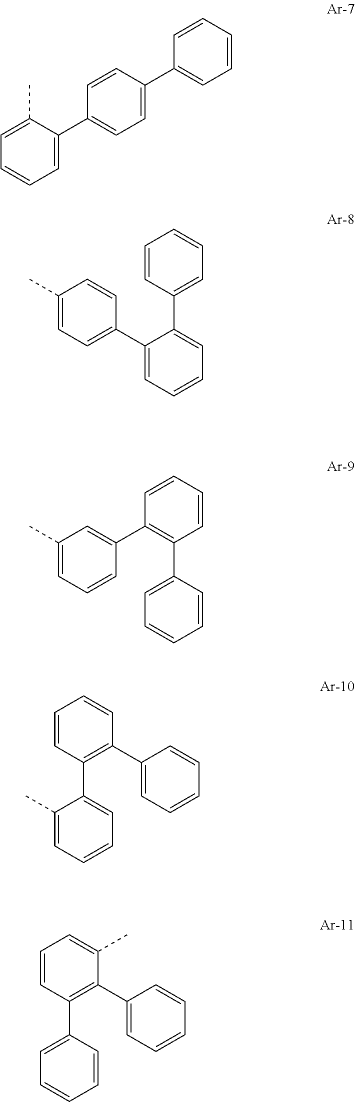

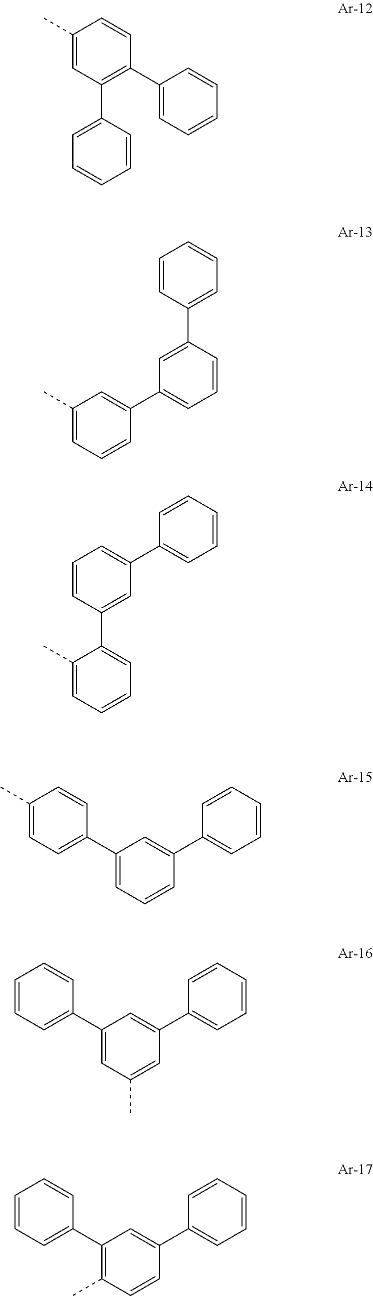

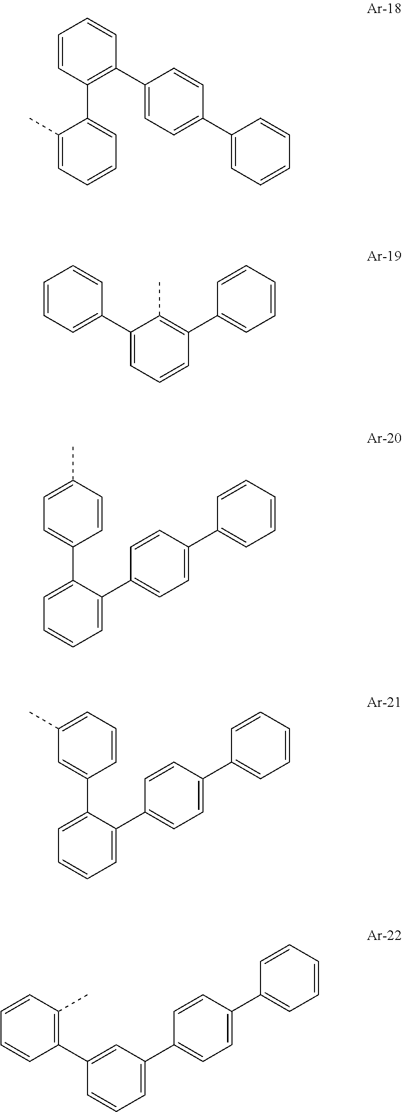

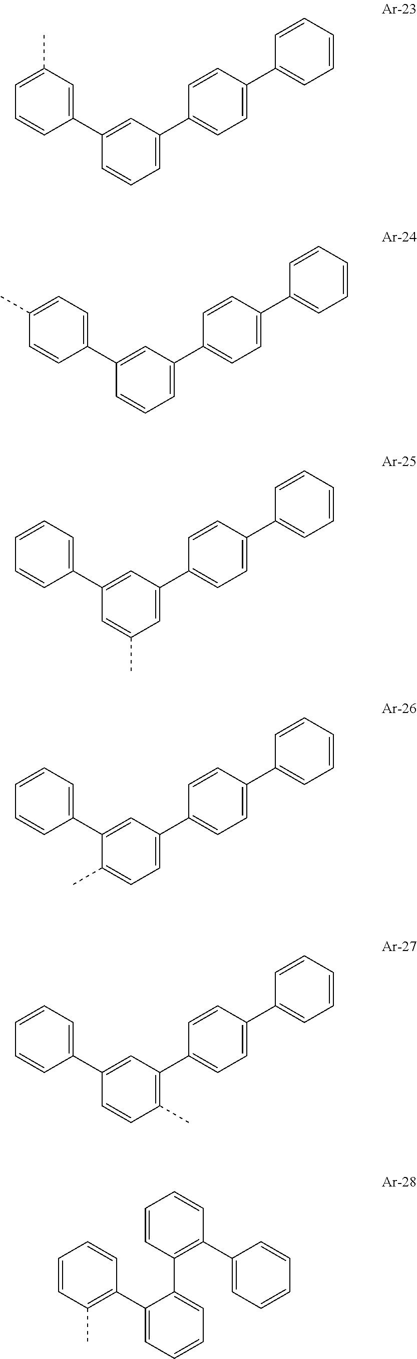

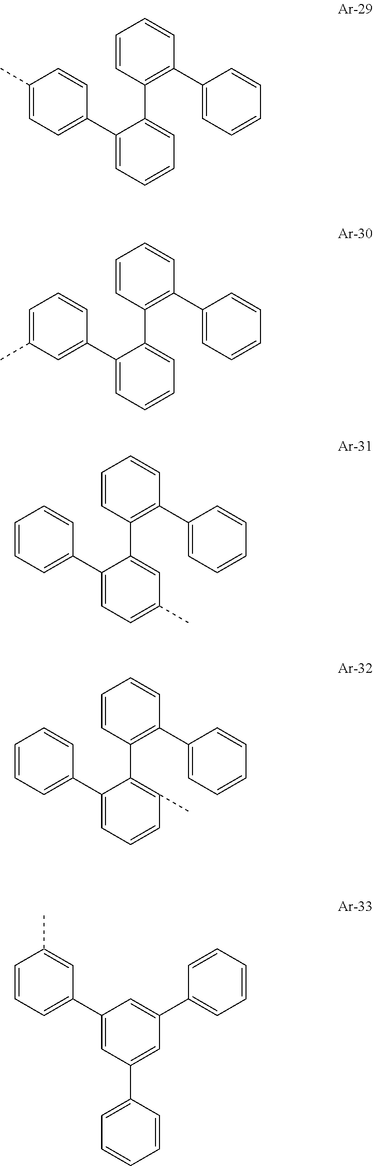

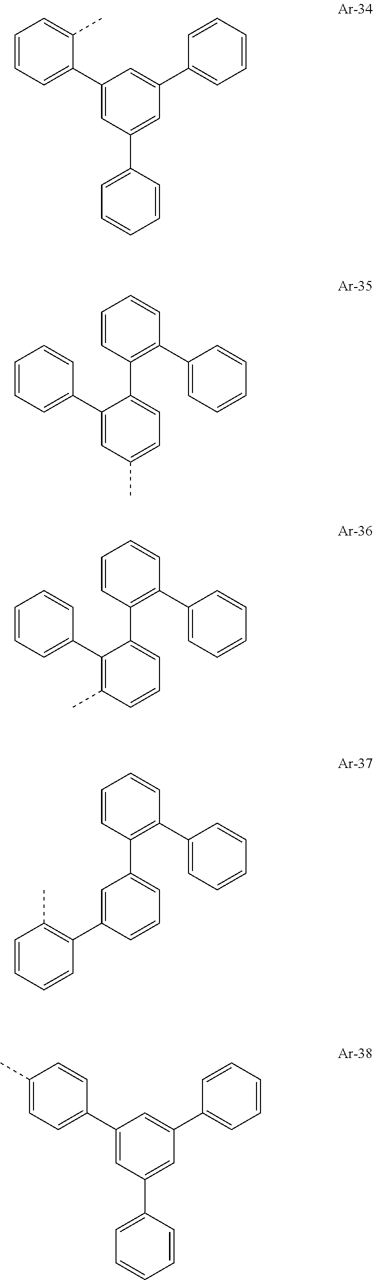

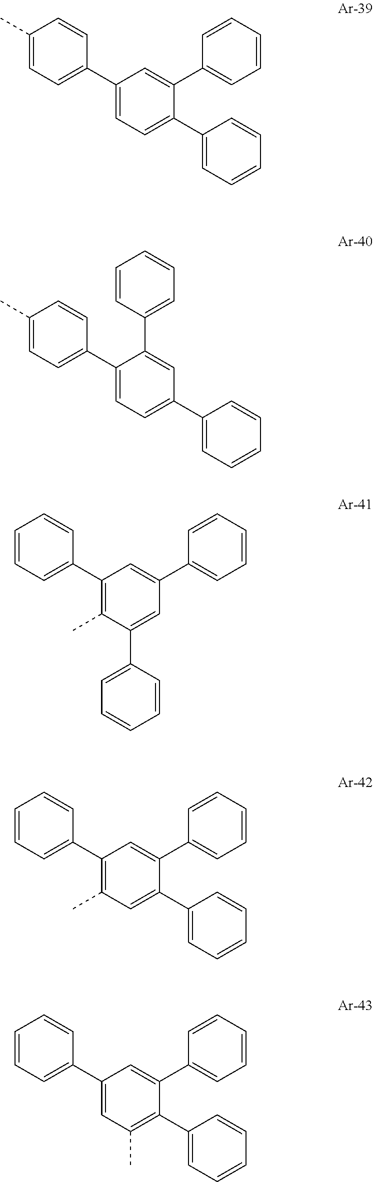

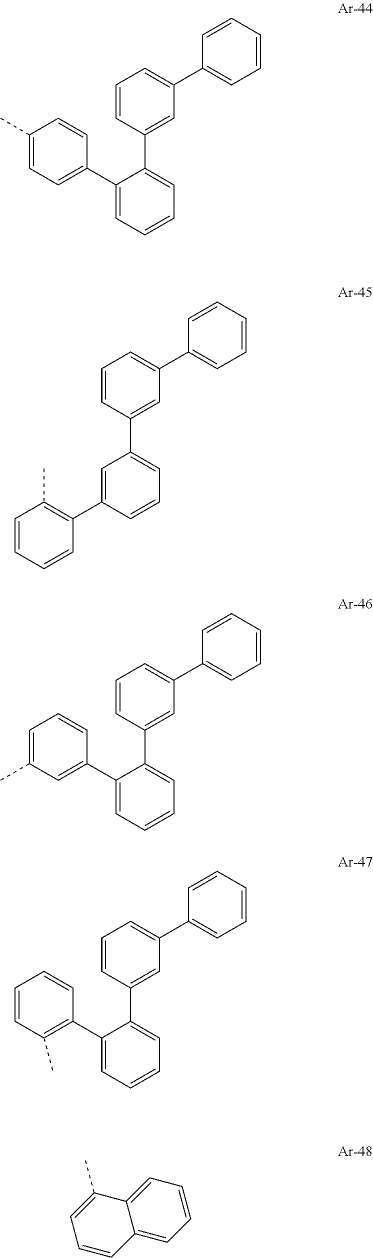

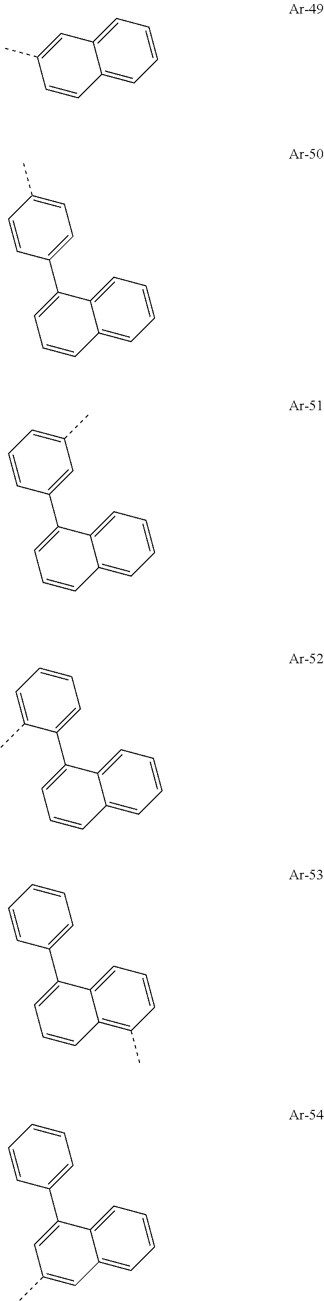

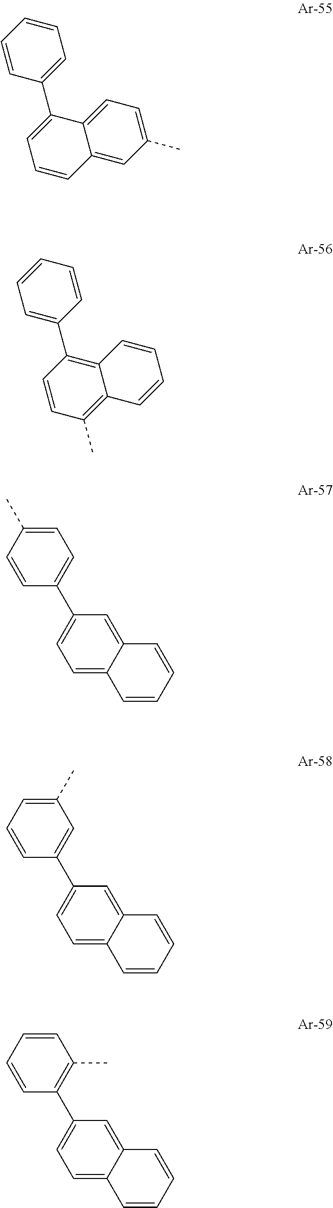

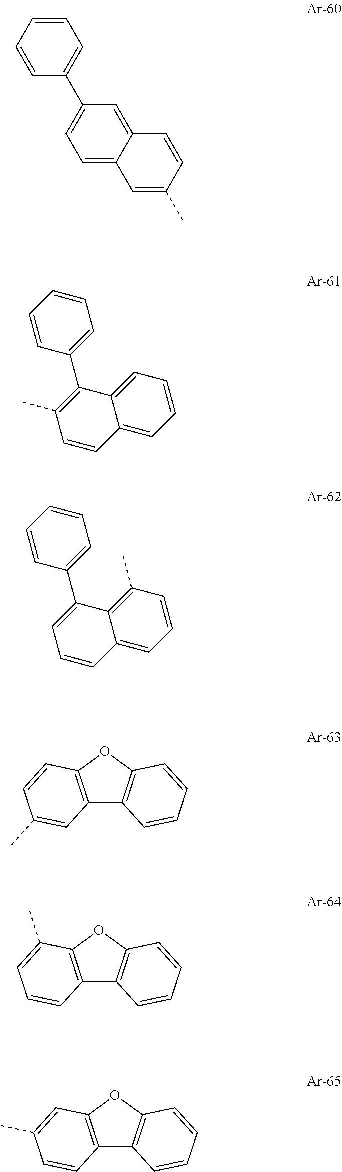

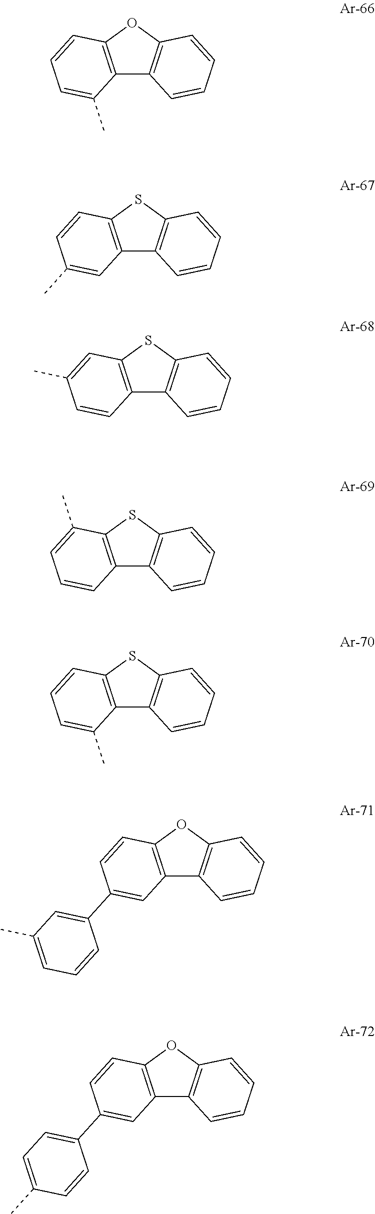

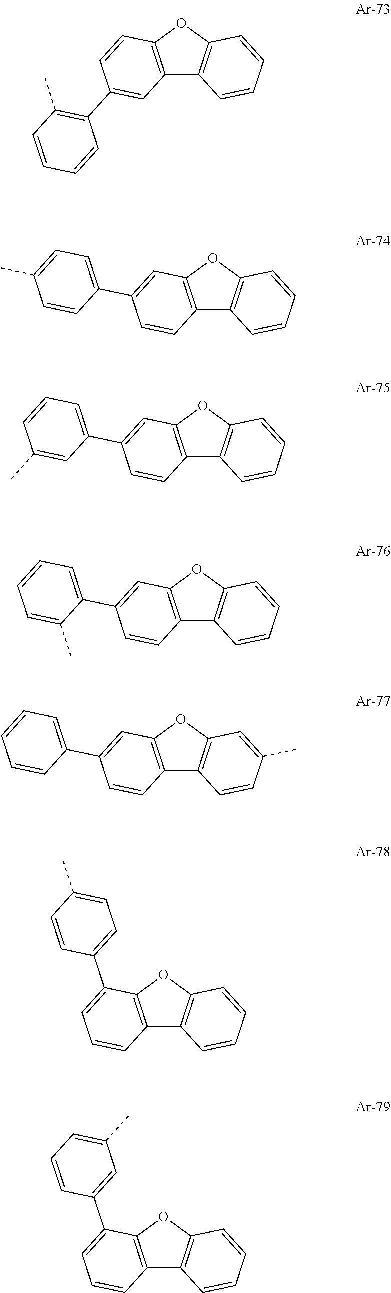

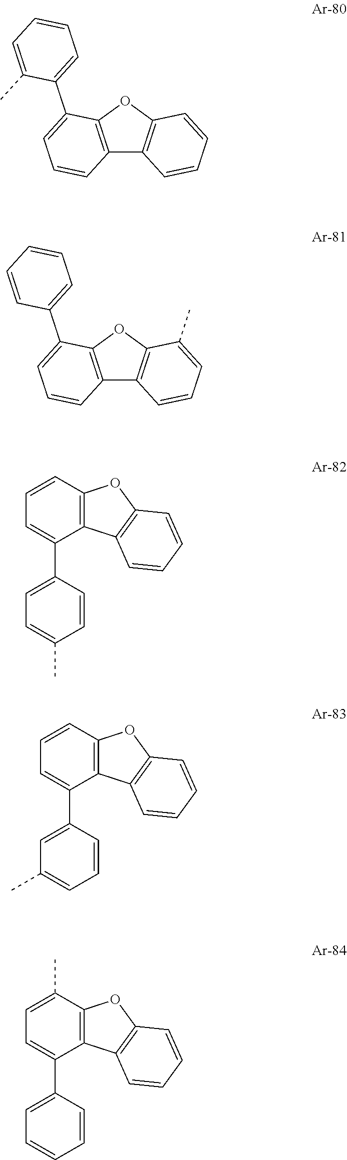

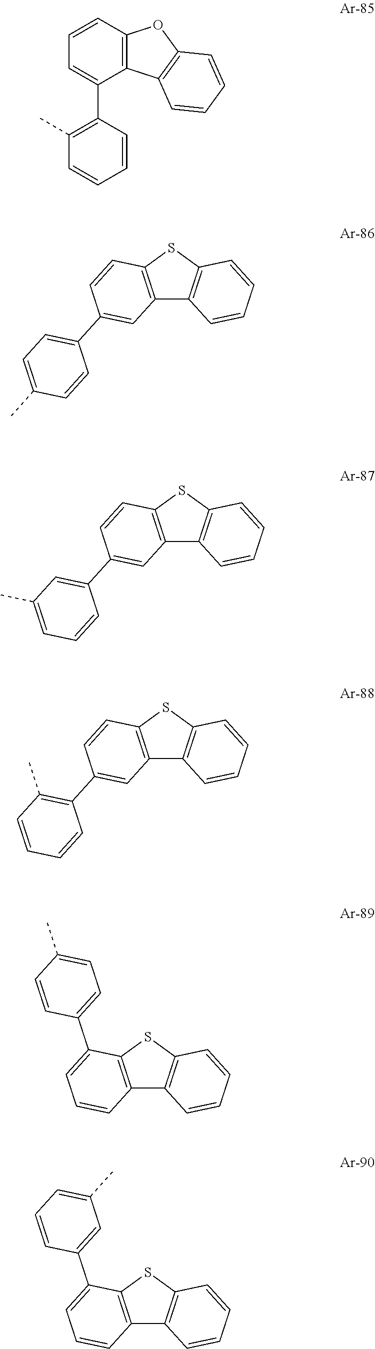

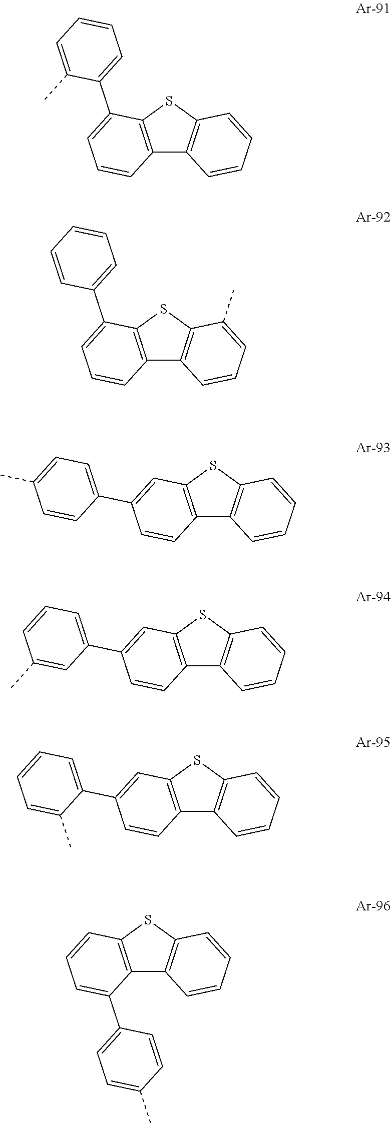

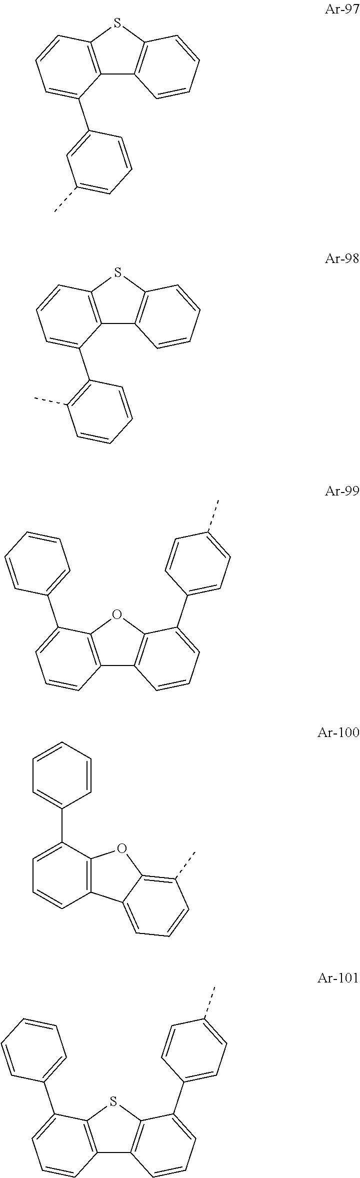

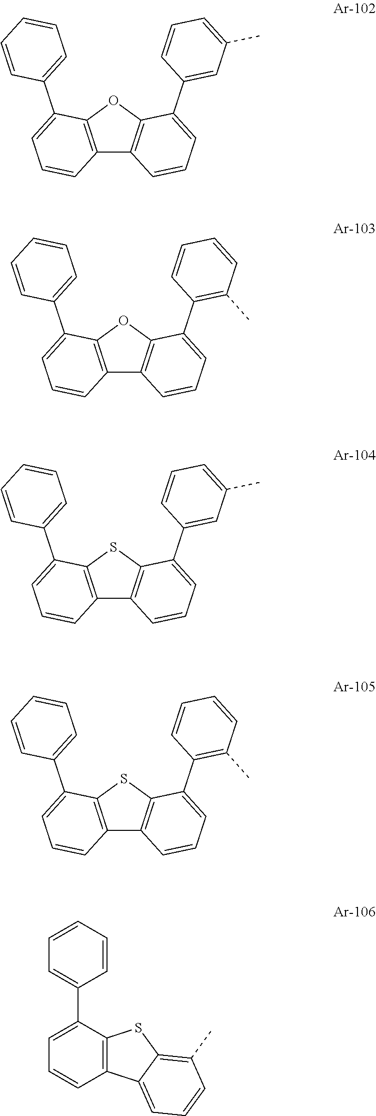

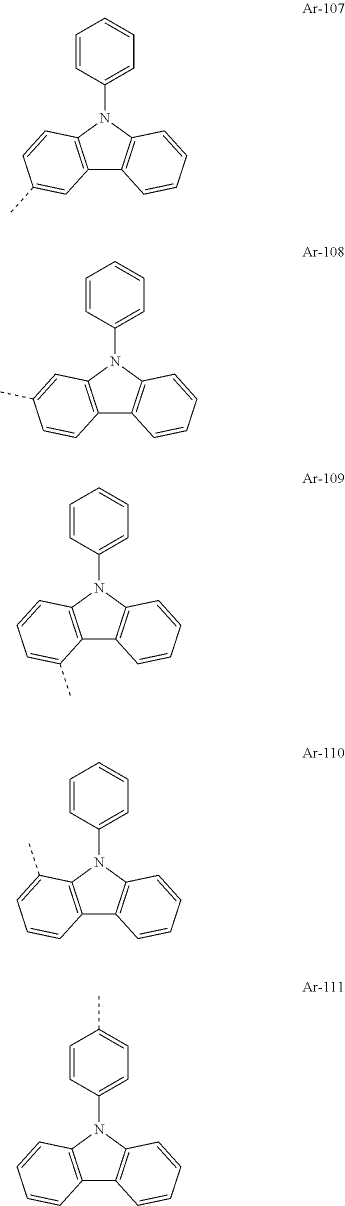

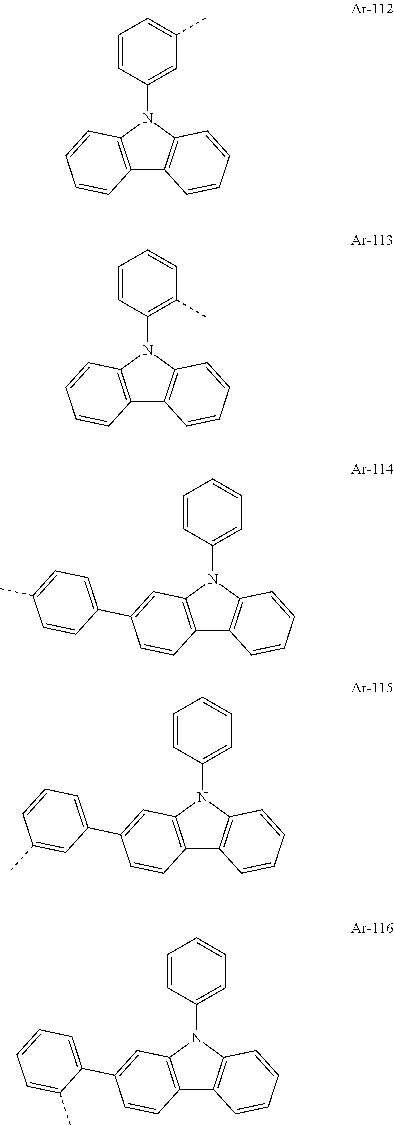

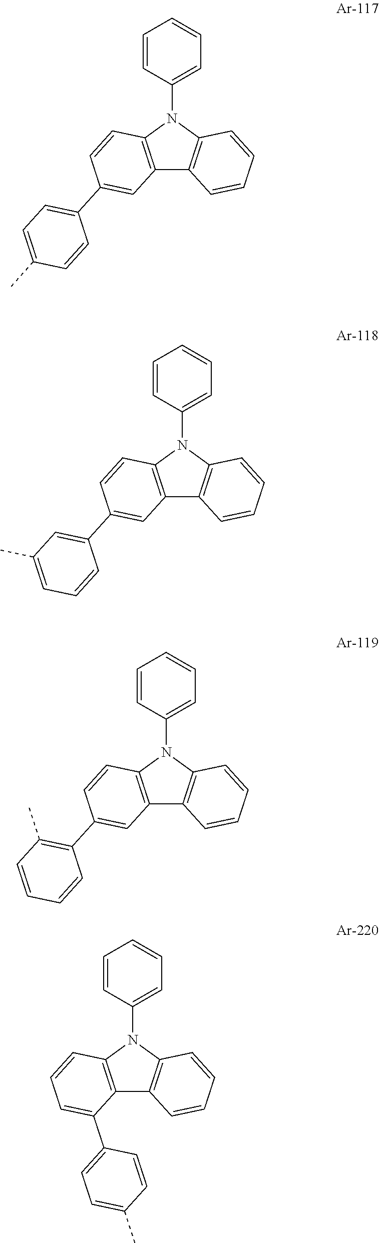

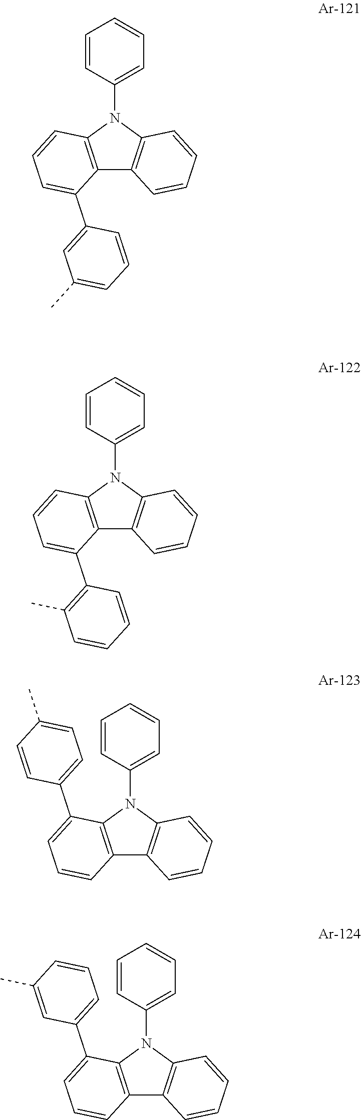

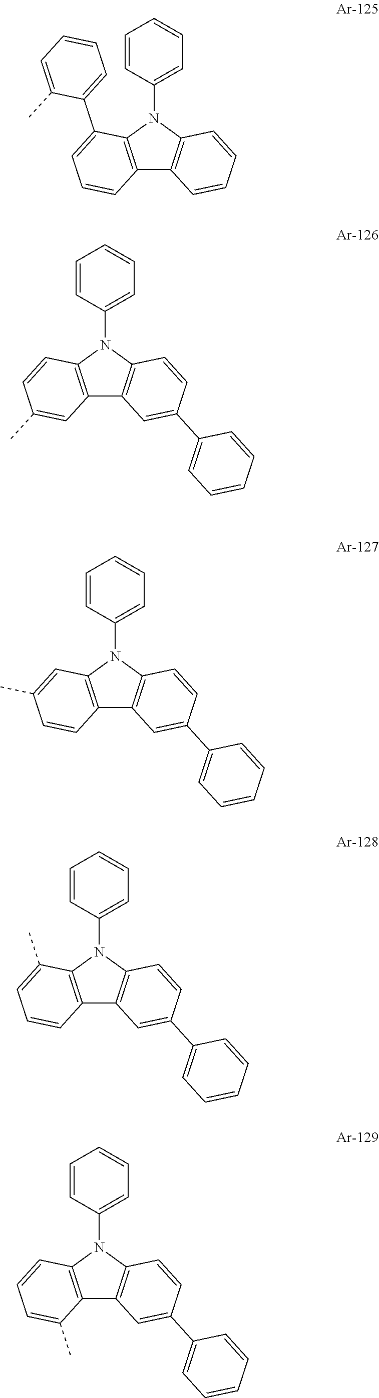

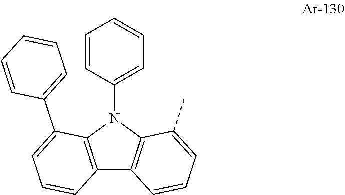

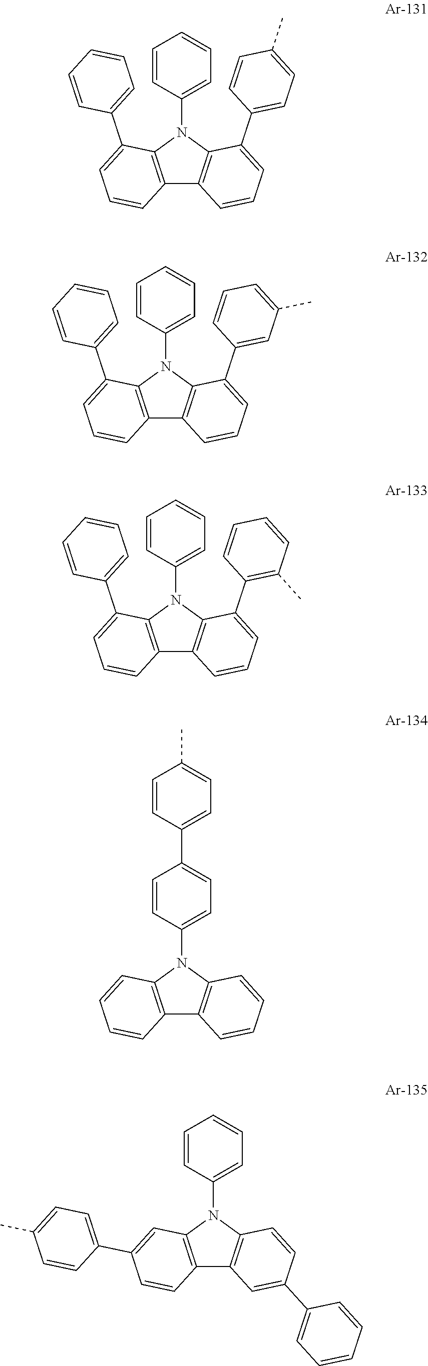

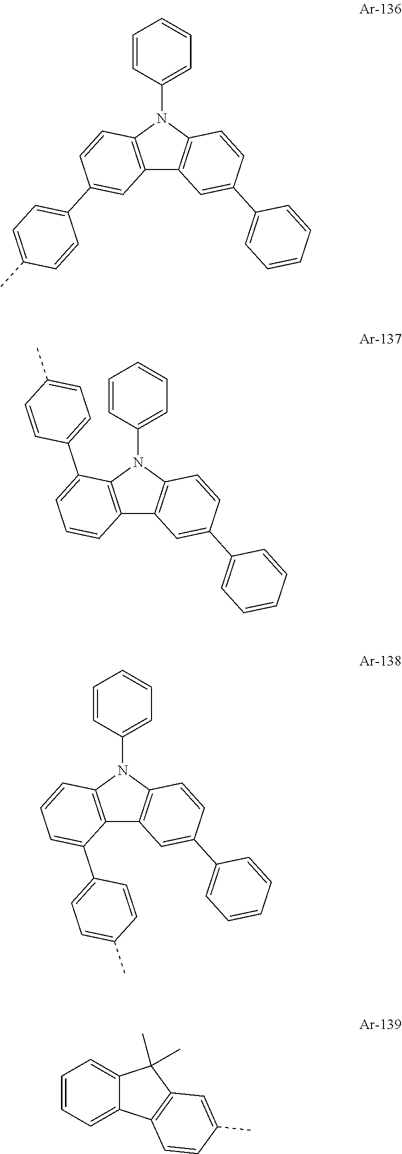

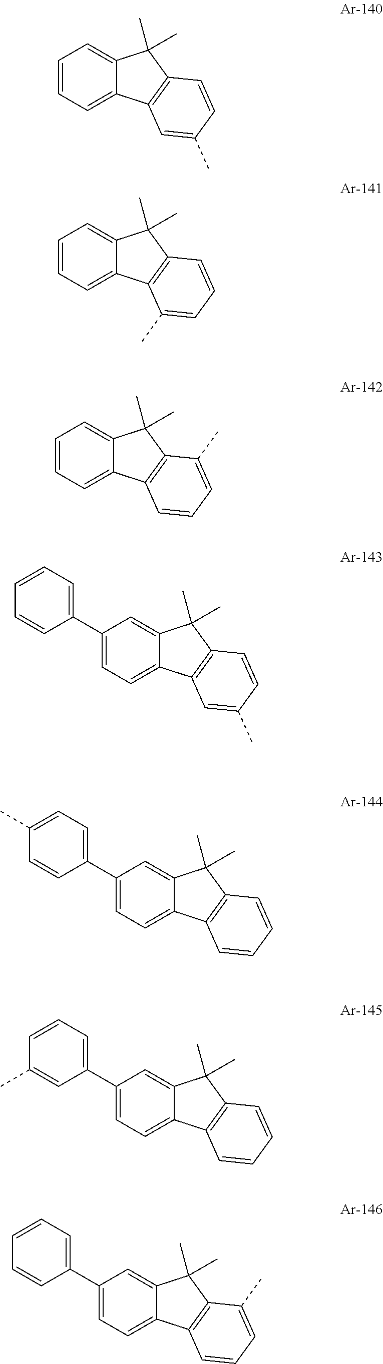

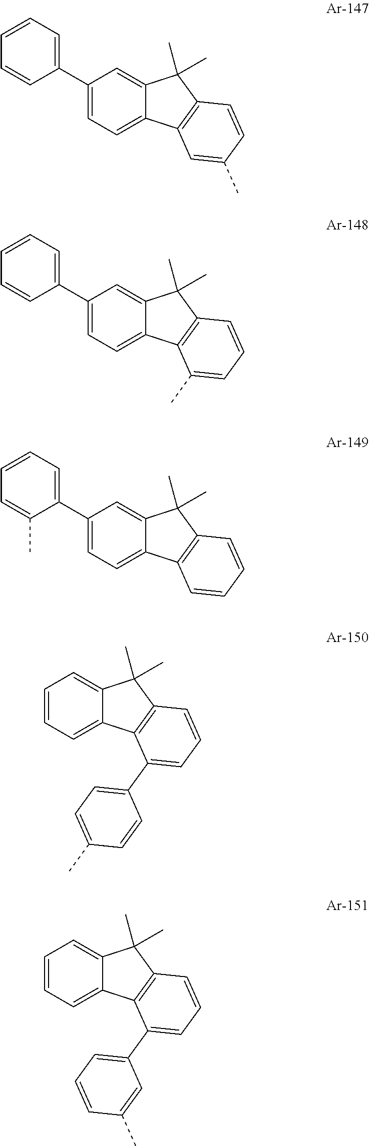

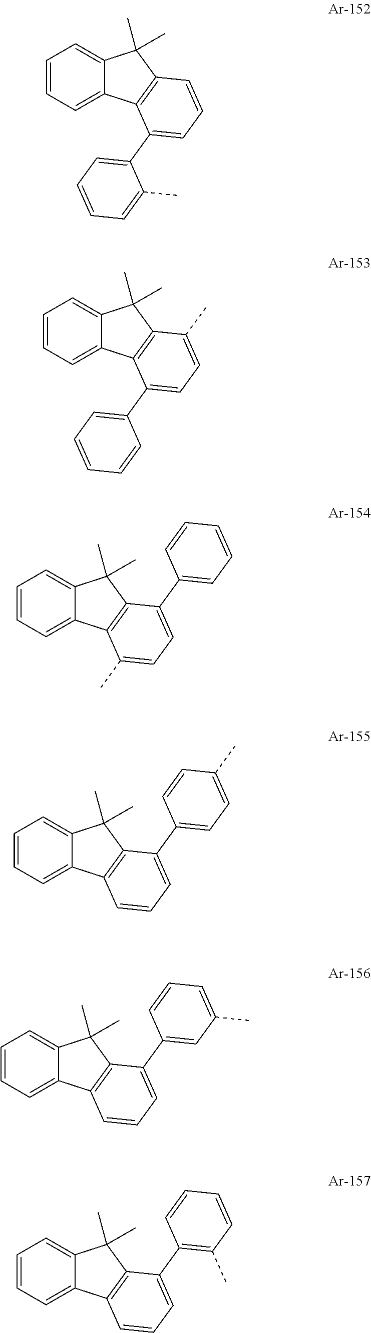

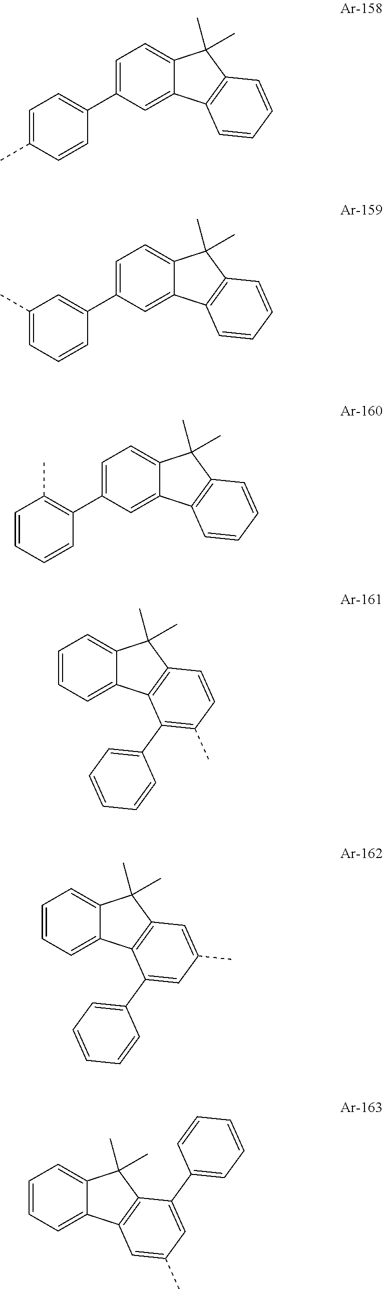

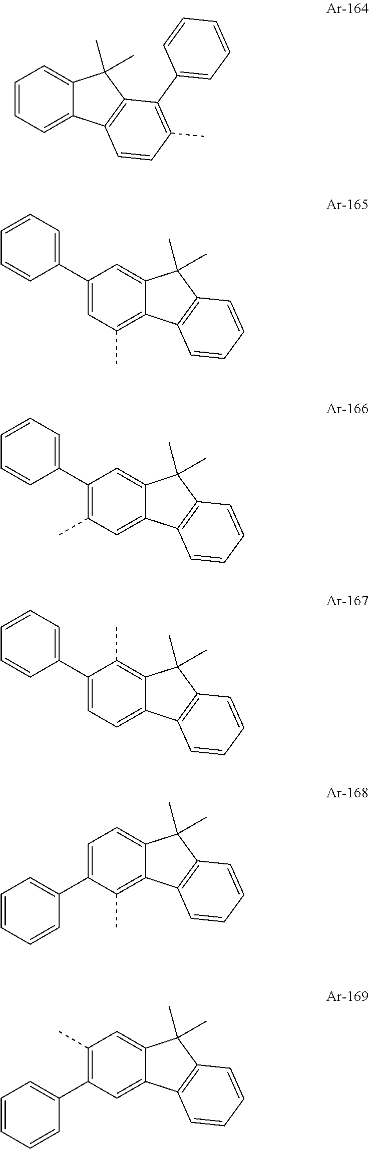

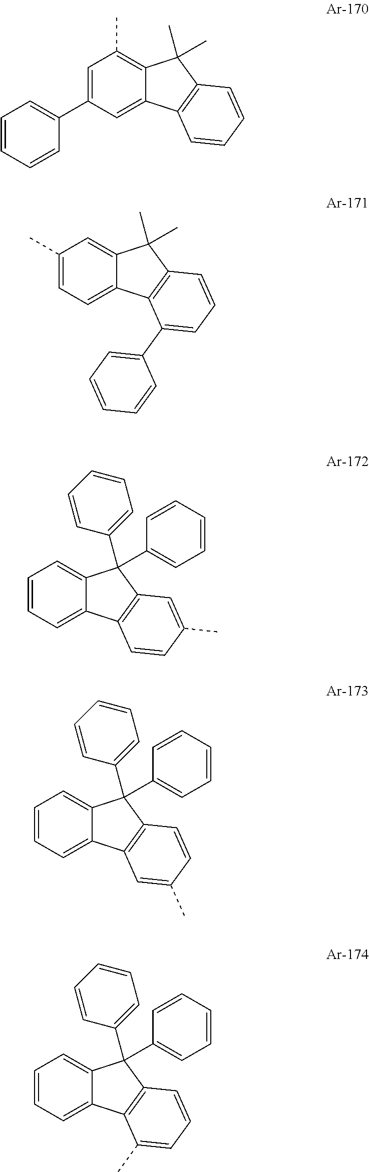

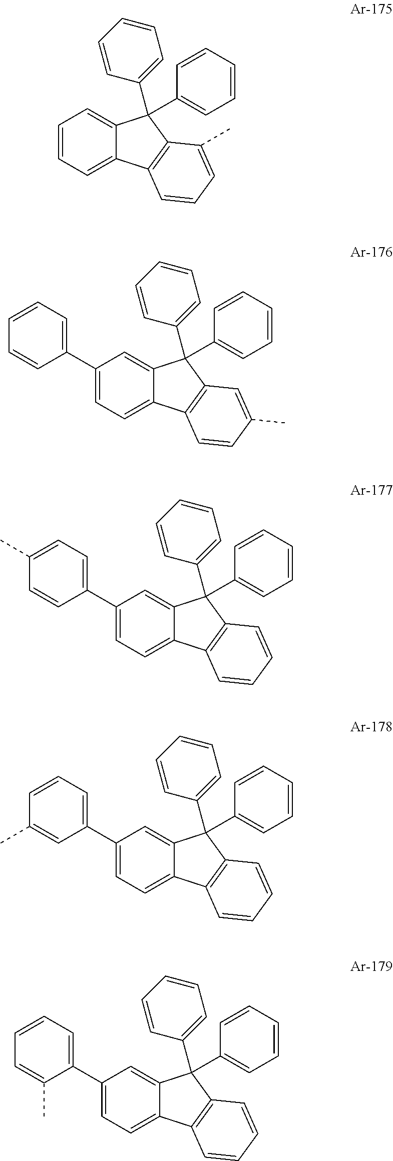

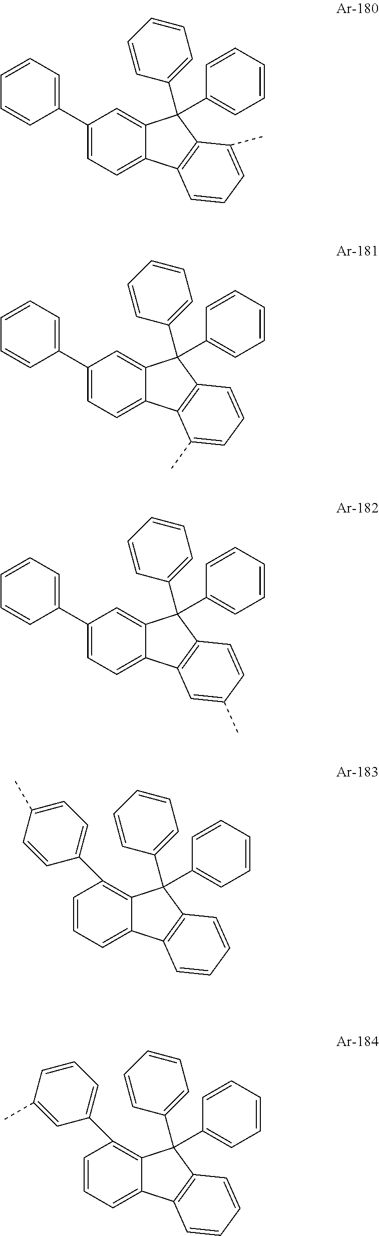

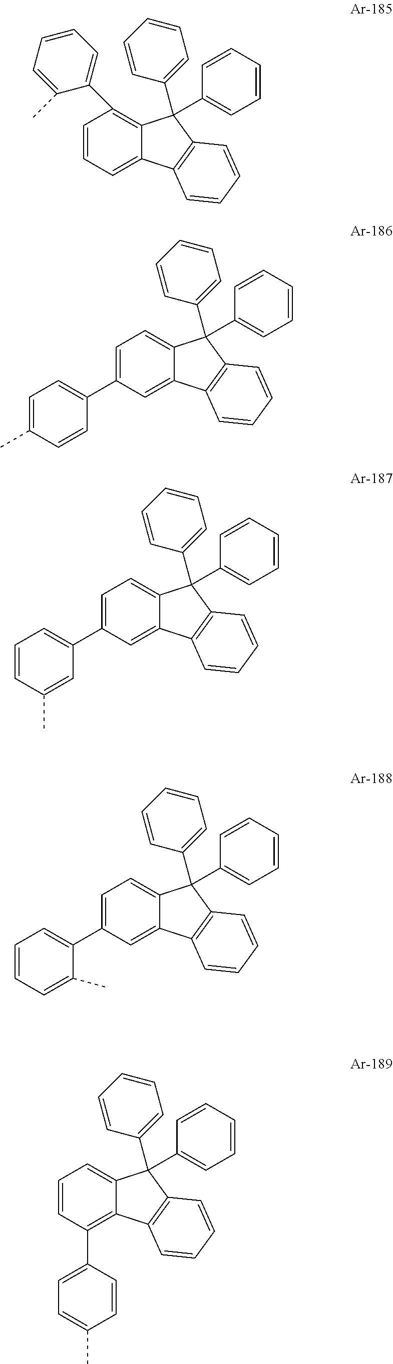

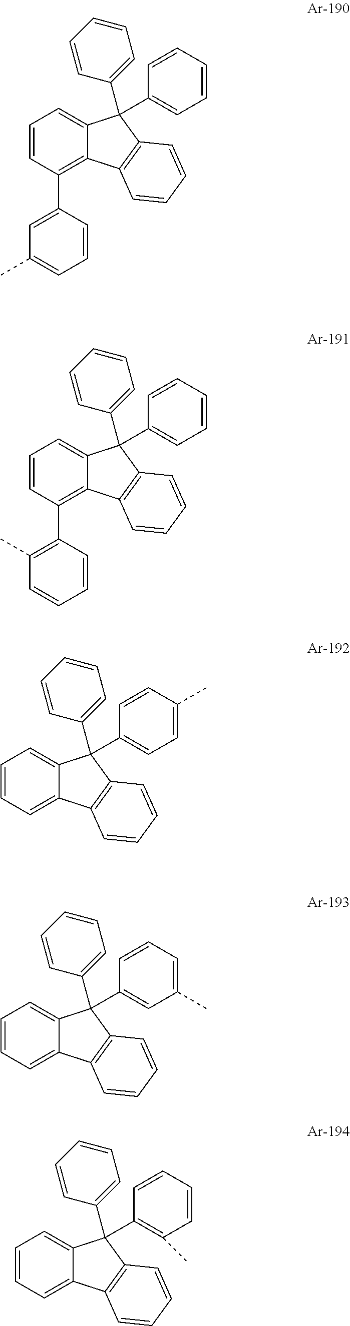

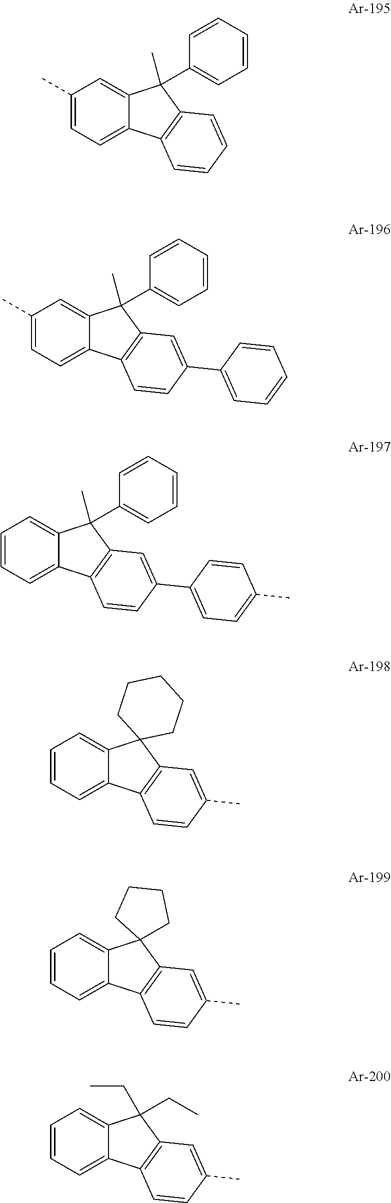

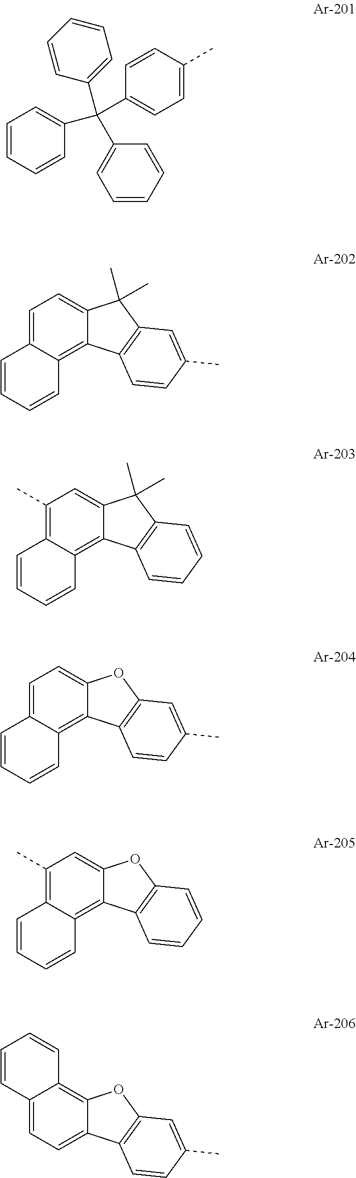

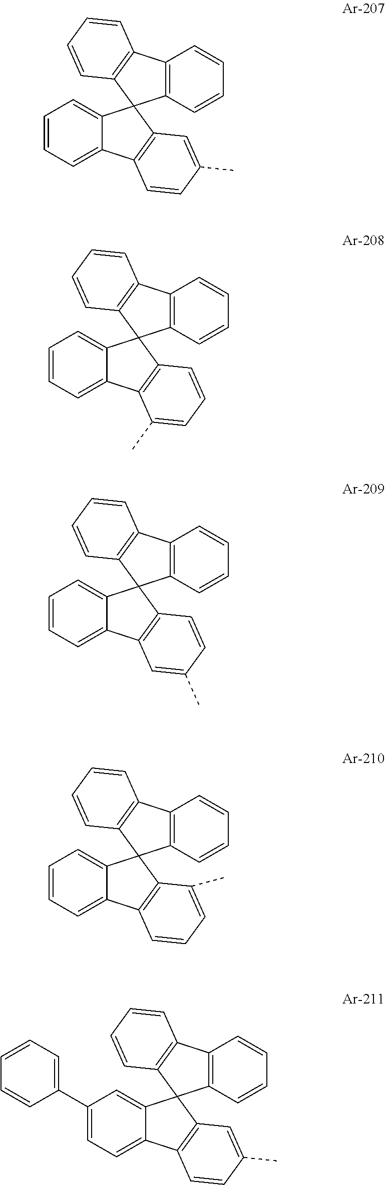

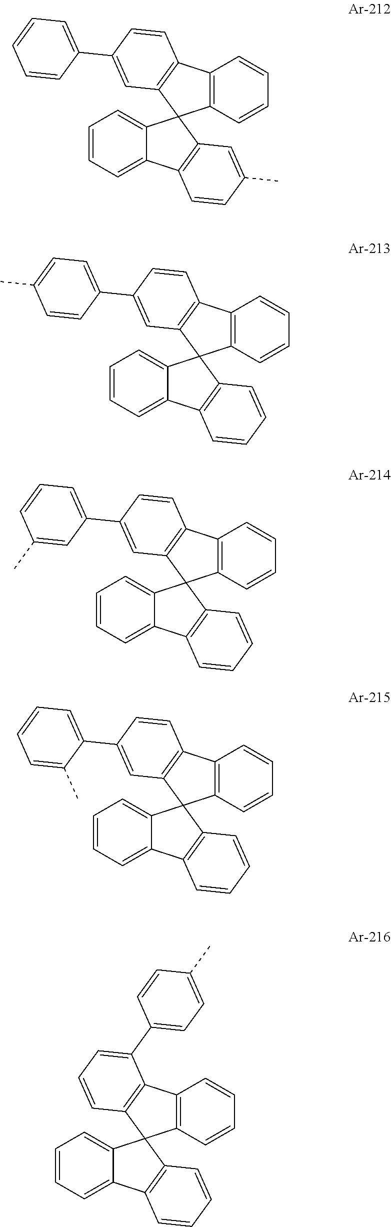

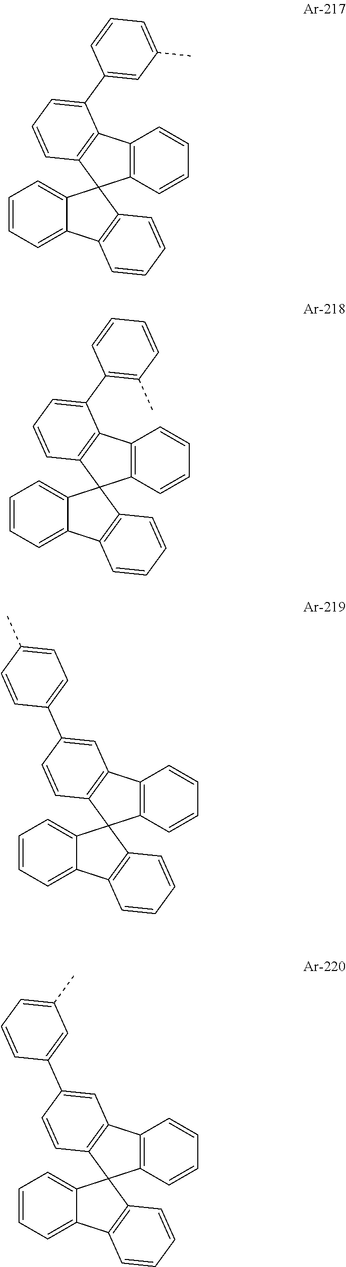

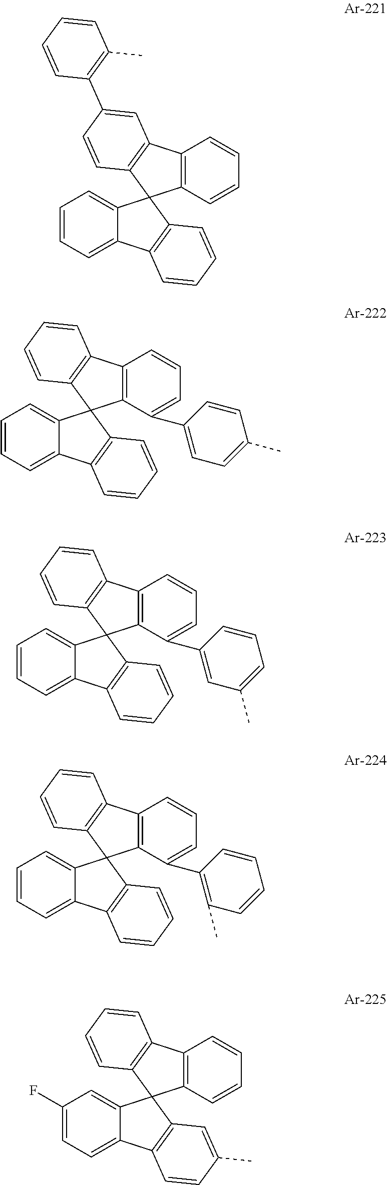

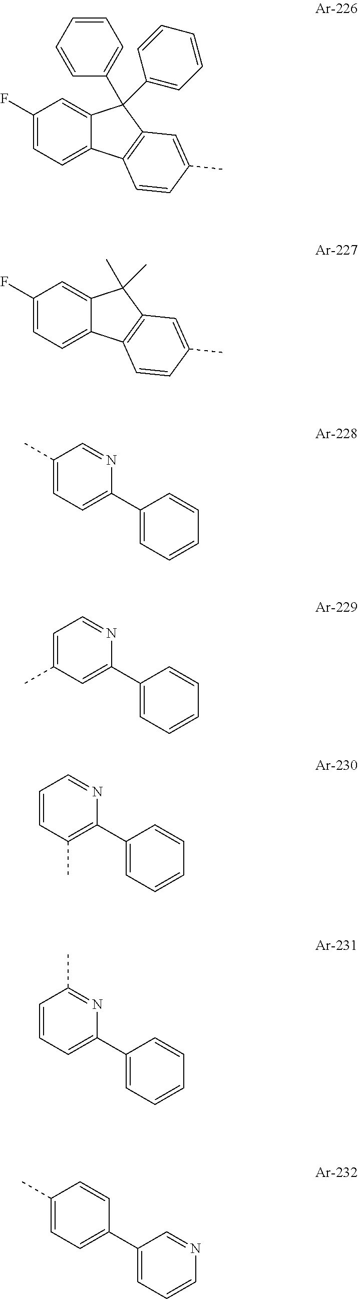

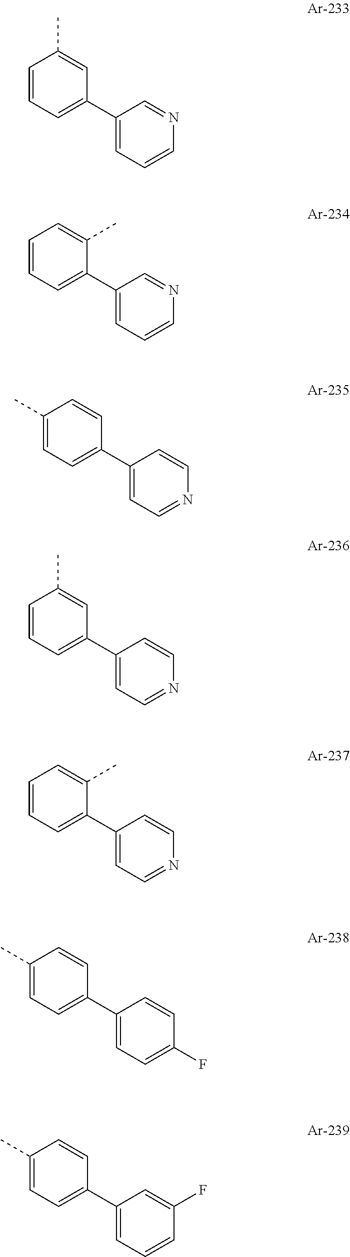

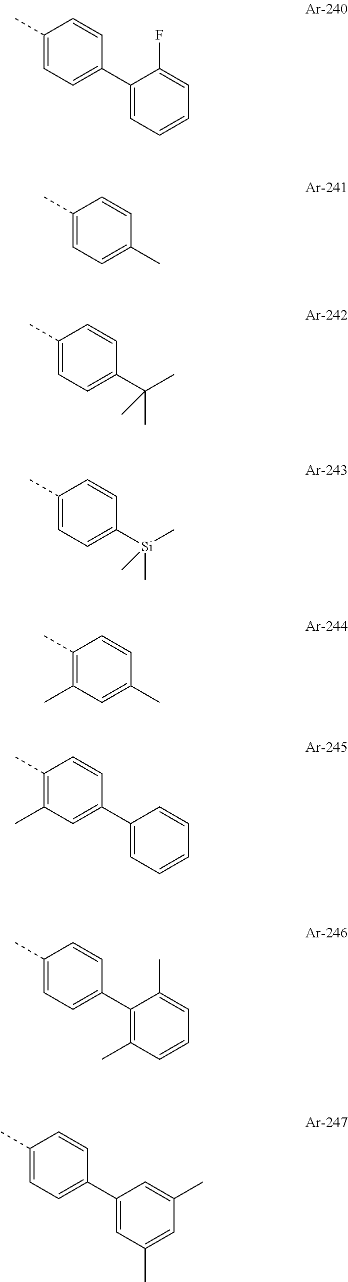

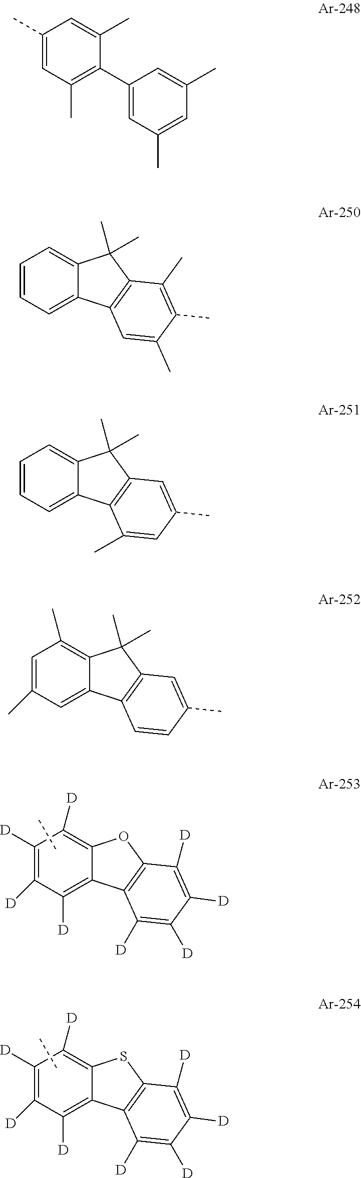

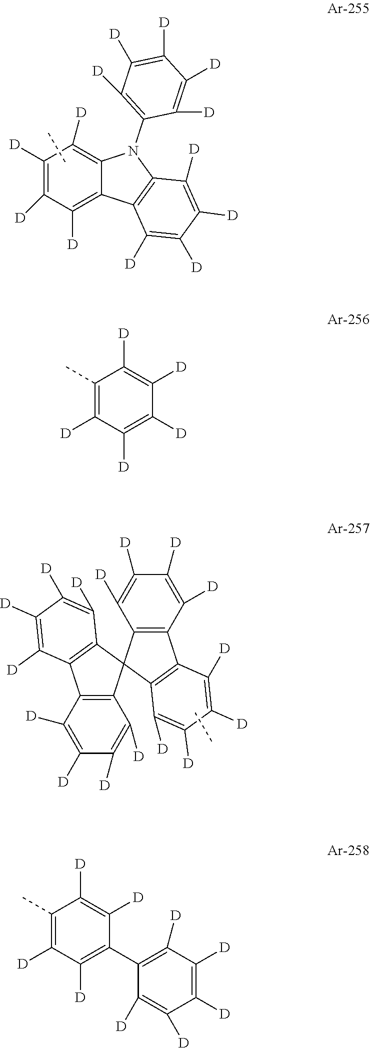

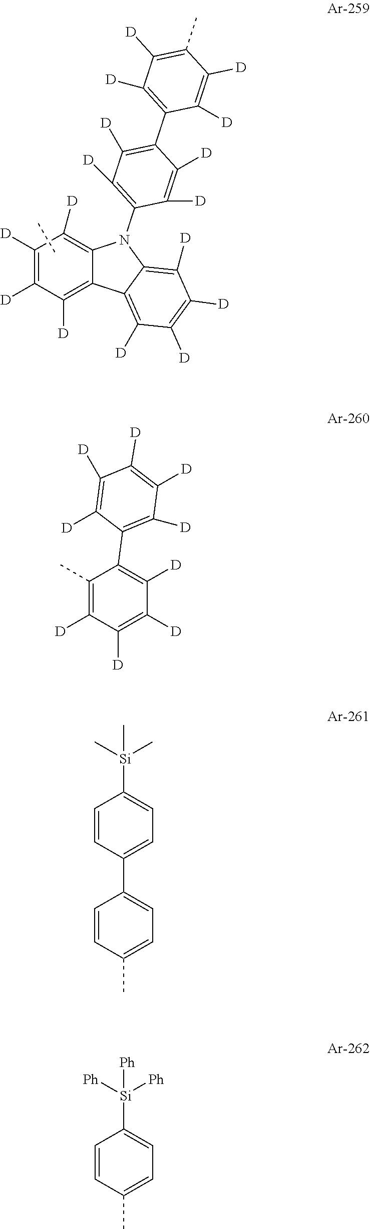

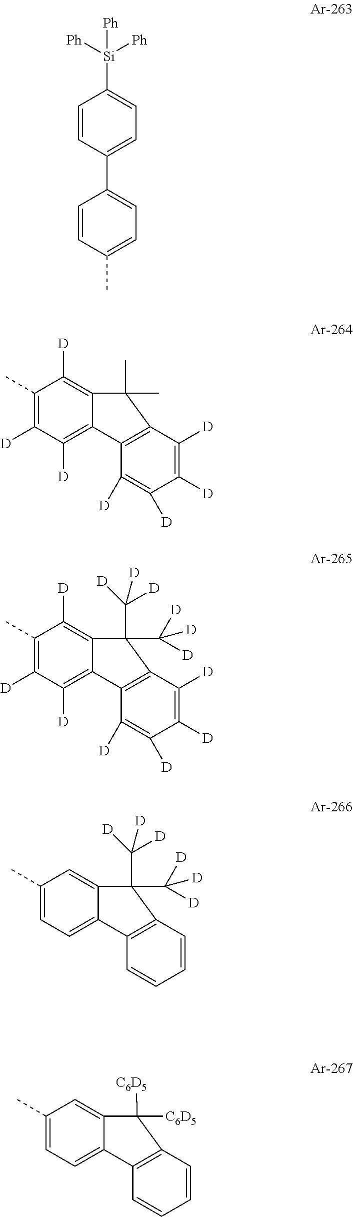

where the groups may be substituted at the free positions with groups R⁴, but are preferably unsubstituted in these positions, and where the dotted line symbolizes the bonding position to the nitrogen atom.

Particularly preferred groups Ar¹ and Ar² are groups which conform to one of the above formulae Ar-1, Ar-2, Ar-4, Ar-5, Ar-74, Ar-78, Ar-82, Ar-117, Ar-134, Ar-139, Ar-150, Ar-172 and Ar-207, with the provision that Ar¹ and Ar² are not identically Ar-1.

Further particularly preferred groups Ar¹ and Ar² are groups which conform to one of the above formulae Ar-253, Ar-254, Ar-255, Ar-256, Ar-257, Ar-258, Ar-259, Ar-260, Ar-261, Ar-261, Ar-262, Ar-263, Ar-264, Ar-265, Ar-266 and Ar-267.

According to a preferred embodiment, index m is 0, meaning that groups Ar¹ and Ar² are not connected by a group E.

According to an alternative embodiment, which may be preferred under certain conditions, index m is 1, meaning that groups Ar¹ and Ar² are connected by a group E.

In the case that groups Ar¹ and Ar² are connected by a group E, it is preferred that groups Ar¹ and Ar² are selected, identically or differently, from phenyl and fluorenyl, each of which may be substituted by one or more groups R⁴. Furthermore, in such case, it is preferred that the group E which connects the groups Ar¹ and Ar² is located on the respective groups Ar¹ and Ar², preferably on the respective groups Ar¹ and Ar² which are phenyl or fluorenyl, in ortho-position to the bond of the groups Ar¹ and Ar² to the amine nitrogen atom. Furthermore, preferably, in such case a six-ring with the amine nitrogen atom is formed of the groups Ar¹ and Ar² and E, if E is selected from C(R⁴)₂, NR⁴, O and S; and a five-ring is formed, if E is a single bond.

In the case that groups Ar¹ and Ar² are connected by a group E, particularly preferred embodiments of the moieties

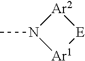

are selected from the following formulae

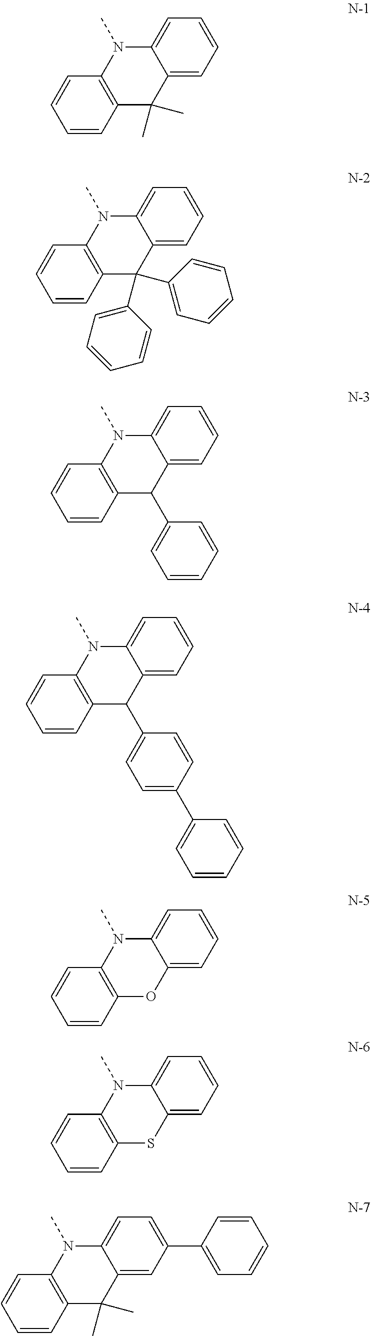

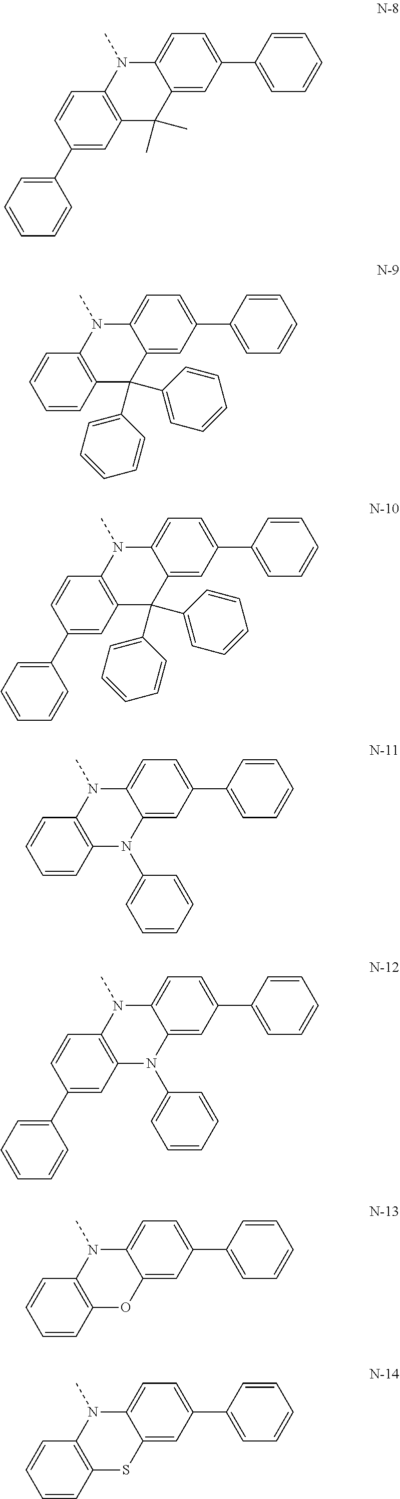

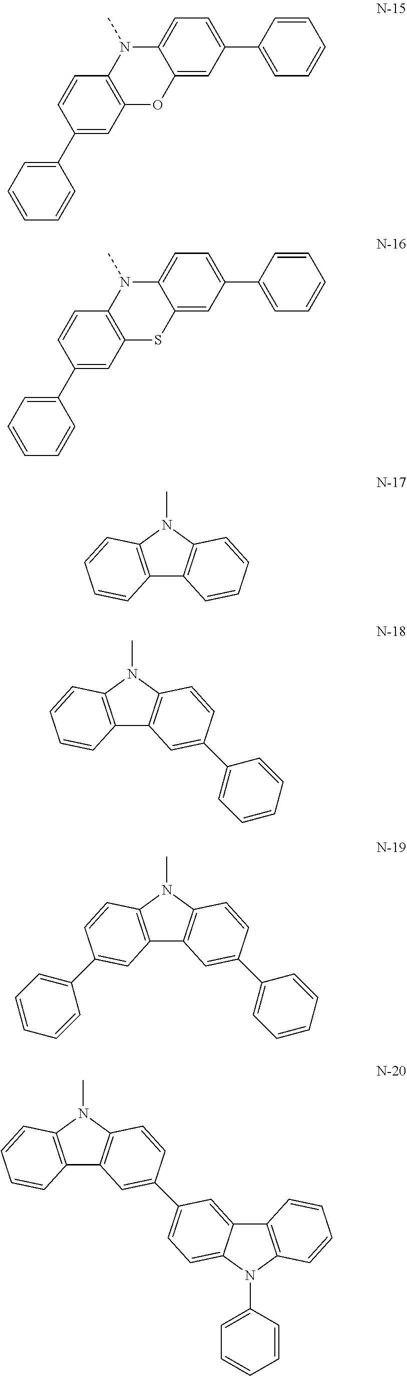

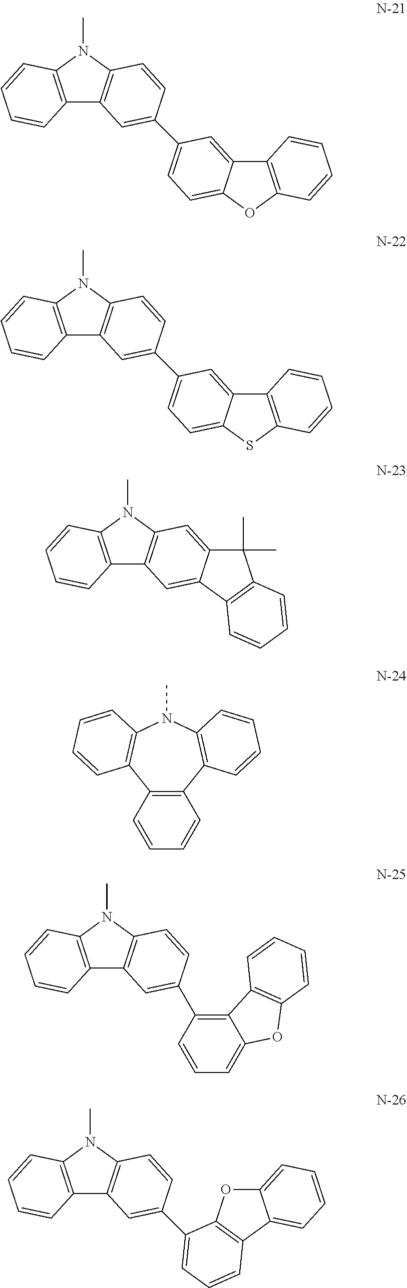

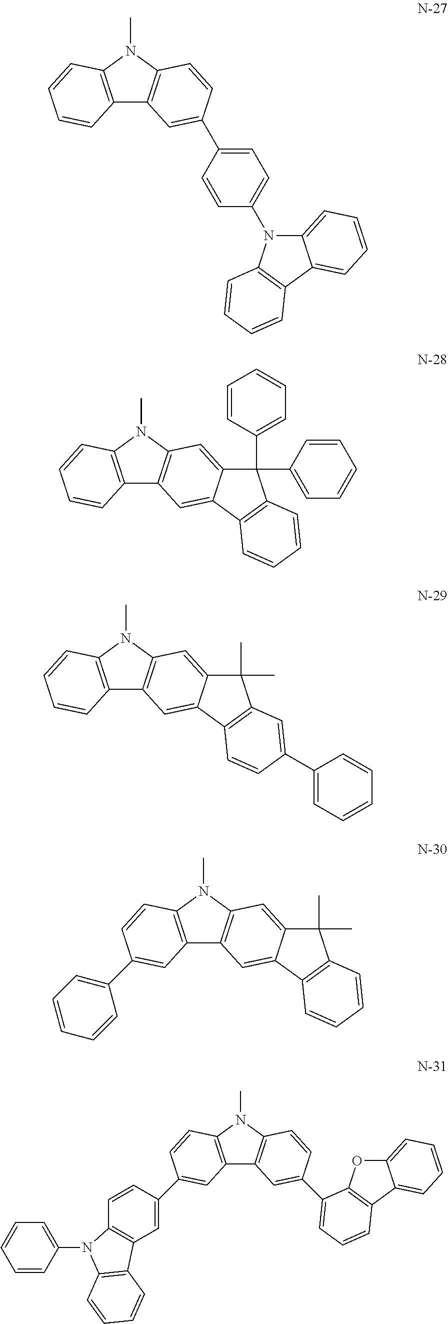

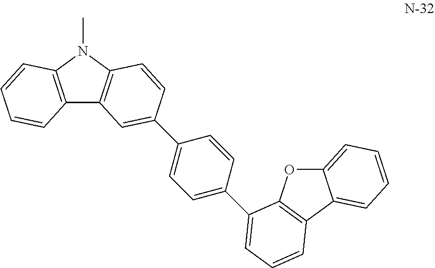 [SMILES: Cn1c2ccccc2c2cc(-c3ccc(-c4cccc5c4oc4ccccc45)cc3)ccc21]

where the groups may be substituted at the free positions with groups R⁴, but are preferably unsubstituted in these positions, and where the dotted line symbolizes the bonding position to the nitrogen atom.

For the case m=0, particularly preferable moieties

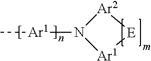

in formula (I) conform to the following formulae

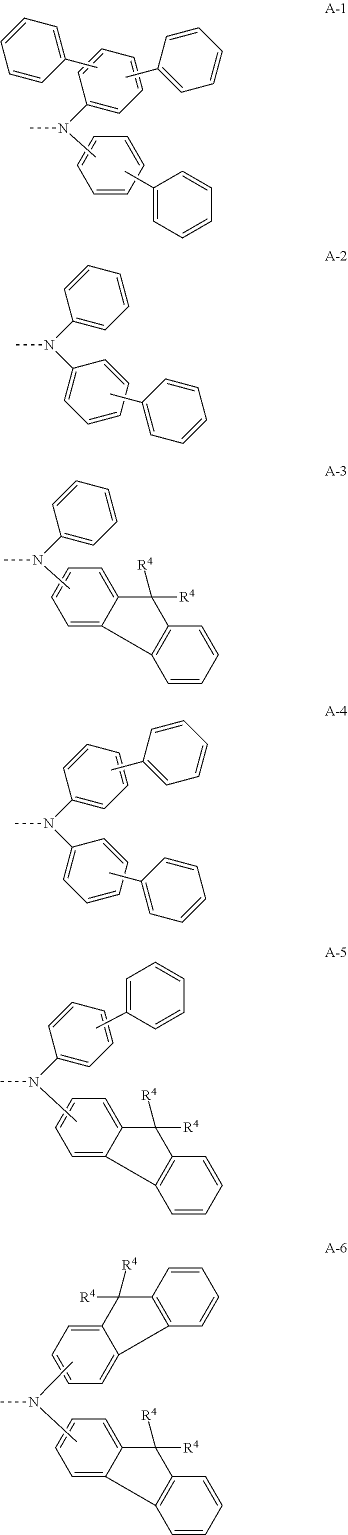

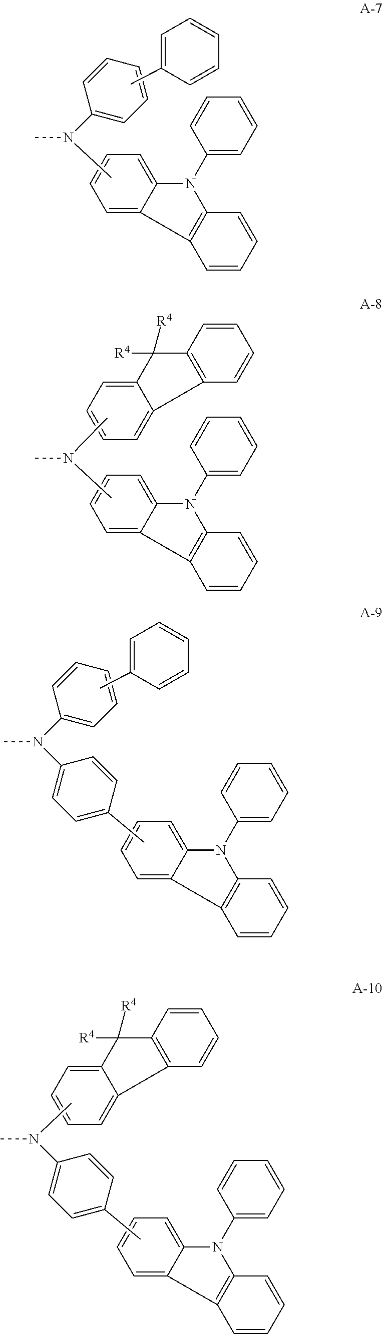

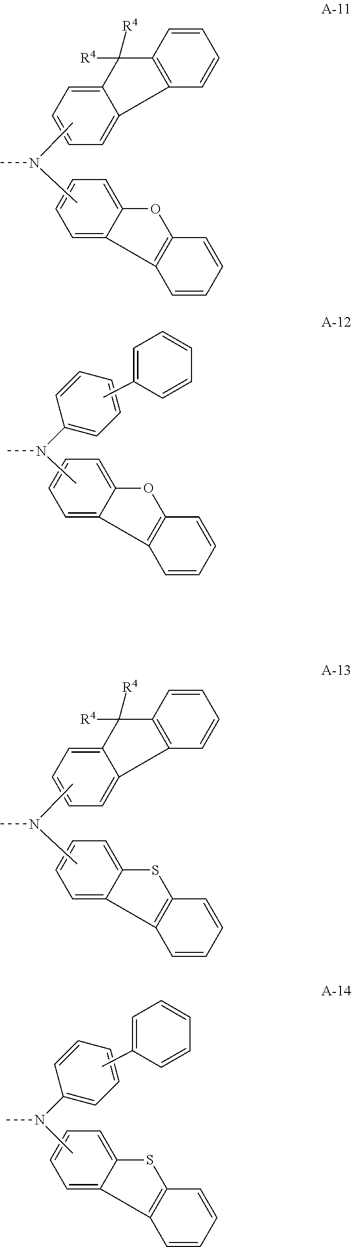

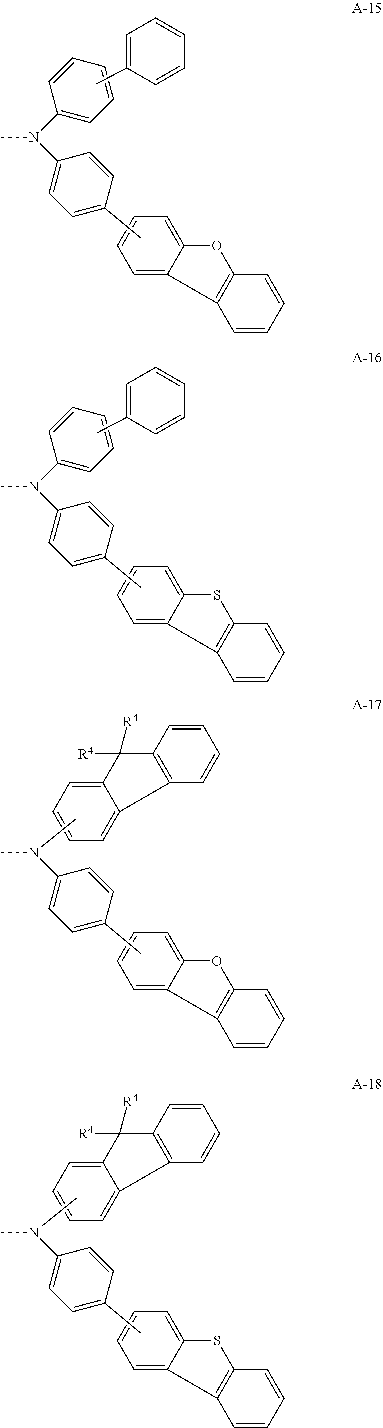

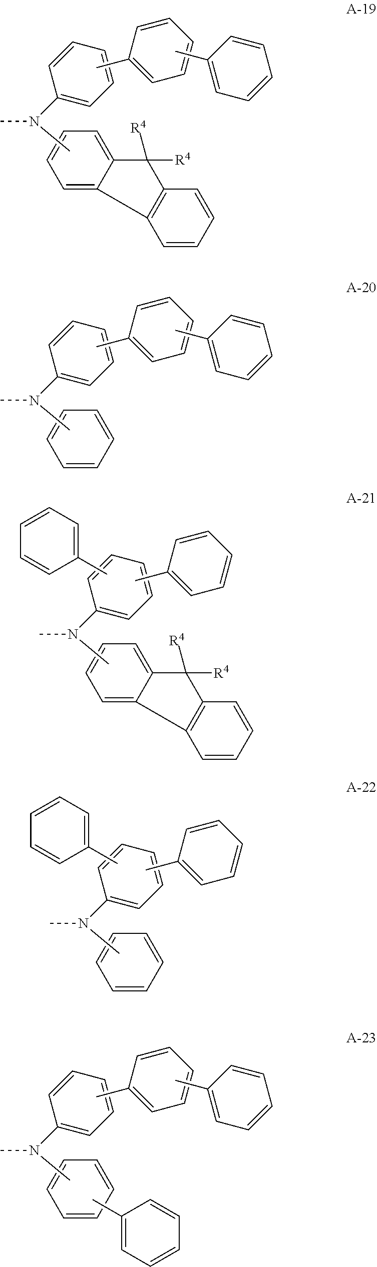

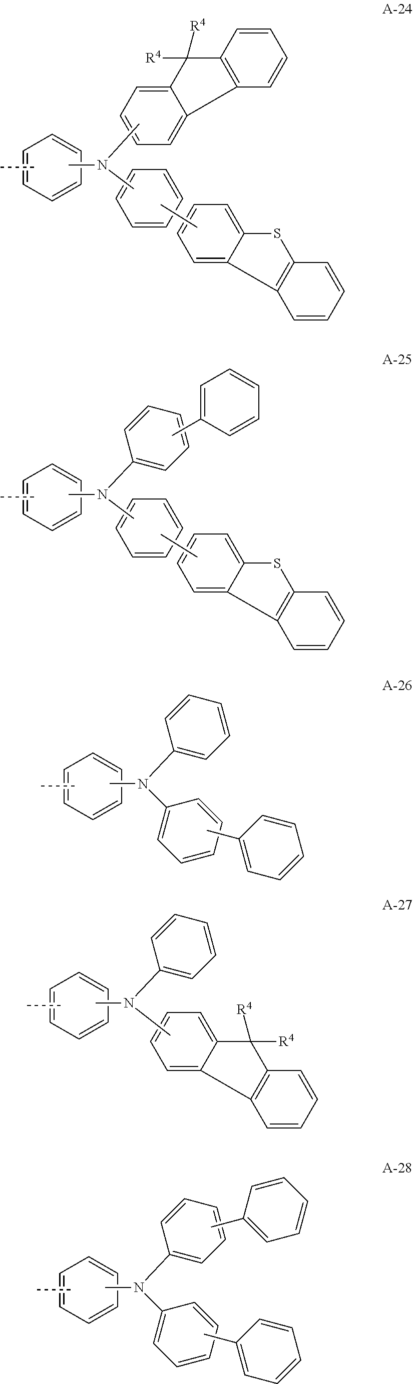

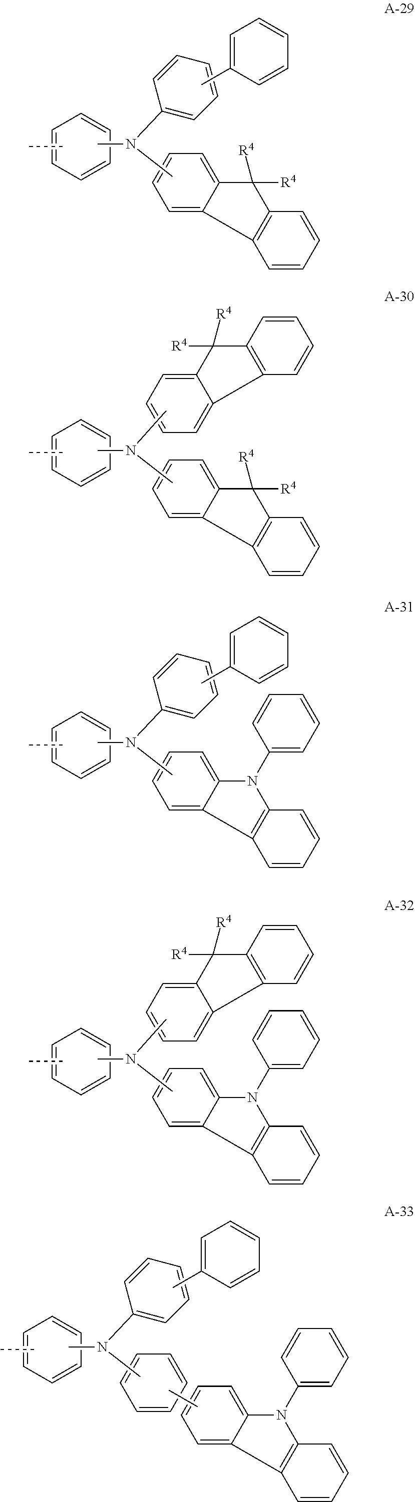

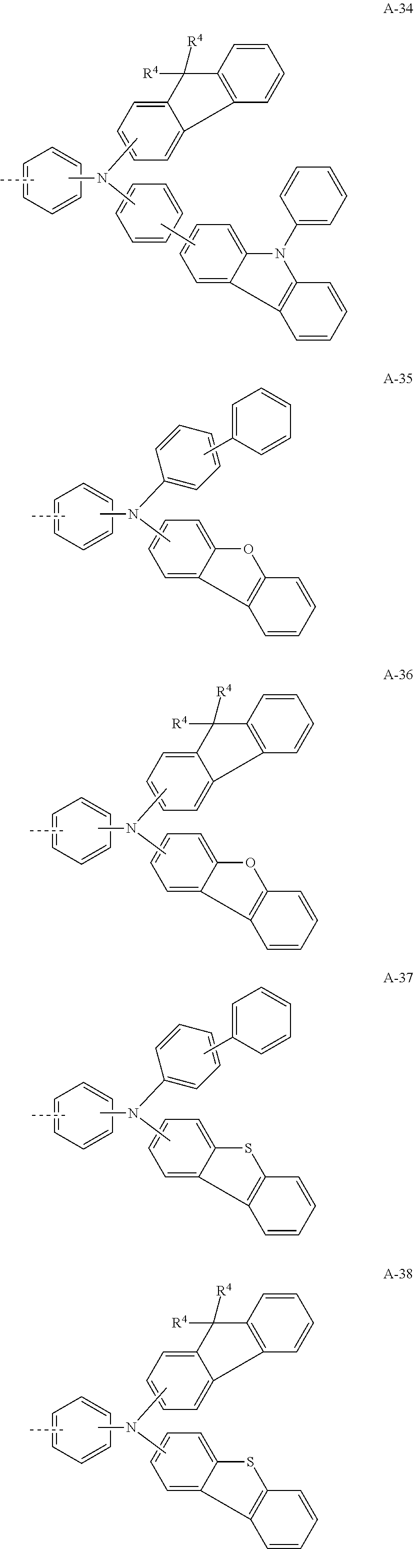

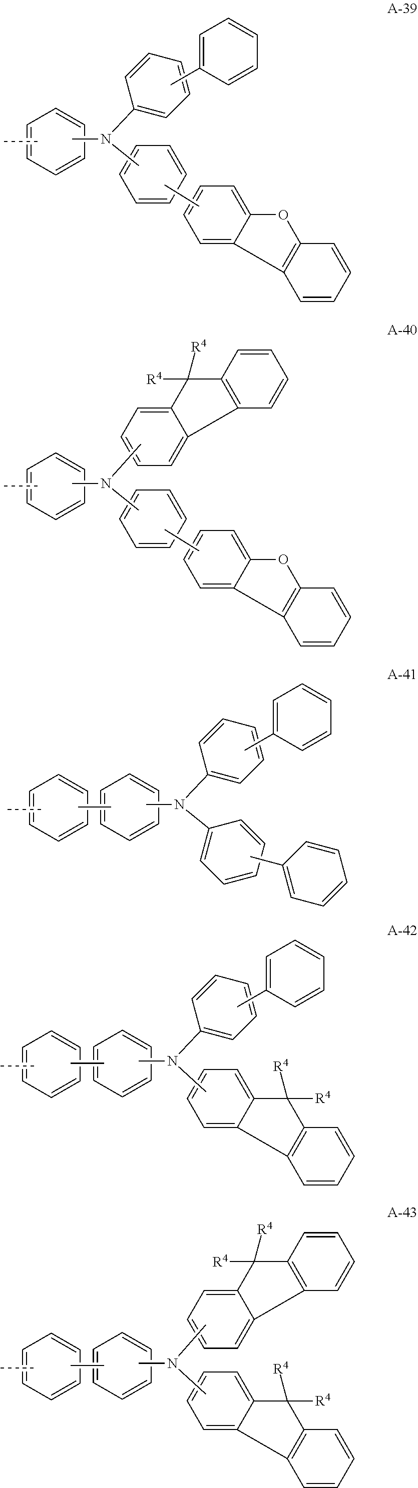

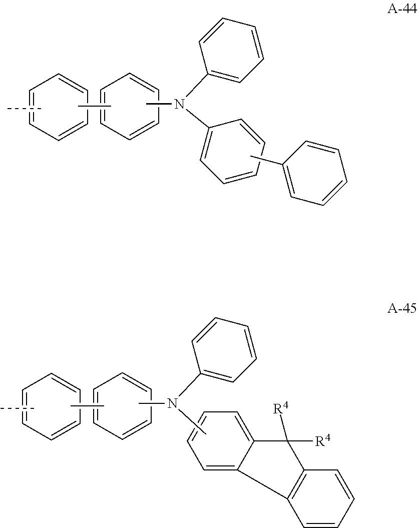

where the groups may be substituted at the free positions with groups R⁴, but are preferably unsubstituted in these positions, and where the dotted line symbolizes the bonding position to the fluorene moiety of formula (I).

Formula (I) preferably conforms to one of formulae (I-A) to (I-G)

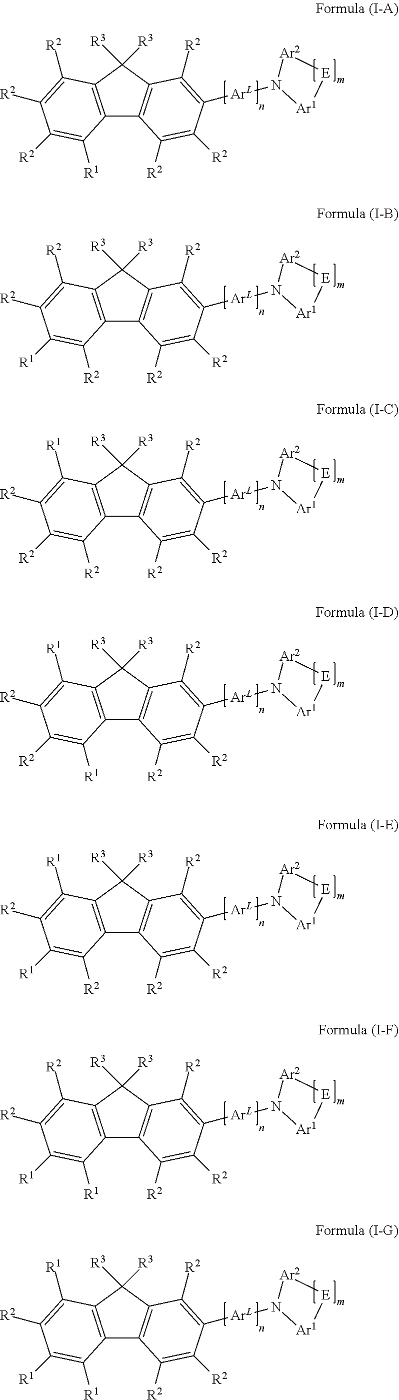

in which the variables occurring are defined as above.

Among formulae (I-A) to (I-G), formulae (I-A) and (I-E) are preferred, and formula (I-A) is particularly preferred.

More preferably, formula (I) conforms to one of formulae (I-A-1) to (I-G-1)

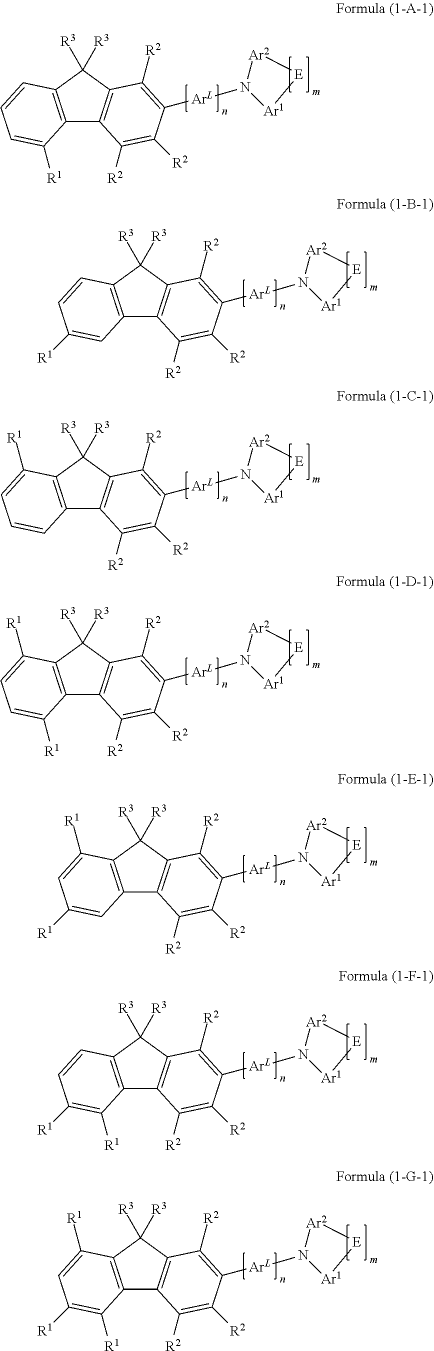

in which the variables occurring are defined as above.

Among formulae (I-A-1) to (I-G-1), formulae (I-A-1) and (I-E-1) are preferred, and formula (I-A-1) is particularly preferred.

Groups R² are preferably selected, identically or differently, from H, F, straight-chain alkyl groups having 1 to 20 C atoms, branched or cyclic alkyl groups having 3 to 20 C atoms, aromatic ring systems having 6 to 30 aromatic ring atoms, and heteroaromatic ring systems having 5 to 30 aromatic ring atoms, where the said alkyl groups, aromatic ring systems and heteroaromatic ring systems may in each case be substituted by one or more radicals R⁵. More preferably, groups R² are selected, identically or differently, from H, F, methyl, tert-butyl, and phenyl, biphenyl, dibenzofurane, dibenzothiophene, terphenyl. Most preferably, groups R² are H and phenyl.

Even more preferably, formula (I) conforms to one of formulae (I-A-2) to (I-K-2)

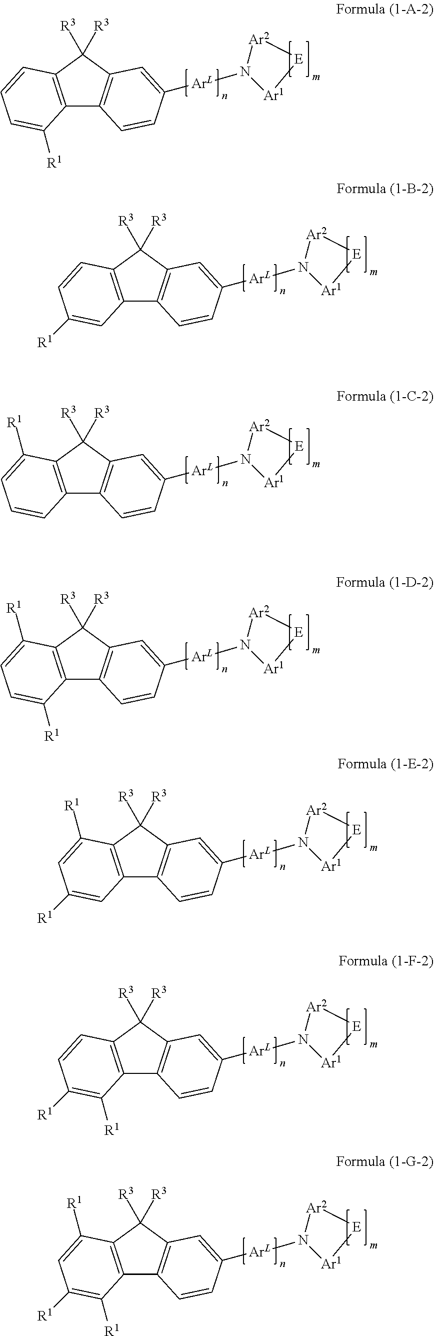

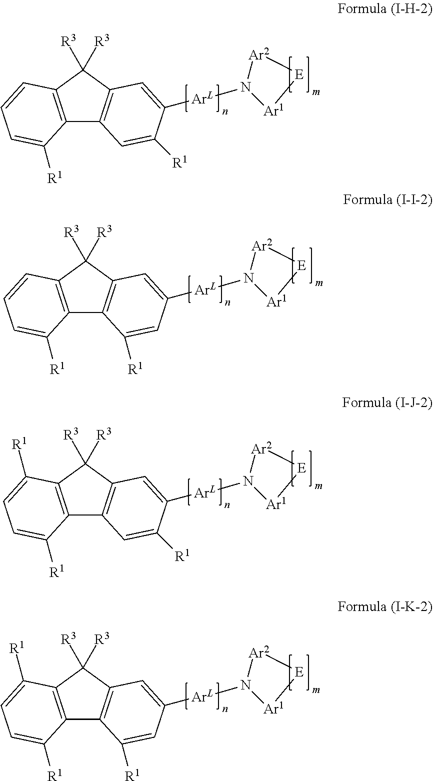

in which the variables occurring are as above.

Among formulae (I-A-2) to (I-K-2), formulae (I-A-2) and (I-E-2) are preferred, and formula (I-A-2) is particularly preferred.

Particularly preferred embodiments of formula (I) conform to one of formulae (I-A-2-1), (I-A-2-2), (I-E-2-1), (I-E-2-2), (I-D-2-1), (I-D-2-2), (I-I-2-1), (I-I-2-2), (I-H-2-1) and (I-H-2-2)

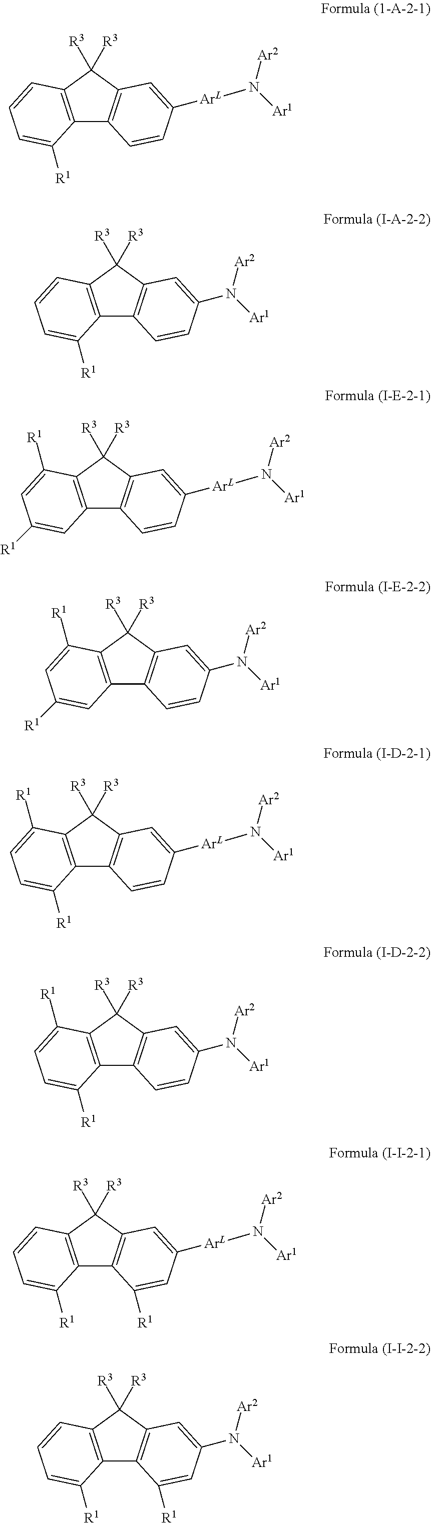

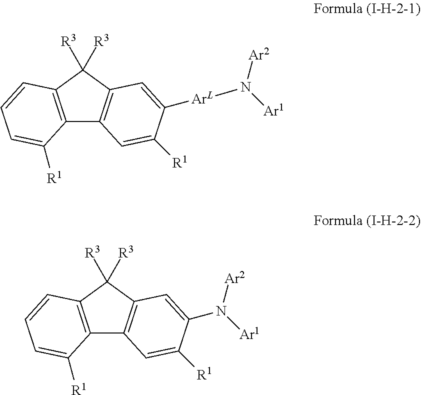

in which the variables occurring are defined as above.

Formulae (I-A-2-1) and (I-A-2-2) are especially preferred.

Preferably, in formulae (I-A-2-1) and (I-E-2-1), Ar^{L} is selected from divalent groups derived from benzene, biphenyl, terphenyl, naphthyl, dibenzofuranyl, dibenzothiophenyl, which may each be substituted by one or more radicals R⁴.

R⁴ is preferably selected, identically or differently, from H, F, CN, Si(R⁵)₃, straight-chain alkyl groups having 1 to 20 C atoms, branched or cyclic alkyl groups having 3 to 20 C atoms, aromatic ring systems having 6 to 40 aromatic ring atoms, and heteroaromatic ring systems having 5 to 40 aromatic ring atoms; where two or more radicals R⁴ may be connected to each other to form a ring; where the said alkyl groups and the said aromatic and heteroaromatic ring systems may in each case be substituted by one or more radicals R⁵.

Preferably, R¹ is selected, identically or differently on each occurrence, from

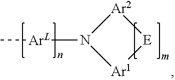

in which m is 1 and E is a single bond, aromatic ring systems having 6 to 30 aromatic ring atoms, and heteroaromatic ring systems having 5 to 30 aromatic ring atoms, where the said aromatic and heteroaromatic ring systems may in each case be substituted by one or more radicals R⁵.

More preferably, R¹ is, identically or differently on each occurrence, selected from

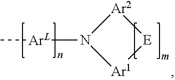

in which m is 1 and E is a single bond, phenyl, biphenyl, terphenyl, quarterphenyl, naphthyl, fluorenyl, especially 9,9′-dimethylfluorenyl and 9,9′-diphenylfluorenyl, benzofluorenyl, spirobifluorenyl, indenofluorenyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, each of which may optionally be substituted by one or more radicals R⁵.

For embodiments

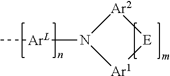

as group R¹, index m is 1 and E is a single bond, but the same preferred embodiments regarding groups Ar^{L}, Ar¹, Ar² and index n apply as mentioned above in the context of group

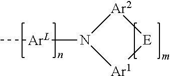

of formula (I).

Accordingly, a five-ring with the amine nitrogen atom is formed of the groups Ar¹, Ar² and E being a single bond.

Preferred specific groups R¹ are groups which conform to the following groups R-1 to R-187

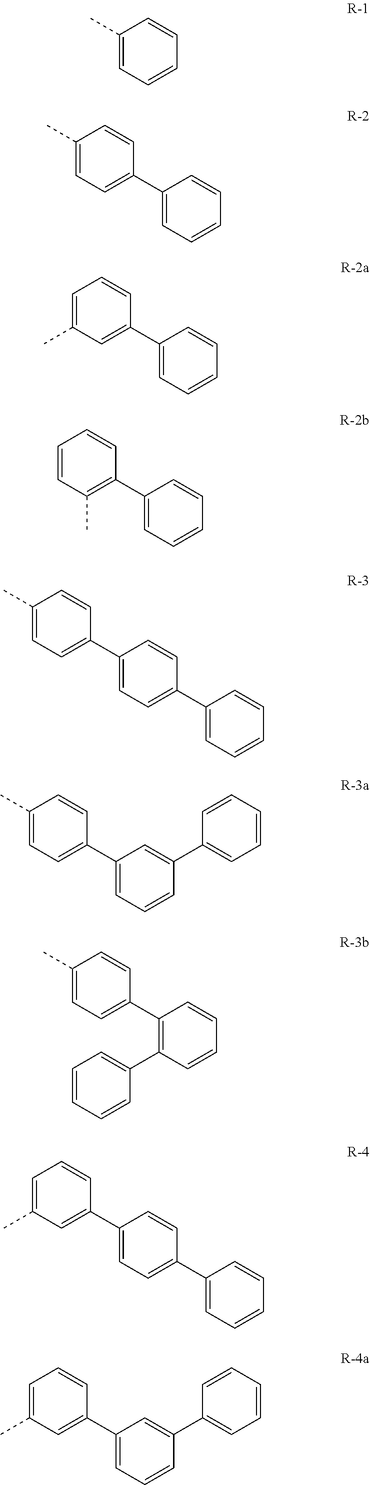

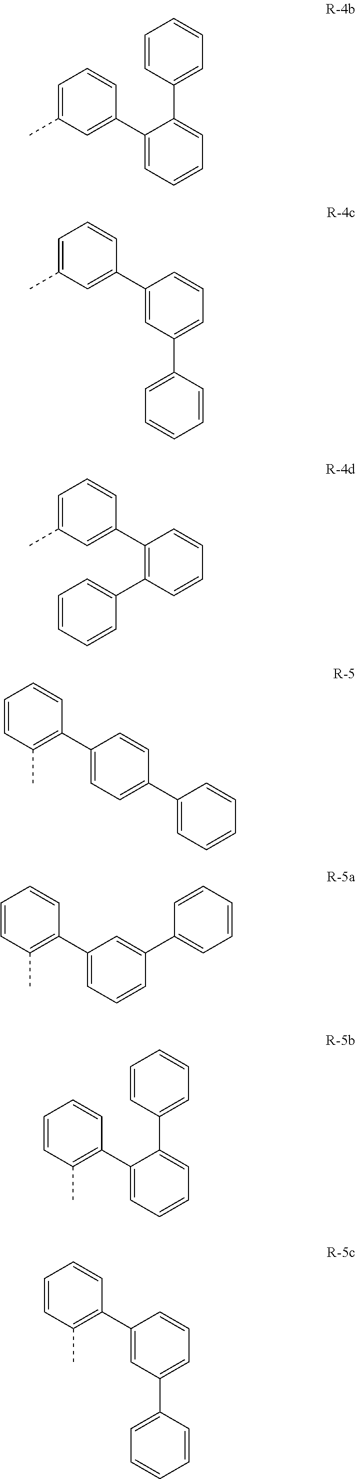

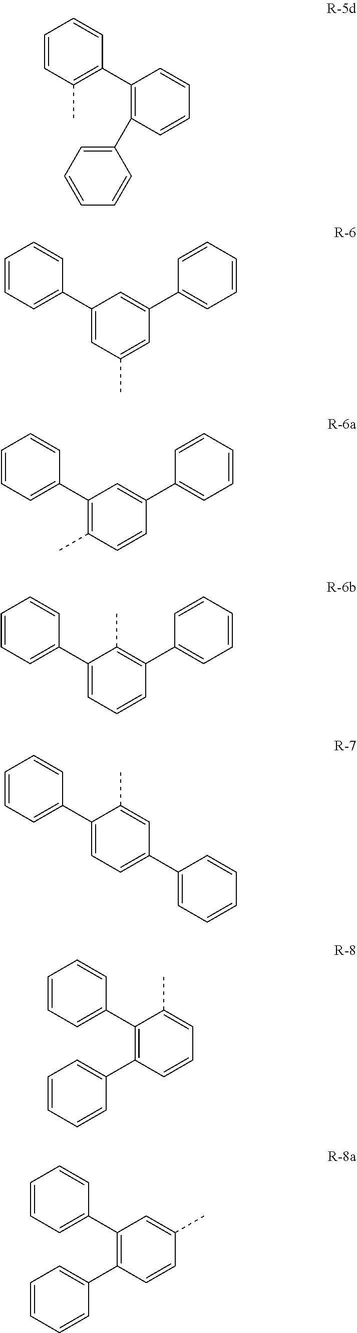

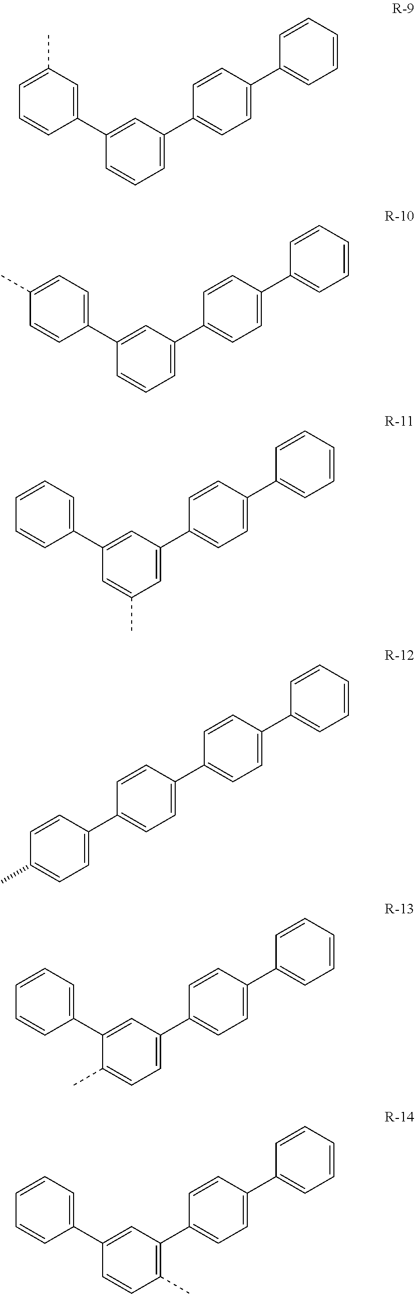

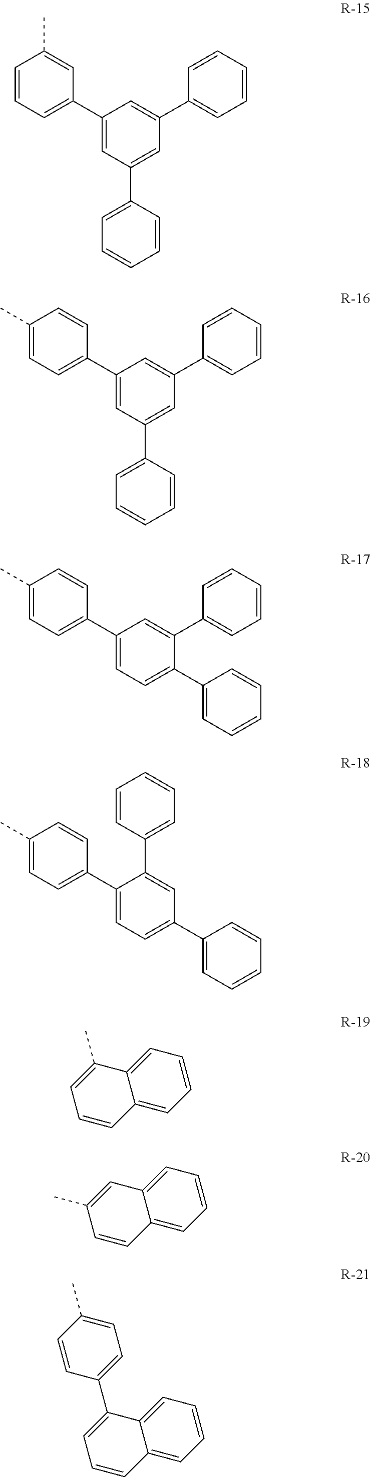

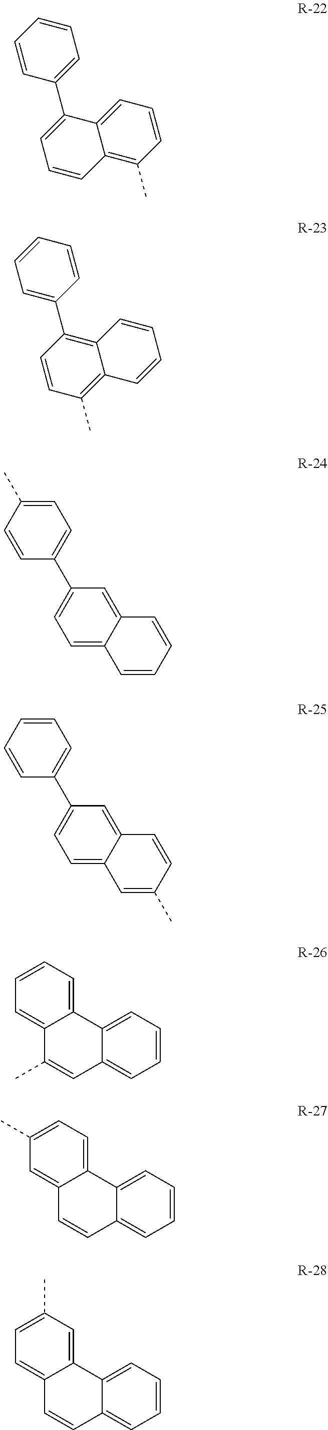

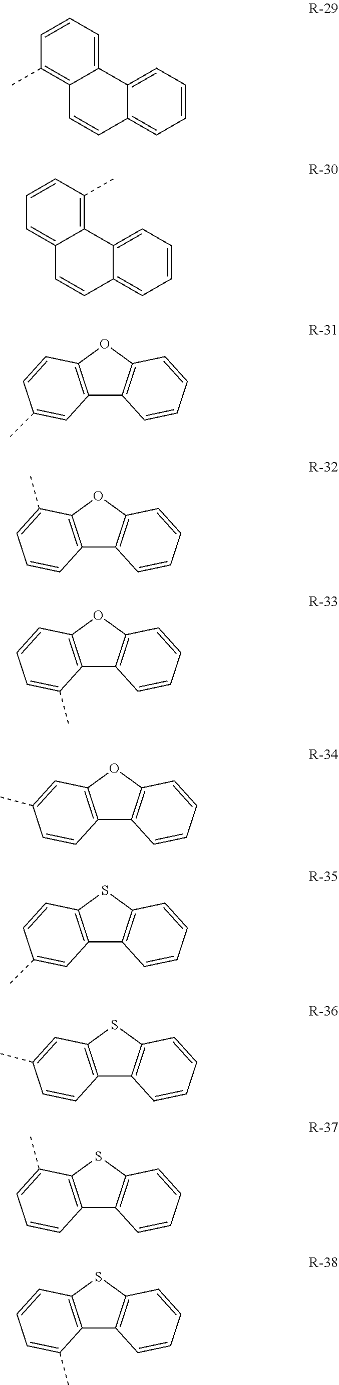

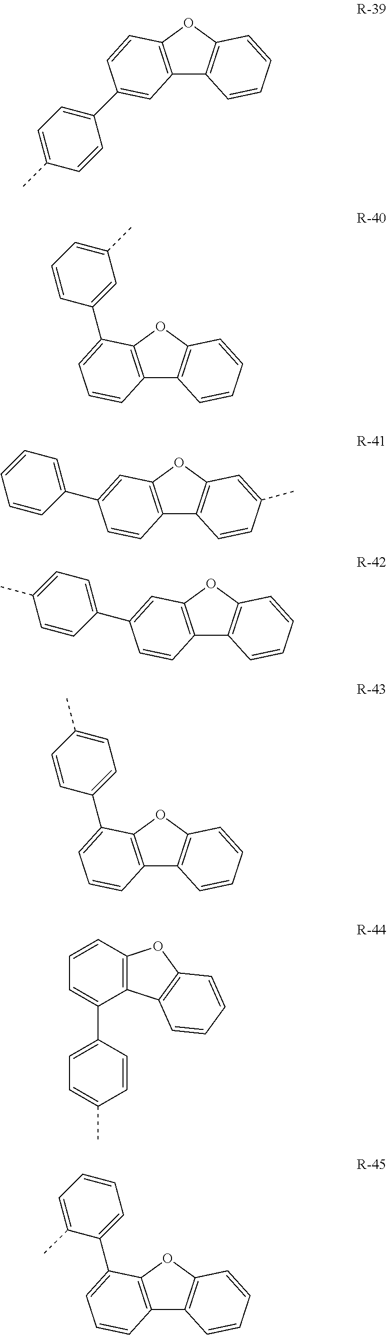

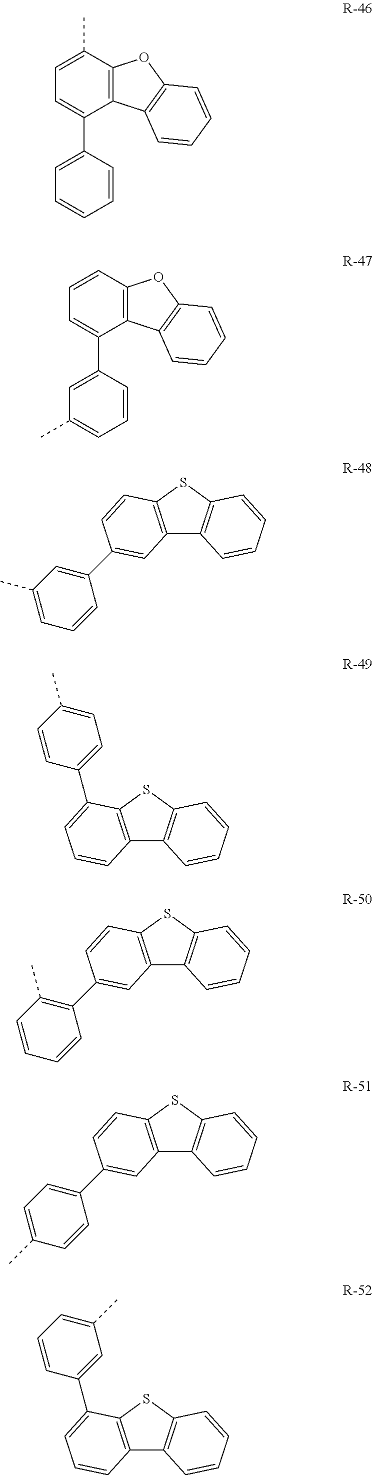

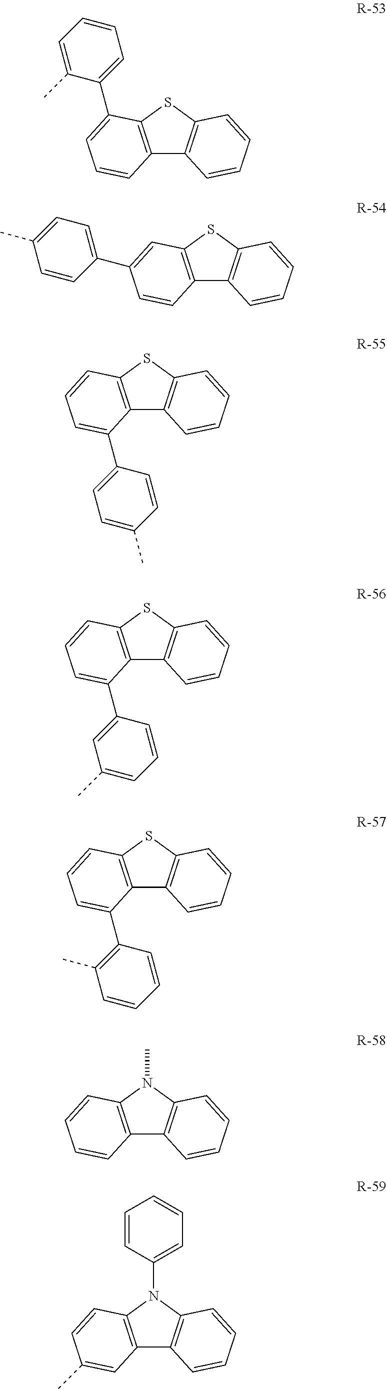

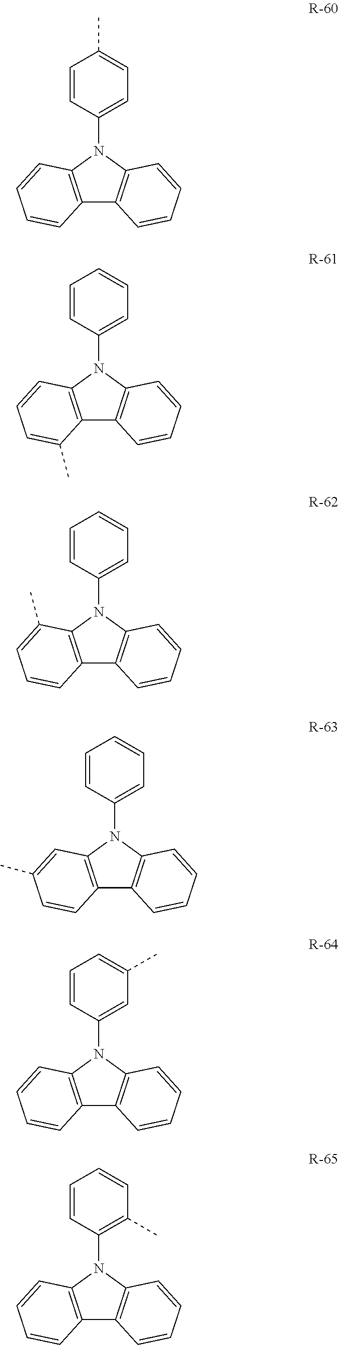

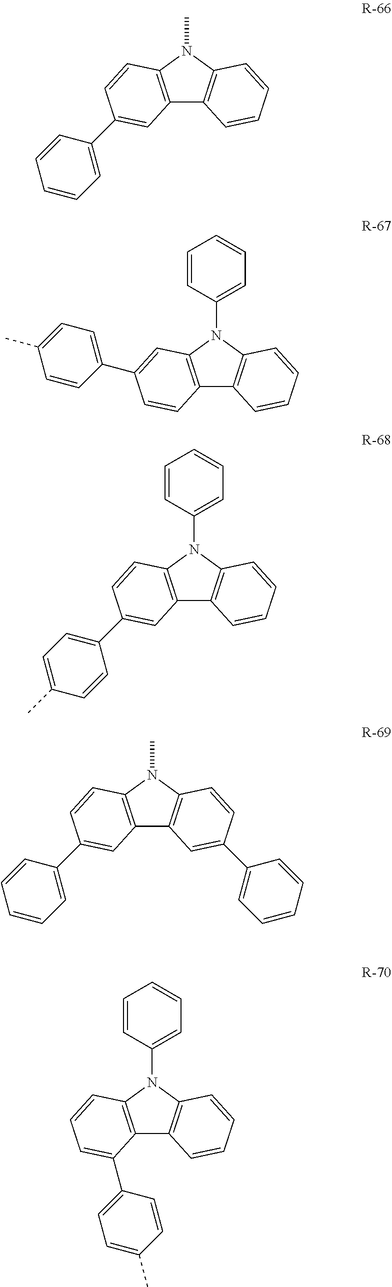

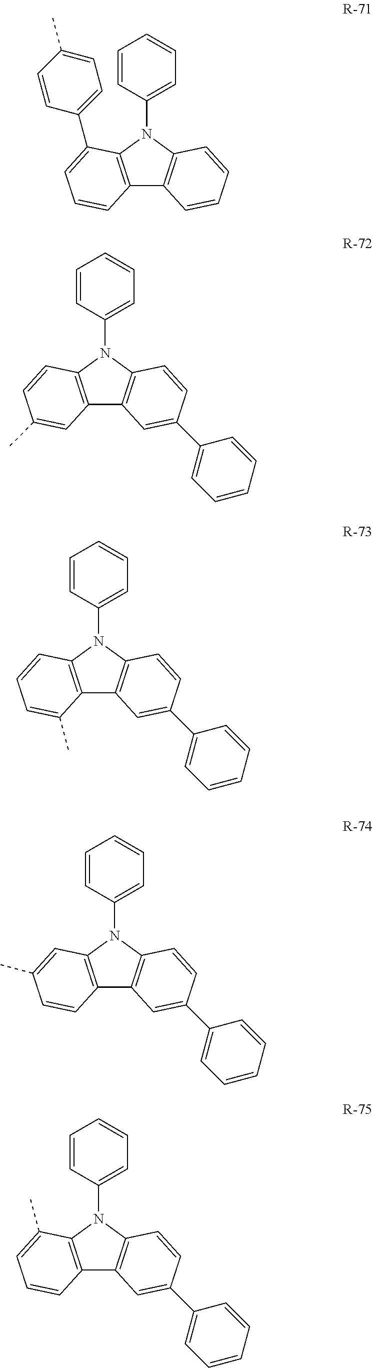

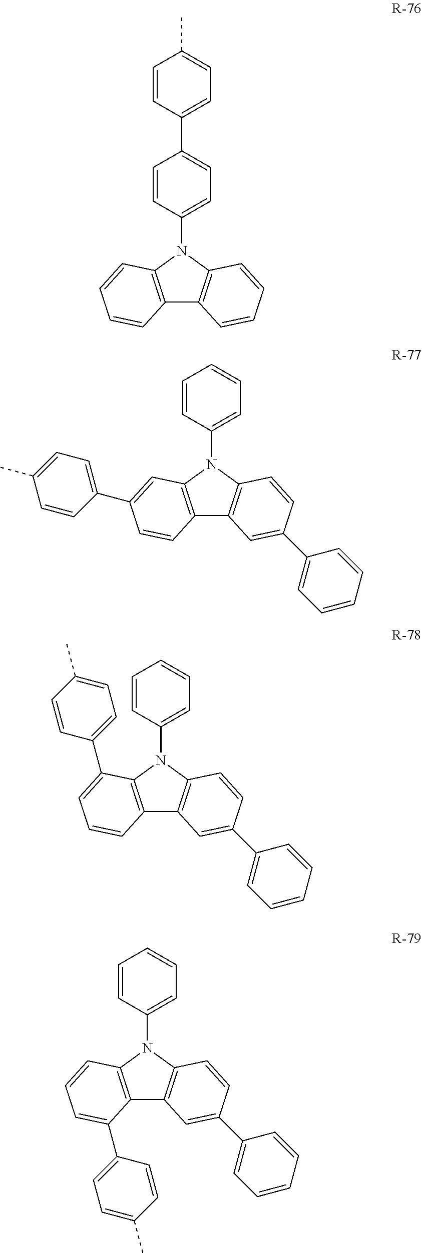

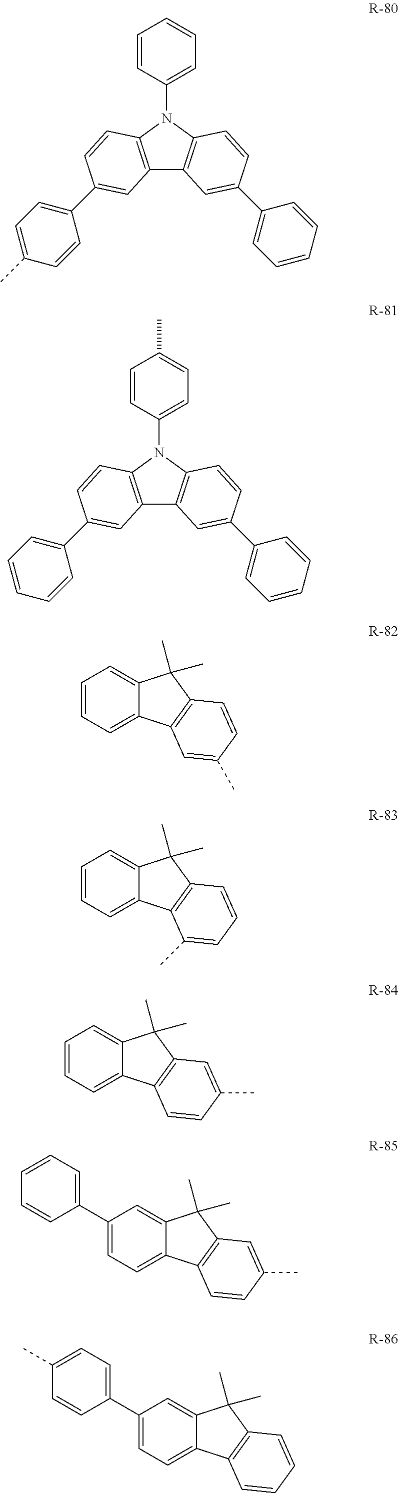

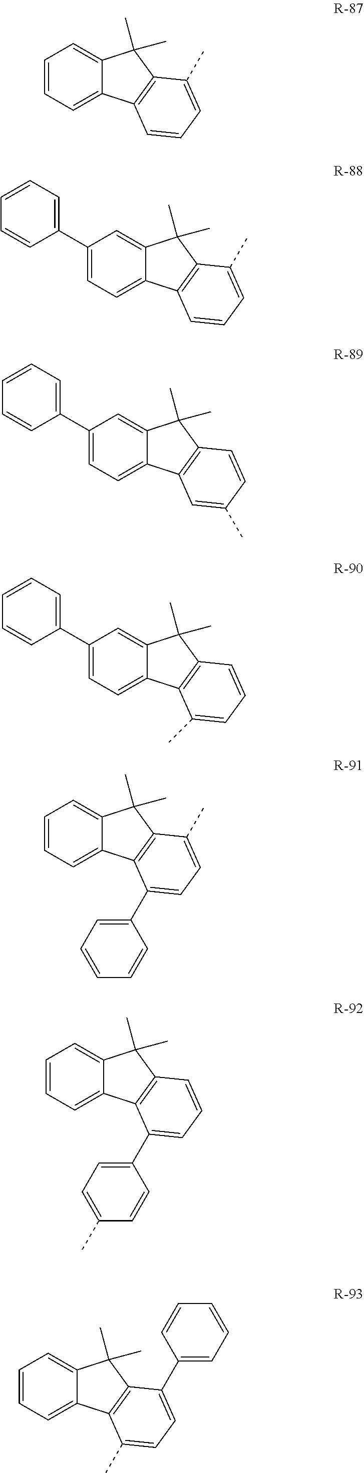

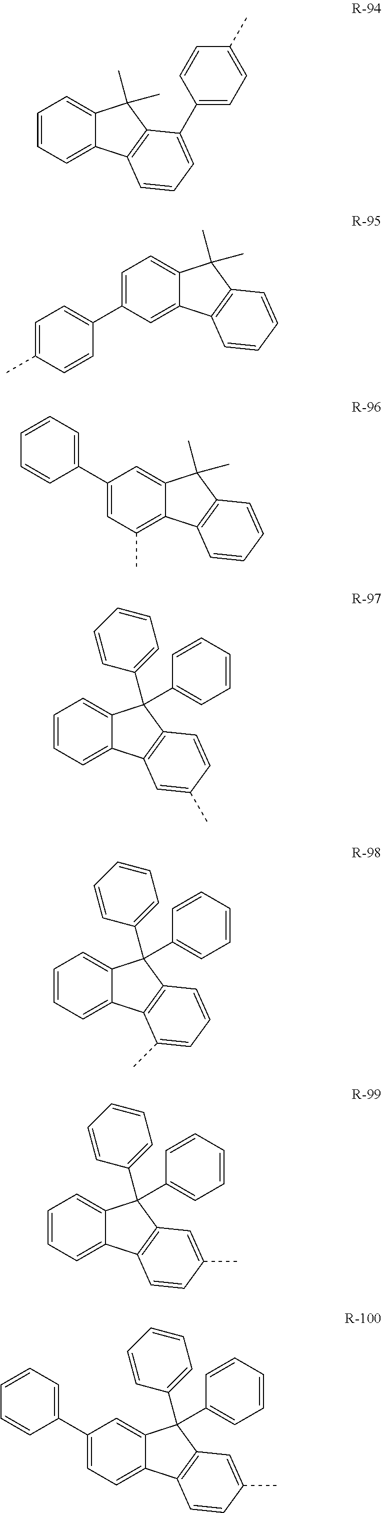

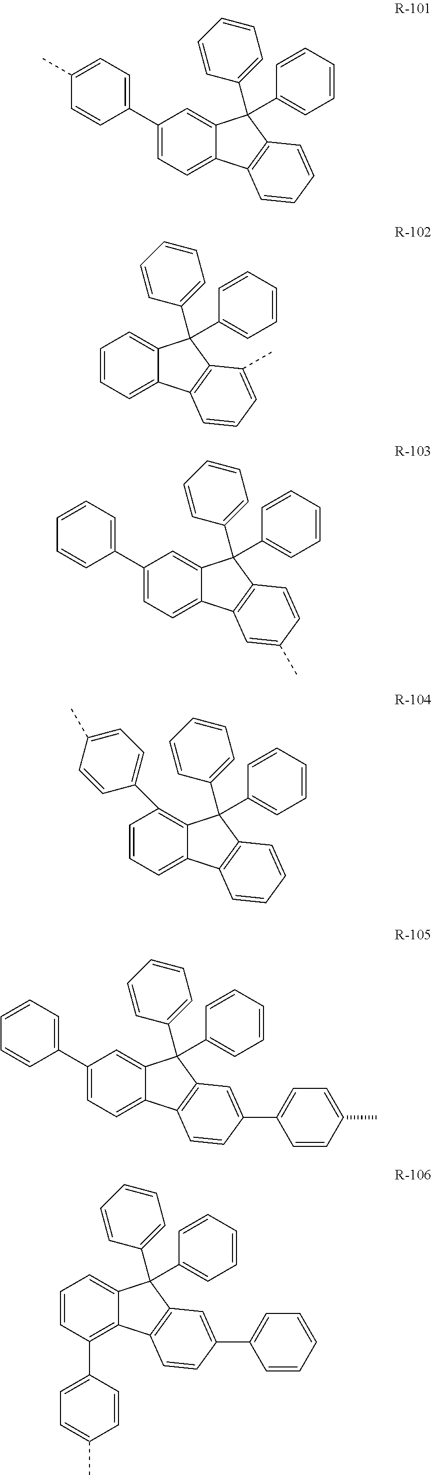

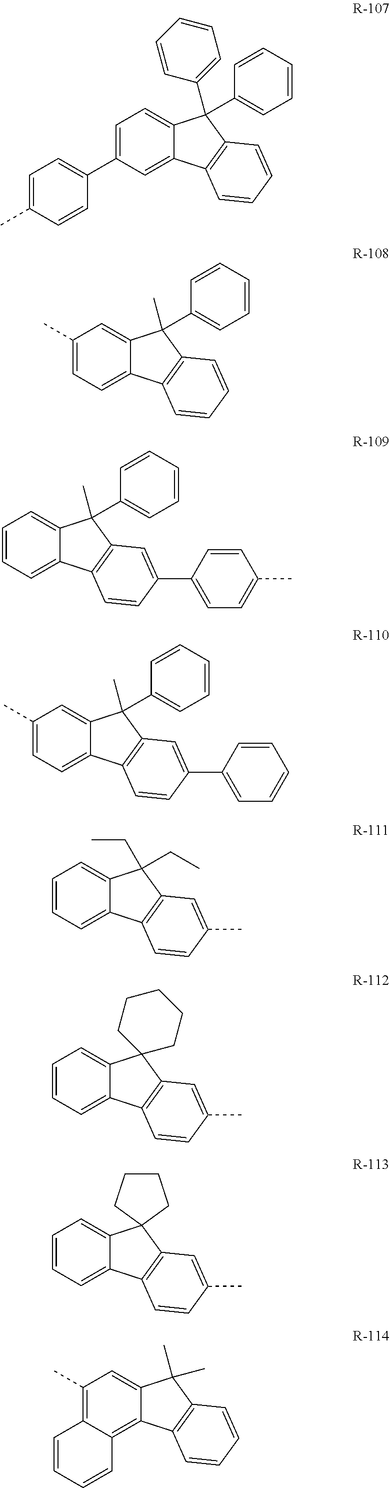

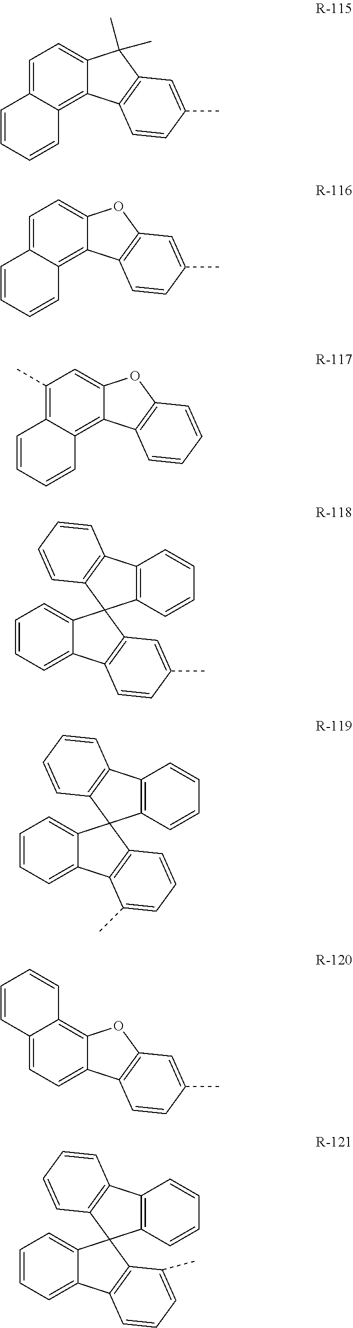

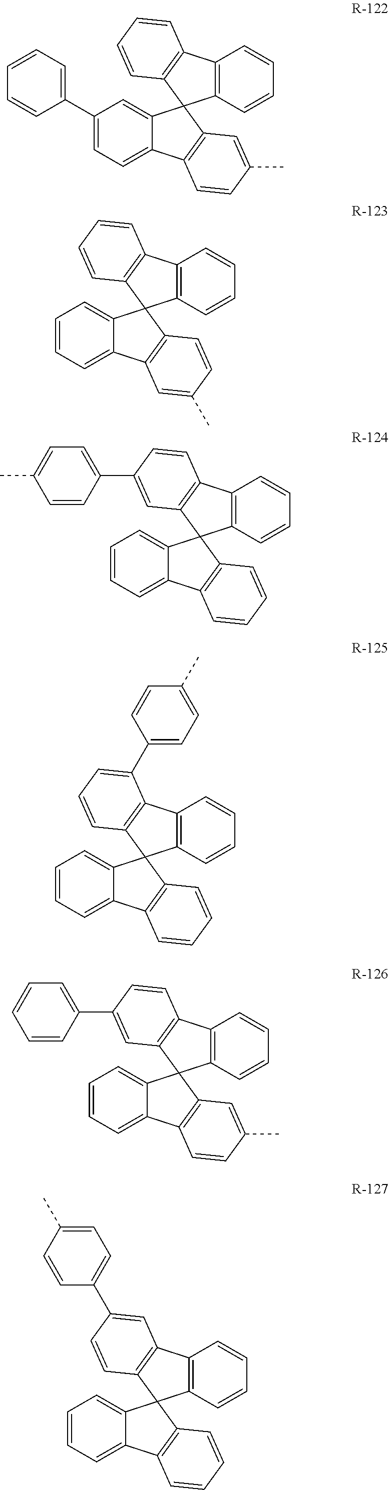

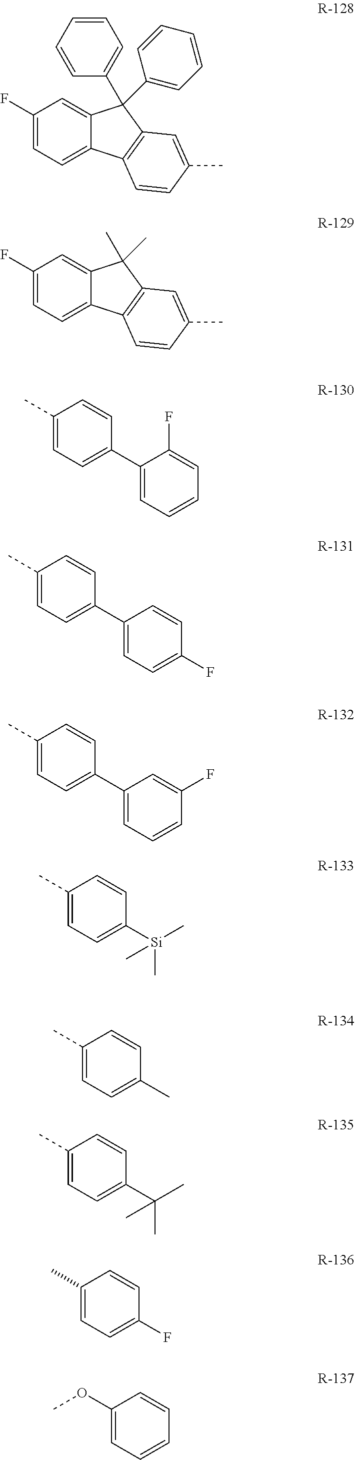

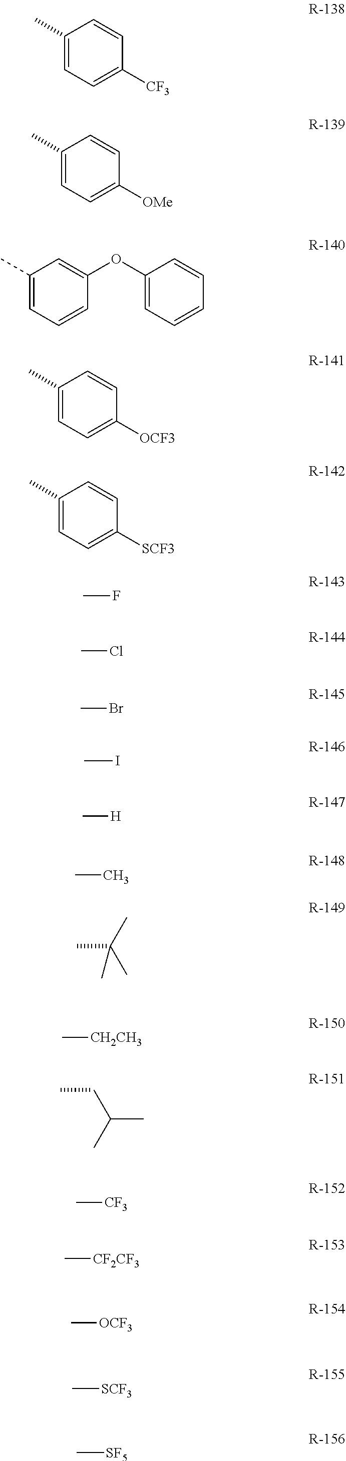

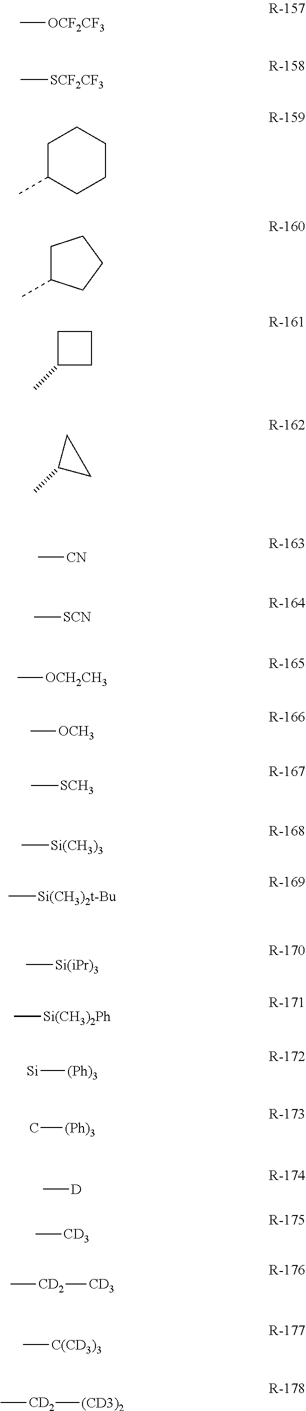

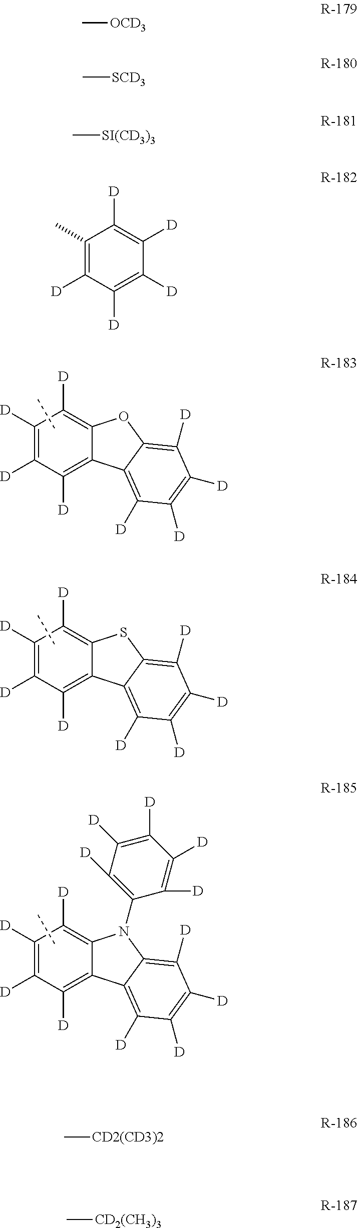

in which the groups may be substituted at the free positions with groups R⁵, but are preferably unsubstituted in these positions, and where the dotted line symbolizes the bonding position to the fluorene moiety of formula (I).

It is particularly preferred according to the present invention that groups R¹ are identical on each occurrence.

In a more preferred embodiment, R¹ is selected from aromatic ring systems having 6 to 24 aromatic ring atoms, which may in each case be substituted by one or more radicals R⁵.

Even more preferred, R¹ is selected from phenyl, biphenyl, terphenyl and quarterphenyl, each of which may optionally be substituted by one or more radicals R⁵.

Particularly preferred, R¹ is selected from phenyl, biphenyl and terphenyl, each of which may optionally be substituted by one or more radicals R⁵.

Very particularly preferred, R¹ is selected from biphenyl and terphenyl, each of which may optionally be substituted by one or more radicals R⁵.

Even more preferred, R¹ is selected from terphenyl, which may optionally be substituted by one or more radicals R⁵.

Groups R¹ conforming to one of formulae R-2 to R-2b and groups R¹ conforming to one of formulae R-3 to R-8a are especially preferred biphenyl and terphenyl groups, respectively.

In a further preferred embodiment, R¹ is selected from aromatic groups having two or more aromatic rings, which may in each case be substituted by one or more radicals R⁵.

In a particularly preferred embodiment of the present invention, in the compound of formula (I) groups R¹ are not selected from moieties

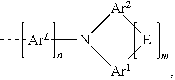

(with index m being 1 and E being a single bond) as defined above for groups R¹.

Furthermore, in a particularly preferred embodiment of the present invention, the compound of formula (I) is characterized in that it is a monoamine compound.

Preferably, R³ is selected, identically or differently on each occurrence, from straight-chain alkyl groups having 1 to 20 C atoms, or cyclic alkyl groups having 3 to 20 C atoms, where the said alkyl groups or cyclic alkyl groups may be substituted by one or more radicals R⁵, or aromatic or heteroaromatic ring systems having 6 to 30 aromatic ring atoms, where the said aromatic and heteroaromatic ring systems may in each case be substituted by one or more radicals R⁵, where the two radicals R³ may be connected to each other to form a ring, so that a spiro-compound is built at position 9 of the fluorene group, where spirobifluorenes are excluded.

More preferably, R³ is identically or different on each occurrence, selected from straight-chain alkyl groups having 1 to 10 C atoms, where the said alkyl groups may be substituted by one or more radicals R⁵, or aromatic ring systems having 6 to 24 aromatic ring atoms, where the said aromatic ring systems may in each case be substituted by one or more radicals R⁵, where the two radicals R³ may be connected to each other to form a ring, so that a spiro compound is built at position 9 of the fluorene group, where spirobifluorenes are excluded.

According to a particularly preferred embodiment of the present invention, two groups R³ are not connected to each other to form a ring.

Further, according to a particularly preferred embodiment of the present invention, groups R³ are identical on each occurrence.

Particularly preferred according to the invention are groups R³ selected from straight chain alkyl groups having 1 to 10 C atoms, wherein even more preferably the alkyl chain is substituted by one or more deuterium atoms and most preferably any of the hydrogen atoms of the alkyl group is replaced by a deuterium. The most preferred alkyl group that comprises deuterium as R³ group is -CD₃.

In another preferred embodiment of the instant invention R³ is a deuterated phenyl group (—C₆D₅).

Compounds according to the invention that exhibit the substitution with deuterium show improved performance data, when used in electronic devices, such as OLEDs. In particular, lifetime of the devices, but also voltage, efficiency and even more shelf life and stability of the compounds can be improved.

Thus, subject of the present invention is a compound of formula (I) comprising at least one group that is deuterated. Preferably the compound of formula (I) comprises at least one deuterated group that is a deuterated methyl group (-CD₃), wherein the deuterated methyl group is most preferably bonded to the carbon atom in position 9 of a fluorene.

Particularly preferred groups R³ are groups which conform to the following groups R-188 to R-202

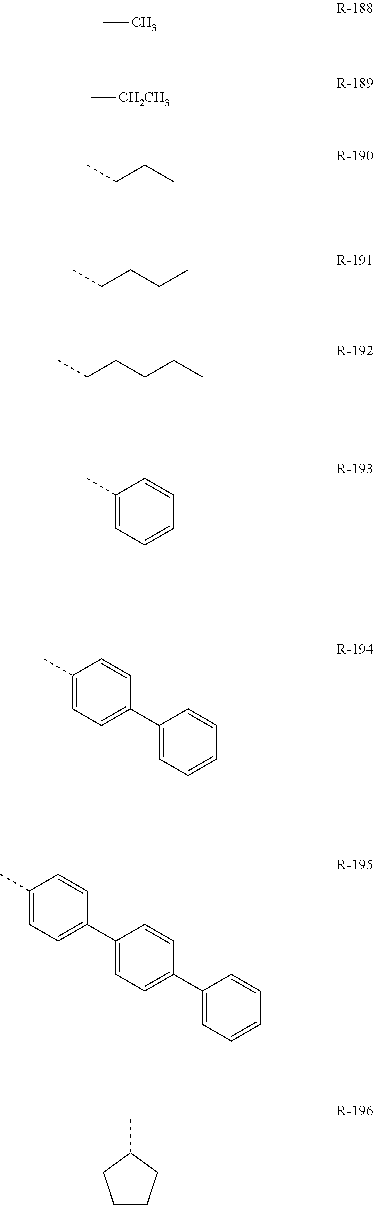

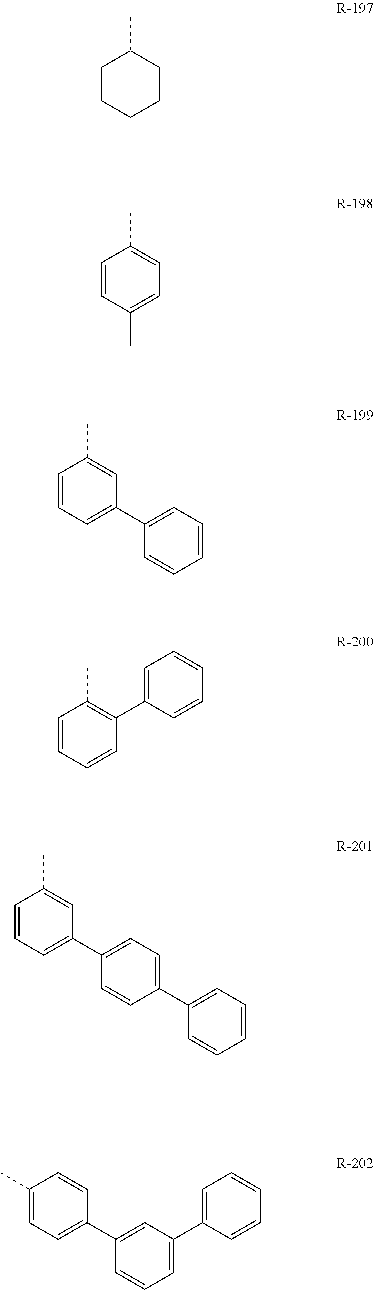

in which the groups may be substituted at the free positions with radicals R⁵, but are preferably unsubstituted in these positions, and where the dotted line symbolizes the bonding position to the fluorene moiety of formula (I).

Among the above illustrated particularly preferred groups R³, groups conforming to formula R-188 (methyl) and to formula R-193 (phenyl) are most preferred groups R³.

R⁵ is preferably selected, identically or differently, from H, F, CN, Si(R⁶)₃, straight-chain alkyl groups having 1 to 20 C atoms, branched or cyclic alkyl groups having 3 to 20 C atoms, aromatic ring systems having 6 to 40 aromatic ring atoms, and heteroaromatic ring systems having 5 to 40 aromatic ring atoms; where two or more radicals R⁵ may be connected to each other to form a ring; where the said alkyl groups and the said aromatic and heteroaromatic ring systems may in each case be substituted by one or more radicals R⁶.

Particularly preferred specific compounds are the following compounds, which conform to the formula (I-A-2-2) above, in which R³, R¹, Ar¹ and Ar² are specified as shown in the list below (formulae Ar-1 to Ar-207 and R-1 to R-66 are as defined above):

| No. | R³ | R¹ | Ar¹ | Ar² |
|---|---|---|---|---|
| C-1 | methyl | R-1 | Ar-1 | Ar-2 |
| C-2 | ″ | ″ | ″ | Ar-4 |
| C-3 | ″ | ″ | ″ | Ar-5 |
| C-4 | ″ | ″ | ″ | Ar-74 |
| C-5 | ″ | ″ | ″ | Ar-78 |
| C-6 | ″ | ″ | ″ | Ar-82 |
| C-7 | ″ | ″ | ″ | Ar-117 |
| C-8 | ″ | ″ | ″ | Ar-134 |
| C-9 | ″ | ″ | ″ | Ar-139 |
| C-10 | ″ | ″ | ″ | Ar-150 |
| C-11 | ″ | ″ | ″ | Ar-172 |
| C-12 | ″ | ″ | ″ | Ar-207 |
| C-13 | ″ | ″ | Ar-2 | Ar-2 |
| C-14 | ″ | ″ | ″ | Ar-4 |
| C-15 | ″ | ″ | ″ | Ar-5 |
| C-16 | ″ | ″ | ″ | Ar-74 |
| C-17 | ″ | ″ | ″ | Ar-78 |
| C-18 | ″ | ″ | ″ | Ar-82 |
| C-19 | ″ | ″ | ″ | Ar-117 |
| C-20 | ″ | ″ | ″ | Ar-134 |
| C-21 | ″ | ″ | ″ | Ar-139 |
| C-22 | ″ | ″ | ″ | Ar-150 |
| C-23 | ″ | ″ | ″ | Ar-172 |
| C-24 | ″ | ″ | ″ | Ar-207 |
| C-25 | ″ | ″ | Ar-4 | Ar-4 |
| C-26 | ″ | ″ | ″ | Ar-5 |
| C-27 | ″ | ″ | ″ | Ar-74 |
| C-28 | ″ | ″ | ″ | Ar-78 |
| C-29 | ″ | ″ | ″ | Ar-82 |
| C-30 | ″ | ″ | ″ | Ar-117 |
| C-31 | ″ | ″ | ″ | Ar-134 |
| C-32 | ″ | ″ | ″ | Ar-139 |
| C-33 | ″ | ″ | ″ | Ar-150 |
| C-34 | ″ | ″ | ″ | Ar-172 |
| C-35 | ″ | ″ | ″ | Ar-207 |
| C-36 | ″ | ″ | Ar-5 | Ar-5 |
| C-37 | ″ | ″ | ″ | Ar-74 |
| C-38 | ″ | ″ | ″ | Ar-78 |
| C-39 | ″ | ″ | ″ | Ar-82 |
| C-40 | ″ | ″ | ″ | Ar-117 |
| C-41 | ″ | ″ | ″ | Ar-134 |
| C-42 | ″ | ″ | ″ | Ar-139 |
| C-43 | ″ | ″ | ″ | Ar-150 |
| C-44 | ″ | ″ | ″ | Ar-172 |
| C-45 | ″ | ″ | ″ | Ar-207 |
| C-46 | ″ | ″ | Ar-74 | Ar-74 |
| C-47 | ″ | ″ | ″ | Ar-78 |
| C-48 | ″ | ″ | ″ | Ar-82 |
| C-49 | ″ | ″ | ″ | Ar-117 |
| C-50 | ″ | ″ | ″ | Ar-134 |
| C-51 | ″ | ″ | ″ | Ar-139 |
| C-52 | ″ | ″ | ″ | Ar-150 |
| C-53 | ″ | ″ | ″ | Ar-172 |
| C-54 | ″ | ″ | ″ | Ar-207 |
| C-55 | ″ | ″ | Ar-78 | Ar-78 |
| C-56 | ″ | ″ | ″ | Ar-82 |
| C-57 | ″ | ″ | ″ | Ar-117 |
| C-58 | ″ | ″ | ″ | Ar-134 |
| C-59 | ″ | ″ | ″ | Ar-139 |
| C-60 | ″ | ″ | ″ | Ar-150 |
| C-61 | ″ | ″ | ″ | Ar-172 |
| C-62 | ″ | ″ | ″ | Ar-207 |
| C-63 | ″ | ″ | Ar-82 | Ar-82 |
| C-64 | ″ | ″ | ″ | Ar-117 |
| C-65 | ″ | ″ | ″ | Ar-134 |
| C-66 | ″ | ″ | ″ | Ar-139 |
| C-67 | ″ | ″ | ″ | Ar-150 |
| C-68 | ″ | ″ | ″ | Ar-172 |
| C-69 | ″ | ″ | ″ | Ar-207 |
| C-70 | ″ | ″ | Ar-117 | Ar-117 |
| C-71 | ″ | ″ | ″ | Ar-134 |
| C-72 | ″ | ″ | ″ | Ar-139 |
| C-73 | ″ | ″ | ″ | Ar-150 |
| C-74 | ″ | ″ | ″ | Ar-172 |
| C-75 | ″ | ″ | ″ | Ar-207 |
| C-76 | ″ | ″ | Ar-134 | Ar-134 |
| C-77 | ″ | ″ | ″ | Ar-139 |
| C-78 | ″ | ″ | ″ | Ar-150 |
| C-79 | ″ | ″ | ″ | Ar-172 |
| C-80 | ″ | ″ | ″ | Ar-207 |
| C-81 | ″ | ″ | Ar-139 | Ar-139 |
| C-82 | ″ | ″ | ″ | Ar-150 |
| C-83 | ″ | ″ | ″ | Ar-172 |
| C-84 | ″ | ″ | ″ | Ar-207 |
| C-85 | ″ | ″ | Ar-150 | Ar-150 |
| C-86 | ″ | ″ | ″ | Ar-172 |
| C-87 | ″ | ″ | ″ | Ar-207 |
| C-88 | ″ | ″ | Ar-172 | Ar-172 |
| C-89 | ″ | ″ | ″ | Ar-207 |
| C-90 | ″ | ″ | Ar-207 | Ar-207 |
| C-91 | ″ | Biphenyl* | Ar-1 | Ar-2 |
| C-92 | ″ | ″ | ″ | Ar-4 |
| C-93 | ″ | ″ | ″ | Ar-5 |
| C-94 | ″ | ″ | ″ | Ar-74 |
| C-95 | ″ | ″ | ″ | Ar-78 |
| C-96 | ″ | ″ | ″ | Ar-82 |
| C-97 | ″ | ″ | ″ | Ar-117 |
| C-98 | ″ | ″ | ″ | Ar-134 |
| C-99 | ″ | ″ | ″ | Ar-139 |
| C-100 | ″ | ″ | ″ | Ar-150 |
| C-101 | ″ | ″ | ″ | Ar-172 |
| C-102 | ″ | ″ | ″ | Ar-207 |
| C-103 | ″ | ″ | Ar-2 | Ar-2 |
| C-104 | ″ | ″ | ″ | Ar-4 |
| C-105 | ″ | ″ | ″ | Ar-5 |
| C-106 | ″ | ″ | ″ | Ar-74 |
| C-107 | ″ | ″ | ″ | Ar-78 |
| C-108 | ″ | ″ | ″ | Ar-82 |
| C-109 | ″ | ″ | ″ | Ar-117 |
| C-110 | ″ | ″ | ″ | Ar-134 |
| C-111 | ″ | ″ | ″ | Ar-139 |
| C-112 | ″ | ″ | ″ | Ar-150 |
| C-113 | ″ | ″ | ″ | Ar-172 |
| C-114 | ″ | ″ | ″ | Ar-207 |
| C-115 | ″ | ″ | Ar-4 | Ar-4 |
| C-116 | ″ | ″ | ″ | Ar-5 |
| C-117 | ″ | ″ | ″ | Ar-74 |
| C-118 | ″ | ″ | ″ | Ar-78 |
| C-119 | ″ | ″ | ″ | Ar-82 |
| C-120 | ″ | ″ | ″ | Ar-117 |
| C-121 | ″ | ″ | ″ | Ar-134 |
| C-122 | ″ | ″ | ″ | Ar-139 |
| C-123 | ″ | ″ | ″ | Ar-150 |
| C-124 | ″ | ″ | ″ | Ar-172 |
| C-125 | ″ | ″ | ″ | Ar-207 |
| C-126 | ″ | ″ | Ar-5 | Ar-5 |
| C-127 | ″ | ″ | ″ | Ar-74 |
| C-128 | ″ | ″ | ″ | Ar-78 |
| C-129 | ″ | ″ | ″ | Ar-82 |
| C-130 | ″ | ″ | ″ | Ar-117 |
| C-131 | ″ | ″ | ″ | Ar-134 |
| C-132 | ″ | ″ | ″ | Ar-139 |
| C-133 | ″ | ″ | ″ | Ar-150 |
| C-134 | ″ | ″ | ″ | Ar-172 |
| C-135 | ″ | ″ | ″ | Ar-207 |
| C-136 | ″ | ″ | Ar-74 | Ar-74 |
| C-137 | ″ | ″ | ″ | Ar-78 |
| C-138 | ″ | ″ | ″ | Ar-82 |
| C-139 | ″ | ″ | ″ | Ar-117 |
| C-140 | ″ | ″ | ″ | Ar-134 |
| C-141 | ″ | ″ | ″ | Ar-139 |
| C-142 | ″ | ″ | ″ | Ar-150 |
| C-143 | ″ | ″ | ″ | Ar-172 |
| C-144 | ″ | ″ | ″ | Ar-207 |
| C-145 | ″ | ″ | Ar-78 | Ar-78 |
| C-146 | ″ | ″ | ″ | Ar-82 |
| C-147 | ″ | ″ | ″ | Ar-117 |
| C-148 | ″ | ″ | ″ | Ar-134 |
| C-149 | ″ | ″ | ″ | Ar-139 |
| C-150 | ″ | ″ | ″ | Ar-150 |
| C-151 | ″ | ″ | ″ | Ar-172 |
| C-152 | ″ | ″ | ″ | Ar-207 |
| C-153 | ″ | ″ | Ar-82 | Ar-82 |
| C-154 | ″ | ″ | ″ | Ar-117 |
| C-155 | ″ | ″ | ″ | Ar-134 |
| C-156 | ″ | ″ | ″ | Ar-139 |
| C-157 | ″ | ″ | ″ | Ar-150 |
| C-158 | ″ | ″ | ″ | Ar-172 |
| C-159 | ″ | ″ | ″ | Ar-207 |
| C-160 | ″ | ″ | Ar-117 | Ar-117 |
| C-161 | ″ | ″ | ″ | Ar-134 |
| C-162 | ″ | ″ | ″ | Ar-139 |
| C-163 | ″ | ″ | ″ | Ar-150 |
| C-164 | ″ | ″ | ″ | Ar-172 |
| C-165 | ″ | ″ | ″ | Ar-207 |
| C-166 | ″ | ″ | Ar-134 | Ar-134 |
| C-167 | ″ | ″ | ″ | Ar-139 |
| C-168 | ″ | ″ | ″ | Ar-150 |
| C-169 | ″ | ″ | ″ | Ar-172 |
| C-170 | ″ | ″ | ″ | Ar-207 |
| C-171 | ″ | ″ | Ar-139 | Ar-139 |
| C-172 | ″ | ″ | ″ | Ar-150 |
| C-173 | ″ | ″ | ″ | Ar-172 |
| C-174 | ″ | ″ | ″ | Ar-207 |
| C-175 | ″ | ″ | Ar-150 | Ar-150 |
| C-176 | ″ | ″ | ″ | Ar-172 |
| C-177 | ″ | ″ | ″ | Ar-207 |
| C-178 | ″ | ″ | Ar-172 | Ar-172 |
| C-179 | ″ | ″ | ″ | Ar-207 |
| C-180 | ″ | ″ | Ar-207 | Ar-207 |
| C-181 | ″ | R-21 | Ar-1 | Ar-2 |
| C-182 | ″ | ″ | ″ | Ar-4 |
| C-183 | ″ | ″ | ″ | Ar-5 |
| C-184 | ″ | ″ | ″ | Ar-74 |
| C-185 | ″ | ″ | ″ | Ar-78 |
| C-186 | ″ | ″ | ″ | Ar-82 |
| C-187 | ″ | ″ | ″ | Ar-117 |
| C-188 | ″ | ″ | ″ | Ar-134 |
| C-189 | ″ | ″ | ″ | Ar-139 |
| C-190 | ″ | ″ | ″ | Ar-150 |
| C-191 | ″ | ″ | ″ | Ar-172 |
| C-192 | ″ | ″ | ″ | Ar-207 |
| C-193 | ″ | ″ | Ar-2 | Ar-2 |
| C-194 | ″ | ″ | ″ | Ar-4 |
| C-195 | ″ | ″ | ″ | Ar-5 |
| C-196 | ″ | ″ | ″ | Ar-74 |
| C-197 | ″ | ″ | ″ | Ar-78 |
| C-198 | ″ | ″ | ″ | Ar-82 |
| C-199 | ″ | ″ | ″ | Ar-117 |
| C-200 | ″ | ″ | ″ | Ar-134 |
| C-201 | ″ | ″ | ″ | Ar-139 |
| C-202 | ″ | ″ | ″ | Ar-150 |
| C-203 | ″ | ″ | ″ | Ar-172 |
| C-204 | ″ | ″ | ″ | Ar-207 |
| C-205 | ″ | ″ | Ar-4 | Ar-4 |
| C-206 | ″ | ″ | ″ | Ar-5 |
| C-207 | ″ | ″ | ″ | Ar-74 |
| C-208 | ″ | ″ | ″ | Ar-78 |
| C-209 | ″ | ″ | ″ | Ar-82 |
| C-210 | ″ | ″ | ″ | Ar-117 |
| C-211 | ″ | ″ | ″ | Ar-134 |
| C-212 | ″ | ″ | ″ | Ar-139 |
| C-213 | ″ | ″ | ″ | Ar-150 |
| C-214 | ″ | ″ | ″ | Ar-172 |
| C-215 | ″ | ″ | ″ | Ar-207 |
| C-216 | ″ | ″ | Ar-5 | Ar-5 |
| C-217 | ″ | ″ | ″ | Ar-74 |
| C-218 | ″ | ″ | ″ | Ar-78 |
| C-219 | ″ | ″ | ″ | Ar-82 |
| C-220 | ″ | ″ | ″ | Ar-117 |
| C-221 | ″ | ″ | ″ | Ar-134 |
| C-222 | ″ | ″ | ″ | Ar-139 |
| C-223 | ″ | ″ | ″ | Ar-150 |
| C-224 | ″ | ″ | ″ | Ar-172 |
| C-225 | ″ | ″ | ″ | Ar-207 |
| C-226 | ″ | ″ | Ar-74 | Ar-74 |
| C-227 | ″ | ″ | ″ | Ar-78 |
| C-228 | ″ | ″ | ″ | Ar-82 |
| C-229 | ″ | ″ | ″ | Ar-117 |
| C-230 | ″ | ″ | ″ | Ar-134 |
| C-231 | ″ | ″ | ″ | Ar-139 |
| C-232 | ″ | ″ | ″ | Ar-150 |
| C-233 | ″ | ″ | ″ | Ar-172 |
| C-234 | ″ | ″ | ″ | Ar-207 |
| C-235 | ″ | ″ | Ar-78 | Ar-78 |
| C-236 | ″ | ″ | ″ | Ar-82 |
| C-237 | ″ | ″ | ″ | Ar-117 |
| C-238 | ″ | ″ | ″ | Ar-134 |
| C-239 | ″ | ″ | ″ | Ar-139 |
| C-240 | ″ | ″ | ″ | Ar-150 |
| C-241 | ″ | ″ | ″ | Ar-172 |
| C-242 | ″ | ″ | ″ | Ar-207 |
| C-243 | ″ | ″ | Ar-82 | Ar-82 |
| C-244 | ″ | ″ | ″ | Ar-117 |
| C-245 | ″ | ″ | ″ | Ar-134 |
| C-246 | ″ | ″ | ″ | Ar-139 |
| C-247 | ″ | ″ | ″ | Ar-150 |
| C-248 | ″ | ″ | ″ | Ar-172 |
| C-249 | ″ | ″ | ″ | Ar-207 |
| C-250 | ″ | ″ | Ar-117 | Ar-117 |
| C-251 | ″ | ″ | ″ | Ar-134 |
| C-252 | ″ | ″ | ″ | Ar-139 |
| C-253 | ″ | ″ | ″ | Ar-150 |
| C-254 | ″ | ″ | ″ | Ar-172 |
| C-255 | ″ | ″ | ″ | Ar-207 |
| C-256 | ″ | ″ | Ar-134 | Ar-134 |
| C-257 | ″ | ″ | ″ | Ar-139 |
| C-258 | ″ | ″ | ″ | Ar-150 |
| C-259 | ″ | ″ | ″ | Ar-172 |
| C-260 | ″ | ″ | ″ | Ar-207 |
| C-261 | ″ | ″ | Ar-139 | Ar-139 |
| C-262 | ″ | ″ | ″ | Ar-150 |
| C-263 | ″ | ″ | ″ | Ar-172 |
| C-264 | ″ | ″ | ″ | Ar-207 |
| C-265 | ″ | ″ | Ar-150 | Ar-150 |
| C-266 | ″ | ″ | ″ | Ar-172 |
| C-267 | ″ | ″ | ″ | Ar-207 |
| C-268 | ″ | ″ | Ar-172 | Ar-172 |
| C-269 | ″ | ″ | ″ | Ar-207 |
| C-270 | ″ | ″ | Ar-207 | Ar-207 |
| C-271 | ″ | R-32 | Ar-1 | Ar-2 |
| C-272 | ″ | ″ | ″ | Ar-4 |
| C-273 | ″ | ″ | ″ | Ar-5 |
| C-274 | ″ | ″ | ″ | Ar-74 |
| C-275 | ″ | ″ | ″ | Ar-78 |
| C-276 | ″ | ″ | ″ | Ar-82 |
| C-277 | ″ | ″ | ″ | Ar-117 |
| C-278 | ″ | ″ | ″ | Ar-134 |
| C-279 | ″ | ″ | ″ | Ar-139 |
| C-280 | ″ | ″ | ″ | Ar-150 |
| C-281 | ″ | ″ | ″ | Ar-172 |
| C-282 | ″ | ″ | ″ | Ar-207 |
| C-283 | ″ | ″ | Ar-2 | Ar-2 |
| C-284 | ″ | ″ | ″ | Ar-4 |
| C-285 | ″ | ″ | ″ | Ar-5 |
| C-286 | ″ | ″ | ″ | Ar-74 |
| C-287 | ″ | ″ | ″ | Ar-78 |
| C-288 | ″ | ″ | ″ | Ar-82 |
| C-289 | ″ | ″ | ″ | Ar-117 |
| C-290 | ″ | ″ | ″ | Ar-134 |
| C-291 | ″ | ″ | ″ | Ar-139 |
| C-292 | ″ | ″ | ″ | Ar-150 |
| C-293 | ″ | ″ | ″ | Ar-172 |
| C-294 | ″ | ″ | ″ | Ar-207 |
| C-295 | ″ | ″ | Ar-4 | Ar-4 |
| C-296 | ″ | ″ | ″ | Ar-5 |
| C-297 | ″ | ″ | ″ | Ar-74 |
| C-298 | ″ | ″ | ″ | Ar-78 |
| C-299 | ″ | ″ | ″ | Ar-82 |
| C-300 | ″ | ″ | ″ | Ar-117 |
| C-301 | ″ | ″ | ″ | Ar-134 |
| C-302 | ″ | ″ | ″ | Ar-139 |
| C-303 | ″ | ″ | ″ | Ar-150 |
| C-304 | ″ | ″ | ″ | Ar-172 |
| C-305 | ″ | ″ | ″ | Ar-207 |
| C-306 | ″ | ″ | Ar-5 | Ar-5 |
| C-307 | ″ | ″ | ″ | Ar-74 |
| C-308 | ″ | ″ | ″ | Ar-78 |
| C-309 | ″ | ″ | ″ | Ar-82 |
| C-310 | ″ | ″ | ″ | Ar-117 |
| C-311 | ″ | ″ | ″ | Ar-134 |
| C-312 | ″ | ″ | ″ | Ar-139 |
| C-313 | ″ | ″ | ″ | Ar-150 |
| C-314 | ″ | ″ | ″ | Ar-172 |
| C-315 | ″ | ″ | ″ | Ar-207 |
| C-316 | ″ | ″ | Ar-74 | Ar-74 |
| C-317 | ″ | ″ | ″ | Ar-78 |
| C-318 | ″ | ″ | ″ | Ar-82 |
| C-319 | ″ | ″ | ″ | Ar-117 |
| C-320 | ″ | ″ | ″ | Ar-134 |
| C-321 | ″ | ″ | ″ | Ar-139 |
| C-322 | ″ | ″ | ″ | Ar-150 |
| C-323 | ″ | ″ | ″ | Ar-172 |
| C-324 | ″ | ″ | ″ | Ar-207 |
| C-325 | ″ | ″ | Ar-78 | Ar-78 |
| C-326 | ″ | ″ | ″ | Ar-82 |
| C-327 | ″ | ″ | ″ | Ar-117 |
| C-328 | ″ | ″ | ″ | Ar-134 |
| C-329 | ″ | ″ | ″ | Ar-139 |
| C-330 | ″ | ″ | ″ | Ar-150 |
| C-331 | ″ | ″ | ″ | Ar-172 |
| C-332 | ″ | ″ | ″ | Ar-207 |
| C-333 | ″ | ″ | Ar-82 | Ar-82 |
| C-334 | ″ | ″ | ″ | Ar-117 |
| C-335 | ″ | ″ | ″ | Ar-134 |
| C-336 | ″ | ″ | ″ | Ar-139 |
| C-337 | ″ | ″ | ″ | Ar-150 |
| C-338 | ″ | ″ | ″ | Ar-172 |
| C-339 | ″ | ″ | ″ | Ar-207 |
| C-340 | ″ | ″ | Ar-117 | Ar-117 |
| C-341 | ″ | ″ | ″ | Ar-134 |
| C-342 | ″ | ″ | ″ | Ar-139 |
| C-343 | ″ | ″ | ″ | Ar-150 |
| C-344 | ″ | ″ | ″ | Ar-172 |
| C-345 | ″ | ″ | ″ | Ar-207 |
| C-346 | ″ | ″ | Ar-134 | Ar-134 |
| C-347 | ″ | ″ | ″ | Ar-139 |
| C-348 | ″ | ″ | ″ | Ar-150 |
| C-349 | ″ | ″ | ″ | Ar-172 |
| C-350 | ″ | ″ | ″ | Ar-207 |
| C-351 | ″ | ″ | Ar-139 | Ar-139 |
| C-352 | ″ | ″ | ″ | Ar-150 |
| C-353 | ″ | ″ | ″ | Ar-172 |
| C-354 | ″ | ″ | ″ | Ar-207 |
| C-355 | ″ | ″ | Ar-150 | Ar-150 |
| C-356 | ″ | ″ | ″ | Ar-172 |
| C-357 | ″ | ″ | ″ | Ar-207 |
| C-358 | ″ | ″ | Ar-172 | Ar-172 |
| C-359 | ″ | ″ | ″ | Ar-207 |
| C-360 | ″ | ″ | Ar-207 | Ar-207 |
| C-361 | ″ | R-58 | Ar-1 | Ar-2 |
| C-362 | ″ | ″ | ″ | Ar-4 |
| C-363 | ″ | ″ | ″ | Ar-5 |
| C-364 | ″ | ″ | ″ | Ar-74 |
| C-365 | ″ | ″ | ″ | Ar-78 |
| C-366 | ″ | ″ | ″ | Ar-82 |
| C-367 | ″ | ″ | ″ | Ar-117 |
| C-368 | ″ | ″ | ″ | Ar-134 |
| C-369 | ″ | ″ | ″ | Ar-139 |
| C-370 | ″ | ″ | ″ | Ar-150 |
| C-371 | ″ | ″ | ″ | Ar-172 |
| C-372 | ″ | ″ | ″ | Ar-207 |
| C-373 | ″ | ″ | Ar-2 | Ar-2 |
| C-374 | ″ | ″ | ″ | Ar-4 |
| C-375 | ″ | ″ | ″ | Ar-5 |
| C-376 | ″ | ″ | ″ | Ar-74 |
| C-377 | ″ | ″ | ″ | Ar-78 |
| C-378 | ″ | ″ | ″ | Ar-82 |
| C-379 | ″ | ″ | ″ | Ar-117 |
| C-380 | ″ | ″ | ″ | Ar-134 |
| C-381 | ″ | ″ | ″ | Ar-139 |
| C-382 | ″ | ″ | ″ | Ar-150 |
| C-383 | ″ | ″ | ″ | Ar-172 |
| C-384 | ″ | ″ | ″ | Ar-207 |
| C-385 | ″ | ″ | Ar-4 | Ar-4 |
| C-386 | ″ | ″ | ″ | Ar-5 |
| C-387 | ″ | ″ | ″ | Ar-74 |
| C-388 | ″ | ″ | ″ | Ar-78 |
| C-389 | ″ | ″ | ″ | Ar-82 |
| C-390 | ″ | ″ | ″ | Ar-117 |
| C-391 | ″ | ″ | ″ | Ar-134 |
| C-392 | ″ | ″ | ″ | Ar-139 |
| C-393 | ″ | ″ | ″ | Ar-150 |
| C-394 | ″ | ″ | ″ | Ar-172 |
| C-395 | ″ | ″ | ″ | Ar-207 |
| C-396 | ″ | ″ | Ar-5 | Ar-5 |
| C-397 | ″ | ″ | ″ | Ar-74 |
| C-398 | ″ | ″ | ″ | Ar-78 |
| C-399 | ″ | ″ | ″ | Ar-82 |
| C-400 | ″ | ″ | ″ | Ar-117 |
| C-401 | ″ | ″ | ″ | Ar-134 |
| C-402 | ″ | ″ | ″ | Ar-139 |
| C-403 | ″ | ″ | ″ | Ar-150 |
| C-404 | ″ | ″ | ″ | Ar-172 |
| C-405 | ″ | ″ | ″ | Ar-207 |
| C-406 | ″ | ″ | Ar-74 | Ar-74 |
| C-407 | ″ | ″ | ″ | Ar-78 |
| C-408 | ″ | ″ | ″ | Ar-82 |
| C-409 | ″ | ″ | ″ | Ar-117 |
| C-410 | ″ | ″ | ″ | Ar-134 |
| C-411 | ″ | ″ | ″ | Ar-139 |
| C-412 | ″ | ″ | ″ | Ar-150 |
| C-413 | ″ | ″ | ″ | Ar-172 |
| C-414 | ″ | ″ | ″ | Ar-207 |
| C-415 | ″ | ″ | Ar-78 | Ar-78 |
| C-416 | ″ | ″ | ″ | Ar-82 |
| C-417 | ″ | ″ | ″ | Ar-117 |
| C-418 | ″ | ″ | ″ | Ar-134 |
| C-419 | ″ | ″ | ″ | Ar-139 |
| C-420 | ″ | ″ | ″ | Ar-150 |
| C-421 | ″ | ″ | ″ | Ar-172 |
| C-422 | ″ | ″ | ″ | Ar-207 |
| C-423 | ″ | ″ | Ar-82 | Ar-82 |
| C-424 | ″ | ″ | ″ | Ar-117 |
| C-425 | ″ | ″ | ″ | Ar-134 |
| C-426 | ″ | ″ | ″ | Ar-139 |
| C-427 | ″ | ″ | ″ | Ar-150 |
| C-428 | ″ | ″ | ″ | Ar-172 |
| C-429 | ″ | ″ | ″ | Ar-207 |
| C-430 | ″ | ″ | Ar-117 | Ar-117 |
| C-431 | ″ | ″ | ″ | Ar-134 |
| C-432 | ″ | ″ | ″ | Ar-139 |
| C-433 | ″ | ″ | ″ | Ar-150 |
| C-434 | ″ | ″ | ″ | Ar-172 |
| C-435 | ″ | ″ | ″ | Ar-207 |
| C-436 | ″ | ″ | Ar-134 | Ar-134 |
| C-437 | ″ | ″ | ″ | Ar-139 |
| C-438 | ″ | ″ | ″ | Ar-150 |
| C-439 | ″ | ″ | ″ | Ar-172 |
| C-440 | ″ | ″ | ″ | Ar-207 |
| C-441 | ″ | ″ | Ar-139 | Ar-139 |
| C-442 | ″ | ″ | ″ | Ar-150 |
| C-443 | ″ | ″ | ″ | Ar-172 |
| C-444 | ″ | ″ | ″ | Ar-207 |
| C-445 | ″ | ″ | Ar-150 | Ar-150 |
| C-446 | ″ | ″ | ″ | Ar-172 |
| C-447 | ″ | ″ | ″ | Ar-207 |
| C-448 | ″ | ″ | Ar-172 | Ar-172 |
| C-449 | ″ | ″ | ″ | Ar-207 |
| C-450 | ″ | ″ | Ar-207 | Ar-207 |
| C-451 | ″ | R-66 | Ar-1 | Ar-2 |
| C-452 | ″ | ″ | ″ | Ar-4 |
| C-453 | ″ | ″ | ″ | Ar-5 |
| C-454 | ″ | ″ | ″ | Ar-74 |
| C-455 | ″ | ″ | ″ | Ar-78 |
| C-456 | ″ | ″ | ″ | Ar-82 |
| C-457 | ″ | ″ | ″ | Ar-117 |
| C-458 | ″ | ″ | ″ | Ar-134 |
| C-459 | ″ | ″ | ″ | Ar-139 |
| C-460 | ″ | ″ | ″ | Ar-150 |
| C-461 | ″ | ″ | ″ | Ar-172 |
| C-462 | ″ | ″ | ″ | Ar-207 |
| C-463 | ″ | ″ | Ar-2 | Ar-2 |
| C-464 | ″ | ″ | ″ | Ar-4 |
| C-465 | ″ | ″ | ″ | Ar-5 |
| C-466 | ″ | ″ | ″ | Ar-74 |
| C-467 | ″ | ″ | ″ | Ar-78 |
| C-468 | ″ | ″ | ″ | Ar-82 |
| C-469 | ″ | ″ | ″ | Ar-117 |
| C-470 | ″ | ″ | ″ | Ar-134 |
| C-471 | ″ | ″ | ″ | Ar-139 |
| C-472 | ″ | ″ | ″ | Ar-150 |
| C-473 | ″ | ″ | ″ | Ar-172 |
| C-474 | ″ | ″ | ″ | Ar-207 |
| C-475 | ″ | ″ | Ar-4 | Ar-4 |
| C-476 | ″ | ″ | ″ | Ar-5 |
| C-477 | ″ | ″ | ″ | Ar-74 |
| C-478 | ″ | ″ | ″ | Ar-78 |
| C-479 | ″ | ″ | ″ | Ar-82 |
| C-480 | ″ | ″ | ″ | Ar-117 |
| C-481 | ″ | ″ | ″ | Ar-134 |
| C-482 | ″ | ″ | ″ | Ar-139 |
| C-483 | ″ | ″ | ″ | Ar-150 |
| C-484 | ″ | ″ | ″ | Ar-172 |
| C-485 | ″ | ″ | ″ | Ar-207 |
| C-486 | ″ | ″ | Ar-5 | Ar-5 |
| C-487 | ″ | ″ | ″ | Ar-74 |
| C-488 | ″ | ″ | ″ | Ar-78 |
| C-489 | ″ | ″ | ″ | Ar-82 |
| C-490 | ″ | ″ | ″ | Ar-117 |
| C-491 | ″ | ″ | ″ | Ar-134 |
| C-492 | ″ | ″ | ″ | Ar-139 |
| C-493 | ″ | ″ | ″ | Ar-150 |
| C-494 | ″ | ″ | ″ | Ar-172 |
| C-495 | ″ | ″ | ″ | Ar-207 |
| C-496 | ″ | ″ | Ar-74 | Ar-74 |
| C-497 | ″ | ″ | ″ | Ar-78 |
| C-498 | ″ | ″ | ″ | Ar-82 |
| C-499 | ″ | ″ | ″ | Ar-117 |
| C-500 | ″ | ″ | ″ | Ar-134 |
| C-501 | ″ | ″ | ″ | Ar-139 |
| C-502 | ″ | ″ | ″ | Ar-150 |
| C-503 | ″ | ″ | ″ | Ar-172 |
| C-504 | ″ | ″ | ″ | Ar-207 |
| C-505 | ″ | ″ | Ar-78 | Ar-78 |
| C-506 | ″ | ″ | ″ | Ar-82 |
| C-507 | ″ | ″ | ″ | Ar-117 |
| C-508 | ″ | ″ | ″ | Ar-134 |
| C-509 | ″ | ″ | ″ | Ar-139 |
| C-510 | ″ | ″ | ″ | Ar-150 |
| C-511 | ″ | ″ | ″ | Ar-172 |
| C-512 | ″ | ″ | ″ | Ar-207 |
| C-513 | ″ | ″ | Ar-82 | Ar-82 |
| C-514 | ″ | ″ | ″ | Ar-117 |
| C-515 | ″ | ″ | ″ | Ar-134 |
| C-516 | ″ | ″ | ″ | Ar-139 |
| C-517 | ″ | ″ | ″ | Ar-150 |
| C-518 | ″ | ″ | ″ | Ar-172 |
| C-519 | ″ | ″ | ″ | Ar-207 |
| C-520 | ″ | ″ | Ar-117 | Ar-117 |
| C-521 | ″ | ″ | ″ | Ar-134 |
| C-522 | ″ | ″ | ″ | Ar-139 |
| C-523 | ″ | ″ | ″ | Ar-150 |
| C-524 | ″ | ″ | ″ | Ar-172 |
| C-525 | ″ | ″ | ″ | Ar-207 |
| C-526 | ″ | ″ | Ar-134 | Ar-134 |
| C-527 | ″ | ″ | ″ | Ar-139 |
| C-528 | ″ | ″ | ″ | Ar-150 |
| C-529 | ″ | ″ | ″ | Ar-172 |
| C-530 | ″ | ″ | ″ | Ar-207 |
| C-531 | ″ | ″ | Ar-139 | Ar-139 |
| C-532 | ″ | ″ | ″ | Ar-150 |
| C-533 | ″ | ″ | ″ | Ar-172 |
| C-534 | ″ | ″ | ″ | Ar-207 |
| C-535 | ″ | ″ | Ar-150 | Ar-150 |
| C-536 | ″ | ″ | ″ | Ar-172 |
| C-537 | ″ | ″ | ″ | Ar-207 |
| C-538 | ″ | ″ | Ar-172 | Ar-172 |
| C-539 | ″ | ″ | ″ | Ar-207 |
| C-540 | ″ | ″ | Ar-207 | Ar-207 |
| C-541 | ″ | Ter-phenyl** | Ar-1 | Ar-2 |
| C-542 | ″ | ″ | ″ | Ar-4 |
| C-543 | ″ | ″ | ″ | Ar-5 |
| C-544 | ″ | ″ | ″ | Ar-74 |
| C-545 | ″ | ″ | ″ | Ar-78 |
| C-546 | ″ | ″ | ″ | Ar-82 |
| C-547 | ″ | ″ | ″ | Ar-117 |
| C-548 | ″ | ″ | ″ | Ar-134 |
| C-549 | ″ | ″ | ″ | Ar-139 |
| C-550 | ″ | ″ | ″ | Ar-150 |
| C-551 | ″ | ″ | ″ | Ar-172 |
| C-552 | ″ | ″ | ″ | Ar-207 |
| C-553 | ″ | ″ | Ar-2 | Ar-2 |
| C-554 | ″ | ″ | ″ | Ar-4 |
| C-555 | ″ | ″ | ″ | Ar-5 |
| C-556 | ″ | ″ | ″ | Ar-74 |
| C-557 | ″ | ″ | ″ | Ar-78 |
| C-558 | ″ | ″ | ″ | Ar-82 |
| C-559 | ″ | ″ | ″ | Ar-117 |
| C-560 | ″ | ″ | ″ | Ar-134 |
| C-561 | ″ | ″ | ″ | Ar-139 |
| C-562 | ″ | ″ | ″ | Ar-150 |
| C-563 | ″ | ″ | ″ | Ar-172 |
| C-564 | ″ | ″ | ″ | Ar-207 |
| C-565 | ″ | ″ | Ar-4 | Ar-4 |
| C-566 | ″ | ″ | ″ | Ar-5 |
| C-567 | ″ | ″ | ″ | Ar-74 |
| C-568 | ″ | ″ | ″ | Ar-78 |
| C-569 | ″ | ″ | ″ | Ar-82 |
| C-570 | ″ | ″ | ″ | Ar-117 |
| C-571 | ″ | ″ | ″ | Ar-134 |
| C-572 | ″ | ″ | ″ | Ar-139 |
| C-573 | ″ | ″ | ″ | Ar-150 |
| C-574 | ″ | ″ | ″ | Ar-172 |
| C-575 | ″ | ″ | ″ | Ar-207 |
| C-576 | ″ | ″ | Ar-5 | Ar-5 |
| C-577 | ″ | ″ | ″ | Ar-74 |
| C-578 | ″ | ″ | ″ | Ar-78 |
| C-579 | ″ | ″ | ″ | Ar-82 |
| C-580 | ″ | ″ | ″ | Ar-117 |
| C-581 | ″ | ″ | ″ | Ar-134 |
| C-582 | ″ | ″ | ″ | Ar-139 |
| C-583 | ″ | ″ | ″ | Ar-150 |
| C-584 | ″ | ″ | ″ | Ar-172 |
| C-585 | ″ | ″ | ″ | Ar-207 |
| C-586 | ″ | ″ | Ar-74 | Ar-74 |
| C-587 | ″ | ″ | ″ | Ar-78 |
| C-588 | ″ | ″ | ″ | Ar-82 |
| C-589 | ″ | ″ | ″ | Ar-117 |
| C-590 | ″ | ″ | ″ | Ar-134 |
| C-591 | ″ | ″ | ″ | Ar-139 |
| C-592 | ″ | ″ | ″ | Ar-150 |
| C-593 | ″ | ″ | ″ | Ar-172 |
| C-594 | ″ | ″ | ″ | Ar-207 |
| C-595 | ″ | ″ | Ar-78 | Ar-78 |
| C-596 | ″ | ″ | ″ | Ar-82 |
| C-597 | ″ | ″ | ″ | Ar-117 |
| C-598 | ″ | ″ | ″ | Ar-134 |
| C-599 | ″ | ″ | ″ | Ar-139 |
| C-600 | ″ | ″ | ″ | Ar-150 |
| C-601 | ″ | ″ | ″ | Ar-172 |
| C-602 | ″ | ″ | ″ | Ar-207 |
| C-603 | ″ | ″ | Ar-82 | Ar-82 |
| C-604 | ″ | ″ | ″ | Ar-117 |
| C-605 | ″ | ″ | ″ | Ar-134 |
| C-606 | ″ | ″ | ″ | Ar-139 |
| C-607 | ″ | ″ | ″ | Ar-150 |
| C-608 | ″ | ″ | ″ | Ar-172 |
| C-609 | ″ | ″ | ″ | Ar-207 |
| C-610 | ″ | ″ | Ar-117 | Ar-117 |
| C-611 | ″ | ″ | ″ | Ar-134 |
| C-612 | ″ | ″ | ″ | Ar-139 |
| C-613 | ″ | ″ | ″ | Ar-150 |
| C-614 | ″ | ″ | ″ | Ar-172 |
| C-615 | ″ | ″ | ″ | Ar-207 |
| C-616 | ″ | ″ | Ar-134 | Ar-134 |
| C-617 | ″ | ″ | ″ | Ar-139 |
| C-618 | ″ | ″ | ″ | Ar-150 |
| C-619 | ″ | ″ | ″ | Ar-172 |
| C-620 | ″ | ″ | ″ | Ar-207 |
| C-621 | ″ | ″ | Ar-139 | Ar-139 |
| C-622 | ″ | ″ | ″ | Ar-150 |
| C-623 | ″ | ″ | ″ | Ar-172 |
| C-624 | ″ | ″ | ″ | Ar-207 |
| C-625 | ″ | ″ | Ar-150 | Ar-150 |
| C-626 | ″ | ″ | ″ | Ar-172 |
| C-627 | ″ | ″ | ″ | Ar-207 |
| C-628 | ″ | ″ | Ar-172 | Ar-172 |
| C-629 | ″ | ″ | ″ | Ar-207 |
| C-630 | ″ | ″ | Ar-207 | Ar-207 |
| C-631 | phenyl | R-1 | Ar-1 | Ar-2 |
| C-632 | ″ | ″ | ″ | Ar-4 |
| C-633 | ″ | ″ | ″ | Ar-5 |
| C-634 | ″ | ″ | ″ | Ar-74 |
| C-635 | ″ | ″ | ″ | Ar-78 |
| C-636 | ″ | ″ | ″ | Ar-82 |
| C-637 | ″ | ″ | ″ | Ar-117 |
| C-638 | ″ | ″ | ″ | Ar-134 |
| C-639 | ″ | ″ | ″ | Ar-139 |
| C-640 | ″ | ″ | ″ | Ar-150 |
| C-641 | ″ | ″ | ″ | Ar-172 |
| C-642 | ″ | ″ | ″ | Ar-207 |
| C-643 | ″ | ″ | Ar-2 | Ar-2 |
| C-644 | ″ | ″ | ″ | Ar-4 |
| C-645 | ″ | ″ | ″ | Ar-5 |
| C-646 | ″ | ″ | ″ | Ar-74 |
| C-647 | ″ | ″ | ″ | Ar-78 |
| C-648 | ″ | ″ | ″ | Ar-82 |
| C-649 | ″ | ″ | ″ | Ar-117 |
| C-650 | ″ | ″ | ″ | Ar-134 |
| C-651 | ″ | ″ | ″ | Ar-139 |
| C-652 | ″ | ″ | ″ | Ar-150 |
| C-653 | ″ | ″ | ″ | Ar-172 |
| C-654 | ″ | ″ | ″ | Ar-207 |
| C-655 | ″ | ″ | Ar-4 | Ar-4 |
| C-656 | ″ | ″ | ″ | Ar-5 |
| C-657 | ″ | ″ | ″ | Ar-74 |
| C-658 | ″ | ″ | ″ | Ar-78 |
| C-659 | ″ | ″ | ″ | Ar-82 |
| C-660 | ″ | ″ | ″ | Ar-117 |
| C-661 | ″ | ″ | ″ | Ar-134 |
| C-662 | ″ | ″ | ″ | Ar-139 |
| C-663 | ″ | ″ | ″ | Ar-150 |
| C-664 | ″ | ″ | ″ | Ar-172 |
| C-665 | ″ | ″ | ″ | Ar-207 |
| C-666 | ″ | ″ | Ar-5 | Ar-5 |
| C-667 | ″ | ″ | ″ | Ar-74 |
| C-668 | ″ | ″ | ″ | Ar-78 |
| C-669 | ″ | ″ | ″ | Ar-82 |
| C-670 | ″ | ″ | ″ | Ar-117 |
| C-671 | ″ | ″ | ″ | Ar-134 |
| C-672 | ″ | ″ | ″ | Ar-139 |
| C-673 | ″ | ″ | ″ | Ar-150 |
| C-674 | ″ | ″ | ″ | Ar-172 |
| C-675 | ″ | ″ | ″ | Ar-207 |
| C-676 | ″ | ″ | Ar-74 | Ar-74 |
| C-677 | ″ | ″ | ″ | Ar-78 |
| C-678 | ″ | ″ | ″ | Ar-82 |
| C-679 | ″ | ″ | ″ | Ar-117 |
| C-680 | ″ | ″ | ″ | Ar-134 |
| C-681 | ″ | ″ | ″ | Ar-139 |
| C-682 | ″ | ″ | ″ | Ar-150 |
| C-683 | ″ | ″ | ″ | Ar-172 |
| C-684 | ″ | ″ | ″ | Ar-207 |
| C-685 | ″ | ″ | Ar-78 | Ar-78 |
| C-686 | ″ | ″ | ″ | Ar-82 |
| C-687 | ″ | ″ | ″ | Ar-117 |
| C-688 | ″ | ″ | ″ | Ar-134 |
| C-689 | ″ | ″ | ″ | Ar-139 |
| C-690 | ″ | ″ | ″ | Ar-150 |
| C-691 | ″ | ″ | ″ | Ar-172 |
| C-692 | ″ | ″ | ″ | Ar-207 |
| C-693 | ″ | ″ | Ar-82 | Ar-82 |
| C-694 | ″ | ″ | ″ | Ar-117 |
| C-695 | ″ | ″ | ″ | Ar-134 |
| C-696 | ″ | ″ | ″ | Ar-139 |
| C-697 | ″ | ″ | ″ | Ar-150 |
| C-698 | ″ | ″ | ″ | Ar-172 |
| C-699 | ″ | ″ | ″ | Ar-207 |
| C-700 | ″ | ″ | Ar-117 | Ar-117 |
| C-701 | ″ | ″ | ″ | Ar-134 |
| C-702 | ″ | ″ | ″ | Ar-139 |
| C-703 | ″ | ″ | ″ | Ar-150 |
| C-704 | ″ | ″ | ″ | Ar-172 |
| C-705 | ″ | ″ | ″ | Ar-207 |
| C-706 | ″ | ″ | Ar-134 | Ar-134 |
| C-707 | ″ | ″ | ″ | Ar-139 |
| C-708 | ″ | ″ | ″ | Ar-150 |
| C-709 | ″ | ″ | ″ | Ar-172 |
| C-710 | ″ | ″ | ″ | Ar-207 |
| C-711 | ″ | ″ | Ar-139 | Ar-139 |
| C-712 | ″ | ″ | ″ | Ar-150 |
| C-713 | ″ | ″ | ″ | Ar-172 |
| C-714 | ″ | ″ | ″ | Ar-207 |
| C-715 | ″ | ″ | Ar-150 | Ar-150 |
| C-716 | ″ | ″ | ″ | Ar-172 |
| C-717 | ″ | ″ | ″ | Ar-207 |
| C-718 | ″ | ″ | Ar-172 | Ar-172 |
| C-719 | ″ | ″ | ″ | Ar-207 |
| C-720 | ″ | ″ | Ar-207 | Ar-207 |
| C-721 | ″ | Biphenyl* | Ar-1 | Ar-2 |
| C-722 | ″ | ″ | ″ | Ar-4 |
| C-723 | ″ | ″ | ″ | Ar-5 |
| C-724 | ″ | ″ | ″ | Ar-74 |
| C-725 | ″ | ″ | ″ | Ar-78 |
| C-726 | ″ | ″ | ″ | Ar-82 |
| C-727 | ″ | ″ | ″ | Ar-117 |
| C-728 | ″ | ″ | ″ | Ar-134 |
| C-729 | ″ | ″ | ″ | Ar-139 |
| C-730 | ″ | ″ | ″ | Ar-150 |
| C-731 | ″ | ″ | ″ | Ar-172 |
| C-732 | ″ | ″ | ″ | Ar-207 |
| C-733 | ″ | ″ | Ar-2 | Ar-2 |
| C-734 | ″ | ″ | ″ | Ar-4 |
| C-735 | ″ | ″ | ″ | Ar-5 |
| C-736 | ″ | ″ | ″ | Ar-74 |
| C-737 | ″ | ″ | ″ | Ar-78 |
| C-738 | ″ | ″ | ″ | Ar-82 |
| C-739 | ″ | ″ | ″ | Ar-117 |
| C-740 | ″ | ″ | ″ | Ar-134 |
| C-741 | ″ | ″ | ″ | Ar-139 |
| C-742 | ″ | ″ | ″ | Ar-150 |
| C-743 | ″ | ″ | ″ | Ar-172 |
| C-744 | ″ | ″ | ″ | Ar-207 |
| C-745 | ″ | ″ | Ar-4 | Ar-4 |
| C-746 | ″ | ″ | ″ | Ar-5 |
| C-747 | ″ | ″ | ″ | Ar-74 |
| C-748 | ″ | ″ | ″ | Ar-78 |
| C-749 | ″ | ″ | ″ | Ar-82 |
| C-750 | ″ | ″ | ″ | Ar-117 |
| C-751 | ″ | ″ | ″ | Ar-134 |
| C-752 | ″ | ″ | ″ | Ar-139 |
| C-753 | ″ | ″ | ″ | Ar-150 |
| C-754 | ″ | ″ | ″ | Ar-172 |
| C-755 | ″ | ″ | ″ | Ar-207 |
| C-756 | ″ | ″ | Ar-5 | Ar-5 |
| C-757 | ″ | ″ | ″ | Ar-74 |
| C-758 | ″ | ″ | ″ | Ar-78 |
| C-759 | ″ | ″ | ″ | Ar-82 |
| C-760 | ″ | ″ | ″ | Ar-117 |
| C-761 | ″ | ″ | ″ | Ar-134 |
| C-762 | ″ | ″ | ″ | Ar-139 |
| C-763 | ″ | ″ | ″ | Ar-150 |
| C-764 | ″ | ″ | ″ | Ar-172 |
| C-765 | ″ | ″ | ″ | Ar-207 |
| C-766 | ″ | ″ | Ar-74 | Ar-74 |
| C-767 | ″ | ″ | ″ | Ar-78 |
| C-768 | ″ | ″ | ″ | Ar-82 |
| C-769 | ″ | ″ | ″ | Ar-117 |
| C-770 | ″ | ″ | ″ | Ar-134 |
| C-771 | ″ | ″ | ″ | Ar-139 |
| C-772 | ″ | ″ | ″ | Ar-150 |
| C-773 | ″ | ″ | ″ | Ar-172 |
| C-774 | ″ | ″ | ″ | Ar-207 |
| C-775 | ″ | ″ | Ar-78 | Ar-78 |
| C-776 | ″ | ″ | ″ | Ar-82 |
| C-777 | ″ | ″ | ″ | Ar-117 |
| C-778 | ″ | ″ | ″ | Ar-134 |
| C-779 | ″ | ″ | ″ | Ar-139 |
| C-780 | ″ | ″ | ″ | Ar-150 |
| C-781 | ″ | ″ | ″ | Ar-172 |
| C-782 | ″ | ″ | ″ | Ar-207 |
| C-783 | ″ | ″ | Ar-82 | Ar-82 |
| C-784 | ″ | ″ | ″ | Ar-117 |
| C-785 | ″ | ″ | ″ | Ar-134 |
| C-786 | ″ | ″ | ″ | Ar-139 |
| C-787 | ″ | ″ | ″ | Ar-150 |
| C-788 | ″ | ″ | ″ | Ar-172 |
| C-789 | ″ | ″ | ″ | Ar-207 |
| C-790 | ″ | ″ | Ar-117 | Ar-117 |
| C-791 | ″ | ″ | ″ | Ar-134 |
| C-792 | ″ | ″ | ″ | Ar-139 |
| C-793 | ″ | ″ | ″ | Ar-150 |
| C-794 | ″ | ″ | ″ | Ar-172 |
| C-795 | ″ | ″ | ″ | Ar-207 |
| C-796 | ″ | ″ | Ar-134 | Ar-134 |
| C-797 | ″ | ″ | ″ | Ar-139 |
| C-798 | ″ | ″ | ″ | Ar-150 |
| C-799 | ″ | ″ | ″ | Ar-172 |
| C-800 | ″ | ″ | ″ | Ar-207 |
| C-801 | ″ | ″ | Ar-139 | Ar-139 |
| C-802 | ″ | ″ | ″ | Ar-150 |
| C-803 | ″ | ″ | ″ | Ar-172 |
| C-804 | ″ | ″ | ″ | Ar-207 |
| C-805 | ″ | ″ | Ar-150 | Ar-150 |
| C-806 | ″ | ″ | ″ | Ar-172 |
| C-807 | ″ | ″ | ″ | Ar-207 |
| C-808 | ″ | ″ | Ar-172 | Ar-172 |
| C-809 | ″ | ″ | ″ | Ar-207 |
| C-810 | ″ | ″ | Ar-207 | Ar-207 |
| C-811 | ″ | R-21 | Ar-1 | Ar-2 |
| C-812 | ″ | ″ | ″ | Ar-4 |
| C-813 | ″ | ″ | ″ | Ar-5 |
| C-814 | ″ | ″ | ″ | Ar-74 |
| C-815 | ″ | ″ | ″ | Ar-78 |
| C-816 | ″ | ″ | ″ | Ar-82 |
| C-817 | ″ | ″ | ″ | Ar-117 |
| C-818 | ″ | ″ | ″ | Ar-134 |
| C-819 | ″ | ″ | ″ | Ar-139 |
| C-820 | ″ | ″ | ″ | Ar-150 |
| C-821 | ″ | ″ | ″ | Ar-172 |
| C-822 | ″ | ″ | ″ | Ar-207 |
| C-823 | ″ | ″ | Ar-2 | Ar-2 |
| C-824 | ″ | ″ | ″ | Ar-4 |
| C-825 | ″ | ″ | ″ | Ar-5 |
| C-826 | ″ | ″ | ″ | Ar-74 |
| C-827 | ″ | ″ | ″ | Ar-78 |
| C-828 | ″ | ″ | ″ | Ar-82 |
| C-829 | ″ | ″ | ″ | Ar-117 |
| C-830 | ″ | ″ | ″ | Ar-134 |
| C-831 | ″ | ″ | ″ | Ar-139 |
| C-832 | ″ | ″ | ″ | Ar-150 |
| C-833 | ″ | ″ | ″ | Ar-172 |
| C-834 | ″ | ″ | ″ | Ar-207 |
| C-835 | ″ | ″ | Ar-4 | Ar-4 |
| C-836 | ″ | ″ | ″ | Ar-5 |
| C-837 | ″ | ″ | ″ | Ar-74 |
| C-838 | ″ | ″ | ″ | Ar-78 |
| C-839 | ″ | ″ | ″ | Ar-82 |
| C-840 | ″ | ″ | ″ | Ar-117 |
| C-841 | ″ | ″ | ″ | Ar-134 |
| C-842 | ″ | ″ | ″ | Ar-139 |
| C-843 | ″ | ″ | ″ | Ar-150 |
| C-844 | ″ | ″ | ″ | Ar-172 |
| C-845 | ″ | ″ | ″ | Ar-207 |
| C-846 | ″ | ″ | Ar-5 | Ar-5 |
| C-847 | ″ | ″ | ″ | Ar-74 |
| C-848 | ″ | ″ | ″ | Ar-78 |
| C-849 | ″ | ″ | ″ | Ar-82 |
| C-850 | ″ | ″ | ″ | Ar-117 |
| C-851 | ″ | ″ | ″ | Ar-134 |
| C-852 | ″ | ″ | ″ | Ar-139 |
| C-853 | ″ | ″ | ″ | Ar-150 |
| C-854 | ″ | ″ | ″ | Ar-172 |
| C-855 | ″ | ″ | ″ | Ar-207 |
| C-856 | ″ | ″ | Ar-74 | Ar-74 |
| C-857 | ″ | ″ | ″ | Ar-78 |
| C-858 | ″ | ″ | ″ | Ar-82 |
| C-859 | ″ | ″ | ″ | Ar-117 |
| C-860 | ″ | ″ | ″ | Ar-134 |
| C-861 | ″ | ″ | ″ | Ar-139 |
| C-862 | ″ | ″ | ″ | Ar-150 |
| C-863 | ″ | ″ | ″ | Ar-172 |
| C-864 | ″ | ″ | ″ | Ar-207 |
| C-865 | ″ | ″ | Ar-78 | Ar-78 |
| C-866 | ″ | ″ | ″ | Ar-82 |
| C-867 | ″ | ″ | ″ | Ar-117 |
| C-868 | ″ | ″ | ″ | Ar-134 |
| C-869 | ″ | ″ | ″ | Ar-139 |
| C-870 | ″ | ″ | ″ | Ar-150 |
| C-871 | ″ | ″ | ″ | Ar-172 |
| C-872 | ″ | ″ | ″ | Ar-207 |
| C-873 | ″ | ″ | Ar-82 | Ar-82 |
| C-874 | ″ | ″ | ″ | Ar-117 |
| C-875 | ″ | ″ | ″ | Ar-134 |
| C-876 | ″ | ″ | ″ | Ar-139 |
| C-877 | ″ | ″ | ″ | Ar-150 |
| C-878 | ″ | ″ | ″ | Ar-172 |
| C-879 | ″ | ″ | ″ | Ar-207 |
| C-880 | ″ | ″ | Ar-117 | Ar-117 |
| C-881 | ″ | ″ | ″ | Ar-134 |
| C-882 | ″ | ″ | ″ | Ar-139 |
| C-883 | ″ | ″ | ″ | Ar-150 |
| C-884 | ″ | ″ | ″ | Ar-172 |
| C-885 | ″ | ″ | ″ | Ar-207 |
| C-886 | ″ | ″ | Ar-134 | Ar-134 |
| C-887 | ″ | ″ | ″ | Ar-139 |
| C-888 | ″ | ″ | ″ | Ar-150 |
| C-889 | ″ | ″ | ″ | Ar-172 |
| C-890 | ″ | ″ | ″ | Ar-207 |
| C-891 | ″ | ″ | Ar-139 | Ar-139 |
| C-892 | ″ | ″ | ″ | Ar-150 |
| C-893 | ″ | ″ | ″ | Ar-172 |
| C-894 | ″ | ″ | ″ | Ar-207 |
| C-895 | ″ | ″ | Ar-150 | Ar-150 |
| C-896 | ″ | ″ | ″ | Ar-172 |
| C-897 | ″ | ″ | ″ | Ar-207 |
| C-898 | ″ | ″ | Ar-172 | Ar-172 |
| C-899 | ″ | ″ | ″ | Ar-207 |
| C-900 | ″ | ″ | Ar-207 | Ar-207 |
| C-901 | ″ | R-32 | Ar-1 | Ar-2 |
| C-902 | ″ | ″ | ″ | Ar-4 |
| C-903 | ″ | ″ | ″ | Ar-5 |
| C-904 | ″ | ″ | ″ | Ar-74 |
| C-905 | ″ | ″ | ″ | Ar-78 |
| C-906 | ″ | ″ | ″ | Ar-82 |
| C-907 | ″ | ″ | ″ | Ar-117 |
| C-908 | ″ | ″ | ″ | Ar-134 |
| C-909 | ″ | ″ | ″ | Ar-139 |
| C-910 | ″ | ″ | ″ | Ar-150 |
| C-911 | ″ | ″ | ″ | Ar-172 |
| C-912 | ″ | ″ | ″ | Ar-207 |
| C-913 | ″ | ″ | Ar-2 | Ar-2 |
| C-914 | ″ | ″ | ″ | Ar-4 |
| C-915 | ″ | ″ | ″ | Ar-5 |
| C-916 | ″ | ″ | ″ | Ar-74 |
| C-917 | ″ | ″ | ″ | Ar-78 |
| C-918 | ″ | ″ | ″ | Ar-82 |
| C-919 | ″ | ″ | ″ | Ar-117 |
| C-920 | ″ | ″ | ″ | Ar-134 |
| C-921 | ″ | ″ | ″ | Ar-139 |
| C-922 | ″ | ″ | ″ | Ar-150 |
| C-923 | ″ | ″ | ″ | Ar-172 |
| C-924 | ″ | ″ | ″ | Ar-207 |
| C-925 | ″ | ″ | Ar-4 | Ar-4 |
| C-926 | ″ | ″ | ″ | Ar-5 |
| C-927 | ″ | ″ | ″ | Ar-74 |
| C-928 | ″ | ″ | ″ | Ar-78 |
| C-929 | ″ | ″ | ″ | Ar-82 |
| C-930 | ″ | ″ | ″ | Ar-117 |
| C-931 | ″ | ″ | ″ | Ar-134 |
| C-932 | ″ | ″ | ″ | Ar-139 |
| C-933 | ″ | ″ | ″ | Ar-150 |
| C-934 | ″ | ″ | ″ | Ar-172 |
| C-935 | ″ | ″ | ″ | Ar-207 |
| C-936 | ″ | ″ | Ar-5 | Ar-5 |
| C-937 | ″ | ″ | ″ | Ar-74 |
| C-938 | ″ | ″ | ″ | Ar-78 |
| C-939 | ″ | ″ | ″ | Ar-82 |
| C-940 | ″ | ″ | ″ | Ar-117 |
| C-941 | ″ | ″ | ″ | Ar-134 |
| C-942 | ″ | ″ | ″ | Ar-139 |
| C-943 | ″ | ″ | ″ | Ar-150 |
| C-944 | ″ | ″ | ″ | Ar-172 |
| C-945 | ″ | ″ | ″ | Ar-207 |
| C-946 | ″ | ″ | Ar-74 | Ar-74 |
| C-947 | ″ | ″ | ″ | Ar-78 |
| C-948 | ″ | ″ | ″ | Ar-82 |
| C-949 | ″ | ″ | ″ | Ar-117 |
| C-950 | ″ | ″ | ″ | Ar-134 |
| C-951 | ″ | ″ | ″ | Ar-139 |
| C-952 | ″ | ″ | ″ | Ar-150 |
| C-953 | ″ | ″ | ″ | Ar-172 |
| C-954 | ″ | ″ | ″ | Ar-207 |
| C-955 | ″ | ″ | Ar-78 | Ar-78 |
| C-956 | ″ | ″ | ″ | Ar-82 |
| C-957 | ″ | ″ | ″ | Ar-117 |
| C-958 | ″ | ″ | ″ | Ar-134 |
| C-959 | ″ | ″ | ″ | Ar-139 |
| C-960 | ″ | ″ | ″ | Ar-150 |
| C-961 | ″ | ″ | ″ | Ar-172 |
| C-962 | ″ | ″ | ″ | Ar-207 |
| C-963 | ″ | ″ | Ar-82 | Ar-82 |
| C-964 | ″ | ″ | ″ | Ar-117 |
| C-965 | ″ | ″ | ″ | Ar-134 |
| C-966 | ″ | ″ | ″ | Ar-139 |
| C-967 | ″ | ″ | ″ | Ar-150 |
| C-968 | ″ | ″ | ″ | Ar-172 |
| C-969 | ″ | ″ | ″ | Ar-207 |
| C-970 | ″ | ″ | Ar-117 | Ar-117 |
| C-971 | ″ | ″ | ″ | Ar-134 |
| C-972 | ″ | ″ | ″ | Ar-139 |
| C-973 | ″ | ″ | ″ | Ar-150 |
| C-974 | ″ | ″ | ″ | Ar-172 |
| C-975 | ″ | ″ | ″ | Ar-207 |
| C-976 | ″ | ″ | Ar-134 | Ar-134 |
| C-977 | ″ | ″ | ″ | Ar-139 |
| C-978 | ″ | ″ | ″ | Ar-150 |
| C-979 | ″ | ″ | ″ | Ar-172 |
| C-980 | ″ | ″ | ″ | Ar-207 |
| C-981 | ″ | ″ | Ar-139 | Ar-139 |
| C-982 | ″ | ″ | ″ | Ar-150 |
| C-983 | ″ | ″ | ″ | Ar-172 |
| C-984 | ″ | ″ | ″ | Ar-207 |
| C-985 | ″ | ″ | Ar-150 | Ar-150 |
| C-986 | ″ | ″ | ″ | Ar-172 |
| C-987 | ″ | ″ | ″ | Ar-207 |
| C-988 | ″ | ″ | Ar-172 | Ar-172 |
| C-989 | ″ | ″ | ″ | Ar-207 |
| C-990 | ″ | ″ | Ar-207 | Ar-207 |
| C-991 | ″ | R-58 | Ar-1 | Ar-2 |
| C-992 | ″ | ″ | ″ | Ar-4 |
| C-993 | ″ | ″ | ″ | Ar-5 |
| C-994 | ″ | ″ | ″ | Ar-74 |
| C-995 | ″ | ″ | ″ | Ar-78 |
| C-996 | ″ | ″ | ″ | Ar-82 |
| C-997 | ″ | ″ | ″ | Ar-117 |
| C-998 | ″ | ″ | ″ | Ar-134 |
| C-999 | ″ | ″ | ″ | Ar-139 |
| C-1000 | ″ | ″ | ″ | Ar-150 |
| C-1001 | ″ | ″ | ″ | Ar-172 |
| C-1002 | ″ | ″ | ″ | Ar-207 |
| C-1003 | ″ | ″ | Ar-2 | Ar-2 |
| C-1004 | ″ | ″ | ″ | Ar-4 |
| C-1005 | ″ | ″ | ″ | Ar-5 |
| C-1006 | ″ | ″ | ″ | Ar-74 |
| C-1007 | ″ | ″ | ″ | Ar-78 |
| C-1008 | ″ | ″ | ″ | Ar-82 |
| C-1009 | ″ | ″ | ″ | Ar-117 |
| C-1010 | ″ | ″ | ″ | Ar-134 |
| C-1011 | ″ | ″ | ″ | Ar-139 |
| C-1012 | ″ | ″ | ″ | Ar-150 |
| C-1013 | ″ | ″ | ″ | Ar-172 |
| C-1014 | ″ | ″ | ″ | Ar-207 |
| C-1015 | ″ | ″ | Ar-4 | Ar-4 |
| C-1016 | ″ | ″ | ″ | Ar-5 |
| C-1017 | ″ | ″ | ″ | Ar-74 |
| C-1018 | ″ | ″ | ″ | Ar-78 |
| C-1019 | ″ | ″ | ″ | Ar-82 |
| C-1020 | ″ | ″ | ″ | Ar-117 |
| C-1021 | ″ | ″ | ″ | Ar-134 |
| C-1022 | ″ | ″ | ″ | Ar-139 |
| C-1023 | ″ | ″ | ″ | Ar-150 |
| C-1024 | ″ | ″ | ″ | Ar-172 |
| C-1025 | ″ | ″ | ″ | Ar-207 |
| C-1026 | ″ | ″ | Ar-5 | Ar-5 |
| C-1027 | ″ | ″ | ″ | Ar-74 |
| C-1028 | ″ | ″ | ″ | Ar-78 |
| C-1029 | ″ | ″ | ″ | Ar-82 |
| C-1030 | ″ | ″ | ″ | Ar-117 |
| C-1031 | ″ | ″ | ″ | Ar-134 |
| C-1032 | ″ | ″ | ″ | Ar-139 |
| C-1033 | ″ | ″ | ″ | Ar-150 |
| C-1034 | ″ | ″ | ″ | Ar-172 |
| C-1035 | ″ | ″ | ″ | Ar-207 |
| C-1036 | ″ | ″ | Ar-74 | Ar-74 |
| C-1037 | ″ | ″ | ″ | Ar-78 |
| C-1038 | ″ | ″ | ″ | Ar-82 |
| C-1039 | ″ | ″ | ″ | Ar-117 |
| C-1040 | ″ | ″ | ″ | Ar-134 |
| C-1041 | ″ | ″ | ″ | Ar-139 |
| C-1042 | ″ | ″ | ″ | Ar-150 |
| C-1043 | ″ | ″ | ″ | Ar-172 |
| C-1044 | ″ | ″ | ″ | Ar-207 |
| C-1045 | ″ | ″ | Ar-78 | Ar-78 |
| C-1046 | ″ | ″ | ″ | Ar-82 |
| C-1047 | ″ | ″ | ″ | Ar-117 |
| C-1048 | ″ | ″ | ″ | Ar-134 |
| C-1049 | ″ | ″ | ″ | Ar-139 |
| C-1050 | ″ | ″ | ″ | Ar-150 |
| C-1051 | ″ | ″ | ″ | Ar-172 |
| C-1052 | ″ | ″ | ″ | Ar-207 |
| C-1053 | ″ | ″ | Ar-82 | Ar-82 |
| C-1054 | ″ | ″ | ″ | Ar-117 |
| C-1055 | ″ | ″ | ″ | Ar-134 |
| C-1056 | ″ | ″ | ″ | Ar-139 |
| C-1057 | ″ | ″ | ″ | Ar-150 |
| C-1058 | ″ | ″ | ″ | Ar-172 |
| C-1059 | ″ | ″ | ″ | Ar-207 |
| C-1060 | ″ | ″ | Ar-117 | Ar-117 |
| C-1061 | ″ | ″ | ″ | Ar-134 |
| C-1062 | ″ | ″ | ″ | Ar-139 |
| C-1063 | ″ | ″ | ″ | Ar-150 |
| C-1064 | ″ | ″ | ″ | Ar-172 |
| C-1065 | ″ | ″ | ″ | Ar-207 |
| C-1066 | ″ | ″ | Ar-134 | Ar-134 |
| C-1067 | ″ | ″ | ″ | Ar-139 |
| C-1068 | ″ | ″ | ″ | Ar-150 |
| C-1069 | ″ | ″ | ″ | Ar-172 |
| C-1070 | ″ | ″ | ″ | Ar-207 |
| C-1071 | ″ | ″ | Ar-139 | Ar-139 |
| C-1072 | ″ | ″ | ″ | Ar-150 |
| C-1073 | ″ | ″ | ″ | Ar-172 |
| C-1074 | ″ | ″ | ″ | Ar-207 |
| C-1075 | ″ | ″ | Ar-150 | Ar-150 |
| C-1076 | ″ | ″ | ″ | Ar-172 |
| C-1077 | ″ | ″ | ″ | Ar-207 |
| C-1078 | ″ | ″ | Ar-172 | Ar-172 |
| C-1079 | ″ | ″ | ″ | Ar-207 |
| C-1080 | ″ | ″ | ″ | Ar-207 |
| C-1081 | ″ | R-66 | Ar-1 | Ar-2 |
| C-1082 | ″ | ″ | ″ | Ar-4 |
| C-1083 | ″ | ″ | ″ | Ar-5 |
| C-1084 | ″ | ″ | ″ | Ar-74 |
| C-1085 | ″ | ″ | ″ | Ar-78 |
| C-1086 | ″ | ″ | ″ | Ar-82 |
| C-1087 | ″ | ″ | ″ | Ar-117 |
| C-1088 | ″ | ″ | ″ | Ar-134 |
| C-1089 | ″ | ″ | ″ | Ar-139 |
| C-1090 | ″ | ″ | ″ | Ar-150 |
| C-1091 | ″ | ″ | ″ | Ar-172 |
| C-1092 | ″ | ″ | ″ | Ar-207 |
| C-1093 | ″ | ″ | Ar-2 | Ar-2 |
| C-1094 | ″ | ″ | ″ | Ar-4 |
| C-1095 | ″ | ″ | ″ | Ar-5 |
| C-1096 | ″ | ″ | ″ | Ar-74 |
| C-1097 | ″ | ″ | ″ | Ar-78 |
| C-1098 | ″ | ″ | ″ | Ar-82 |
| C-1099 | ″ | ″ | ″ | Ar-117 |
| C-1100 | ″ | ″ | ″ | Ar-134 |
| C-1101 | ″ | ″ | ″ | Ar-139 |
| C-1102 | ″ | ″ | ″ | Ar-150 |
| C-1103 | ″ | ″ | ″ | Ar-172 |
| C-1104 | ″ | ″ | ″ | Ar-207 |
| C-1105 | ″ | ″ | Ar-4 | Ar-4 |
| C-1106 | ″ | ″ | ″ | Ar-5 |
| C-1107 | ″ | ″ | ″ | Ar-74 |
| C-1108 | ″ | ″ | ″ | Ar-78 |
| C-1109 | ″ | ″ | ″ | Ar-82 |
| C-1110 | ″ | ″ | ″ | Ar-117 |
| C-1111 | ″ | ″ | ″ | Ar-134 |
| C-1112 | ″ | ″ | ″ | Ar-139 |
| C-1113 | ″ | ″ | ″ | Ar-150 |
| C-1114 | ″ | ″ | ″ | Ar-172 |
| C-1115 | ″ | ″ | ″ | Ar-207 |
| C-1116 | ″ | ″ | Ar-5 | Ar-5 |
| C-1117 | ″ | ″ | ″ | Ar-74 |
| C-1118 | ″ | ″ | ″ | Ar-78 |
| C-1119 | ″ | ″ | ″ | Ar-82 |
| C-1120 | ″ | ″ | ″ | Ar-117 |
| C-1121 | ″ | ″ | ″ | Ar-134 |
| C-1122 | ″ | ″ | ″ | Ar-139 |
| C-1123 | ″ | ″ | ″ | Ar-150 |
| C-1124 | ″ | ″ | ″ | Ar-172 |
| C-1125 | ″ | ″ | ″ | Ar-207 |
| C-1126 | ″ | ″ | Ar-74 | Ar-74 |
| C-1127 | ″ | ″ | ″ | Ar-78 |
| C-1128 | ″ | ″ | ″ | Ar-82 |
| C-1129 | ″ | ″ | ″ | Ar-117 |
| C-1130 | ″ | ″ | ″ | Ar-134 |
| C-1131 | ″ | ″ | ″ | Ar-139 |
| C-1132 | ″ | ″ | ″ | Ar-150 |
| C-1133 | ″ | ″ | ″ | Ar-172 |
| C-1134 | ″ | ″ | ″ | Ar-207 |
| C-1135 | ″ | ″ | Ar-78 | Ar-78 |
| C-1136 | ″ | ″ | ″ | Ar-82 |
| C-1137 | ″ | ″ | ″ | Ar-117 |
| C-1138 | ″ | ″ | ″ | Ar-134 |
| C-1139 | ″ | ″ | ″ | Ar-139 |
| C-1140 | ″ | ″ | ″ | Ar-150 |
| C-1141 | ″ | ″ | ″ | Ar-172 |
| C-1142 | ″ | ″ | ″ | Ar-207 |
| C-1143 | ″ | ″ | Ar-82 | Ar-82 |
| C-1144 | ″ | ″ | ″ | Ar-117 |
| C-1145 | ″ | ″ | ″ | Ar-134 |
| C-1146 | ″ | ″ | ″ | Ar-139 |
| C-1147 | ″ | ″ | ″ | Ar-150 |
| C-1148 | ″ | ″ | ″ | Ar-172 |
| C-1149 | ″ | ″ | ″ | Ar-207 |
| C-1150 | ″ | ″ | Ar-117 | Ar-117 |
| C-1151 | ″ | ″ | ″ | Ar-134 |
| C-1152 | ″ | ″ | ″ | Ar-139 |
| C-1153 | ″ | ″ | ″ | Ar-150 |
| C-1154 | ″ | ″ | ″ | Ar-172 |
| C-1155 | ″ | ″ | ″ | Ar-207 |
| C-1156 | ″ | ″ | Ar-134 | Ar-134 |
| C-1157 | ″ | ″ | ″ | Ar-139 |
| C-1158 | ″ | ″ | ″ | Ar-150 |
| C-1159 | ″ | ″ | ″ | Ar-172 |
| C-1160 | ″ | ″ | ″ | Ar-207 |
| C-1161 | ″ | ″ | Ar-139 | Ar-139 |
| C-1162 | ″ | ″ | ″ | Ar-150 |
| C-1163 | ″ | ″ | ″ | Ar-172 |
| C-1164 | ″ | ″ | ″ | Ar-207 |
| C-1165 | ″ | ″ | Ar-150 | Ar-150 |
| C-1166 | ″ | ″ | ″ | Ar-172 |
| C-1167 | ″ | ″ | ″ | Ar-207 |
| C-1168 | ″ | ″ | Ar-172 | Ar-172 |
| C-1169 | ″ | ″ | ″ | Ar-207 |
| C-1170 | ″ | ″ | Ar-207 | Ar-207 |
| C-1171 | ″ | Ter-phenyl** | Ar-1 | Ar-2 |
| C-1172 | ″ | ″ | ″ | Ar-4 |
| C-1173 | ″ | ″ | ″ | Ar-5 |
| C-1174 | ″ | ″ | ″ | Ar-74 |
| C-1175 | ″ | ″ | ″ | Ar-78 |
| C-1176 | ″ | ″ | ″ | Ar-82 |
| C-1177 | ″ | ″ | ″ | Ar-117 |
| C-1178 | ″ | ″ | ″ | Ar-134 |
| C-1179 | ″ | ″ | ″ | Ar-139 |
| C-1180 | ″ | ″ | ″ | Ar-150 |
| C-1181 | ″ | ″ | ″ | Ar-172 |
| C-1182 | ″ | ″ | ″ | Ar-207 |
| C-1183 | ″ | ″ | Ar-2 | Ar-2 |
| C-1184 | ″ | ″ | ″ | Ar-4 |
| C-1185 | ″ | ″ | ″ | Ar-5 |
| C-1186 | ″ | ″ | ″ | Ar-74 |
| C-1187 | ″ | ″ | ″ | Ar-78 |
| C-1188 | ″ | ″ | ″ | Ar-82 |
| C-1189 | ″ | ″ | ″ | Ar-117 |
| C-1190 | ″ | ″ | ″ | Ar-134 |
| C-1191 | ″ | ″ | ″ | Ar-139 |
| C-1192 | ″ | ″ | ″ | Ar-150 |
| C-1193 | ″ | ″ | ″ | Ar-172 |
| C-1194 | ″ | ″ | ″ | Ar-207 |
| C-1195 | ″ | ″ | Ar-4 | Ar-4 |
| C-1196 | ″ | ″ | ″ | Ar-5 |
| C-1197 | ″ | ″ | ″ | Ar-74 |
| C-1198 | ″ | ″ | ″ | Ar-78 |
| C-1199 | ″ | ″ | ″ | Ar-82 |
| C-1200 | ″ | ″ | ″ | Ar-117 |
| C-1201 | ″ | ″ | ″ | Ar-134 |
| C-1202 | ″ | ″ | ″ | Ar-139 |
| C-1203 | ″ | ″ | ″ | Ar-150 |
| C-1204 | ″ | ″ | ″ | Ar-172 |
| C-1205 | ″ | ″ | ″ | Ar-207 |
| C-1206 | ″ | ″ | Ar-5 | Ar-5 |
| C-1207 | ″ | ″ | ″ | Ar-74 |
| C-1208 | ″ | ″ | ″ | Ar-78 |
| C-1209 | ″ | ″ | ″ | Ar-82 |
| C-1210 | ″ | ″ | ″ | Ar-117 |
| C-1211 | ″ | ″ | ″ | Ar-134 |
| C-1212 | ″ | ″ | ″ | Ar-139 |
| C-1213 | ″ | ″ | ″ | Ar-150 |
| C-1214 | ″ | ″ | ″ | Ar-172 |
| C-1215 | ″ | ″ | ″ | Ar-207 |
| C-1216 | ″ | ″ | Ar-74 | Ar-74 |
| C-1217 | ″ | ″ | ″ | Ar-78 |
| C-1218 | ″ | ″ | ″ | Ar-82 |
| C-1219 | ″ | ″ | ″ | Ar-117 |
| C-1220 | ″ | ″ | ″ | Ar-134 |
| C-1221 | ″ | ″ | ″ | Ar-139 |
| C-1222 | ″ | ″ | ″ | Ar-150 |
| C-1223 | ″ | ″ | ″ | Ar-172 |
| C-1224 | ″ | ″ | ″ | Ar-207 |
| C-1225 | ″ | ″ | Ar-78 | Ar-78 |
| C-1226 | ″ | ″ | ″ | Ar-82 |
| C-1227 | ″ | ″ | ″ | Ar-117 |
| C-1228 | ″ | ″ | ″ | Ar-134 |
| C-1229 | ″ | ″ | ″ | Ar-139 |
| C-1230 | ″ | ″ | ″ | Ar-150 |
| C-1231 | ″ | ″ | ″ | Ar-172 |
| C-1232 | ″ | ″ | ″ | Ar-207 |
| C-1233 | ″ | ″ | Ar-82 | Ar-82 |
| C-1234 | ″ | ″ | ″ | Ar-117 |
| C-1235 | ″ | ″ | ″ | Ar-134 |
| C-1236 | ″ | ″ | ″ | Ar-139 |
| C-1237 | ″ | ″ | ″ | Ar-150 |
| C-1238 | ″ | ″ | ″ | Ar-172 |
| C-1239 | ″ | ″ | ″ | Ar-207 |
| C-1240 | ″ | ″ | Ar-117 | Ar-117 |
| C-1241 | ″ | ″ | ″ | Ar-134 |
| C-1242 | ″ | ″ | ″ | Ar-139 |
| C-1243 | ″ | ″ | ″ | Ar-150 |
| C-1244 | ″ | ″ | ″ | Ar-172 |
| C-1245 | ″ | ″ | ″ | Ar-207 |
| C-1246 | ″ | ″ | Ar-134 | Ar-134 |
| C-1247 | ″ | ″ | ″ | Ar-139 |
| C-1248 | ″ | ″ | ″ | Ar-150 |
| C-1249 | ″ | ″ | ″ | Ar-172 |
| C-1250 | ″ | ″ | ″ | Ar-207 |
| C-1251 | ″ | ″ | Ar-139 | Ar-139 |
| C-1252 | ″ | ″ | ″ | Ar-150 |
| C-1253 | ″ | ″ | ″ | Ar-172 |
| C-1254 | ″ | ″ | ″ | Ar-207 |
| C-1255 | ″ | ″ | Ar-150 | Ar-150 |
| C-1256 | ″ | ″ | ″ | Ar-172 |
| C-1257 | ″ | ″ | ″ | Ar-207 |
| C-1258 | ″ | ″ | Ar-172 | Ar-172 |
| C-1259 | ″ | ″ | ″ | Ar-207 |
| C-1260 | ″ | ″ | Ar-207 | Ar-207 |

(*biphenyl group conforming to one of formulae R2, R2a and R2b as defined above;
**terphenyl group conforming to one of formulae R-3 to R-8a as defined above)

Further preferred specific compounds are the compounds C-1 to C-1260 of the preceding table wherein R³ is -CD₃. The technical effect that can be observed with such compounds is described above.

Further preferred are compounds that correspond to the compounds C-1 to C-1260 listed above, with the exception that they are derived from formula (I-A-2-1) shown above, in which Ar^{L} is phenylene, preferably 1,4-phenylene, and in which R³, R¹, Ar¹ and Ar² are specified as shown for the corresponding compounds C-1 to C-1260.

Further preferred are compounds that correspond to the compounds C-1 to C-1260 listed above, with the exception that they are derived from formula (I-E-2-2) shown above, in which R³, R¹, Ar¹ and Ar² are specified as shown for the corresponding compounds C-1 to C-1260 and in which both groups R¹ are identical.

In a further preferred embodiment of the instant invention, the compound of formula (I) comprises two fluorene groups. Even more preferred is a compound of formula (I) that contains precisely two fluorene groups, i.e. the compound contains no further fluorene group. Preference is given to a compound of formula (I) wherein Z¹ and Z² are defined as being CR² (forming the first fluorene group) and wherein m=0 and only one of either Ar¹ or Ar² comprises a or is a fluorene group (the second fluorene group), very preferably only one of either Ar¹ or Ar² is selected from the groups Ar-139 to Ar-200, Ar-202, Ar-203, Ar-226, Ar-227, Ar-250 to Ar-252 and Ar-264 to Ar-266, which may be substituted at any free positions with groups R⁴.

It Is further preferred if the compound of formula (I) comprising the two fluorene groups show identical substitution in position 9 of the two fluorene groups. Particularly preferred substituents for the overall four groups in position 9 of the two fluorene groups are selected from —CH₃, -CD₃, phenyl (—C₆H₅) and —C₆D₅.

In a further preferred embodiment of the instant invention, the compound of formula (I) comprises two fluorene groups and one dibenzofurane group. Even more preferred is a compound of formula (I) that contains precisely two fluorene groups and one dibenzofurane group, i.e. the compound contains no further fluorene or dibenzofuranre group. Preference is given to a compound of formula (I) wherein Z¹ and Z² are defined as being CR² (forming the first fluorene group) and wherein m=0 and wherein Ar¹ comprises a or is a fluorene group (the second fluorene group) and wherein Ar² comprises a or is a fluorene group, very preferably Ar¹ is selected from the groups Ar-139 to Ar-200, Ar-202, Ar-203, Ar-226, Ar-227, Ar-250 to Ar-252 and Ar-264 to Ar-266 and Ar² is selected from Ar-63 to Ar-66, Ar-71 to Ar-85, Ar-99, Ar-100, Ar-102, Ar-103, Ar-204, Ar-205, Ar-206, very preferably Ar² is selected from Ar-63 to Ar-66, Ar-71 to Ar-85, Ar-99, Ar-100, Ar-102, Ar-103, particularly preferably Ar² is selected from Ar-71 to Ar-83 and Ar-85, very particularly preferably Ar² is Ar-78 and wherein both Ar¹ and Ar² may be substituted at any free positions with groups R⁴.

It Is further preferred if the compound of formula (I) comprising the two fluorene groups and the one dibenzofurane group show identical substitution in position 9 of the two fluorene groups. Particularly preferred substituents for the overall four groups in position 9 of the two fluorene groups are selected from —CH₃, -CD₃, phenyl (—C₆H₅) and —C₆D₅.

In yet another preferred embodiment of the instant invention, the compound of formula (I) comprises one fluorene group and two dibenzofurane groups. Even more preferred is a compound of formula (I) that contains only one fluorene group and two dibenzofurane group, i.e. the compound contains no further fluorene or dibenzofuranre group. Preference is given to a compound of formula (I) wherein Z¹ and Z² are defined as being CR² (forming the first fluorene group) and wherein m=0 and wherein Ar¹ comprises a or is a dibenzofurane group (the first dibenzofurane group) and wherein Ar² comprises a or is a dibenzofurane group (the second dibenzofurane group), very preferably Ar¹ and Ar² are, the same or different from each other, selected from the groups Ar-63 to Ar-66, Ar-71 to Ar-85, Ar-99, Ar-100, Ar-102, Ar-103, Ar-204, Ar-205, Ar-206, very preferably from Ar-63 to Ar-66, Ar-71 to Ar-85, Ar-99, Ar-100, Ar-102, Ar-103, particularly preferably from Ar-71 to Ar-83 and Ar-85 and very particularly preferably Ar¹ and Ar² are both Ar-78 and wherein both Ar¹ and Ar² may be substituted at any free positions with groups R⁴.

It Is further preferred if the compound of formula (I) comprises the one fluorene group and two dibenzofurane groups, wherein particularly preferred substituents for the two groups in position 9 of the fluorene group are selected from —CH₃, -CD₃, phenyl (—C₆H₅) and —C₆D₅.

In yet another preferred embodiment of the instant invention, the compound of formula (I) comprises one fluorene group and one dibenzofurane group. Even more preferred is a compound of formula (I) that contains only one fluorene group and one dibenzofurane group, i.e. the compound contains no further fluorene or dibenzofuranre group. Preference is given to a compound of formula (I) wherein Z¹ and Z² are defined as being CR² (forming the first fluorene group) and wherein m=0 and wherein only one of either Ar¹ or Ar² comprises a or is a dibenzofurane group, very preferably only one of either Ar¹ or Ar² are selected from the groups Ar-63 to Ar-66, Ar-71 to Ar-85, Ar-99, Ar-100, Ar-102, Ar-103, Ar-204, Ar-205, Ar-206, very preferably from Ar-63 to Ar-66, Ar-71 to Ar-85, Ar-99, Ar-100, Ar-102, Ar-103, particularly preferably from Ar-71 to Ar-83 and Ar-85 and very particularly preferably only one of either Ar¹ or Ar² is Ar-78 and wherein both Ar¹ and Ar² may be substituted at any free positions with groups R⁴.

It Is further preferred if the compound of formula (I) comprises the one fluorene group and the one dibenzofurane group, wherein particularly preferred substituents for the two groups in position 9 of the fluorene group are selected from —CH₃, -CD₃, phenyl (—C₆H₅) and —C₆D₅.

Preferred compounds according to formula (I) are shown in the following table:

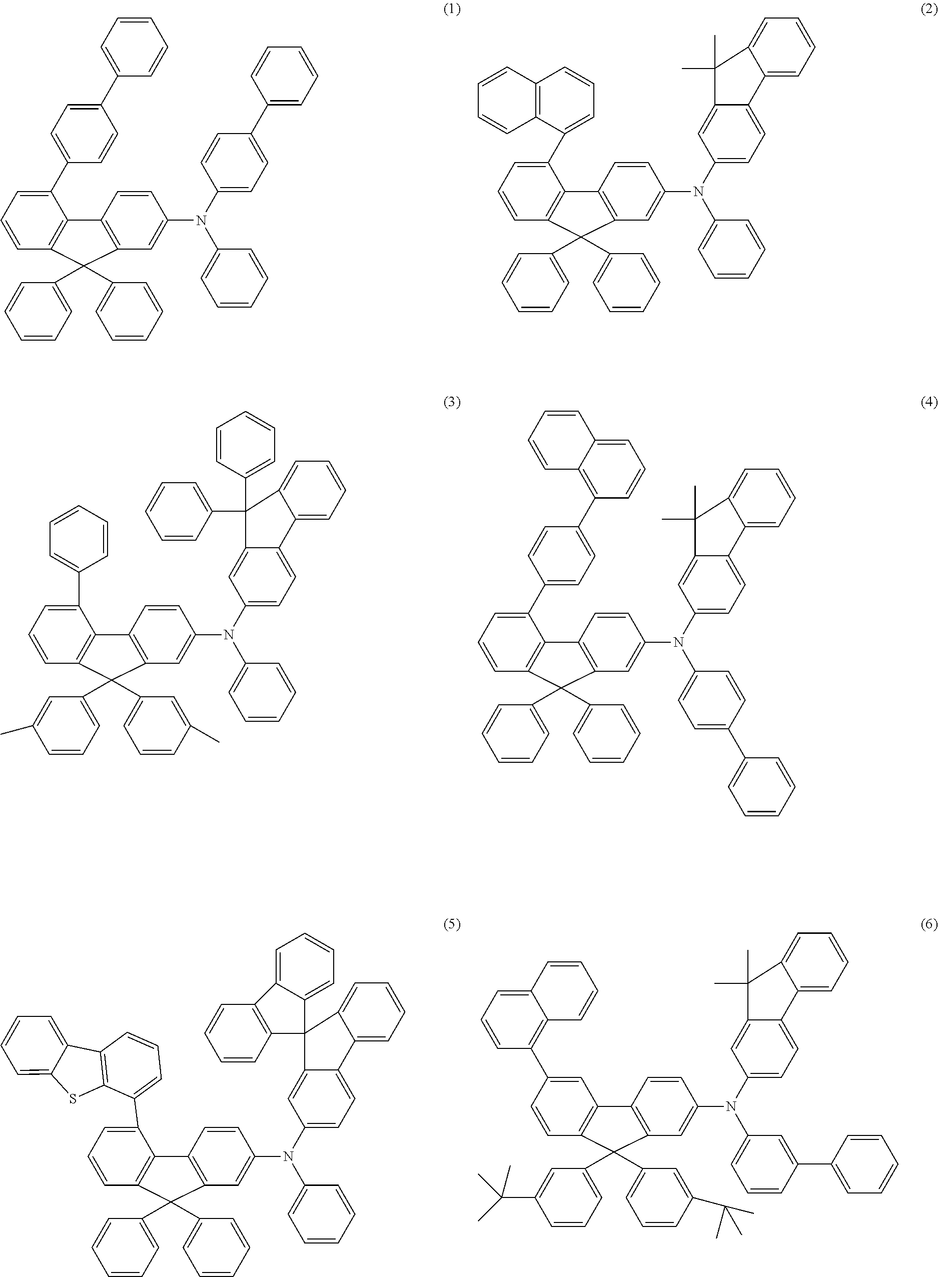

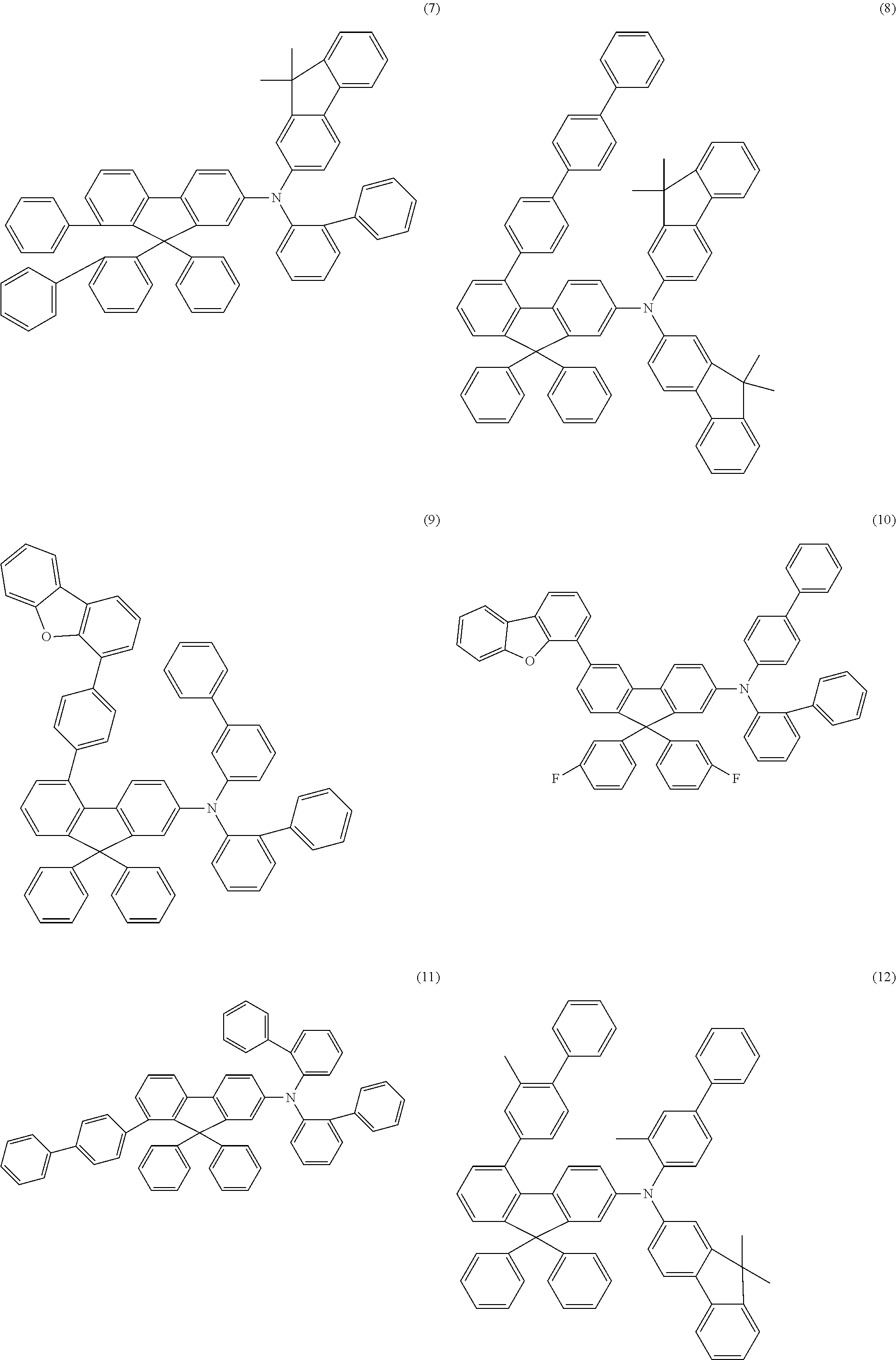

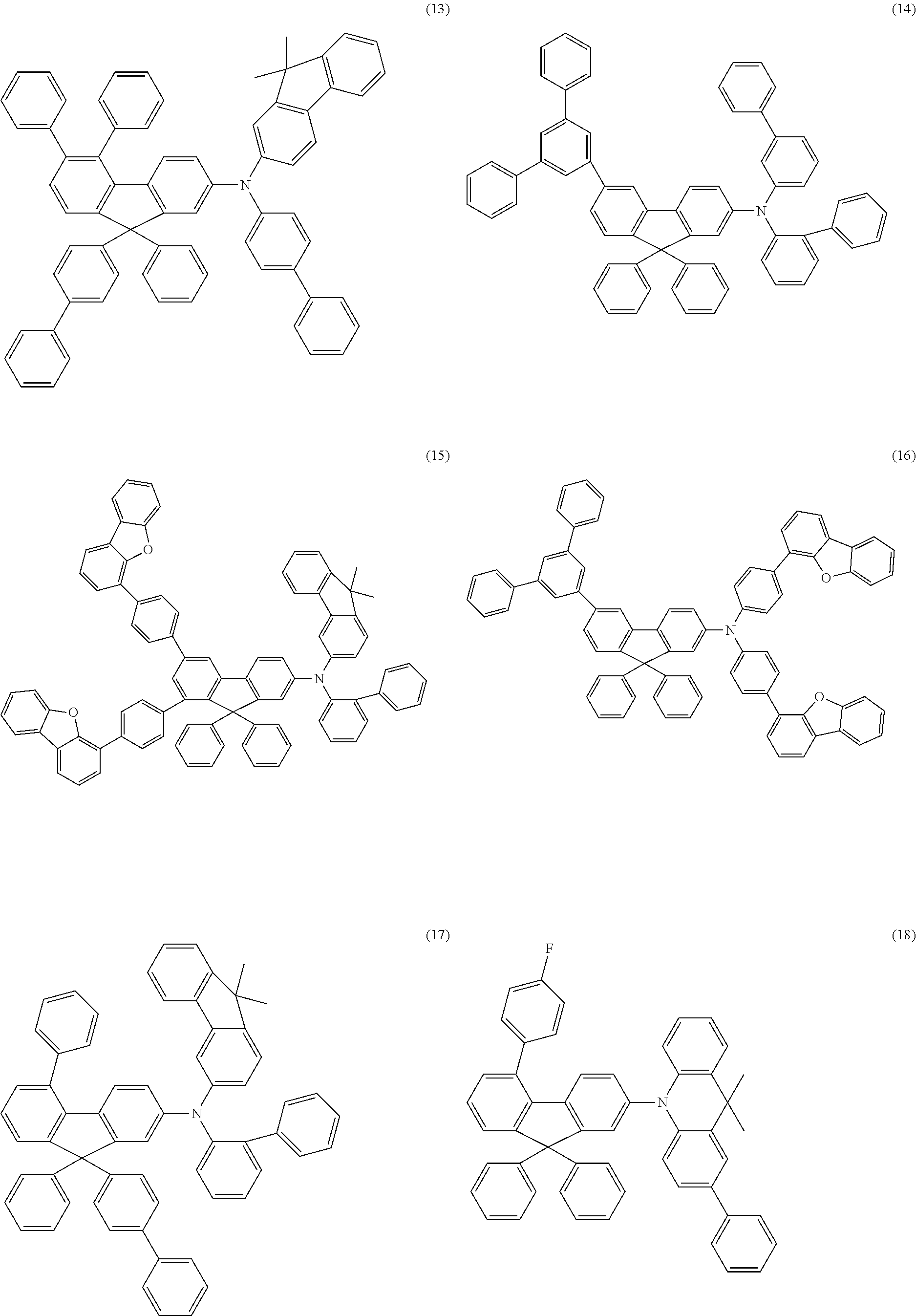

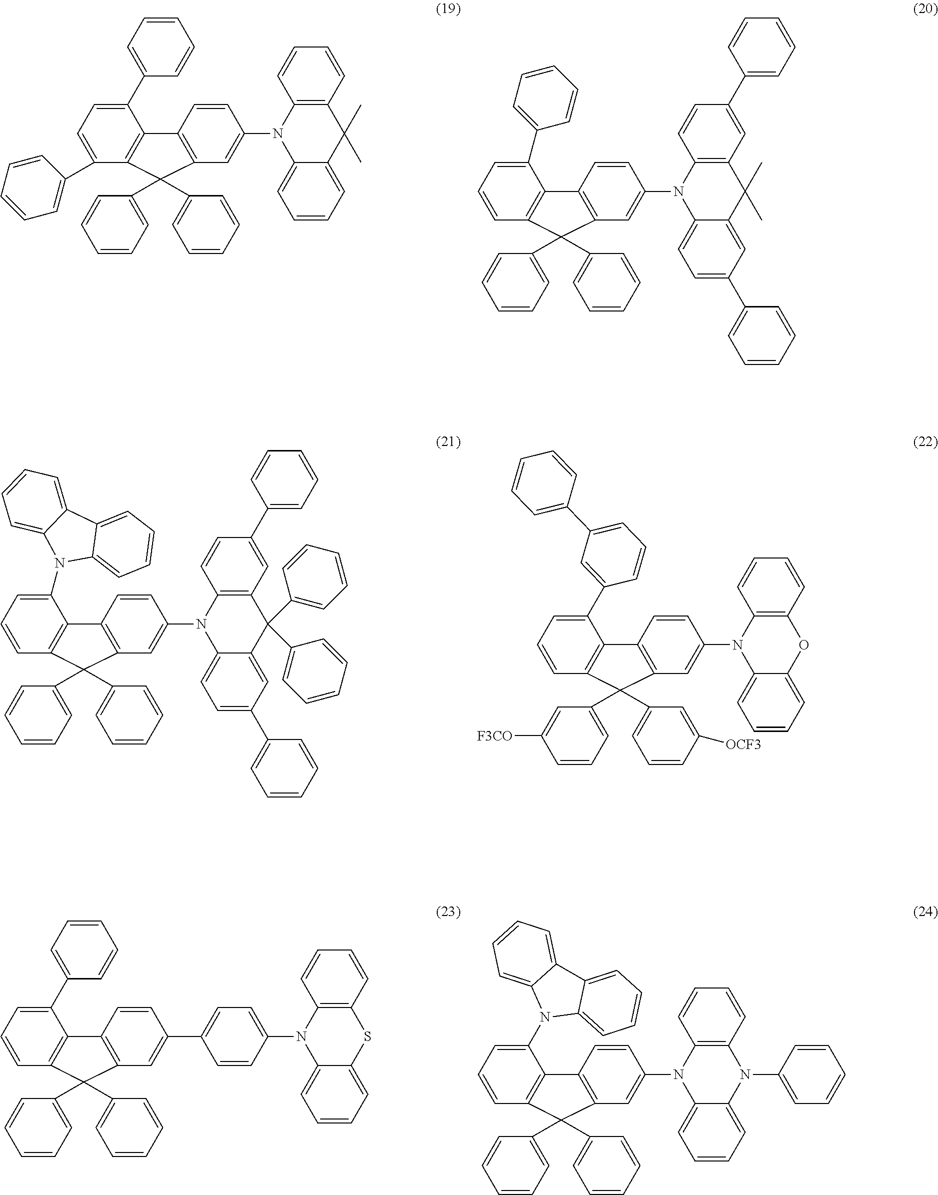

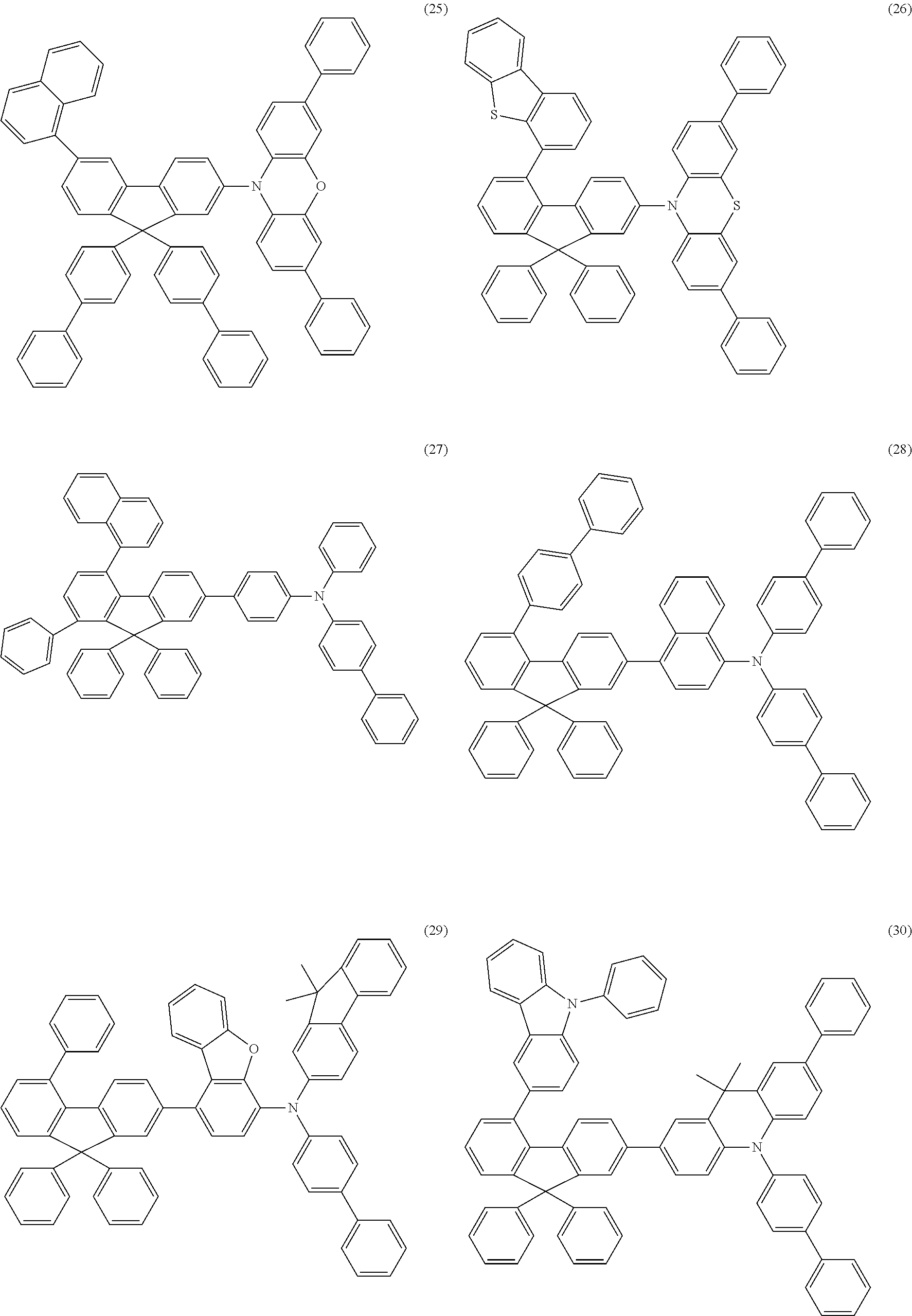

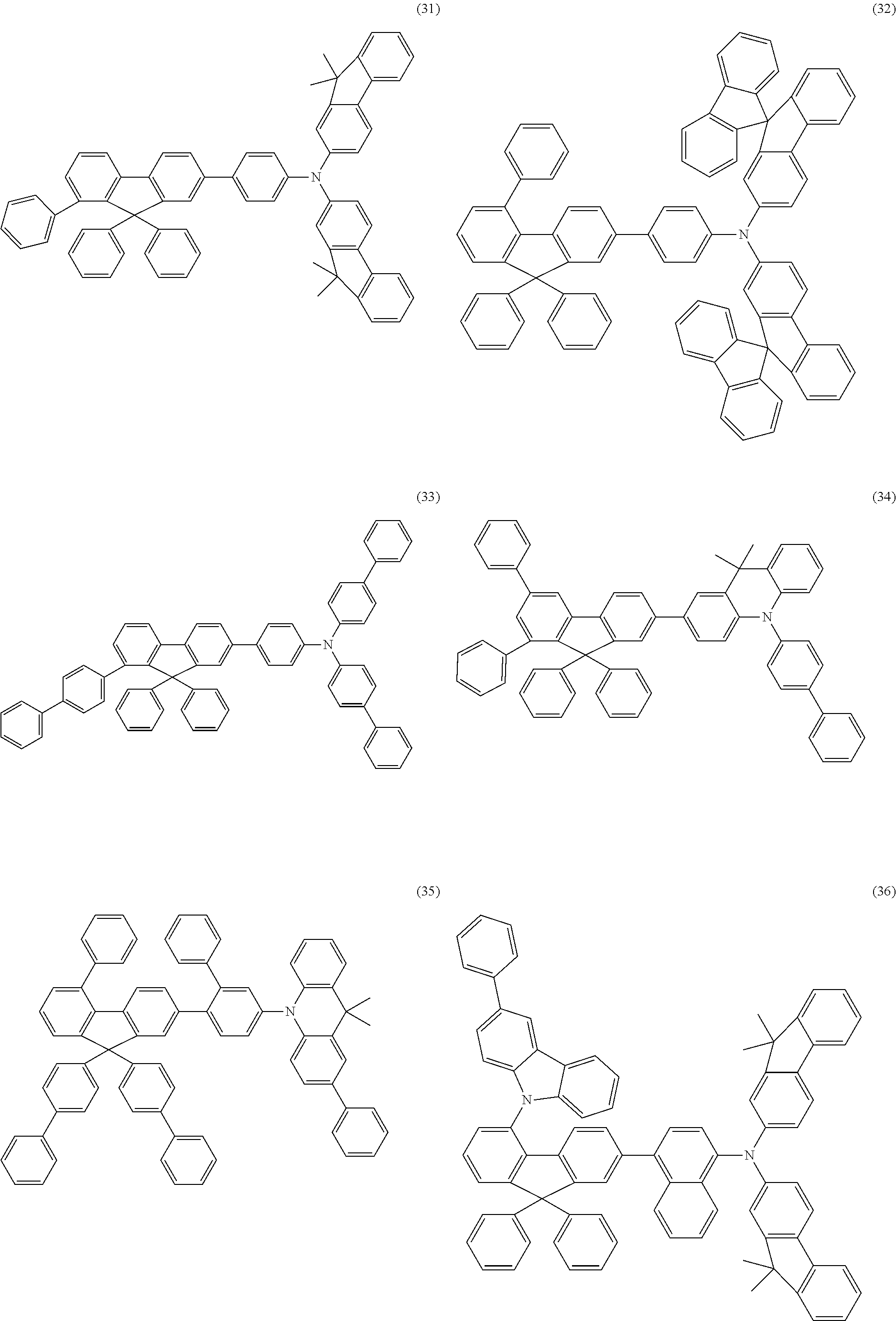

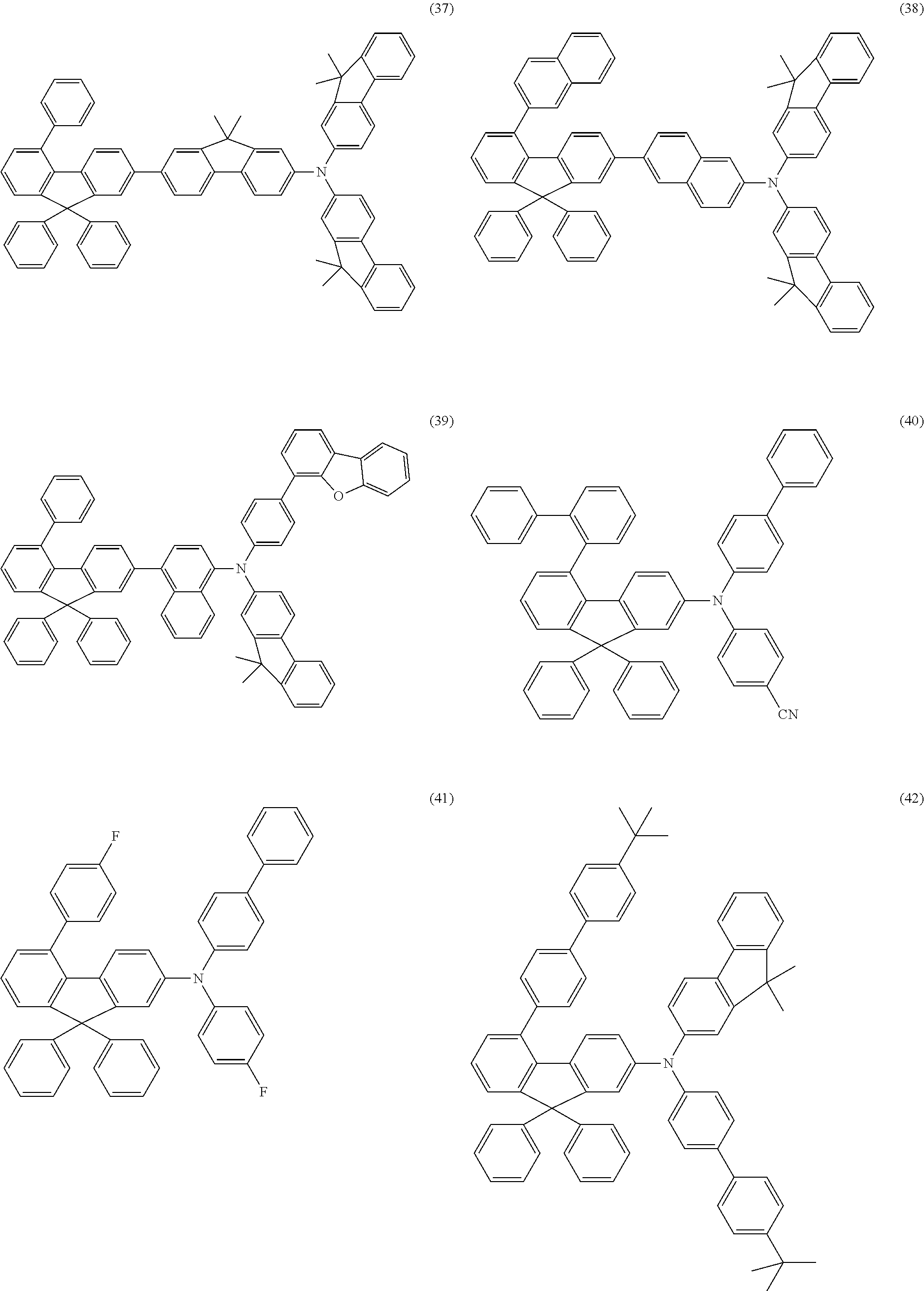

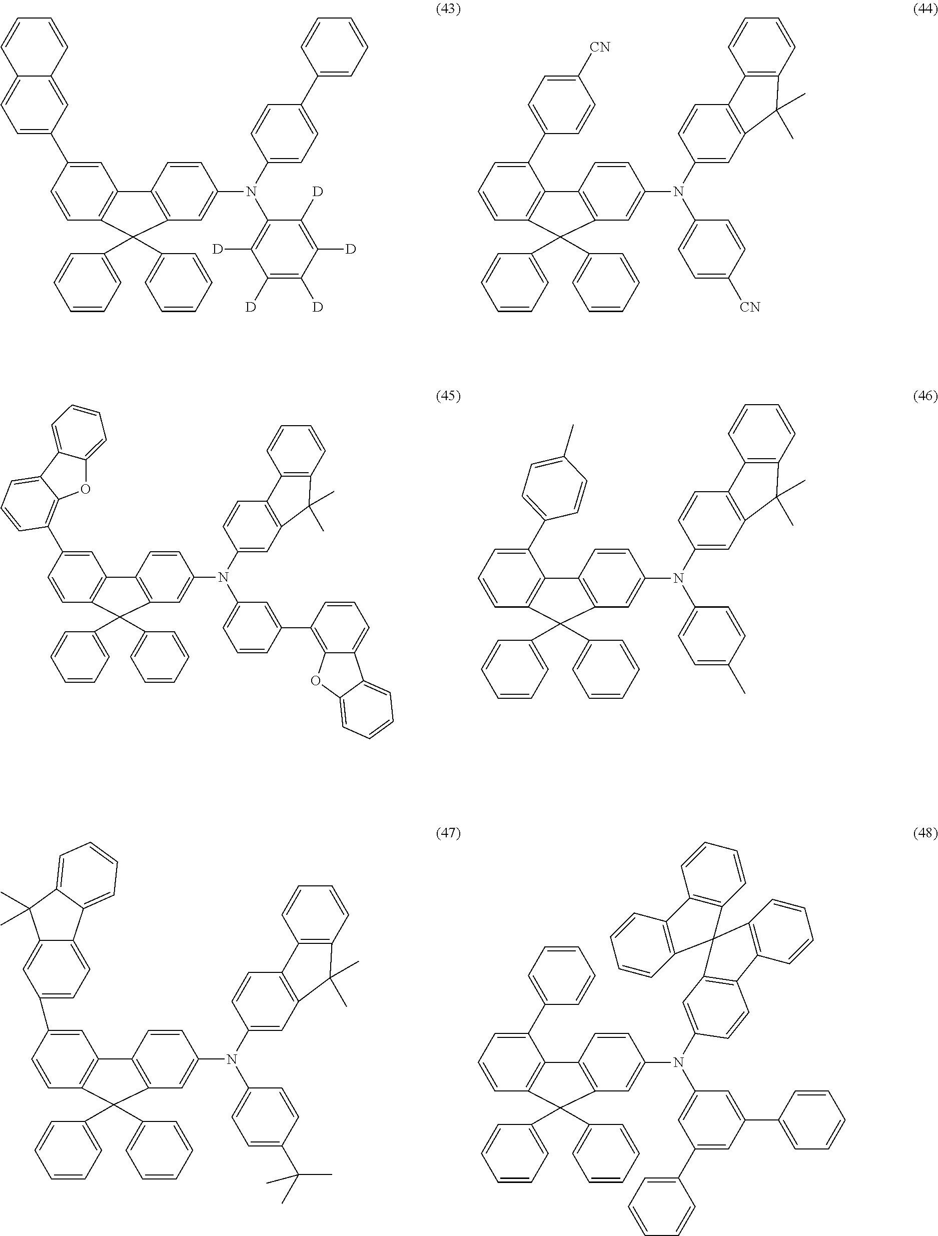

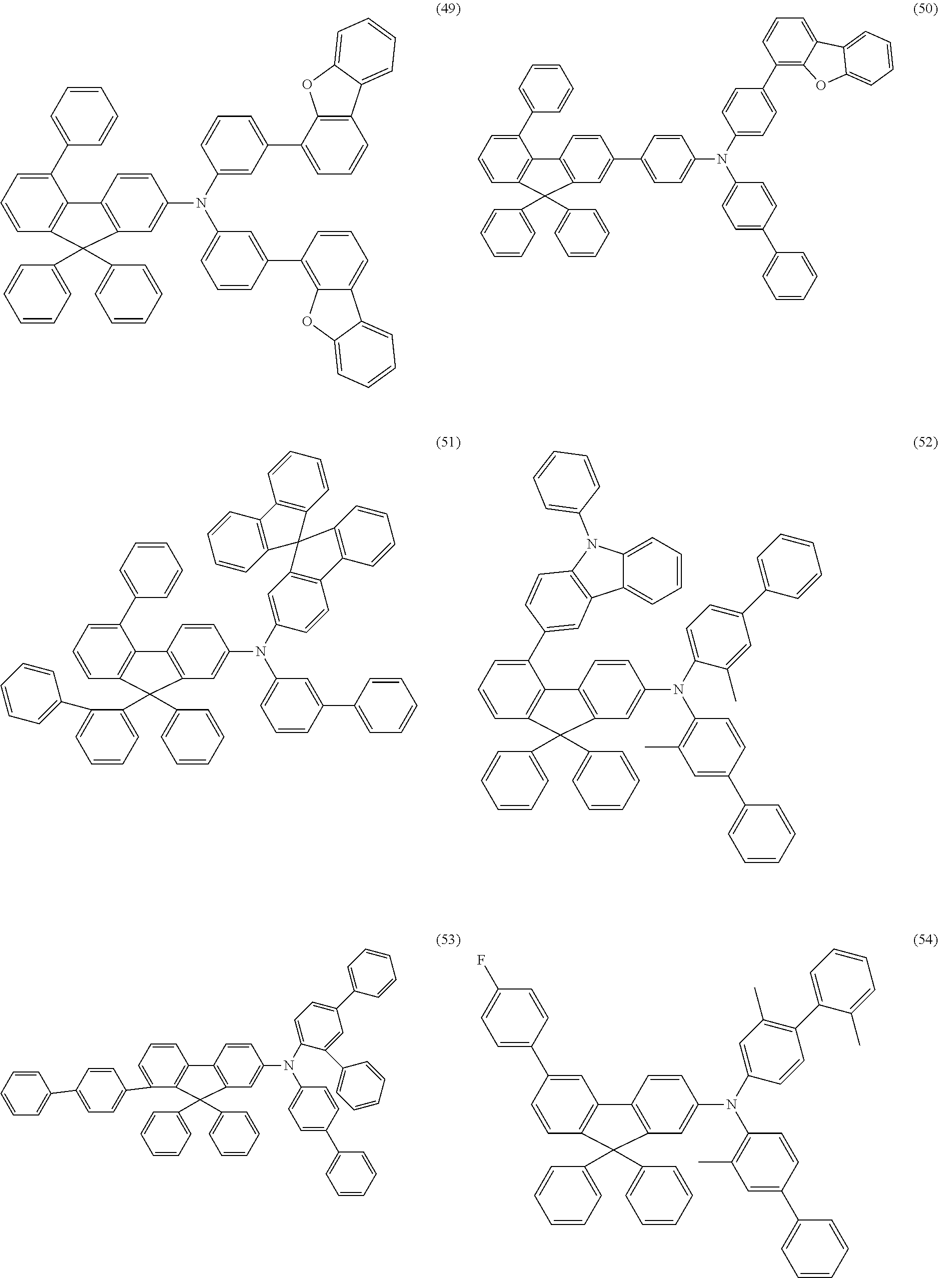

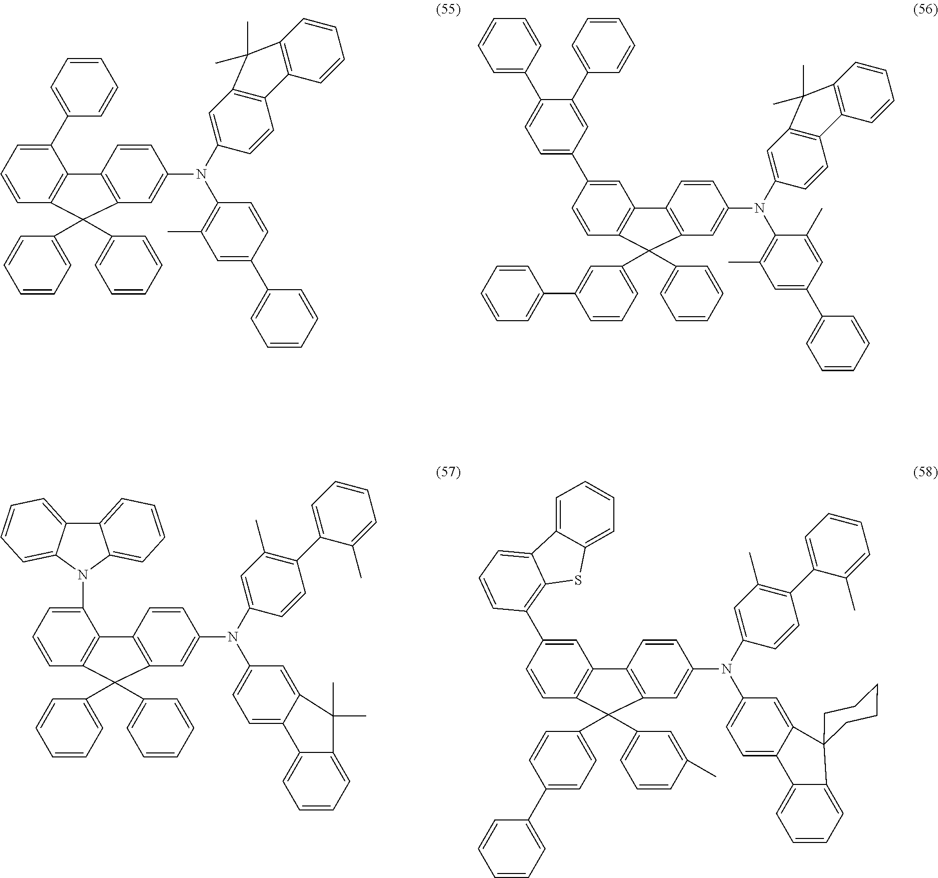

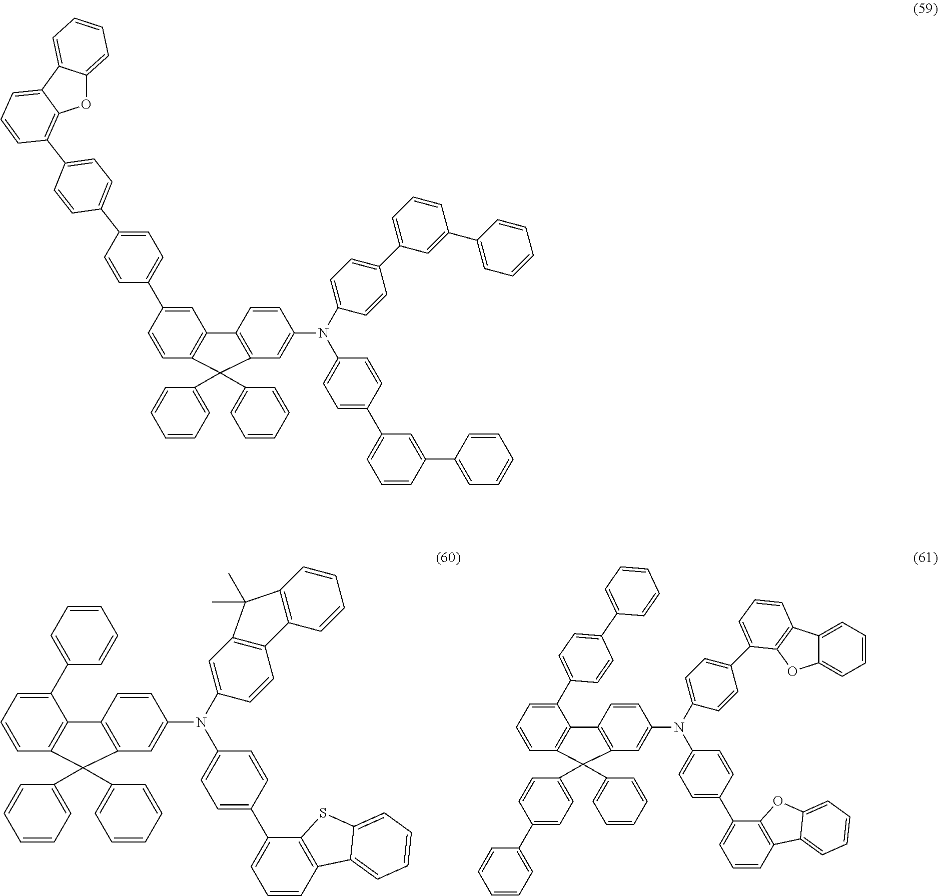 [SMILES: c1ccc(-c2cccc(-c3ccc(N(c4ccc(-c5cccc(-c6ccccc6)c5)cc4)c4ccc5c(c4)C(c4ccccc4)(c4ccccc4)c4ccc(-c6ccc(-c7ccc(-c8cccc9c8oc8ccccc89)cc7)cc6)cc4-5)cc3)c2)cc1 ; CC1(C)c2ccccc2-c2ccc(N(c3ccc(-c4cccc5c4sc4ccccc45)cc3)c3ccc4c(c3)C(c3ccccc3)(c3ccccc3)c3cccc(-c5ccccc5)c3-4)cc21 ; c1ccc(-c2ccc(-c3cccc4c3-c3ccc(N(c5ccc(-c6cccc7c6oc6ccccc67)cc5)c5ccc(-c6cccc7c6oc6ccccc67)cc5)cc3C4(c3ccccc3)c3ccc(-c4ccccc4)cc3)cc2)cc1]

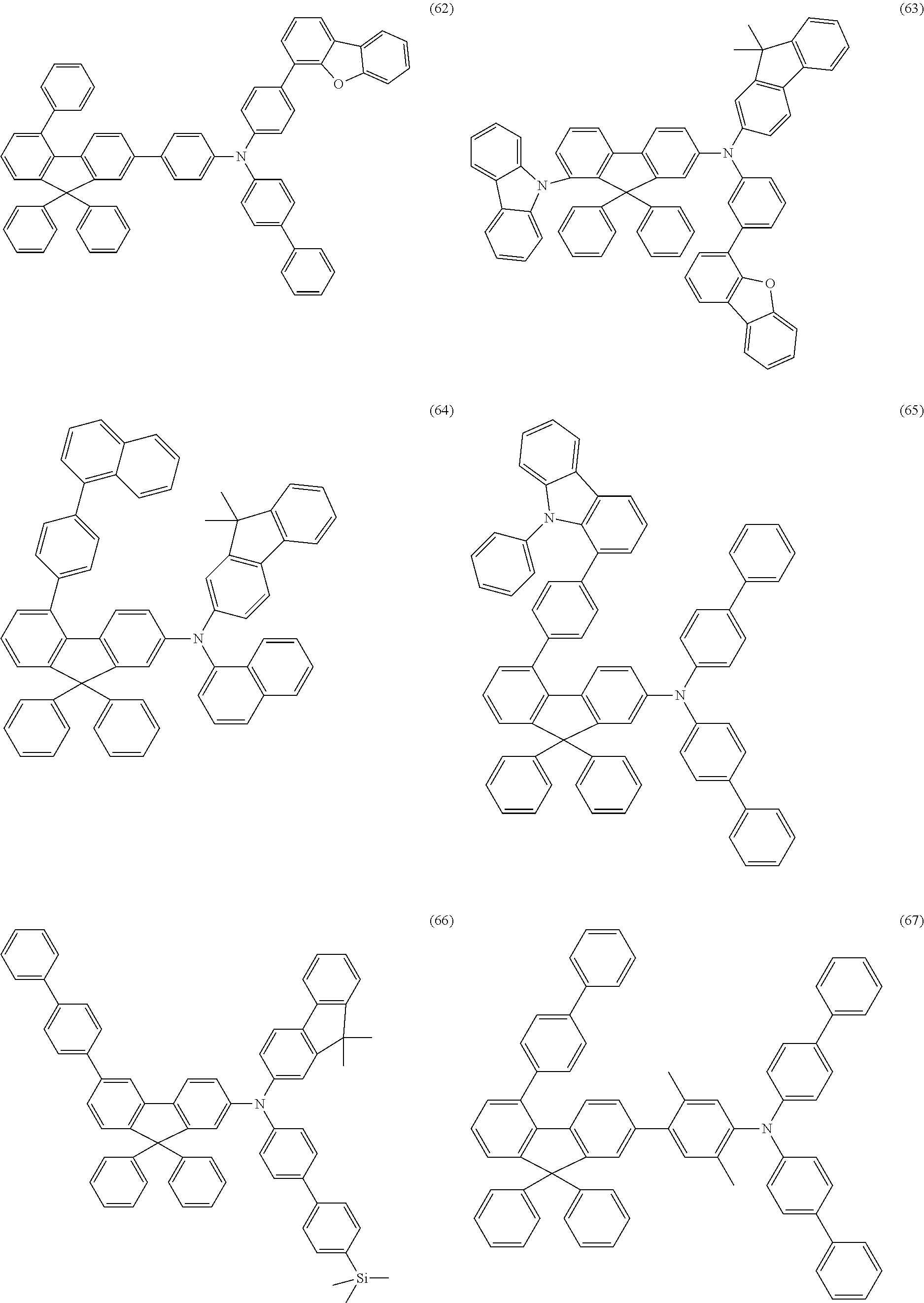

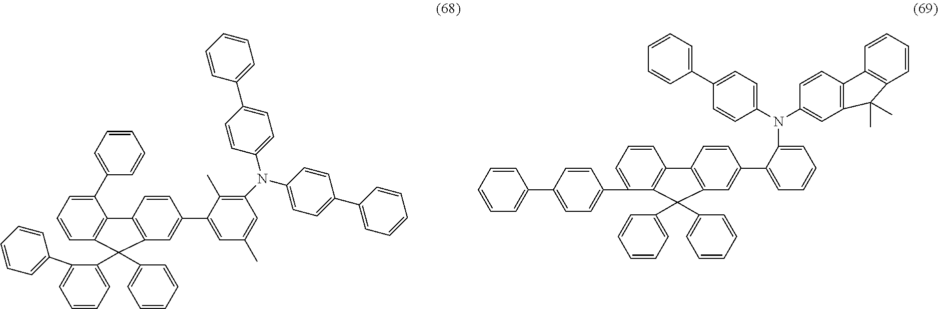 [SMILES: Cc1cc(-c2ccc3c(c2)C(c2ccccc2)(c2ccccc2-c2ccccc2)c2cccc(-c4ccccc4)c2-3)c(C)c(N(c2ccc(-c3ccccc3)cc2)c2ccc(-c3ccccc3)cc2)c1 ; CC1(C)c2ccccc2-c2ccc(N(c3ccc(-c4ccccc4)cc3)c3ccccc3-c3ccc4c(c3)C(c3ccccc3)(c3ccccc3)c3c(-c5ccc(-c6ccccc6)cc5)cccc3-4)cc21]

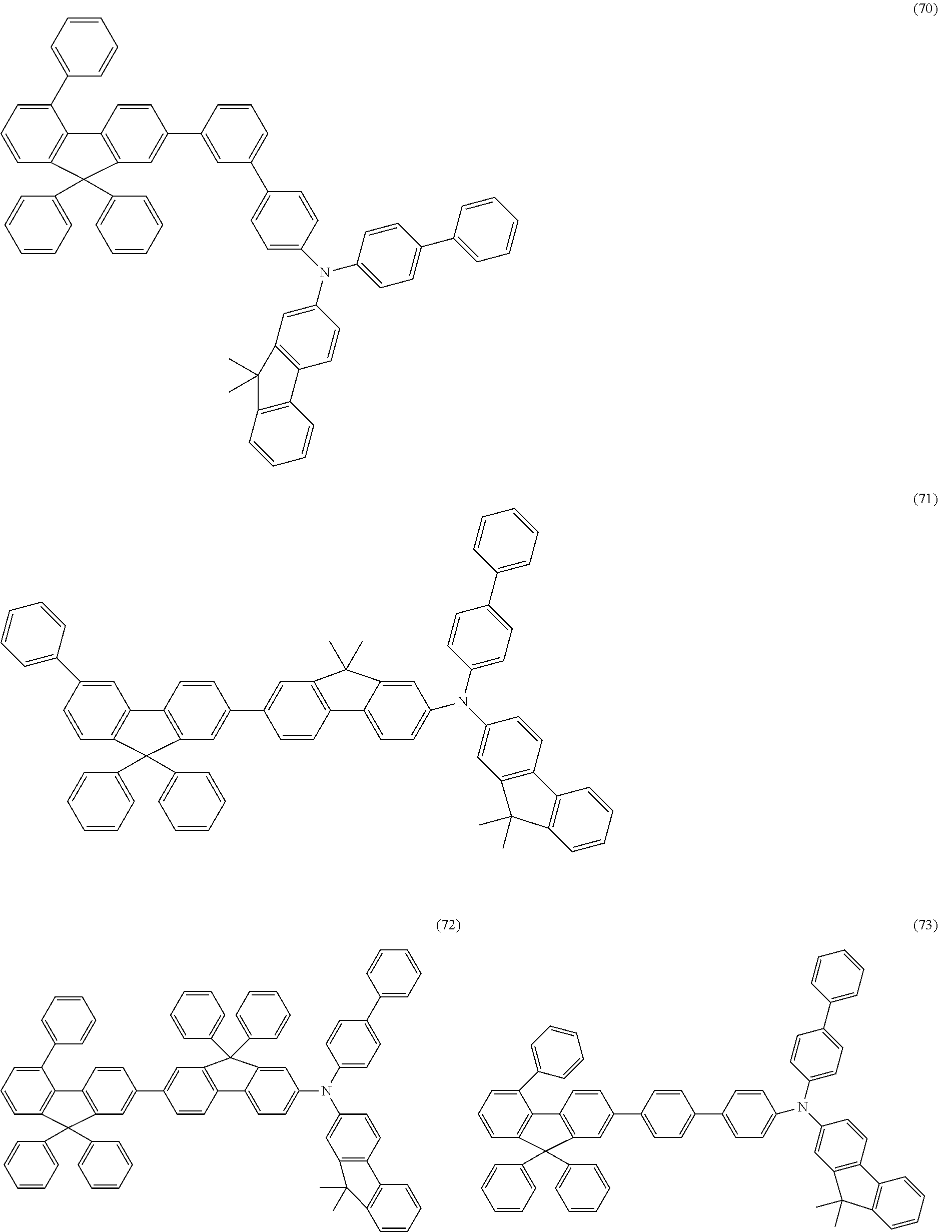

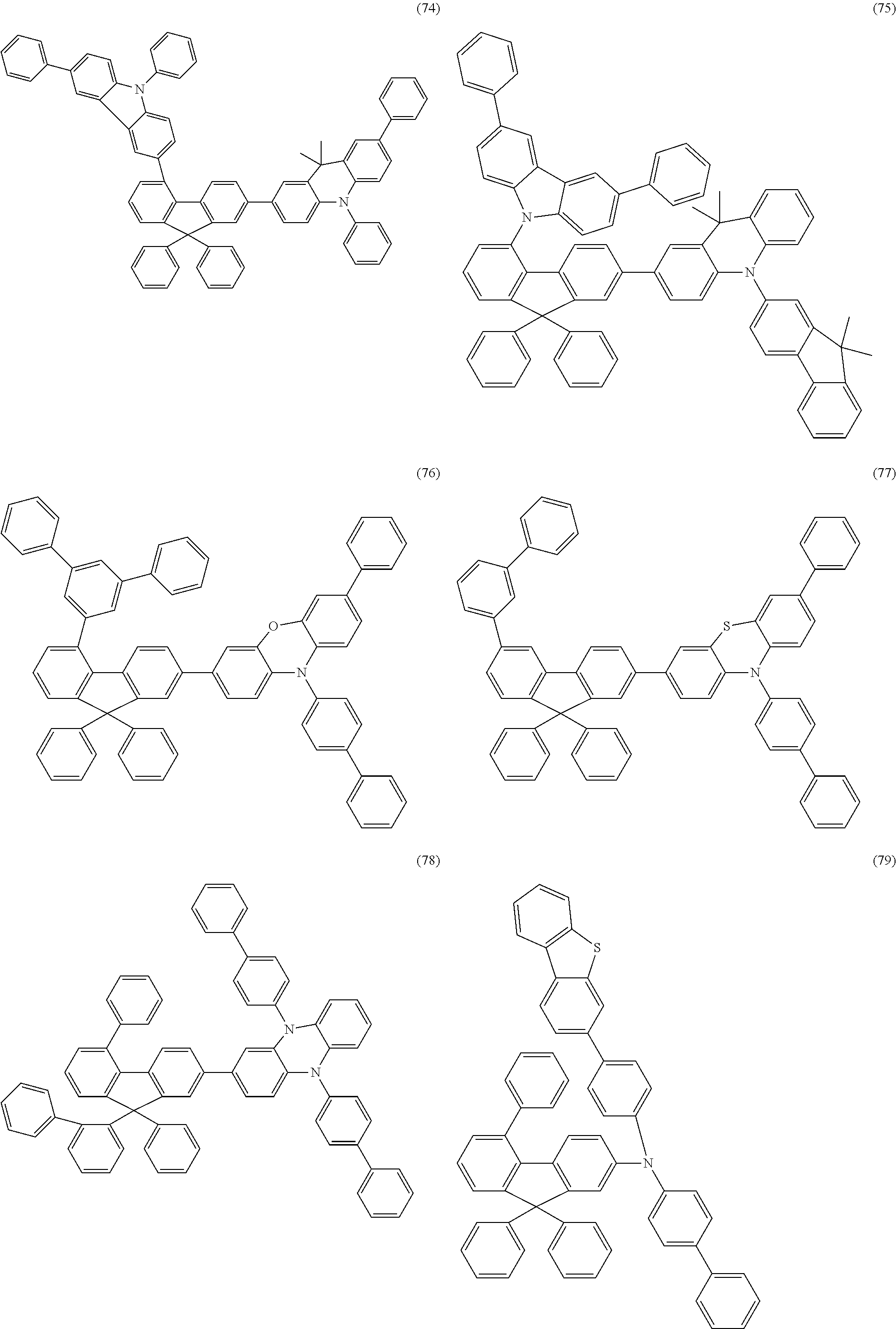

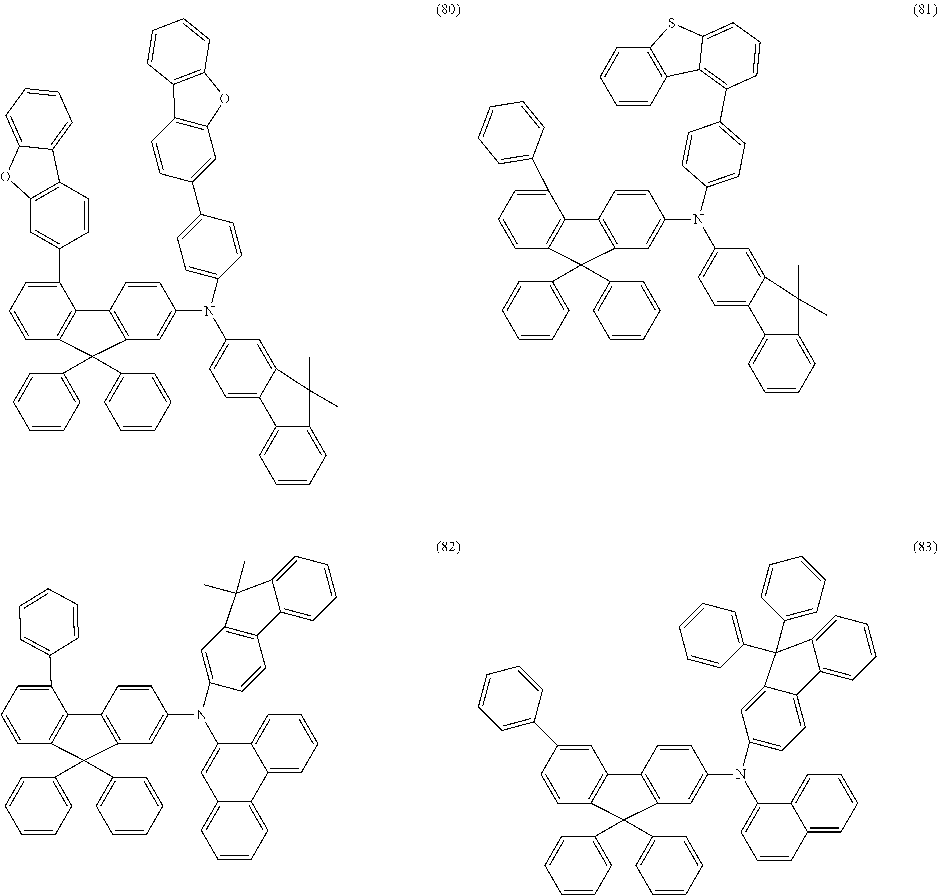 [SMILES: CC1(C)c2ccccc2-c2ccc(N(c3ccc(-c4ccc5c(c4)oc4ccccc45)cc3)c3ccc4c(c3)C(c3ccccc3)(c3ccccc3)c3cccc(-c5ccc6c(c5)oc5ccccc56)c3-4)cc21 ; CC1(C)c2ccccc2-c2ccc(N(c3ccc(-c4cccc5sc6ccccc6c45)cc3)c3ccc4c(c3)C(c3ccccc3)(c3ccccc3)c3cccc(-c5ccccc5)c3-4)cc21 ; CC1(C)c2ccccc2-c2ccc(N(c3ccc4c(c3)C(c3ccccc3)(c3ccccc3)c3cccc(-c5ccccc5)c3-4)c3cc4ccccc4c4ccccc34)cc21 ; c1ccc(-c2ccc3c(c2)-c2ccc(N(c4ccc5c(c4)C(c4ccccc4)(c4ccccc4)c4ccccc4-5)c4cccc5ccccc45)cc2C3(c2ccccc2)c2ccccc2)cc1]

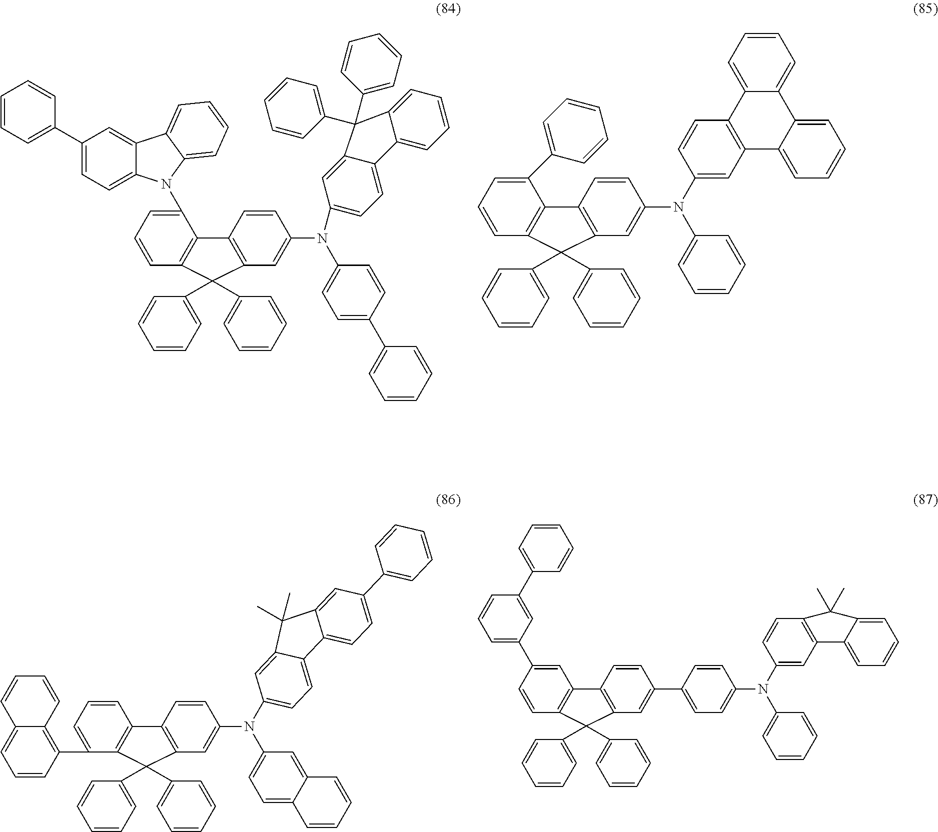 [SMILES: c1ccc(-c2ccc(N(c3ccc4c(c3)C(c3ccccc3)(c3ccccc3)c3ccccc3-4)c3ccc4c(c3)C(c3ccccc3)(c3ccccc3)c3cccc(-n5c6ccccc6c6cc(-c7ccccc7)ccc65)c3-4)cc2)cc1 ; c1ccc(-c2cccc3c2-c2ccc(N(c4ccccc4)c4ccc5c6ccccc6c6ccccc6c5c4)cc2C3(c2ccccc2)c2ccccc2)cc1 ; CC1(C)c2cc(-c3ccccc3)ccc2-c2ccc(N(c3ccc4c(c3)C(c3ccccc3)(c3ccccc3)c3c-4cccc3-c3cccc4ccccc34)c3ccc4ccccc4c3)cc21 ; CC1(C)c2ccccc2-c2cc(N(c3ccccc3)c3ccc(-c4ccc5c(c4)C(c4ccccc4)(c4ccccc4)c4ccc(-c6cccc(-c7ccccc7)c6)cc4-5)cc3)ccc21]

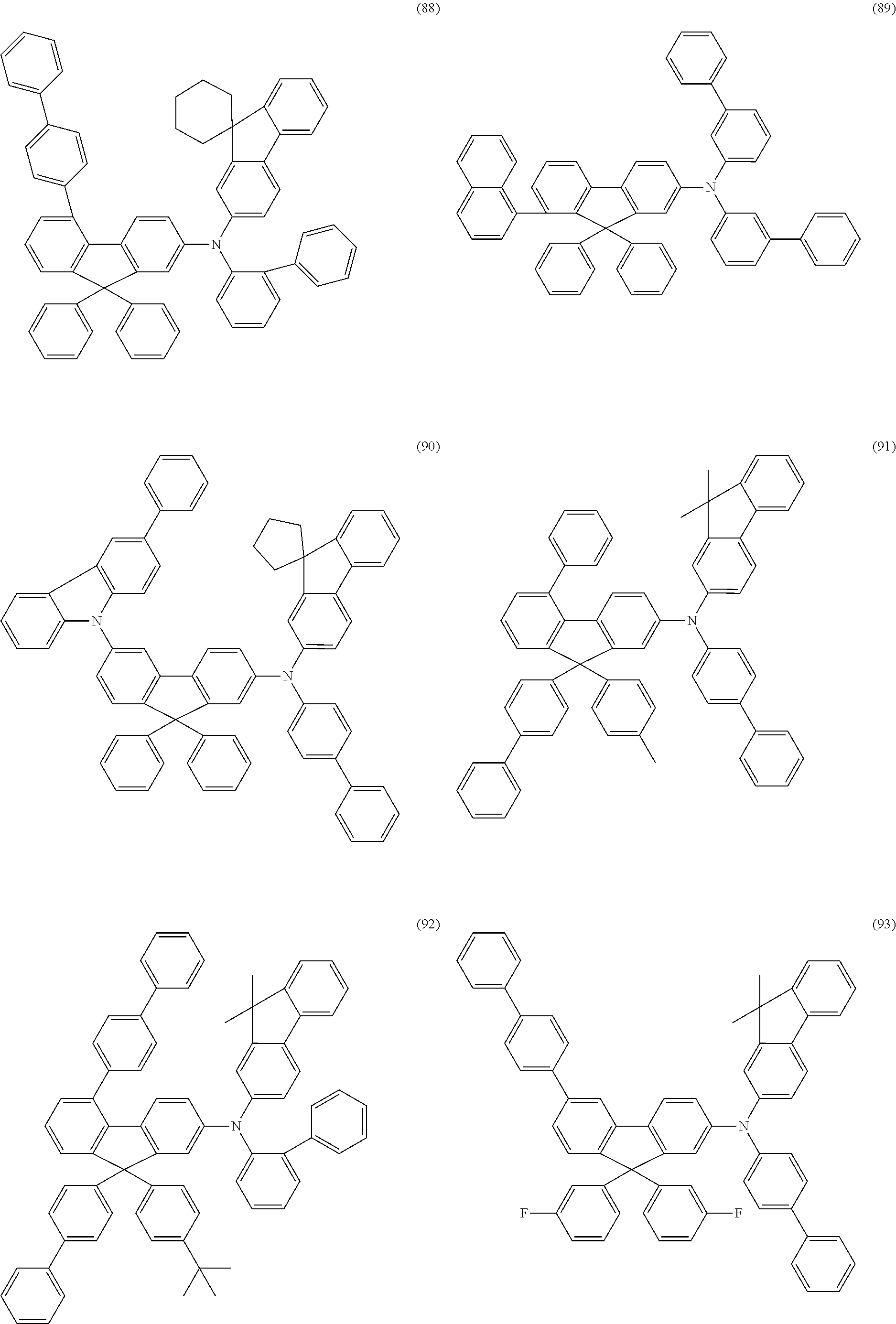

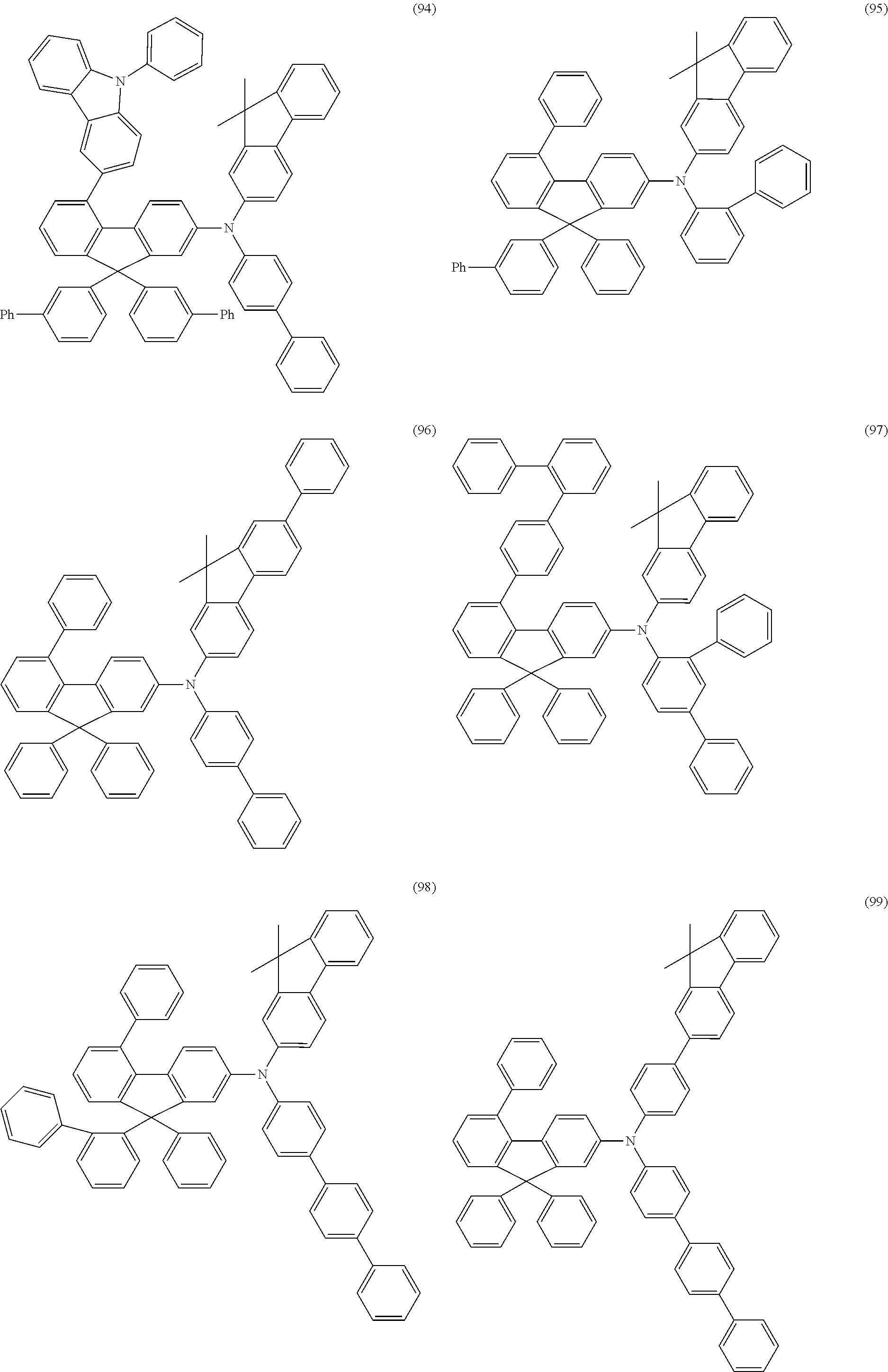

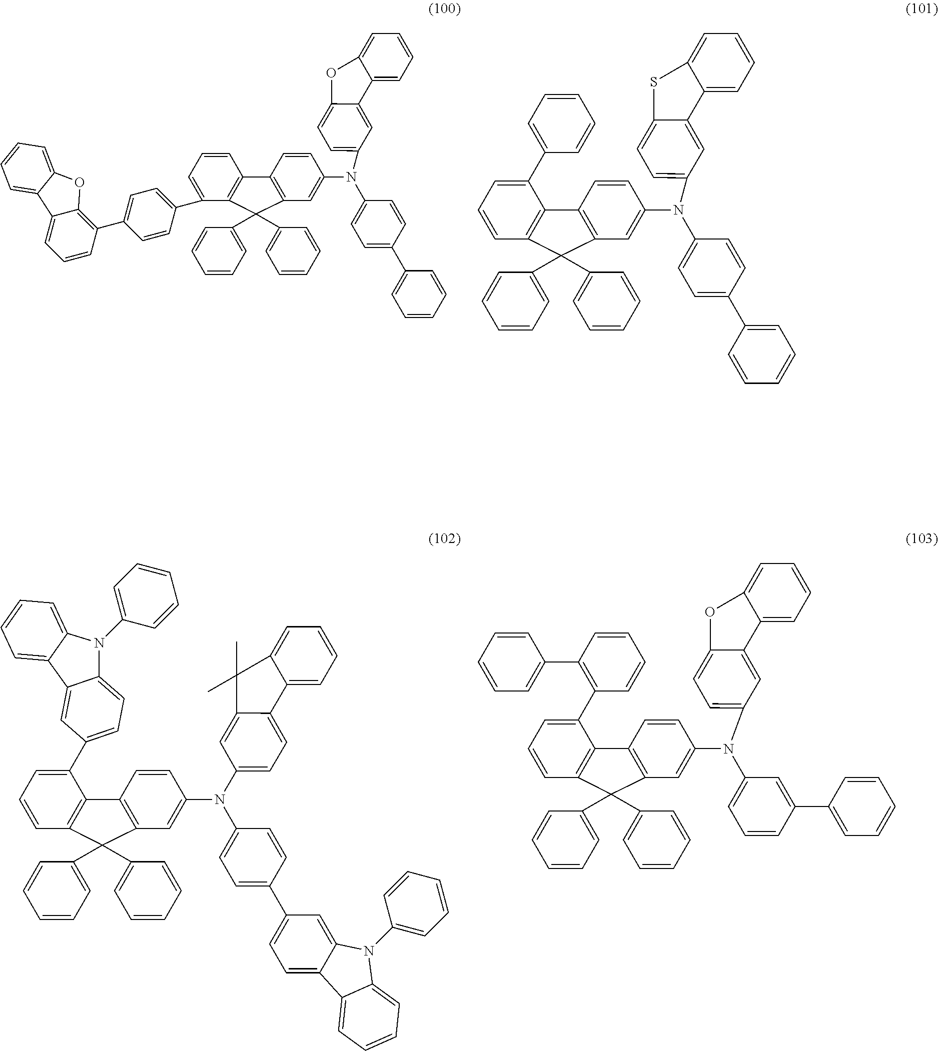

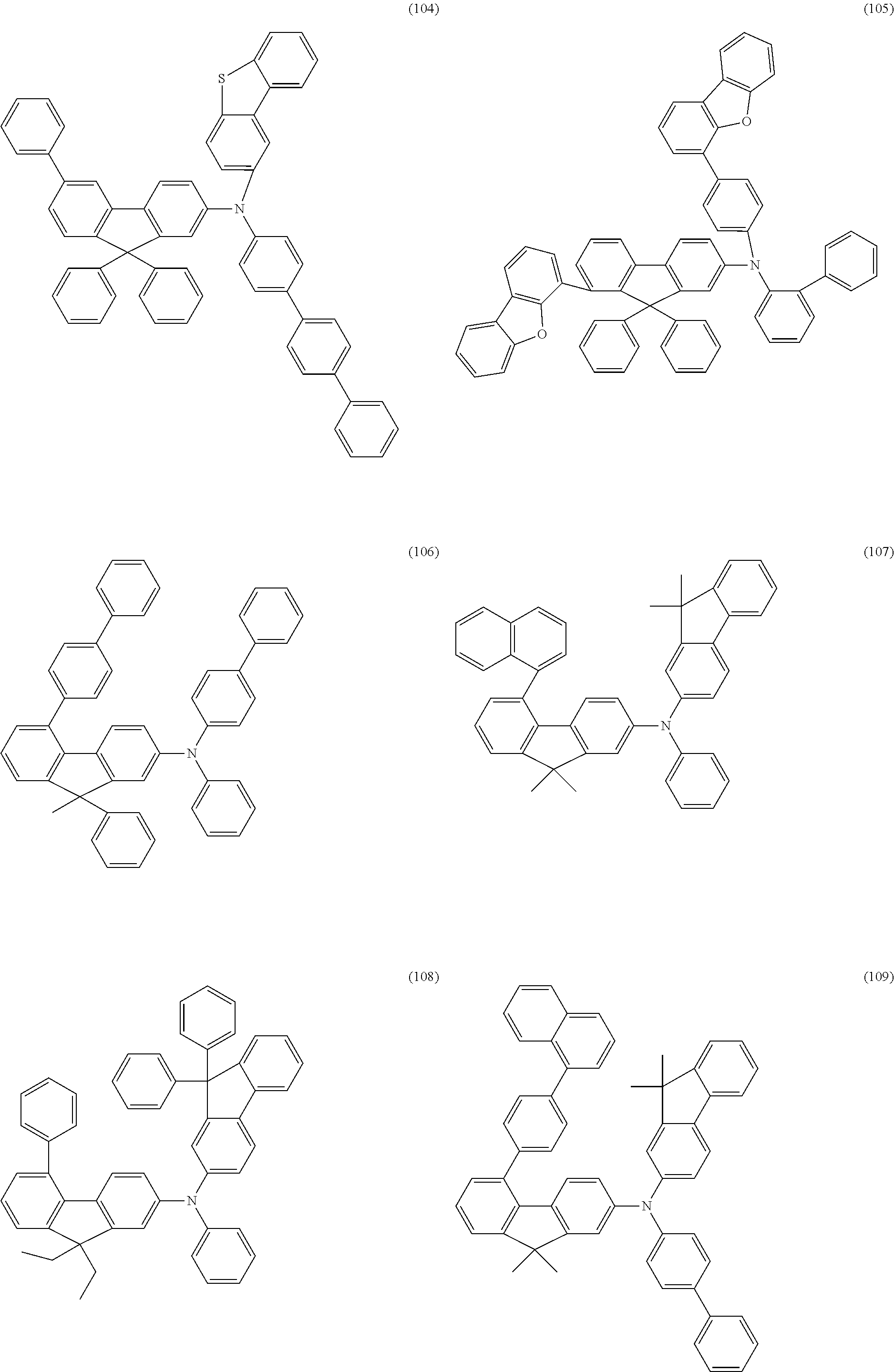

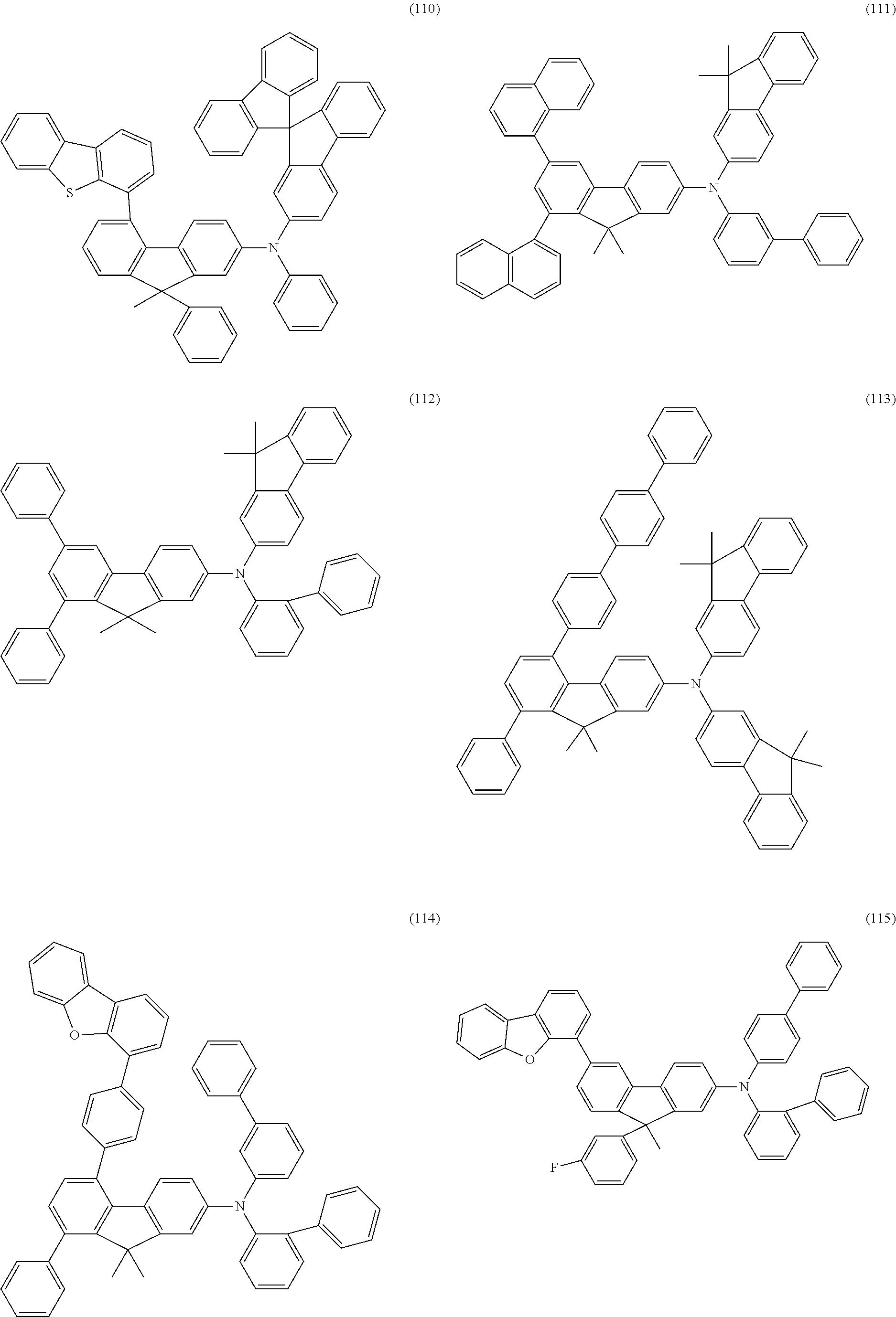

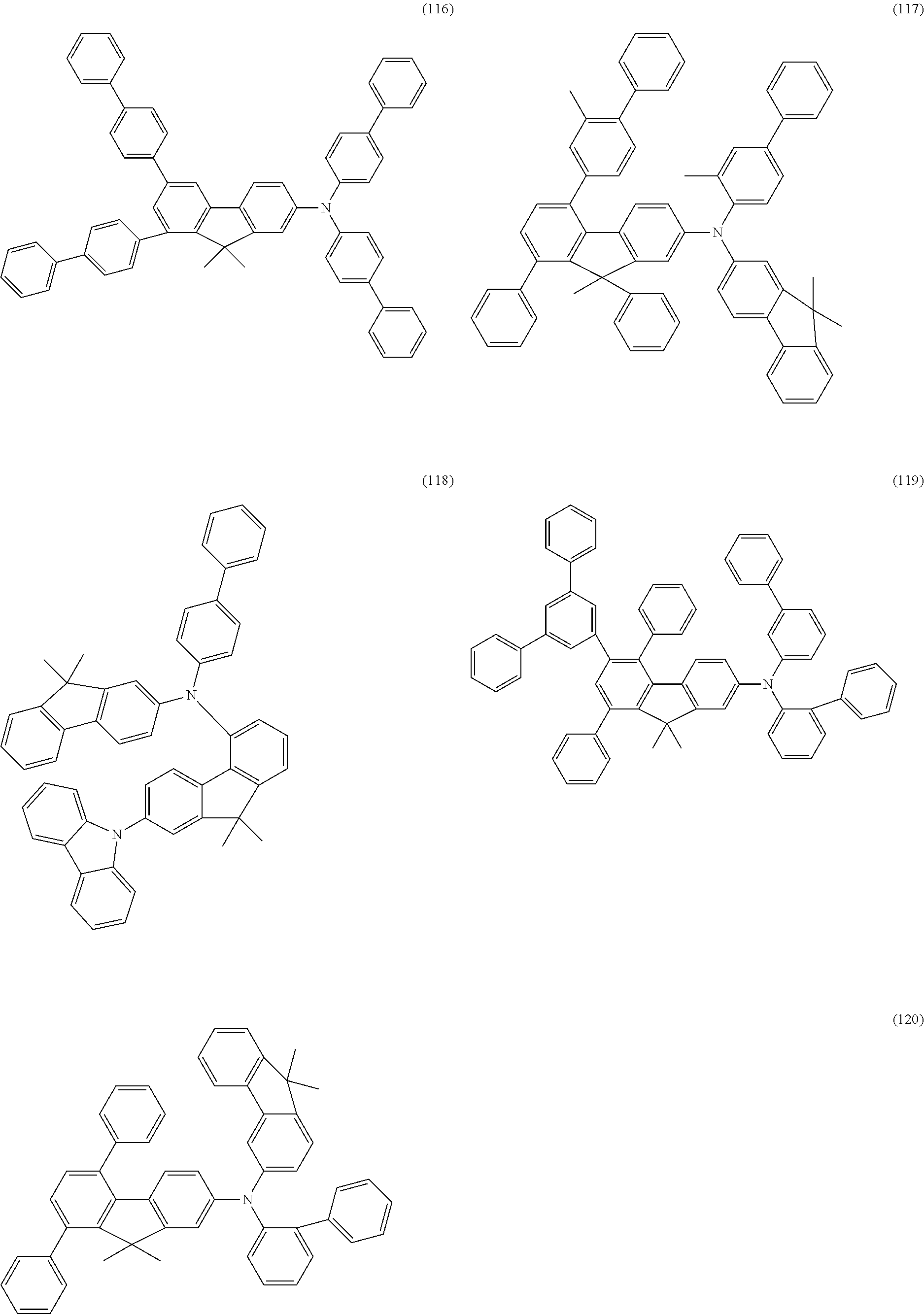

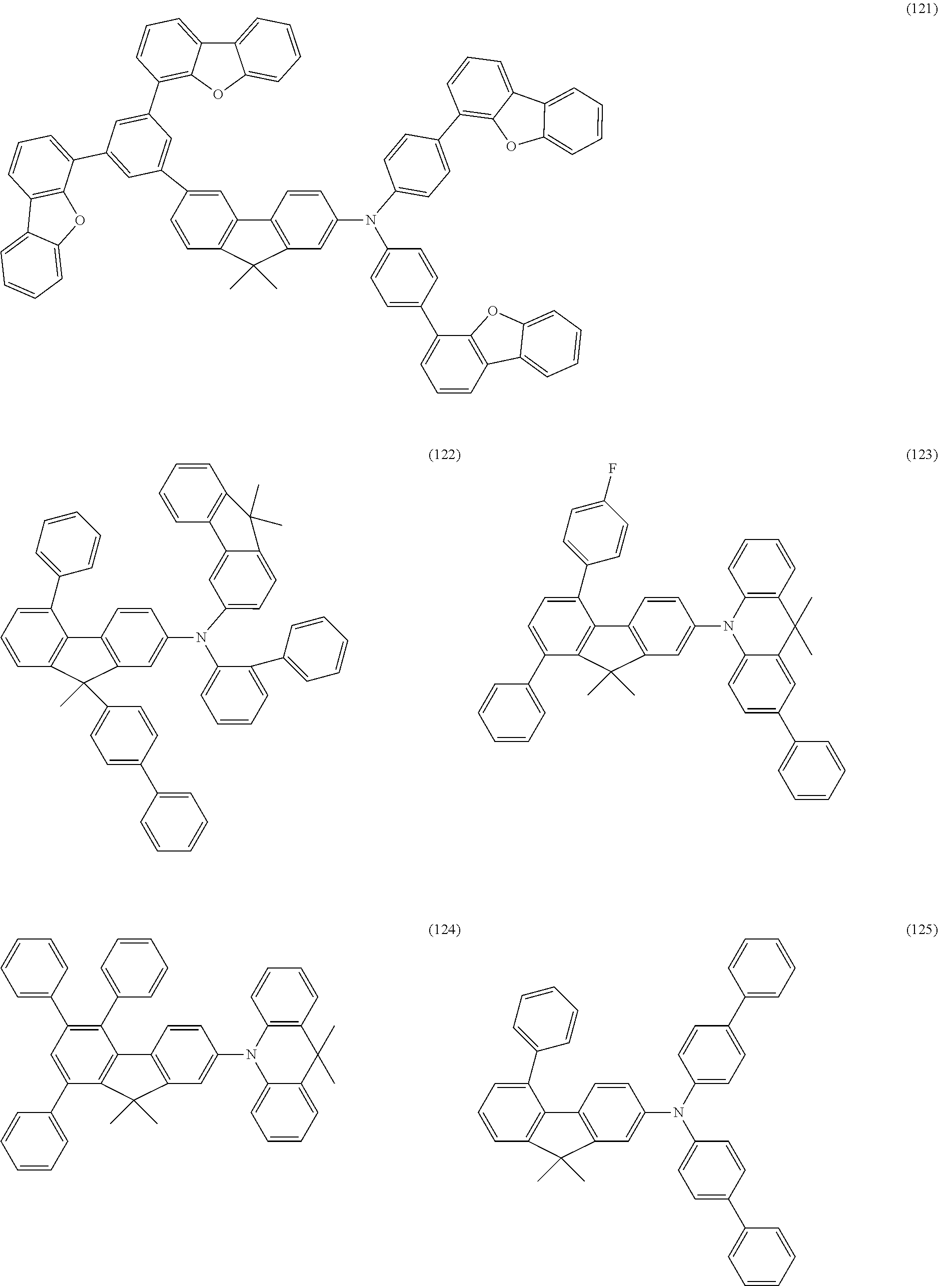

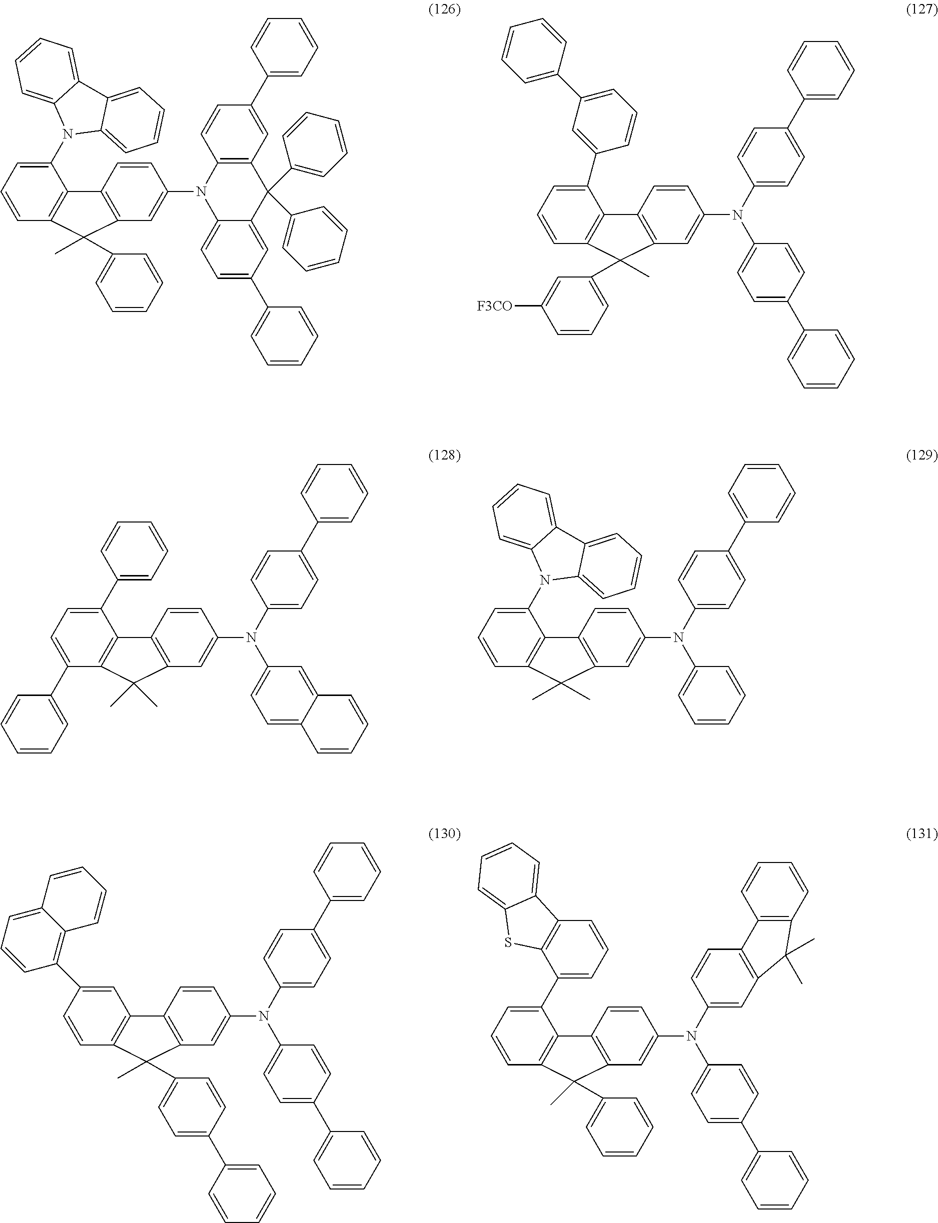

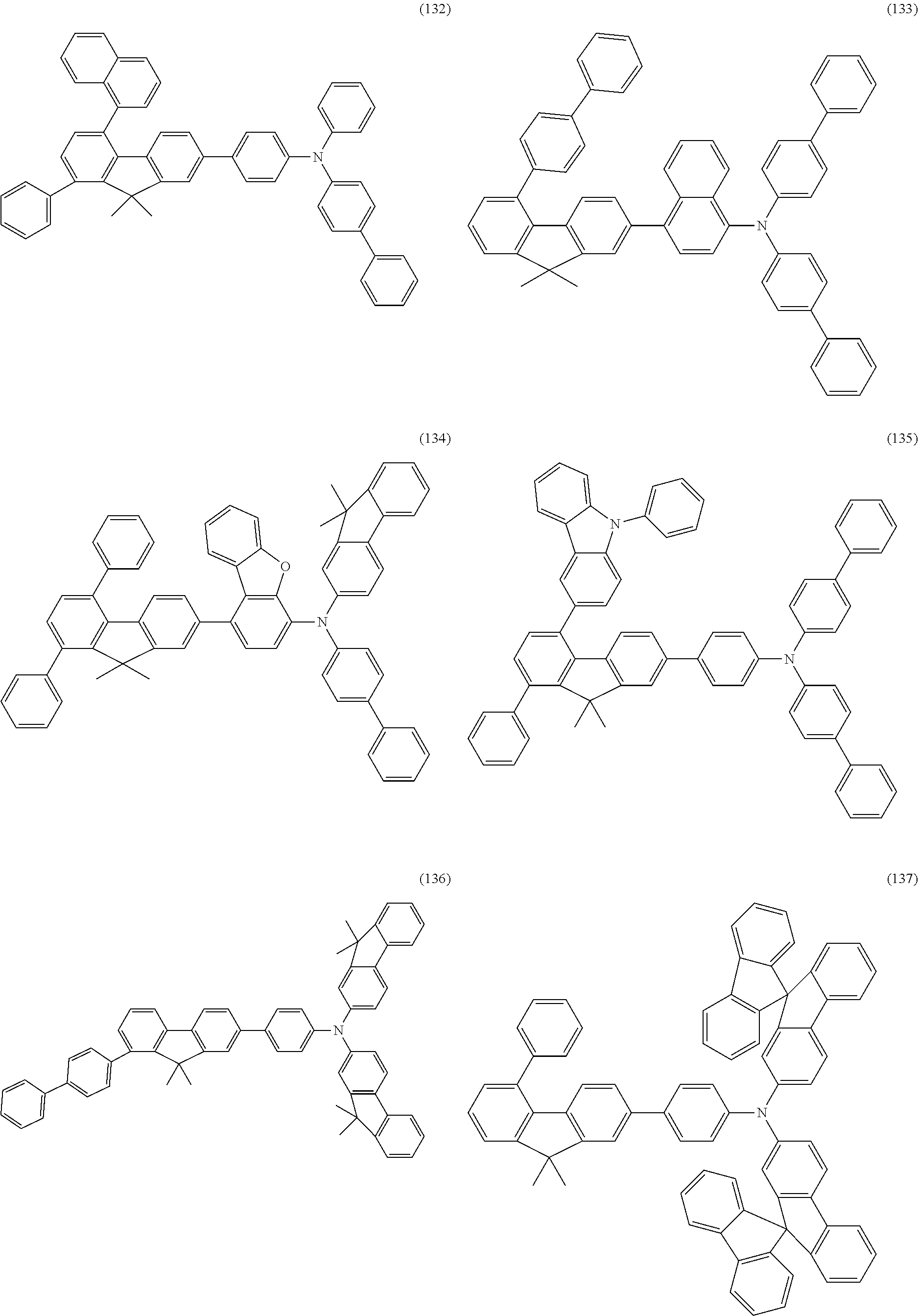

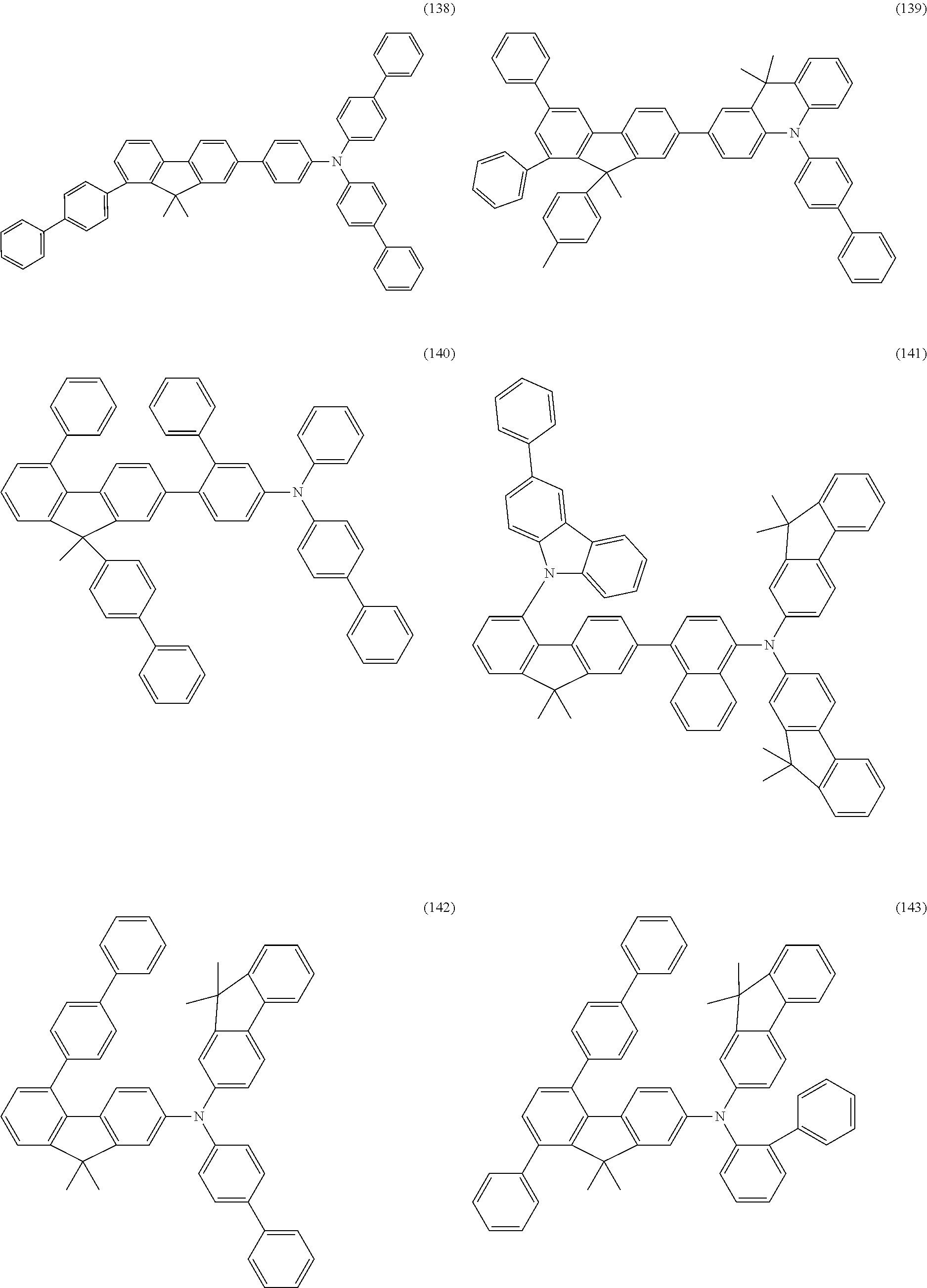

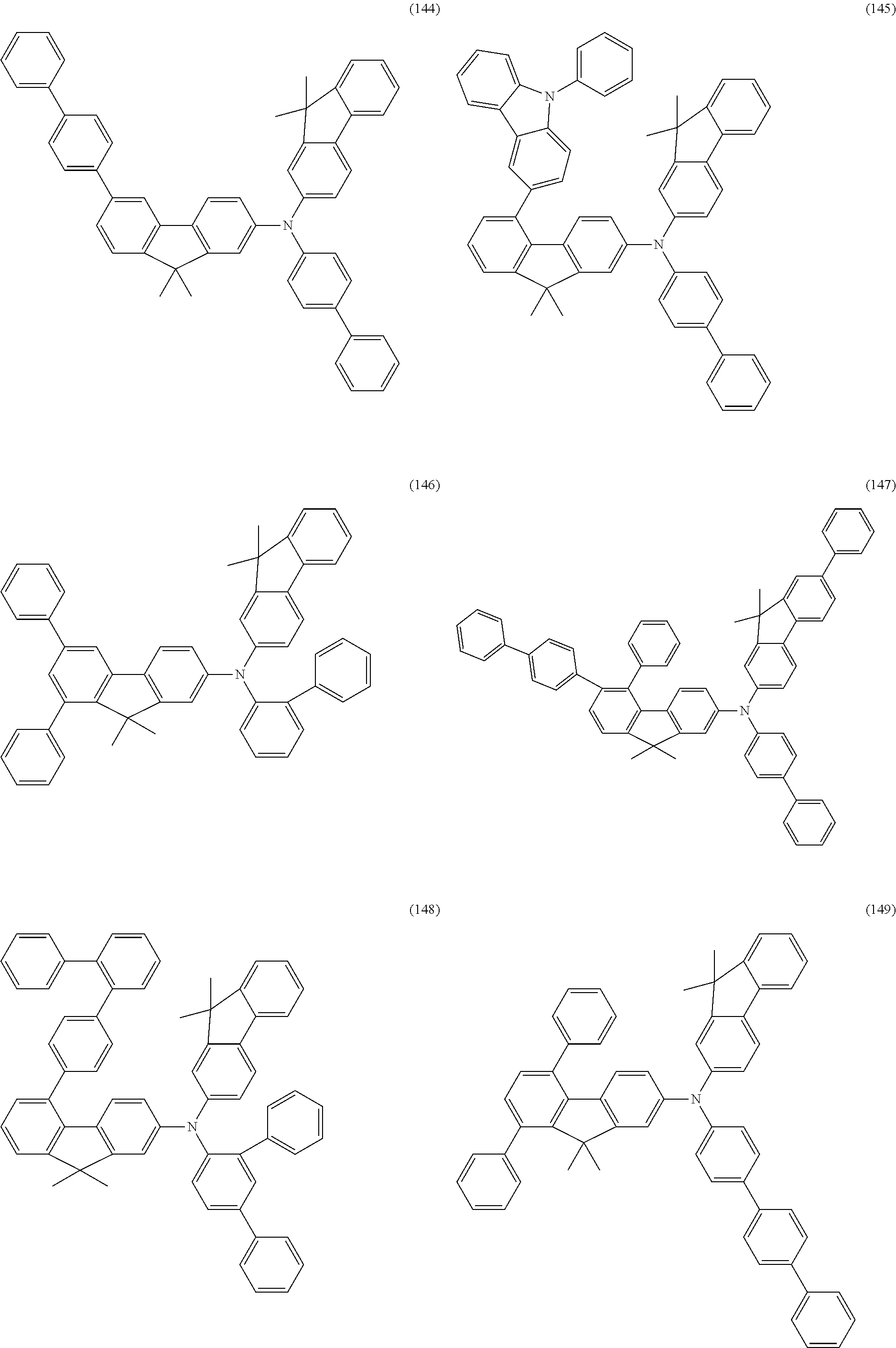

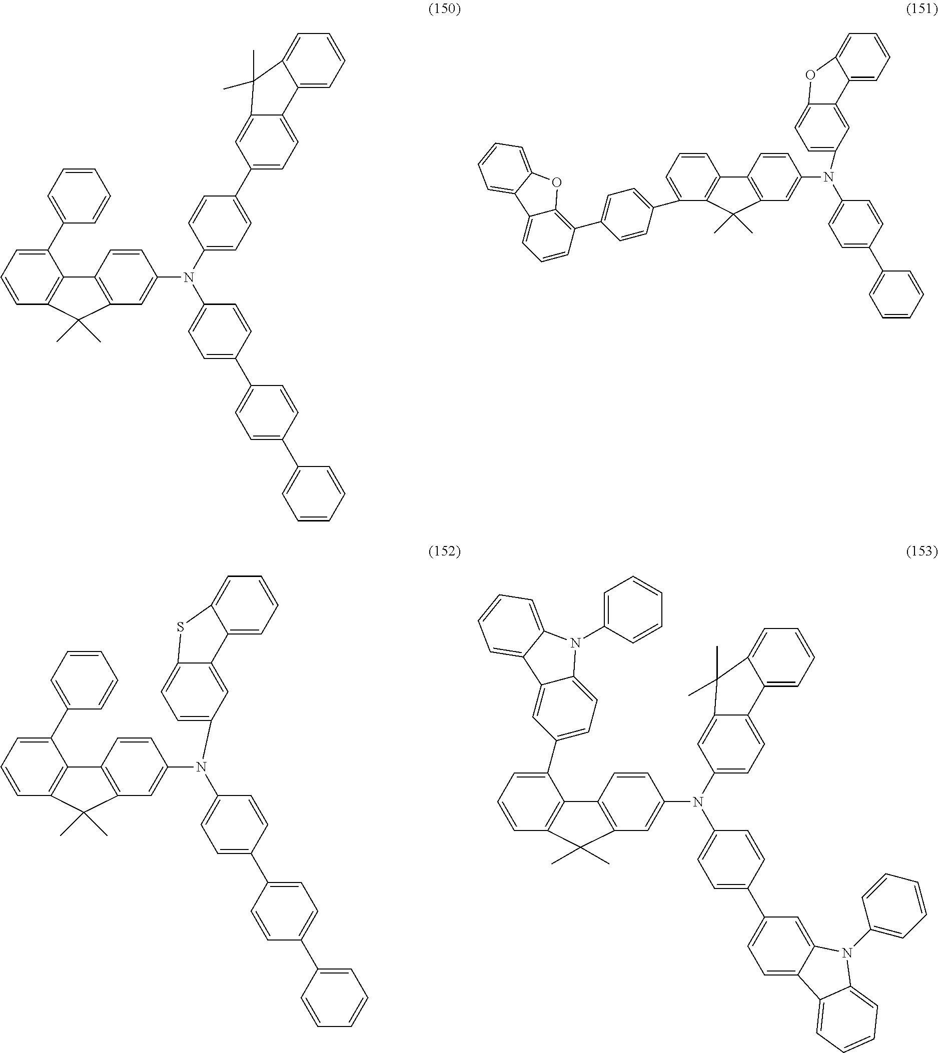 [SMILES: CC1(C)c2ccccc2-c2ccc(-c3ccc(N(c4ccc(-c5ccc(-c6ccccc6)cc5)cc4)c4ccc5c(c4)C(C)(C)c4cccc(-c6ccccc6)c4-5)cc3)cc21 ; CC1(C)c2cc(N(c3ccc(-c4ccccc4)cc3)c3ccc4oc5ccccc5c4c3)ccc2-c2cccc(-c3ccc(-c4cccc5c4oc4ccccc45)cc3)c21 ; CC1(C)c2cc(N(c3ccc(-c4ccc(-c5ccccc5)cc4)cc3)c3ccc4sc5ccccc5c4c3)ccc2-c2c(-c3ccccc3)cccc21 ; CC1(C)c2ccccc2-c2ccc(N(c3ccc(-c4ccc5c6ccccc6n(-c6ccccc6)c5c4)cc3)c3ccc4c(c3)C(C)(C)c3cccc(-c5ccc6c(c5)c5ccccc5n6-c5ccccc5)c3-4)cc21]

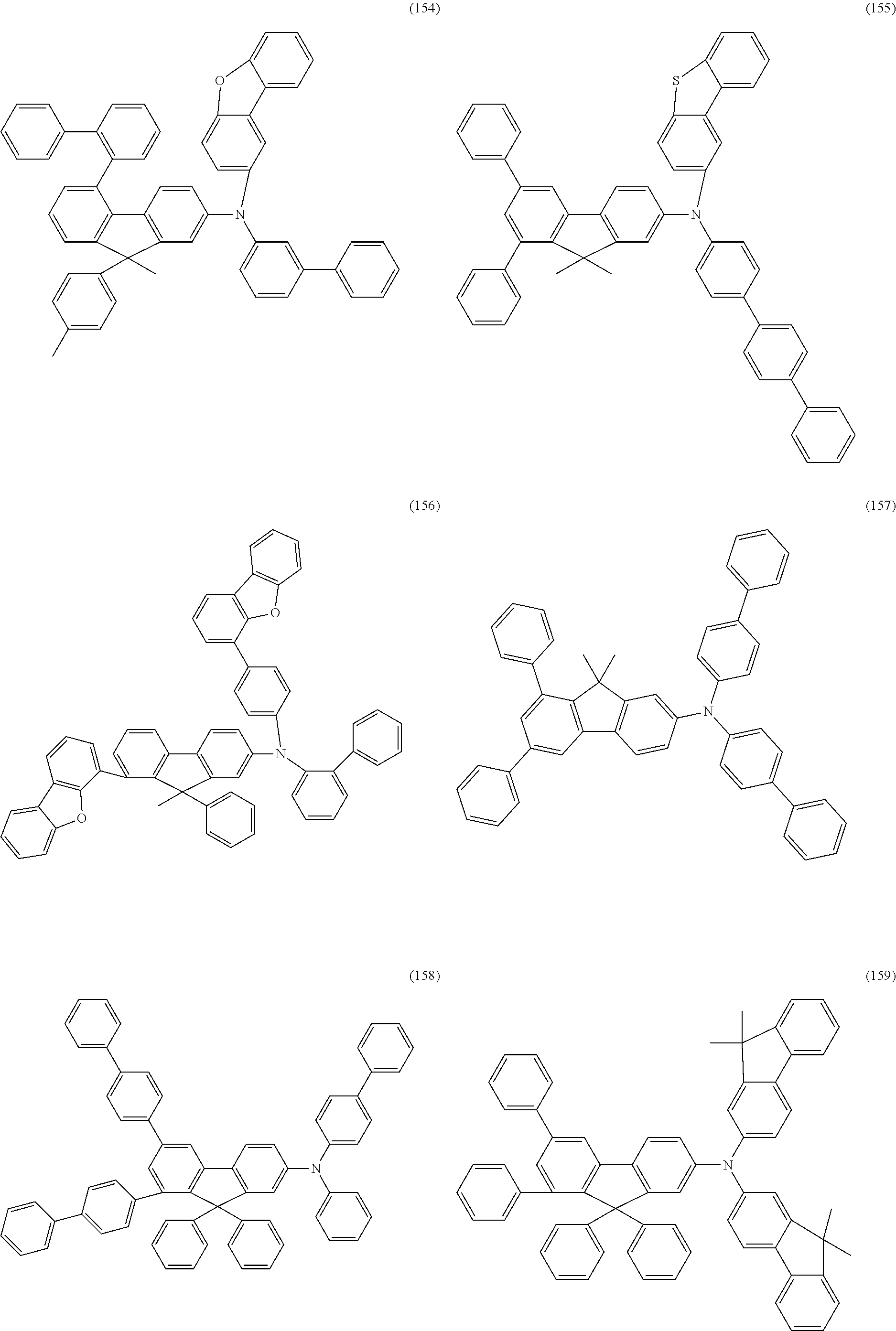

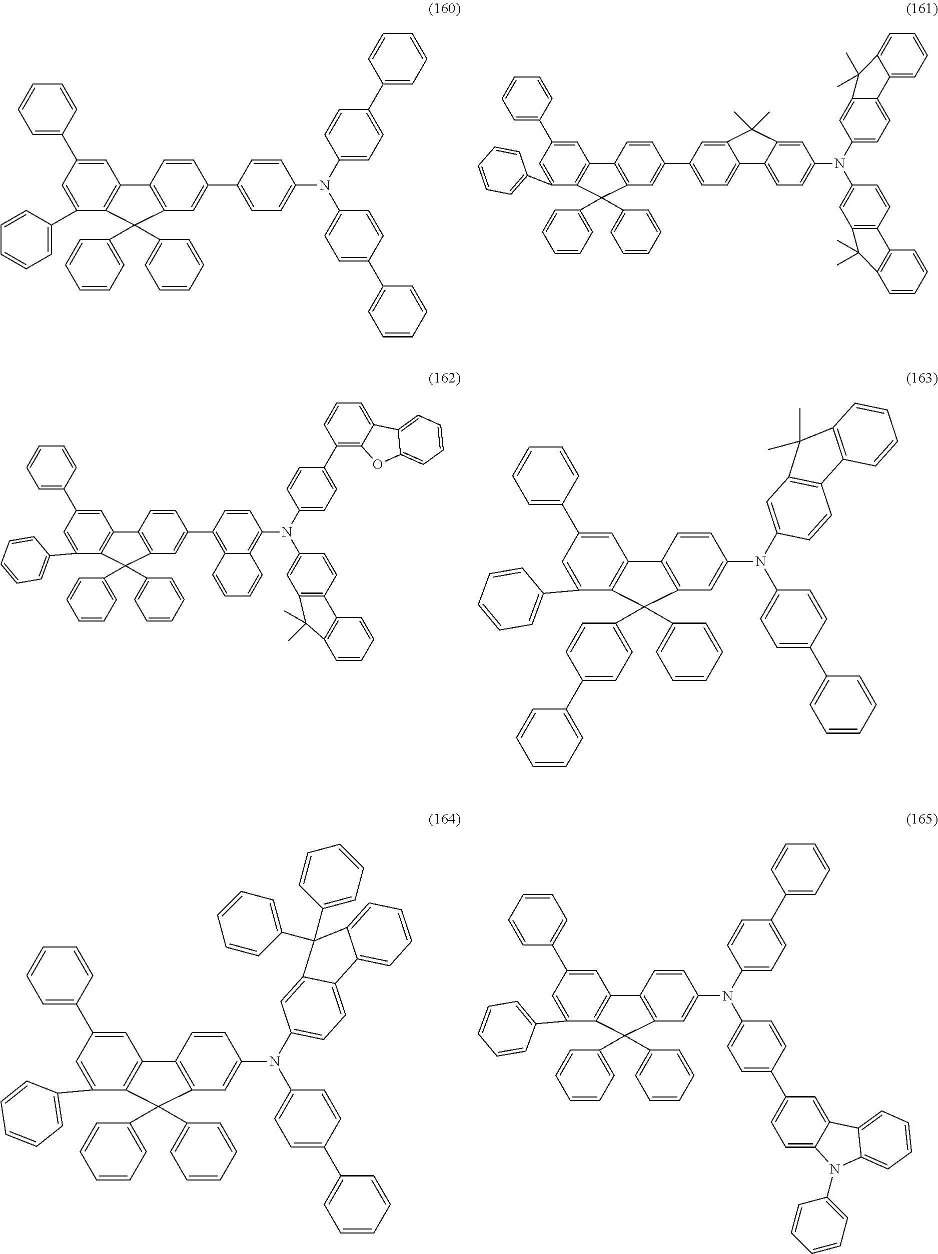

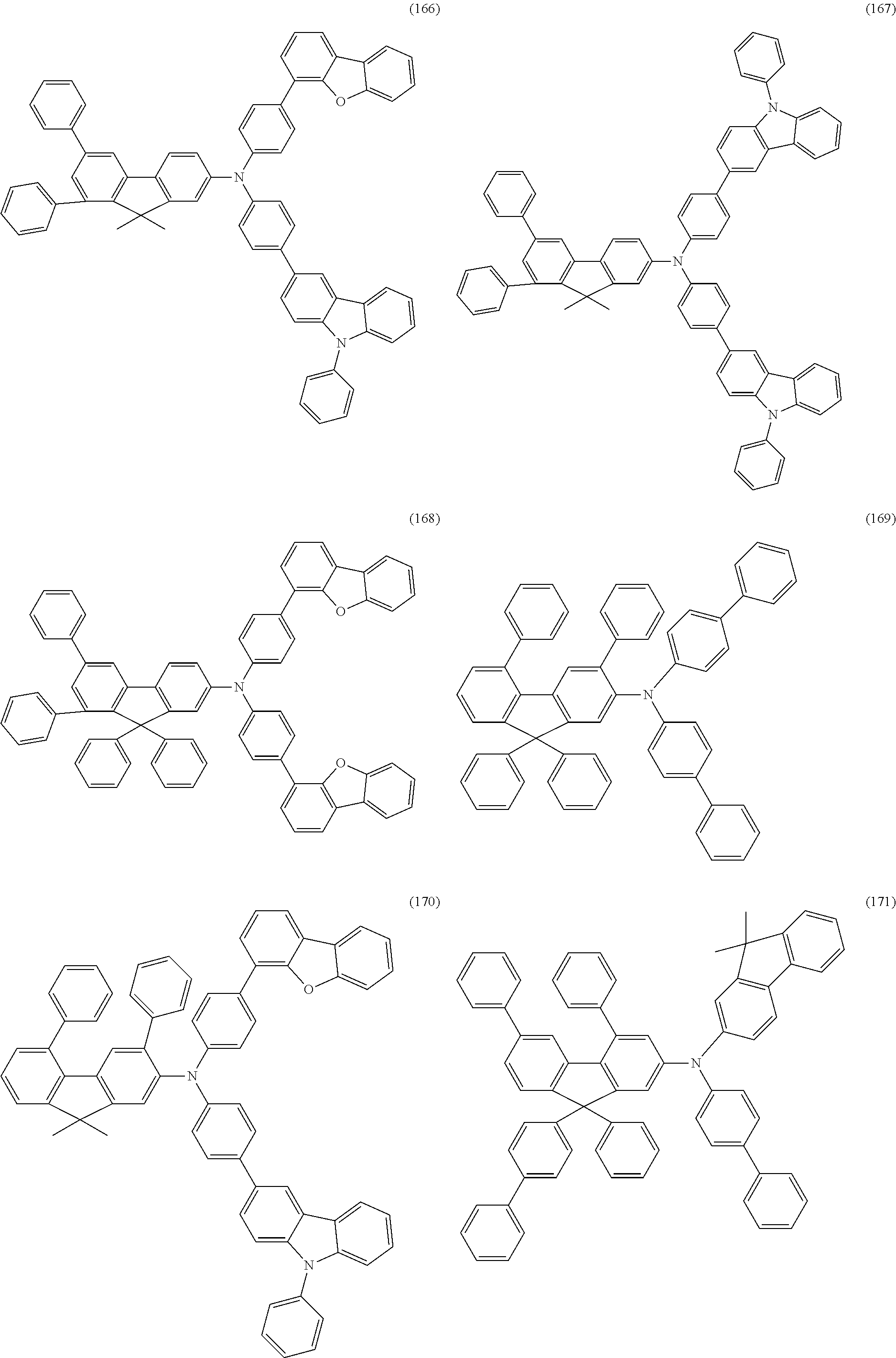

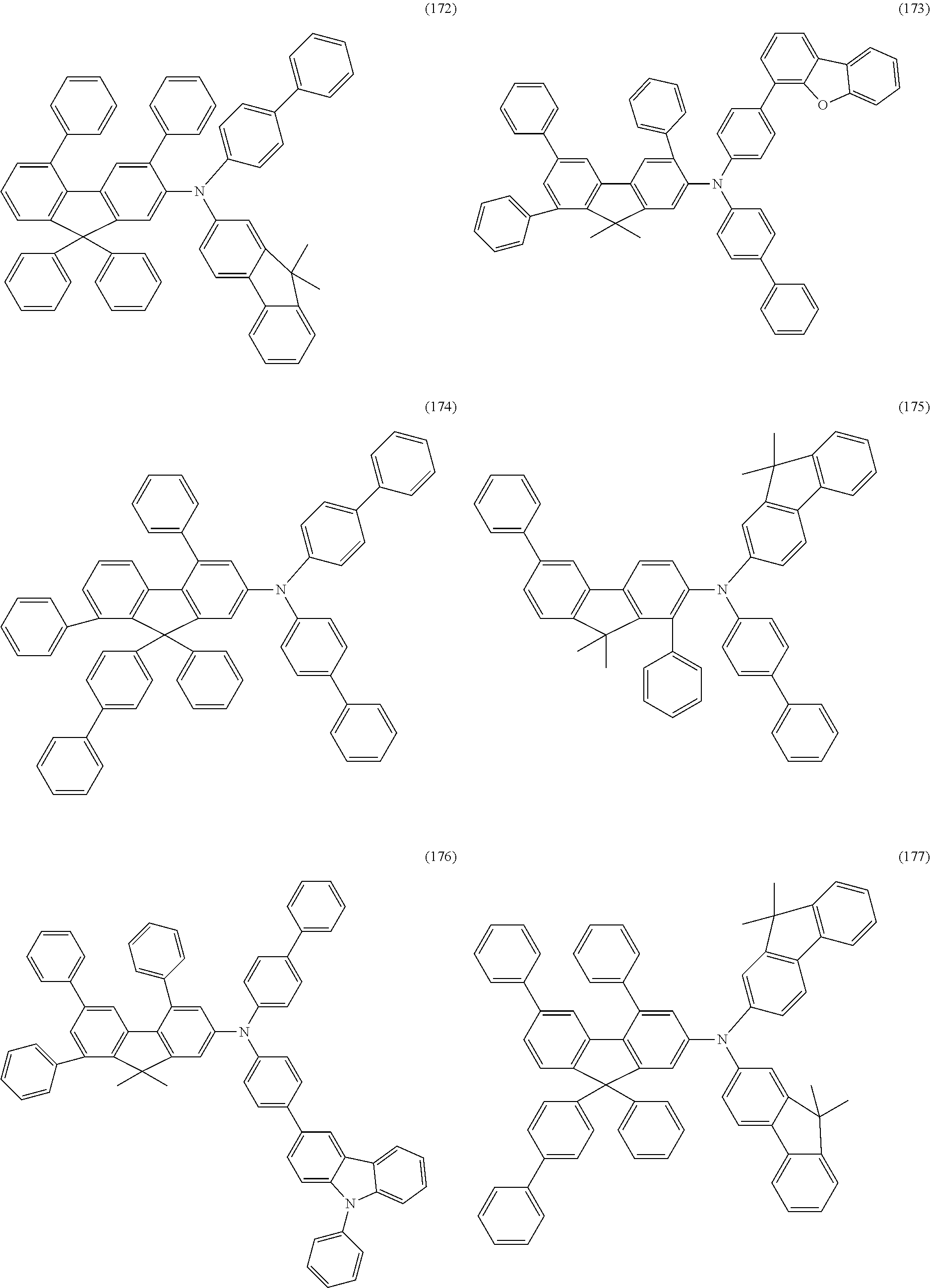

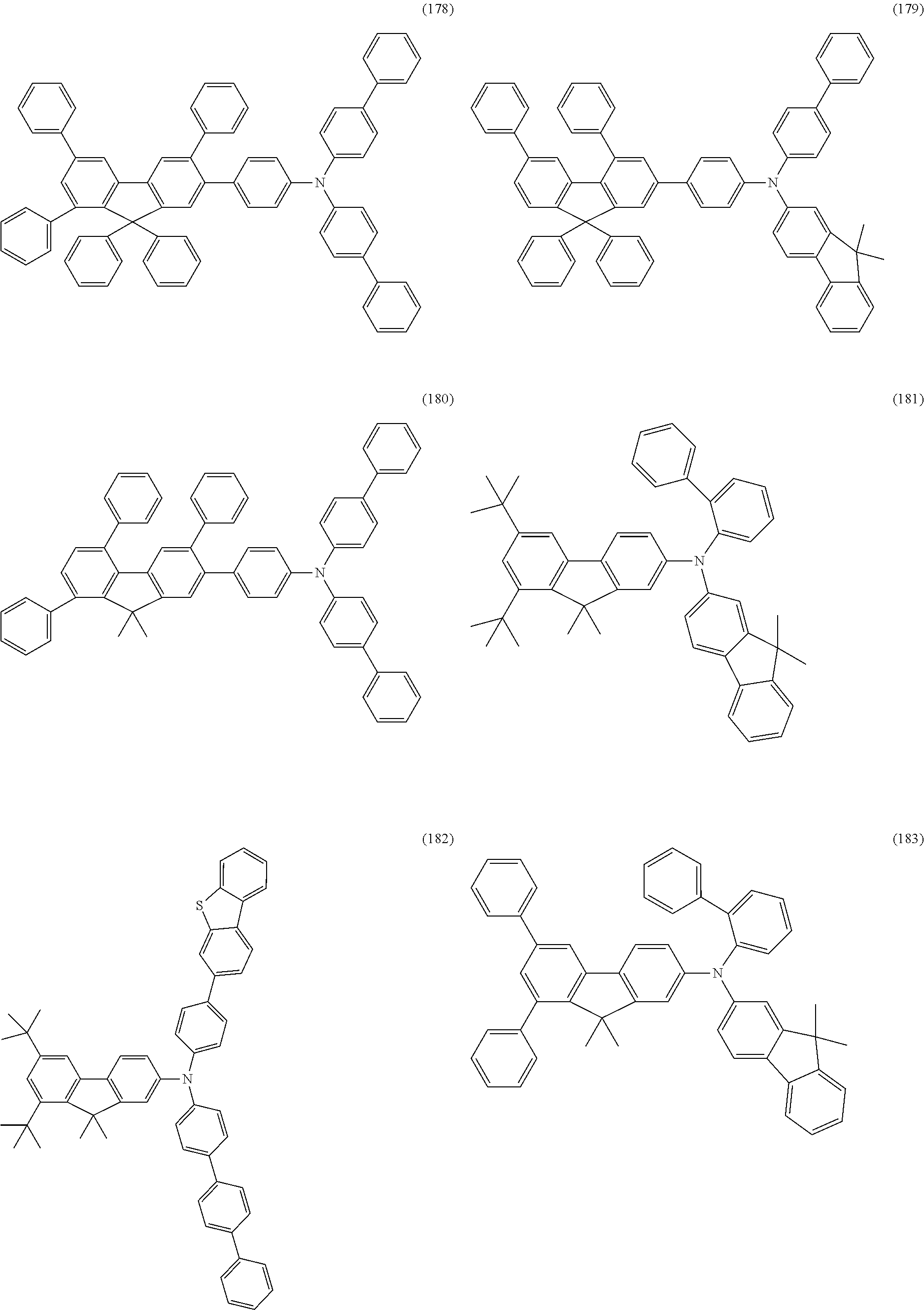

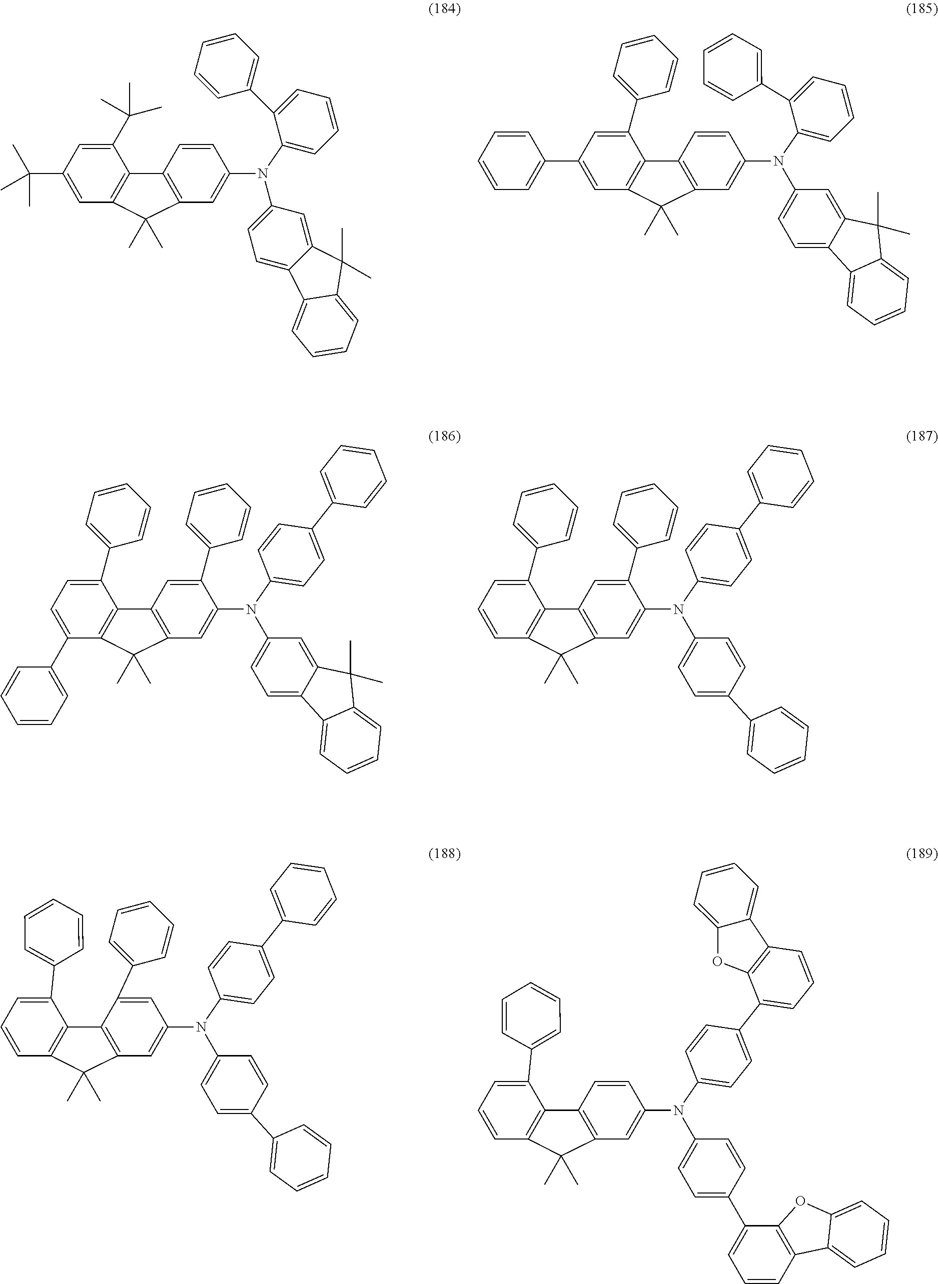

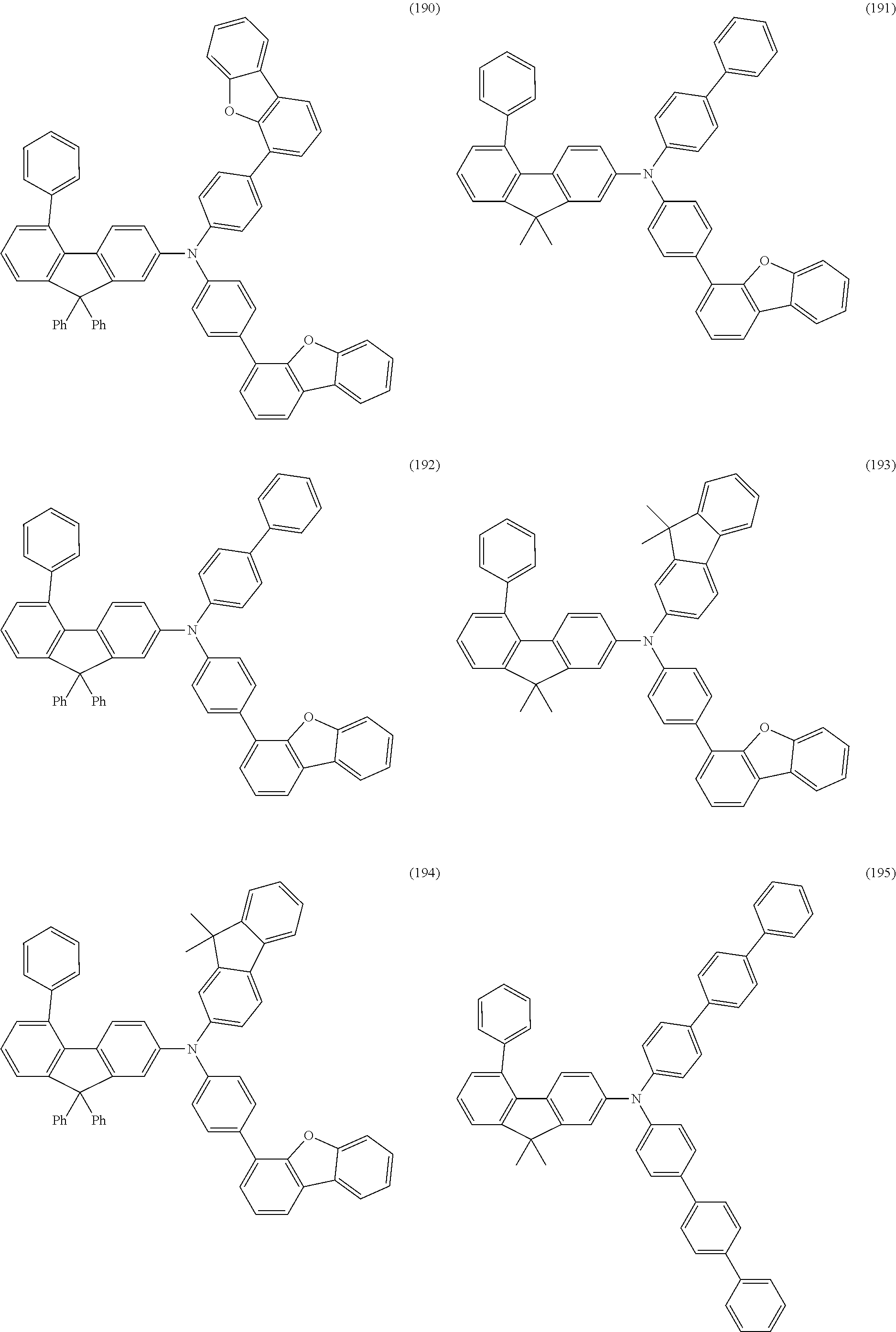

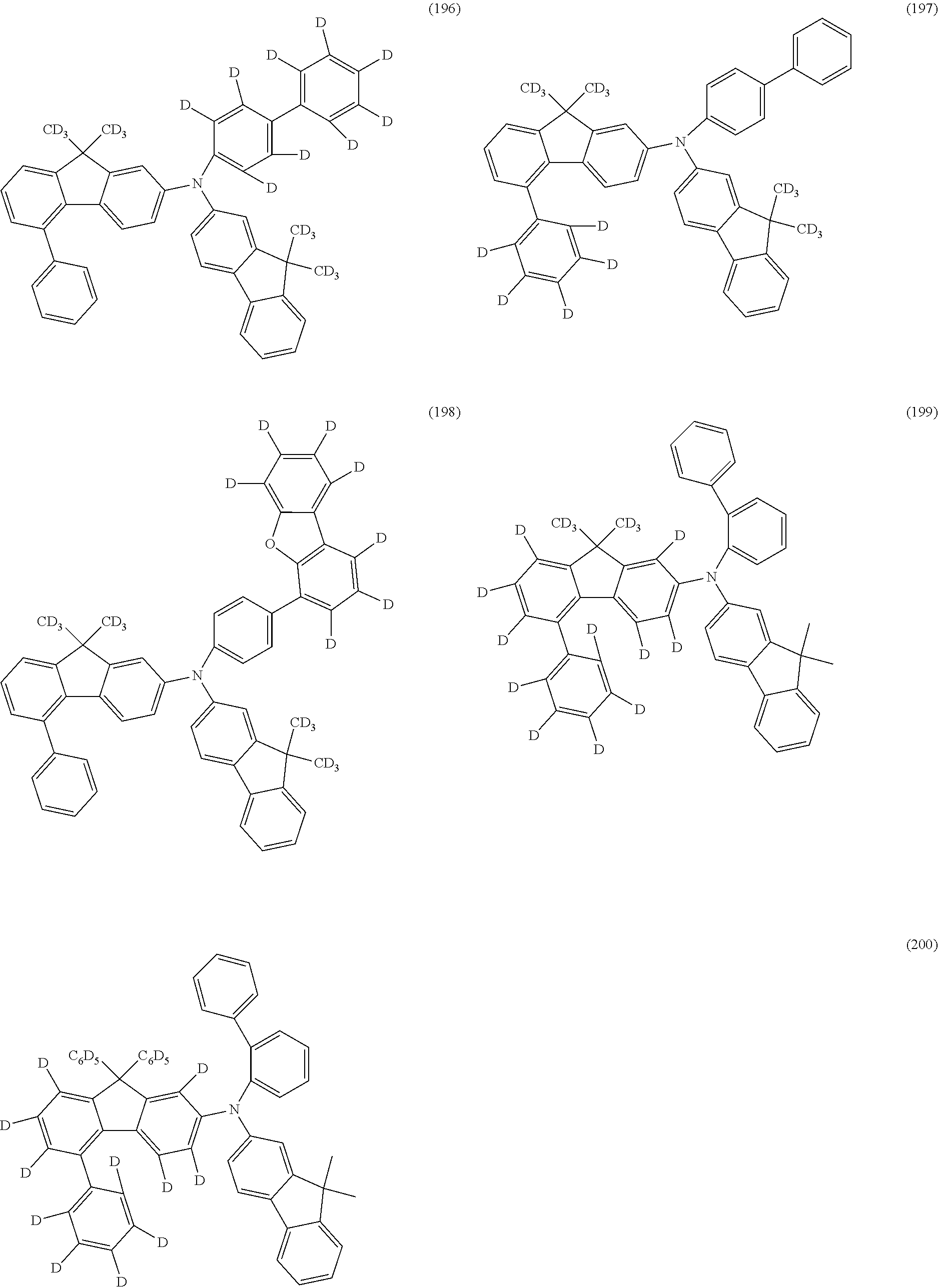

The compounds according to the present application are prepared by using standard methods known in the art of organic synthesis, such as halogenation and metal catalyzed coupling reactions, in particular Suzuki reactions and Buchwald reactions.

The following scheme shows a preferred synthesis process for the synthesis of compounds according to formula (I) of the present application. According to this synthesis process, a fluorene derivative A, which has a leaving group preferably at 2-position, is reacted via a C—N coupling reaction, preferably a Buchwald coupling reaction, with a diarylamino derivative B of formula Ar²—NH—Ar¹:

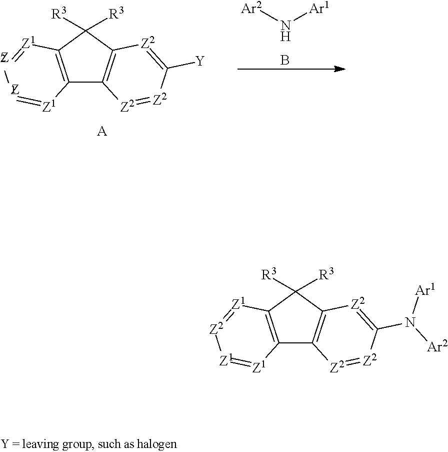

The following scheme shows another preferred synthesis process for synthesizing compounds of formula (I) according to the present application. Here, fluorene derivative C, which has a leaving group preferably at 5- and 2-position, respectively, is reacted via a Suzuki coupling reaction with a boronic acid D of the formula R¹—B(OH)₂. A following Buchwald coupling reaction of the resulting intermediate Eat 2-position with a diarylamino derivative of formula Ar²—NH—Ar¹ results in corresponding compounds according to formula (I) of the present application:

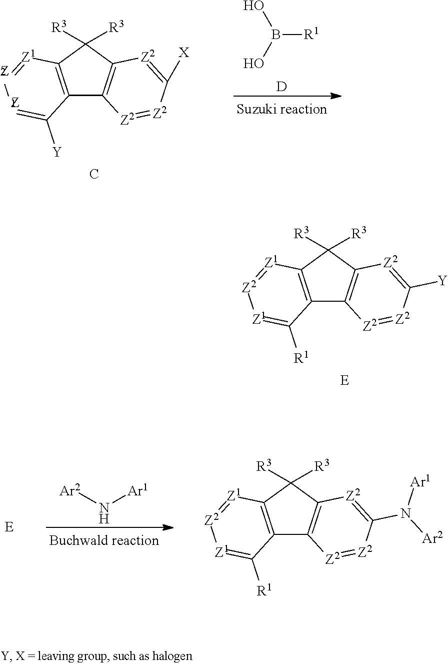

The variables occurring in these schemes are defined as above.

As a result of the above-mentioned reactions, compounds according to formula (I) of the present application are obtained.

A further embodiment of the present invention is therefore a process for preparation of a compound according to formula (I), comprising introducing a diarylamino group by a C—N coupling reaction between a fluorene derivative, which is halogenated at 2-position, and a diarylamine derivative.

Synthesis processes for obtaining fluorine derivatives A and C and diarylamine derivative C used for synthesizing the compounds according to the present invention are known to those skilled in the art.

In particular, compounds according to formula (I) of the present invention can be prepared by reacting an alkyl 5-halo-2-iodobenzoate with an arylboronic acid as the starting compounds via a Suzuki coupling reaction.

Particularly preferred, the process for preparing compounds according to formula (I) of the present invention comprises the following reaction steps:
a) Reacting a methyl 5-halo-2-iodobenzoate of general formula (II)
b)

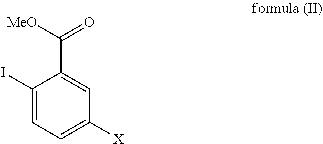

(in which X=Cl or Br) with an arylboronic acid derivative which conforms to one of formulae (III-1) to (III-5)

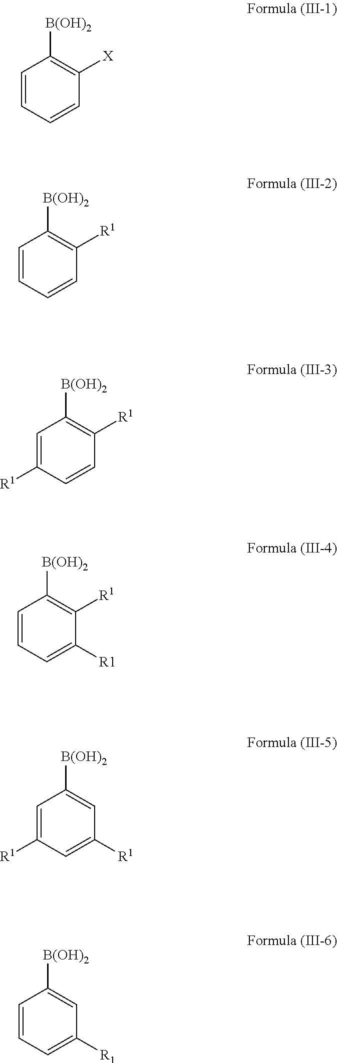

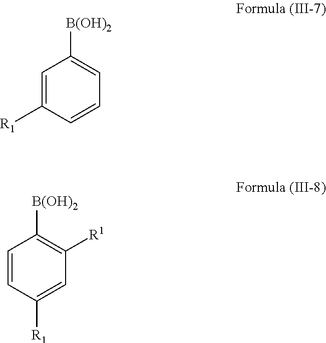

in which
R¹ is, identically or differently on each occurrence, as defined above, but is preferably selected from phenyl, biphenyl, terphenyl or quaterphenyl, each of which may optionally be substituted by one or more radicals R⁵ as defined above; and
X is Cl or Br;
to obtain a 5-halobenzoate methyl ester derivative, and subsequently
c) converting the ester derivative to a tertiary alcohol by using an alkyl- or aryl-magnesium halide, and subsequently
d) performing acid-catalyzed cyclisation to obtain a fluorene derivative, which is halogenated at 2-position, and subsequently
e) reacting the fluorene derivative with a diarylamine derivative to obtain a compound of formula (I).

The alkyl- or aryl-magnesium halide in step b) is preferably a methyl- or phenyl-magnesium chloride as commonly used for a Grignard reaction, without being limited thereto. As the acid for catalyzing the cyclisation in step c), for example BF₃.Et₂O can be used. As the catalyst for the Suzuki coupling reaction in step a), Pd(P(Ph₃))₄ may be used, without being limited thereto. The reaction conditions for performing a Suzuki coupling reaction, a Grignard reaction and the cyclization are known to a person skilled in the art.

Specific examples of arylboronic acid that may be used compounds are:

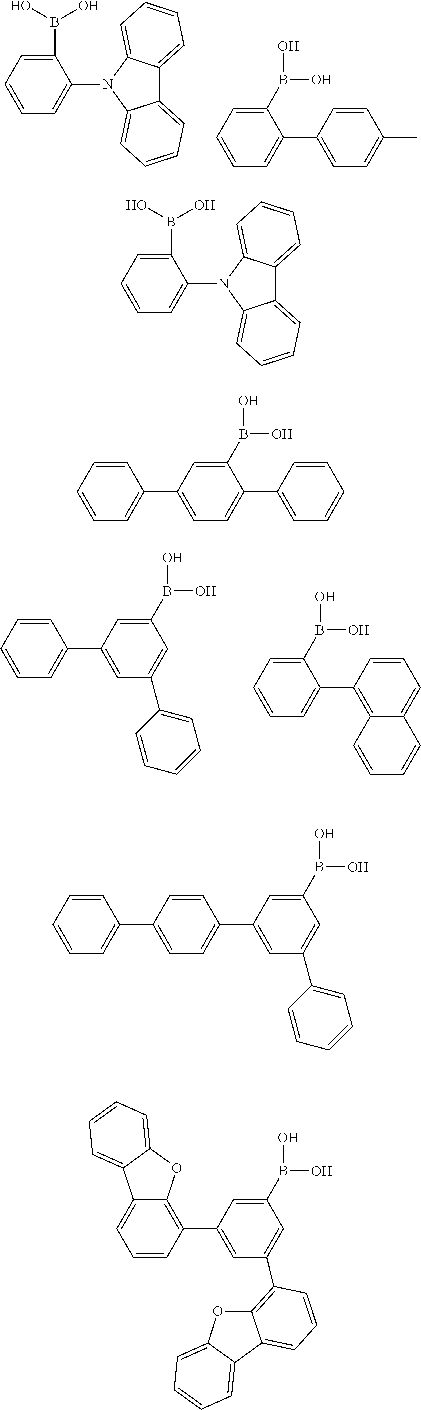

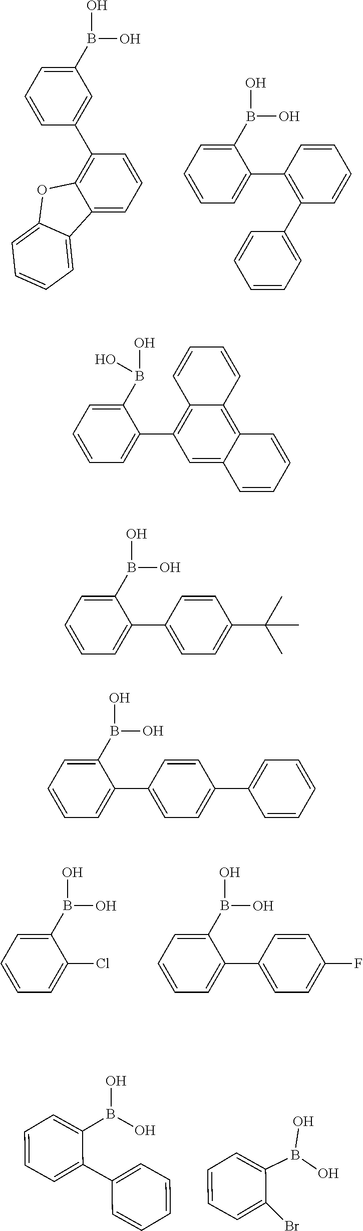

In an alternatively preferred process, compounds according to formula (I) of the present invention can be prepared by the following reaction steps:
a-1) reacting biphenyl, which is halogenated at least in 2- and 4-position, with a diaryl, dialkyl or arylalkyl ketone derivative, for example a benzophenone derivative, using a organometallic compound, and subsequently
b-1) performing acid-catalyzed cyclication to obtain a fluorene derivative, which is halogenated at 2-position, and subsequently
c-1) reacting the fluorene derivative with a diarylamine derivative to obtain a compound of formula (I).

Fluorene derivatives, which are halogenated at 2-position, can be prepared following reaction steps a) to c) or steps a-1) to b-1) described above, or are obtainable or can be obtained or isolated from reaction step c) or b-1) described above.

The present invention thus further provides to fluorene derivatives which conform to one of formulae (IV-A) to (IV-L)

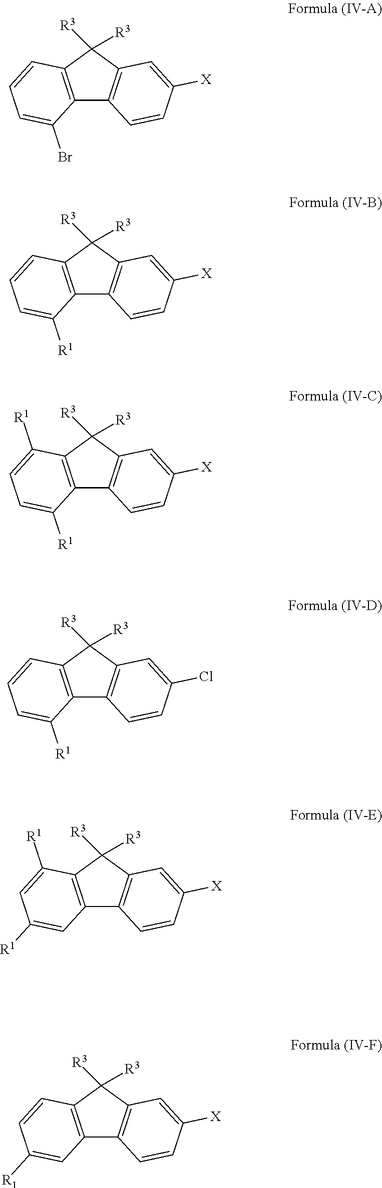

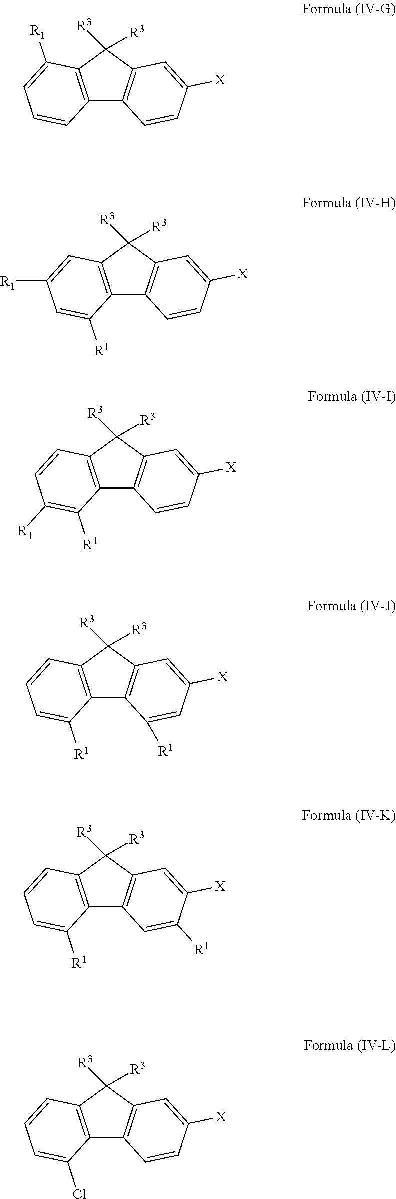

in which
R¹ is selected, identically or differently on each occurrence, from phenyl, biphenyl, terphenyl or quaterphenyl, each of which may optionally be substituted by one or more radicals R⁵ as defined above;
R³ is selected, identically or differently on each occurrence, from methyl -CD₃, and phenyl or deuterated phenyl (C₆D₅), each of which may optionally be substituted by one or more radicals R⁵ as defined above; and
X is Cl or Br.

Particularly preferred fluorene derivatives of the present invention conform to one of the following formulae:

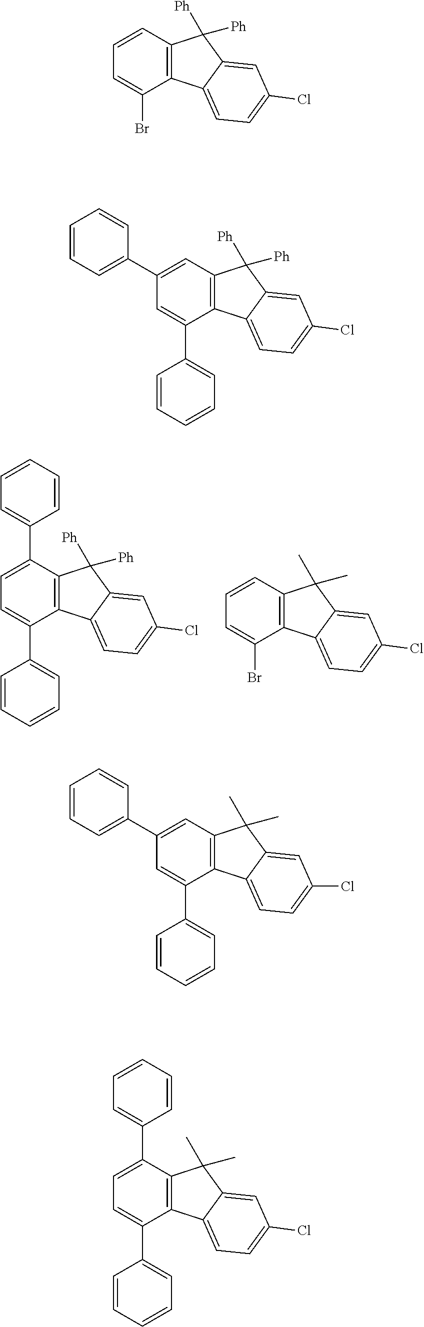

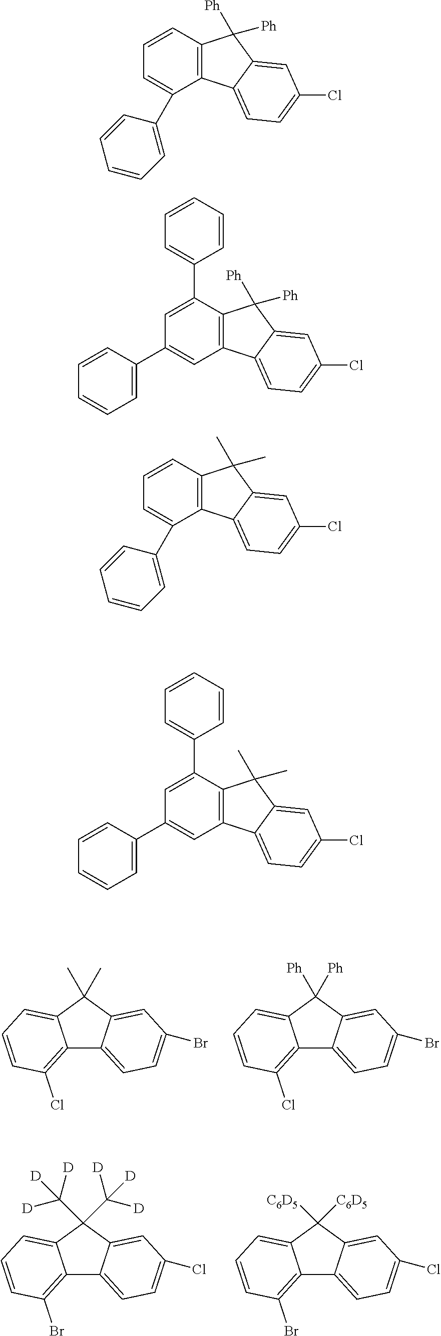

The above-described compounds of formula (I), especially compounds of formula (I) substituted by reactive leaving groups, such as bromine, iodine, chlorine, boronic acid or boronic ester, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C═C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of formula (I), wherein the bond(s) to the polymer, oligomer or dendrimer may be localized at any desired positions substituted by R¹, R², R³, R⁴, R⁵ or R⁶ in formula (I). According to the linkage of the compound of formula (I), the compound is part of a side chain of the oligomer or polymer or part of the main chain. An oligomer in the context of this invention is understood to mean a compound formed from at least three monomer units. A polymer in the context of the invention is understood to mean a compound formed from at least ten monomer units. The polymers, oligomers or dendrimers of the invention may be conjugated, partly conjugated or non-conjugated. The oligomers or polymers of the invention may be linear, branched or dendritic. In the structures having linear linkage, the units of formula (I) may be joined directly to one another, or they may be joined to one another via a bivalent group, for example via a substituted or unsubstituted alkylene group, via a heteroatom or via a bivalent aromatic or heteroaromatic group. In branched and dendritic structures, it is possible, for example, for three or more units of formula (I) to be joined via a trivalent or higher-valency group, for example via a trivalent or higher-valency aromatic or heteroaromatic group, to give a branched or dendritic oligomer or polymer.

For the repeat units of formula (I) in oligomers, dendrimers and polymers, the same preferences apply as described above for compounds of formula (I).

In order to prepare the oligomers or polymers, the monomers of the invention are homopolymerized or copolymerized with further monomers. Suitable and preferred comonomers are chosen from fluorenes (for example according to EP 842208 or WO 2000/22026), spirobifluorenes (for example according to EP 707020, EP 894107 or WO 2006/061181), paraphenylenes (for example according to WO 1992/18552), carbazoles (for example according to WO 2004/070772 or WO 2004/113468), thiophenes (for example according to EP 1028136), dihydrophenanthrenes (for example according to WO 2005/014689 or WO 2007/006383), cis- and trans-indenofluorenes (for example according to WO 2004/041901 or WO 2004/113412), ketones (for example according to WO 2005/040302), phenanthrenes (for example according to WO 2005/104264 or WO 2007/017066) or else a plurality of these units. The polymers, oligomers and dendrimers typically contain still further units, for example emitting (fluorescent or phosphorescent) units, for example vinyltriarylamines (for example according to WO 2007/068325) or phosphorescent metal complexes (for example according to WO 2006/003000), and/or charge transport units, especially those based on triarylamines.

The polymers and oligomers of the invention are generally prepared by polymerization of one or more monomer types, of which at least one monomer leads to repeat units of the formula (I) in the polymer. Suitable polymerization reactions are known to those skilled in the art and are described in the literature. Particularly suitable and preferred polymerization reactions which lead to formation of C—C or C—N bonds are the Suzuki polymerization, the Yamamoto polymerization, the Stille polymerization and the Hartwig-Buchwald polymerization.

The compounds according to the present invention may be used or applied together with further organic functional materials, which are commonly used in electronic devices according to the prior art. A great variety of suitable organic functional materials is known to those skilled in the art in the field of electronic devices. The present invention therefore further provides for a composition comprising one or more compounds of formula (I), or one or more polymers, oligomers or dendrimers containing one or more compounds of formula (I), and at least one further organic functional material selected from the group consisting of fluorescent emitters, phosphorescent emitters, host materials, matrix materials, electron transporting materials, electron injecting materials, hole transporting materials, hole injecting materials, electron blocking materials, hole blocking materials, wide band gap materials, delayed fluorescent emitters and delayed fluorescent hosts.

Delayed fluorescent emitters and delayed fluorescent hosts are well known in the art and disclosed in, e.g., Ye Tao et al., Adv. Mater. 2014, 26, 7931-7958, M. Y. Wong et al., Adv. Mater. 2017, 29, 1605444, WO 2011/070963, WO 2012/133188, WO 2015/022974 and WO 2015/098975. Typically, the delayed fluorescent materials (emitters and/or hosts) are characterized in that they exhibit a rather small gap between their singlet energy (S₁) and triplet energy (T₁). Preferably ΔE_{ST} is equal to or smaller than 0.5 eV, very preferably equal to or smaller than 0.3 eV, particularly preferably equal to or smaller than 0.2 eV and most preferably equal to or small than 0.1 eV, wherein ΔE_{ST} represents the difference between the singlet energy (S₁) and the triplet energy (T₁).

Within the present invention, wide band gap materials are understood to mean a material as disclosed in U.S. Pat. No. 7,294,849, which is characterized in having a band gap of at least 3 eV, preferably at least 3.5 eV and very preferably at least 4.0 eV, wherein the term “band gap” means the energy gap between the highest occupied molecular orbital (HOMO) and the lowest unoccupied molecular orbital (LUMO). Such systems exhibit particularly advantageous performance characteristics in electroluminescent devices.

For the processing of the compounds and compositions of the invention from a liquid phase, for example by spin-coating or by printing methods, formulations of the compounds and compositions of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, a-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane or mixtures of these solvents.

The invention therefore further provides a formulation, especially a solution, dispersion or emulsion, comprising at least one compound of formula (I), or oligomers, polymers or dendrimers containing one or more compounds of formula (I), or at least one composition comprising one or more compounds of formula (I) and at least one further organic functional material, as described above, and at least one solvent, preferably an organic solvent. The way in which such solutions can be prepared is known to those skilled in the art and is described, for example, in WO 2002/072714, WO 2003/019694 and the literature cited therein.

The compounds of the invention are suitable for use in electronic devices, especially in organic electroluminescent devices such as OLEDs. Depending on the substitution, the compounds are used in different functions and layers.

The invention therefore further provides for the use of the compound of formula (I), or an oligomer, polymers or dendrimer containing one or more compounds of formula (I), or a composition comprising one or more compounds of formula (I) and at least one further organic functional material, as described above, in an electronic device. This electronic device is preferably selected from the group consisting of organic integrated circuits (OICs), organic field-effect transistors (OFETs), organic thin-film transistors (OTFTs), organic solar cells (OSCs), organic optical detectors, organic photoreceptors and, more preferably, organic electroluminescent devices (EL devices). Preferred EL devices are organic light-emitting transistors (OLETs), organic field-quench devices (OFQDs), organic light-emitting electrochemical cells (OLECs, LECs, LEECs), organic laser diodes (O-lasers) and organic light emitting diodes (OLEDs), of which OLEDs are most preferred.

The invention further provides, as already set out above, an electronic device comprising at least one compound of formula (I). This electronic device is preferably selected from the abovementioned devices.

Particularly preferably, the electronic device is an organic light emitting diode (OLED) comprising anode, cathode and at least one emitting layer, characterized in that at least one organic layer, which may be an emitting layer, a hole transport layer or another layer, preferably an emitting layer or a hole transport layer, particularly preferably a hole transport layer, comprises at least one compound of formula (I).

Within the present invention, the term “organic layer” is understood to mean any layer of an electronic device which comprises one or more organic compounds as functional materials.

Apart from the cathode, anode and emitting layer, the organic light emitting diode may also comprise further layers. These are selected, for example, from in each case one or more hole injection layers, hole transport layers, hole blocking layers, electron transport layers, electron injection layers, electron blocking layers, exciton blocking layers, interlayers, charge generation layers (IDMC 2003, Taiwan; Session 21 OLED (5), T. Matsumoto, T. Nakada, J. Endo, K. Mori, N. Kawamura, A. Yokoi, J. Kido, Multiphoton Organic EL Device Having Charge Generation Layer) and/or organic or inorganic p/n junctions.

The sequence of the layers of the organic light emitting diode comprising the compound of the formula (I) is preferably as follows:

anode-hole injection layer-hole transport layer-optionally further hole transport layer(s)-optionally electron blocking layer-emitting layer-optionally hole blocking layer-electron transport layer-electron injection layer-cathode. It is additionally possible for further layers to be present in the OLED.

The organic light emitting diode of the invention may contain two or more emitting layers. More preferably, these emission layers in this case have several emission maxima between 380 nm and 750 nm overall, such that the overall result is white emission; in other words, various emitting compounds which may fluoresce or phosphoresce and which emit blue, green, yellow, orange or red light are used in the emitting layers. Especially preferred are three-layer systems, i.e. systems having three emitting layers, where the three layers show blue, green and orange or red emission (for the basic construction see, for example, WO 2005/011013). The compounds of the invention are preferably present in the hole transport layer, hole injection layer or electron blocking layer, most preferably in the electron blocking layer.

It is preferable in accordance with the invention when the compound of formula (I) is used in an electronic device comprising one or more phosphorescent emitting compounds. In this case, the compound may be present in different layers, preferably in a hole transport layer, an electron blocking layer, a hole injection layer or in an emitting layer.

The term “phosphorescent emitting compounds” typically encompasses compounds where the emission of light is effected through a spin-forbidden transition, for example a transition from an excited triplet state or a state having a higher spin quantum number, for example a quintet state.

Suitable phosphorescent emitting compounds (=triplet emitters) are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitting compounds, compounds containing copper, molybdenum, tungsten, rhenium, ruthenium, osmium, rhodium, iridium, palladium, platinum, silver, gold or europium, especially compounds containing iridium, platinum or copper. In the context of the present invention, all luminescent iridium, platinum or copper complexes are considered to be phosphorescent emitting compounds.

Examples of the above-described emitting compounds can be found in applications WO 00/70655, WO 01/41512, WO 02/02714, WO 02/15645, EP 1191613, EP 1191612, EP 1191614, WO 05/033244, WO 05/019373 and US 2005/0258742. In general, all phosphorescent complexes as used for phosphorescent OLEDs according to the prior art and as known to those skilled in the art in the field of organic electroluminescent devices are suitable. It is also possible for the person skilled in the art, without exercising inventive skill, to use further phosphorescent complexes in combination with the compounds of formula (I) in organic electroluminescent devices. Further examples are listed in a table which follows.

It is also possible in accordance with the invention to use the compound of formula (I) in an electronic device comprising one or more fluorescent emitting compounds.

In a preferred embodiment of the invention, the compounds of formula (I) are used as hole-transporting material. In that case, the compounds are preferably present in a hole transport layer, an electron blocking layer or a hole injection layer. Particular preference is given to use in an electron blocking layer.

A hole transport layer according to the present application is a layer having a hole-transporting function between the anode and emitting layer.

Hole injection layers and electron blocking layers are understood in the context of the present application to be specific embodiments of hole transport layers. A hole injection layer, in the case of a plurality of hole transport layers between the anode and emitting layer, is a hole transport layer which directly adjoins the anode or is separated therefrom only by a single coating of the anode. An electron blocking layer, in the case of a plurality of hole transport layers between the anode and emitting layer, is that hole transport layer which directly adjoins the emitting layer on the anode side. Preferably, the OLED of the invention comprises two, three or four hole-transporting layers between the anode and emitting layer, at least one of which preferably contains a compound of formula (I), and more preferably exactly one or two contain a compound of formula (I).

If the compound of formula (I) is used as hole transport material in a hole transport layer, a hole injection layer or an electron blocking layer, the compound can be used as pure material, i.e. in a proportion of 100%, in the hole transport layer, or it can be used in combination with one or more further compounds. In a preferred embodiment, the organic layer comprising the compound of the formula (I) then additionally contains one or more p-dopants. p-Dopants used according to the present invention are preferably those organic electron acceptor compounds capable of oxidizing one or more of the other compounds in the mixture.

Particularly preferred embodiments of p-dopants are the compounds disclosed in WO 2011/073149, EP 1968131, EP 2276085, EP 2213662, EP 1722602, EP 2045848, DE 102007031220, U.S. Pat. Nos. 8,044,390, 8,057,712, WO 2009/003455, WO 2010/094378, WO 2011/120709, US 2010/0096600, WO 2012/095143 and DE 102012209523.

Particularly preferred p-dopants are quinodimethane compounds, azaindenofluorenediones, azaphenalenes, azatriphenylenes, I₂, metal halides, preferably transition metal halides, metal oxides, preferably metal oxides containing at least one transition metal or a metal of main group 3, and transition metal complexes, preferably complexes of Cu, Co, Ni, Pd and Pt with ligands containing at least one oxygen atom as bonding site. Preference is further given to transition metal oxides as dopants, preferably oxides of rhenium, molybdenum and tungsten, more preferably Re₂O₇, MoO₃, WO₃ and ReO₃.

The p-dopants are preferably in substantially homogeneous distribution in the p-doped layers. This can be achieved, for example, by coevaporation of the p-dopant and the hole transport material matrix.

Preferred p-dopants are especially the following compounds:

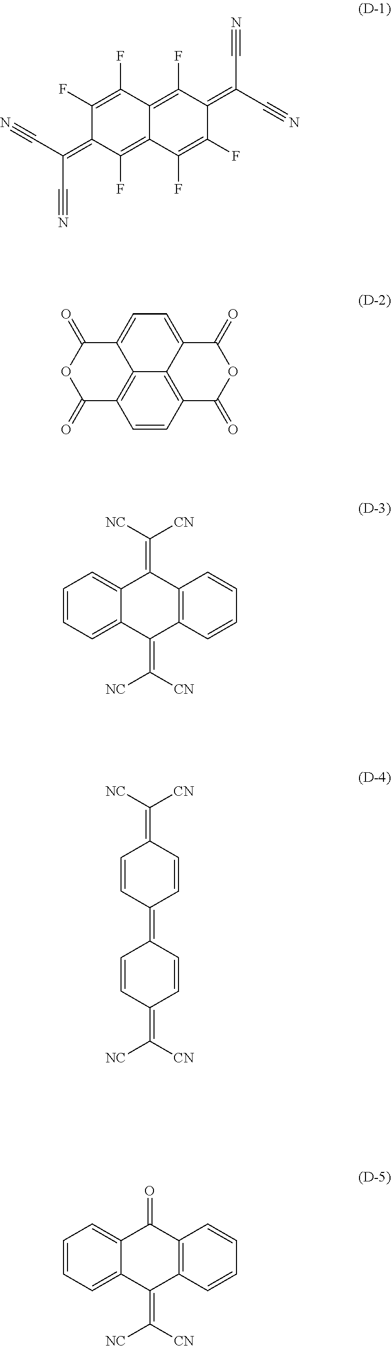 [SMILES: N#CC(C#N)=c1c(F)c(F)c2c(F)c(=C(C#N)C#N)c(F)c(F)c2c1F ; O=C1OC(=O)c2ccc3c4c(ccc1c24)C(=O)OC3=O ; N#CC(C#N)=c1c2ccccc2c(=C(C#N)C#N)c2ccccc12 ; N#CC(C#N)=c1ccc(=c2ccc(=C(C#N)C#N)cc2)cc1 ; N#CC(C#N)=C1c2ccccc2C(=O)c2ccccc21]

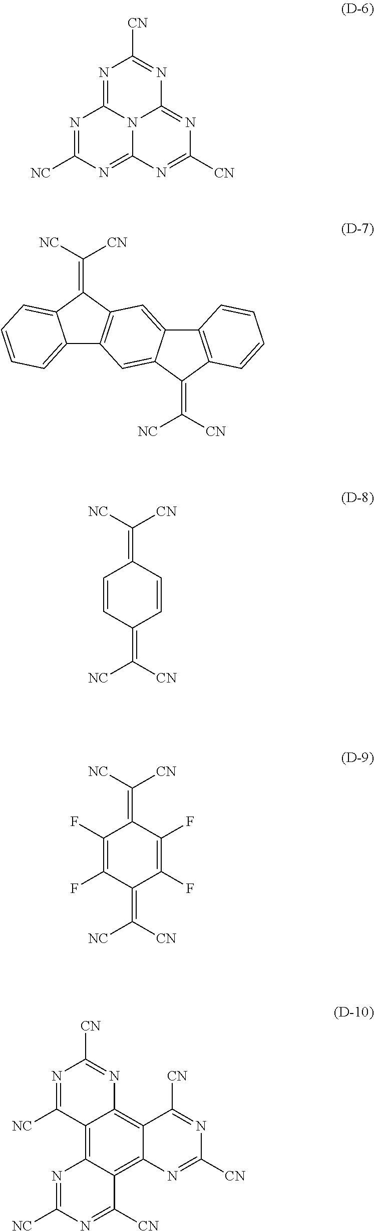 [SMILES: N#CC1=NC2=NC(C#N)=NC3=NC(C#N)=NC(=N1)N23 ; N#CC(C#N)=C1c2ccccc2-c2cc3c(cc21)-c1ccccc1C3=C(C#N)C#N ; N#CC(C#N)=c1ccc(=C(C#N)C#N)cc1 ; N#CC(C#N)=c1c(F)c(F)c(=C(C#N)C#N)c(F)c1F ; N#Cc1nc(C#N)c2c(n1)c1c(C#N)nc(C#N)nc1c1c(C#N)nc(C#N)nc21]

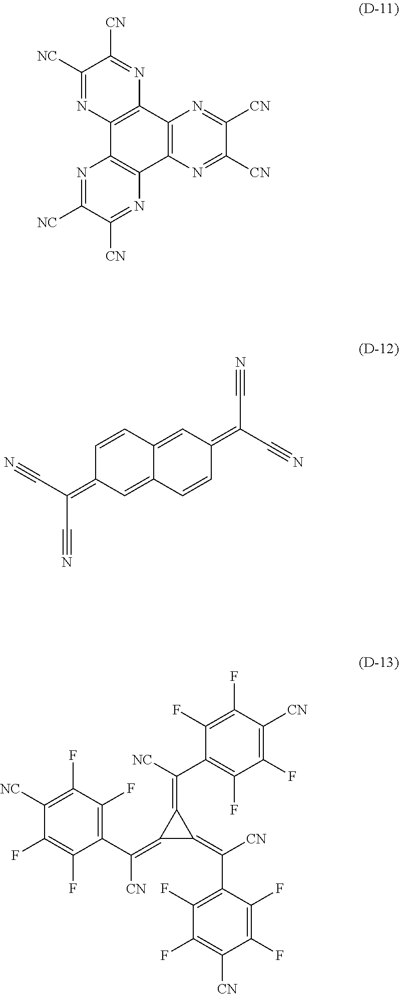 [SMILES: N#Cc1nc2c3nc(C#N)c(C#N)nc3c3nc(C#N)c(C#N)nc3c2nc1C#N ; N#CC(C#N)=c1ccc2cc(=C(C#N)C#N)ccc2c1 ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

In a further preferred embodiment of the invention, the compound of formula (I) is used as hole transport material in combination with a hexaazatriphenylene derivative as described in US 2007/0092755. Particular preference is given here to using the hexaazatriphenylene derivative in a separate layer.

Further hole transport materials that can be used in any of the layers that require materials with hole transporting capabilities, e.g. hole injection layer (HIL), hole transport layer (HTL), electron blocking layer (EBL) or the emissive layer (EML) are listed in the following table. The compounds can be prepared easily according to the disclosure cited for each of the compounds. The compounds (1) to (22) exhibit excellent stability and electronic devices comprising the compounds show high efficiencies, low voltages and improved lifetimes.

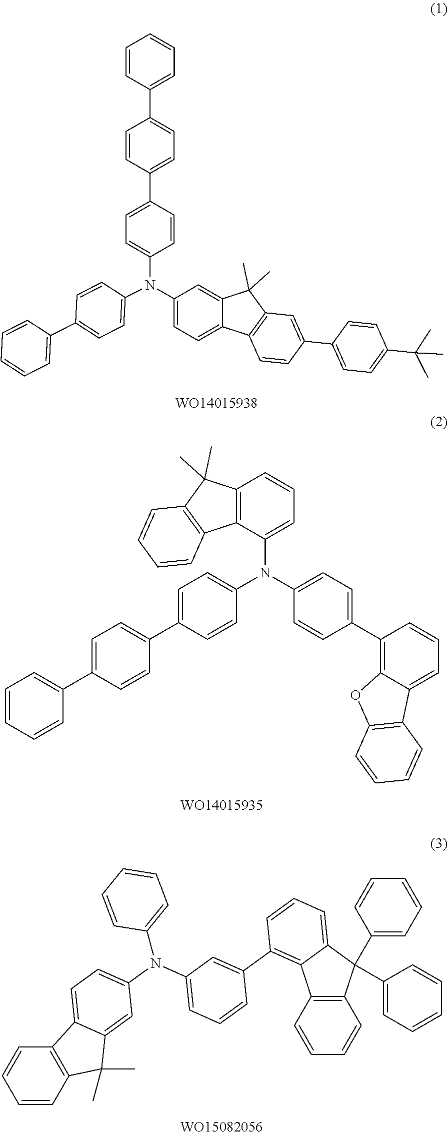 [SMILES: CC(C)(C)c1ccc(-c2ccc3c(c2)C(C)(C)c2cc(N(c4ccc(-c5ccccc5)cc4)c4ccc(-c5ccc(-c6ccccc6)cc5)cc4)ccc2-3)cc1 ; CC1(C)c2ccccc2-c2c(N(c3ccc(-c4ccc(-c5ccccc5)cc4)cc3)c3ccc(-c4cccc5c4oc4ccccc45)cc3)cccc21 ; CC1(C)c2ccccc2-c2ccc(N(c3ccccc3)c3cccc(-c4cccc5c4-c4ccccc4C5(c4ccccc4)c4ccccc4)c3)cc21]

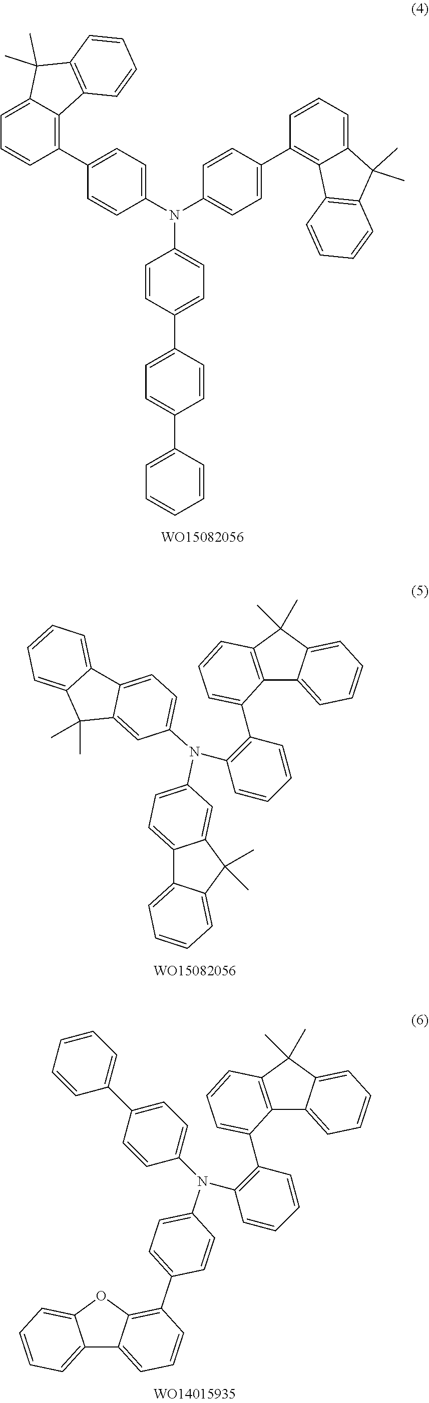 [SMILES: CC1(C)c2ccccc2-c2c(-c3ccc(N(c4ccc(-c5ccc(-c6ccccc6)cc5)cc4)c4ccc(-c5cccc6c5-c5ccccc5C6(C)C)cc4)cc3)cccc21 ; CC1(C)c2ccccc2-c2ccc(N(c3ccc4c(c3)C(C)(C)c3ccccc3-4)c3ccccc3-c3cccc4c3-c3ccccc3C4(C)C)cc21 ; CC1(C)c2ccccc2-c2c(-c3ccccc3N(c3ccc(-c4ccccc4)cc3)c3ccc(-c4cccc5c4oc4ccccc45)cc3)cccc21]

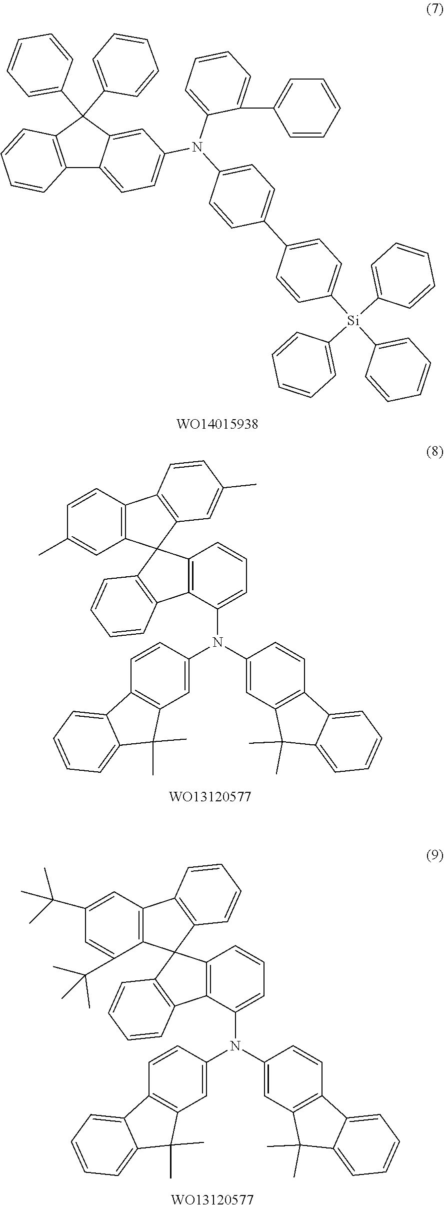 [SMILES: c1ccc(-c2ccccc2N(c2ccc(-c3ccc([Si](c4ccccc4)(c4ccccc4)c4ccccc4)cc3)cc2)c2ccc3c(c2)C(c2ccccc2)(c2ccccc2)c2ccccc2-3)cc1 ; Cc1ccc2c(c1)C1(c3cc(C)ccc3-2)c2ccccc2-c2c(N(c3ccc4c(c3)C(C)(C)c3ccccc3-4)c3ccc4c(c3)C(C)(C)c3ccccc3-4)cccc21 ; CC(C)(C)c1cc2c(c(C(C)(C)C)c1)C1(c3ccccc3-2)c2ccccc2-c2c(N(c3ccc4c(c3)C(C)(C)c3ccccc3-4)c3ccc4c(c3)C(C)(C)c3ccccc3-4)cccc21]

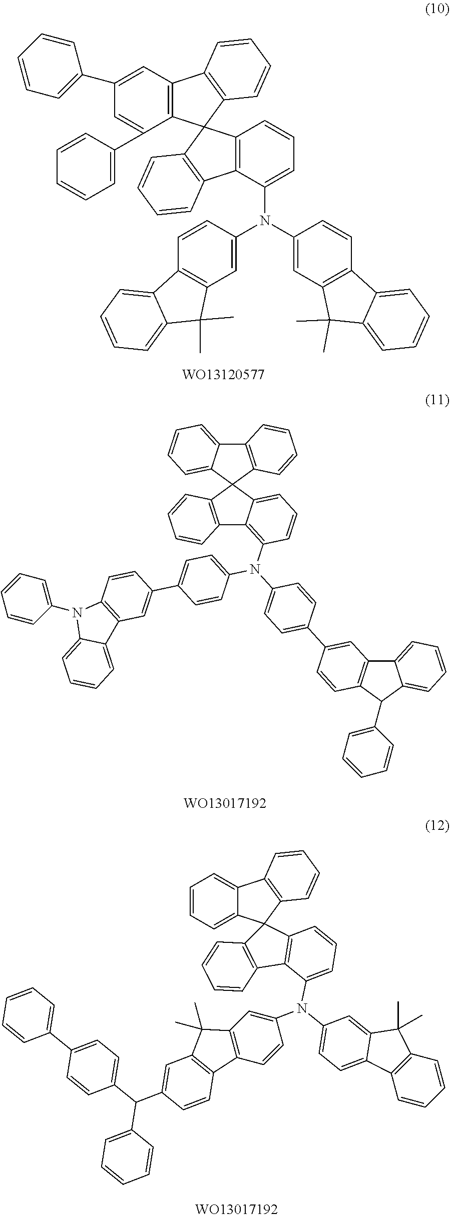 [SMILES: CC1(C)c2ccccc2-c2ccc(N(c3ccc4c(c3)C(C)(C)c3ccccc3-4)c3cccc4c3-c3ccccc3C43c4ccccc4-c4cc(-c5ccccc5)cc(-c5ccccc5)c43)cc21 ; c1ccc(C2c3ccccc3-c3cc(-c4ccc(N(c5ccc(-c6ccc7c(c6)c6ccccc6n7-c6ccccc6)cc5)c5cccc6c5-c5ccccc5C65c6ccccc6-c6ccccc65)cc4)ccc32)cc1 ; CC1(C)c2ccccc2-c2ccc(N(c3ccc4c(c3)C(C)(C)c3cc(C(c5ccccc5)c5ccc(-c6ccccc6)cc5)ccc3-4)c3cccc4c3-c3ccccc3C43c4ccccc4-c4ccccc43)cc21]

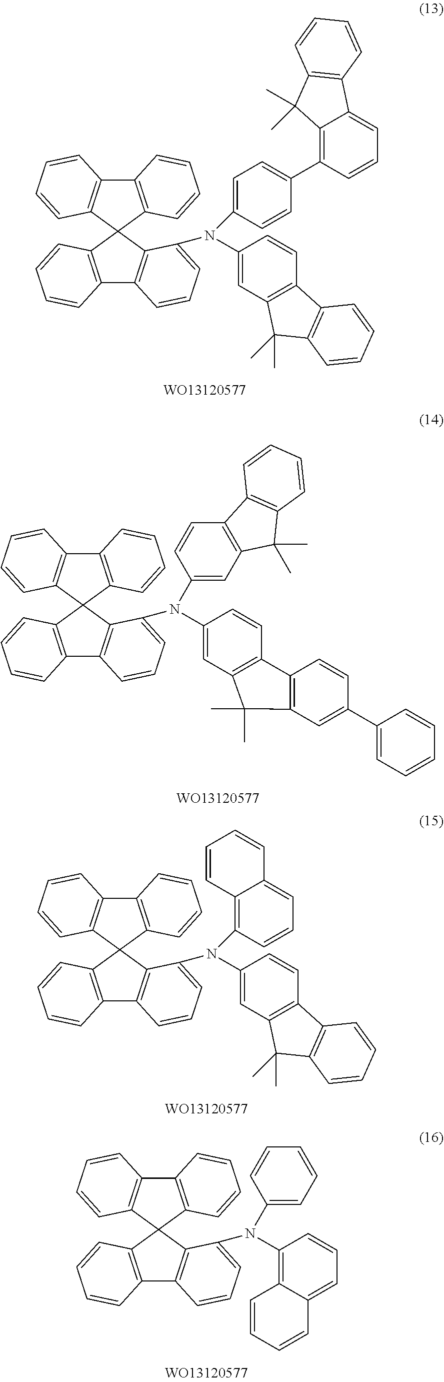 [SMILES: CC1(C)c2ccccc2-c2ccc(N(c3ccc(-c4cccc5c4C(C)(C)c4ccccc4-5)cc3)c3cccc4c3C3(c5ccccc5-c5ccccc53)c3ccccc3-4)cc21 ; CC1(C)c2ccccc2-c2ccc(N(c3ccc4c(c3)C(C)(C)c3cc(-c5ccccc5)ccc3-4)c3cccc4c3C3(c5ccccc5-c5ccccc53)c3ccccc3-4)cc21 ; CC1(C)c2ccccc2-c2ccc(N(c3cccc4c3C3(c5ccccc5-c5ccccc53)c3ccccc3-4)c3cccc4ccccc34)cc21 ; c1ccc(N(c2cccc3c2C2(c4ccccc4-c4ccccc42)c2ccccc2-3)c2cccc3ccccc23)cc1]

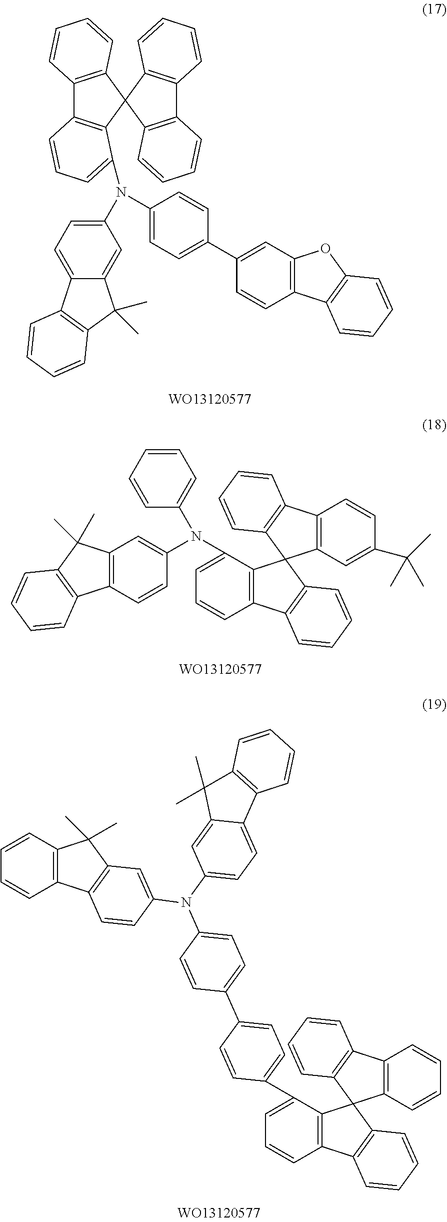 [SMILES: CC1(C)c2ccccc2-c2ccc(N(c3ccc(-c4ccc5c(c4)oc4ccccc45)cc3)c3cccc4c3C3(c5ccccc5-c5ccccc53)c3ccccc3-4)cc21 ; CC(C)(C)c1ccc2c(c1)C1(c3ccccc3-2)c2ccccc2-c2cccc(N(c3ccccc3)c3ccc4c(c3)C(C)(C)c3ccccc3-4)c21 ; CC1(C)c2ccccc2-c2ccc(N(c3ccc(-c4ccc(-c5cccc6c5C5(c7ccccc7-c7ccccc75)c5ccccc5-6)cc4)cc3)c3ccc4c(c3)C(C)(C)c3ccccc3-4)cc21]

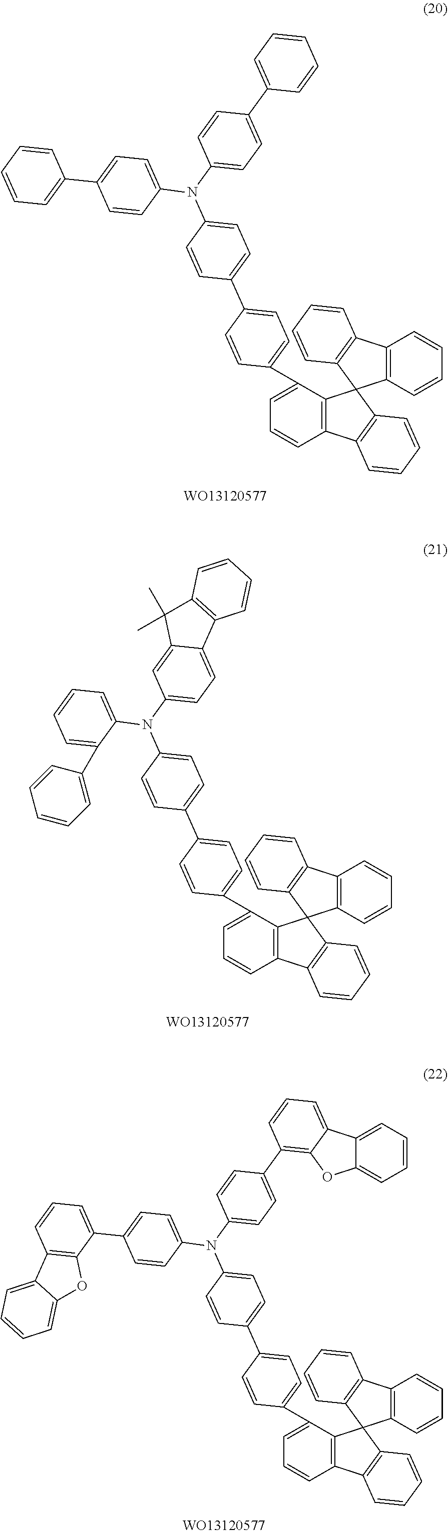 [SMILES: c1ccc(-c2ccc(N(c3ccc(-c4ccccc4)cc3)c3ccc(-c4ccc(-c5cccc6c5C5(c7ccccc7-c7ccccc75)c5ccccc5-6)cc4)cc3)cc2)cc1 ; CC1(C)c2ccccc2-c2ccc(N(c3ccc(-c4ccc(-c5cccc6c5C5(c7ccccc7-c7ccccc75)c5ccccc5-6)cc4)cc3)c3ccccc3-c3ccccc3)cc21 ; c1ccc2c(c1)-c1ccccc1C21c2ccccc2-c2cccc(-c3ccc(-c4ccc(N(c5ccc(-c6cccc7c6oc6ccccc67)cc5)c5ccc(-c6cccc7c6oc6ccccc67)cc5)cc4)cc3)c21]

In a further embodiment of the present invention, the compound of the formula (I) is used in an emitting layer as matrix material in combination with one or more emitting compounds, preferably phosphorescent emitting compounds.

The proportion of the matrix material in the emitting layer in this case is between 50.0% and 99.9% by volume, preferably between 80.0% and 99.5% by volume, and more preferably between 92.0% and 99.5% by volume for fluorescent emitting layers and between 85.0% and 97.0% by volume for phosphorescent emitting layers.

Correspondingly, the proportion of the emitting compound is between 0.1% and 50.0% by volume, preferably between 0.5% and 20.0% by volume, and more preferably between 0.5% and 8.0% by volume for fluorescent emitting layers and between 3.0% and 15.0% by volume for phosphorescent emitting layers.

An emitting layer of an organic light emitting diode may also comprise systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds of formula (I) are used as a component of mixed matrix systems. The mixed matrix systems preferably comprise two or three different matrix materials, more preferably two different matrix materials. Preferably, in this case, one of the two materials is a material having hole-transporting properties and the other material is a material having electron-transporting properties. The compound of the formula (I) is preferably the matrix material having hole-transporting properties. The desired electron-transporting and hole-transporting properties of the mixed matrix components may, however, also be combined mainly or entirely in a single mixed matrix component, in which case the further mixed matrix component(s) fulfill(s) other functions. The two different matrix materials may be present in a ratio of 1:50 to 1:1, preferably 1:20 to 1:1, more preferably 1:10 to 1:1 and most preferably 1:4 to 1:1. Preference is given to using mixed matrix systems in phosphorescent organic light emitting diode. One source of more detailed information about mixed matrix systems is the application WO 2010/108579.

The mixed matrix systems may comprise one or more emitting compounds, preferably one or more phosphorescent emitting compounds. In general, mixed matrix systems are preferably used in phosphorescent organic light emitting diode.

Particularly suitable matrix materials which can be used in combination with the compounds of the invention as matrix components of a mixed matrix system are selected from the preferred matrix materials specified below for phosphorescent emitting compounds or the preferred matrix materials for fluorescent emitting compounds, according to what type of emitting compound is used in the mixed matrix system.

Preferred phosphorescent emitting compounds for use in mixed matrix systems are the same as detailed further up as generally preferred phosphorescent emitter materials.

Preferred embodiments of the different functional materials in the electronic device are listed hereinafter.

Preferred phosphorescent emitting compounds are the following ones:

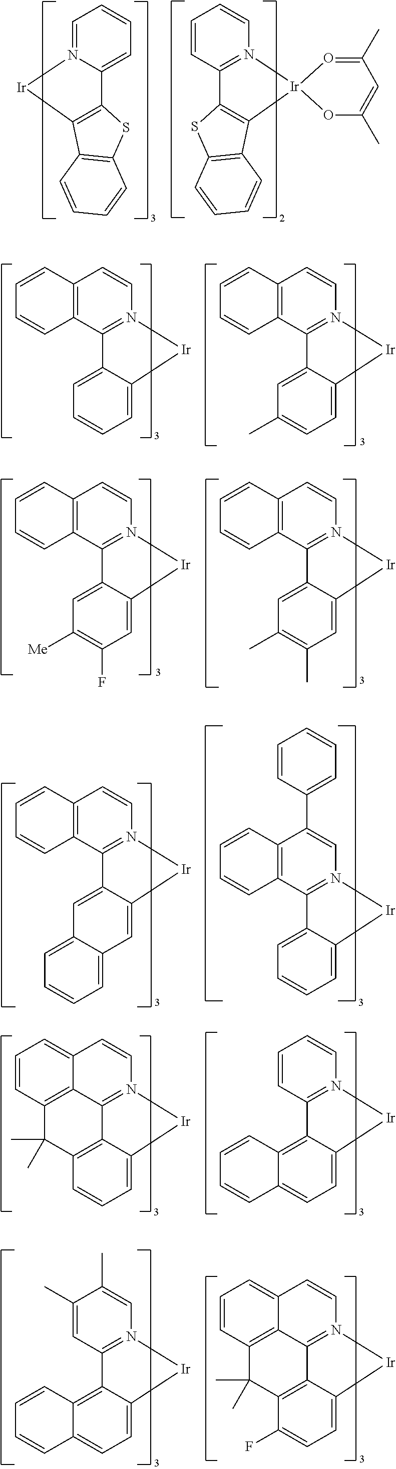

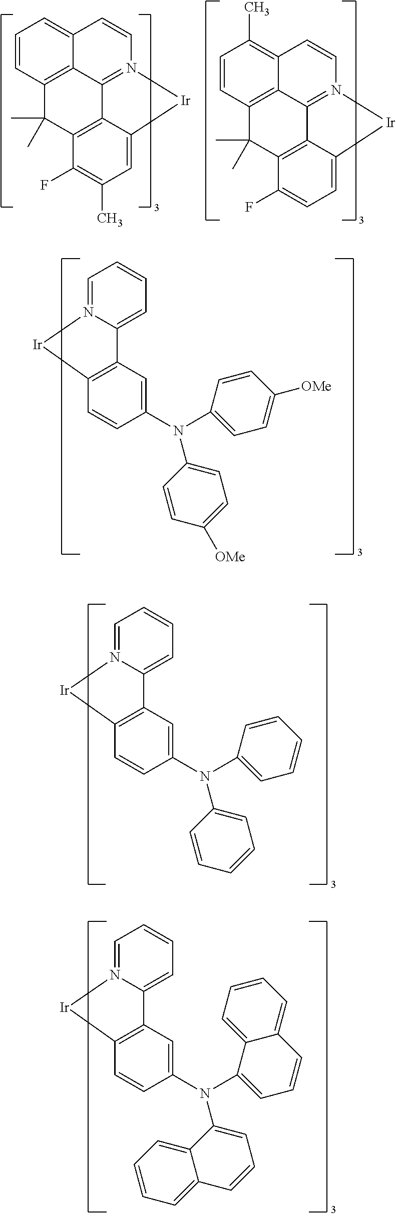

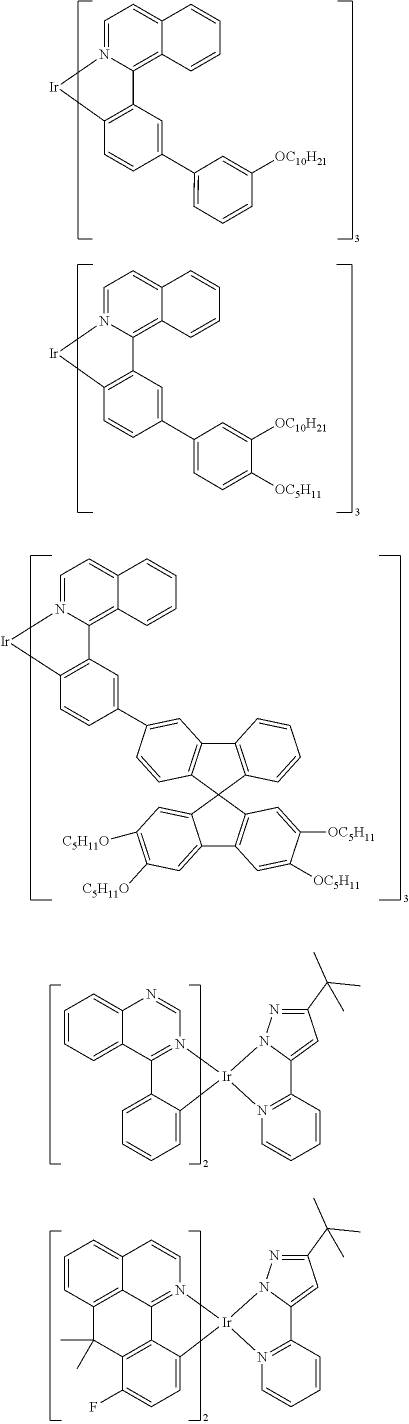

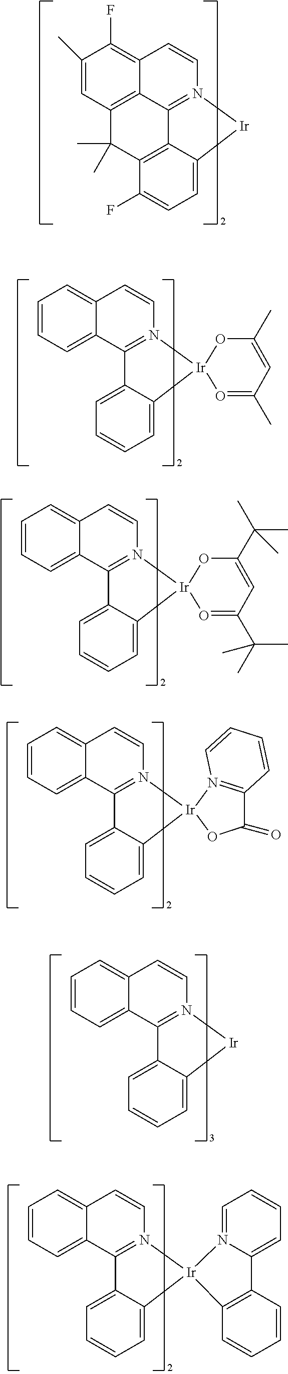

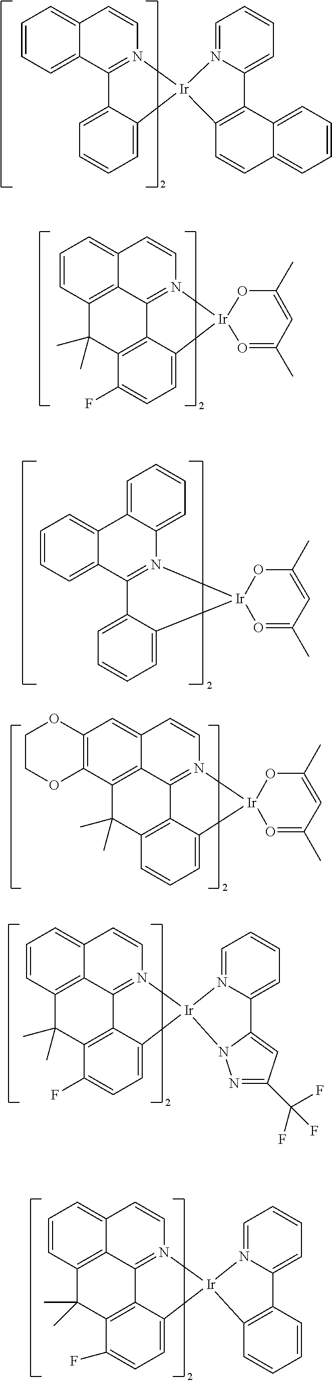

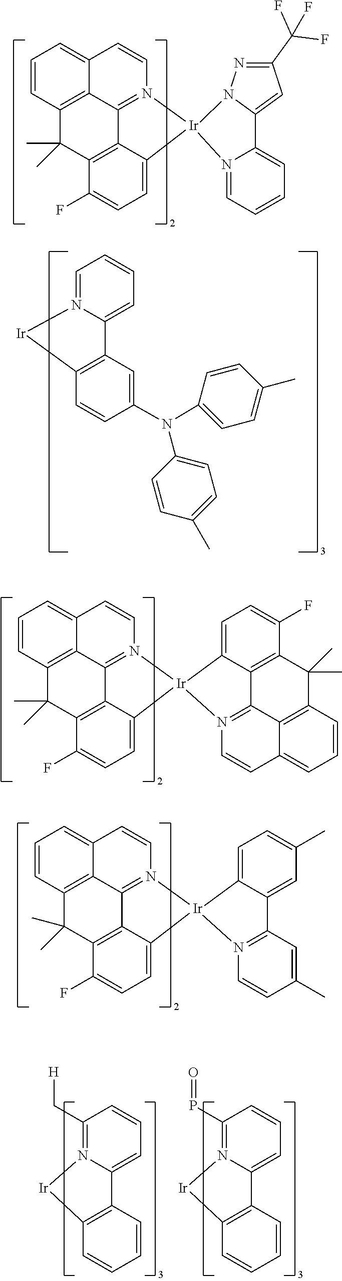

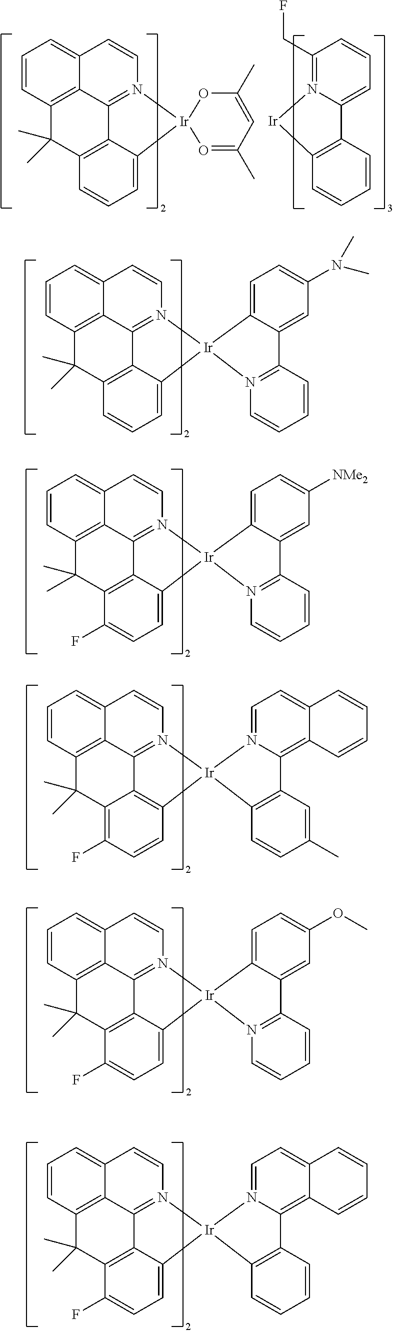

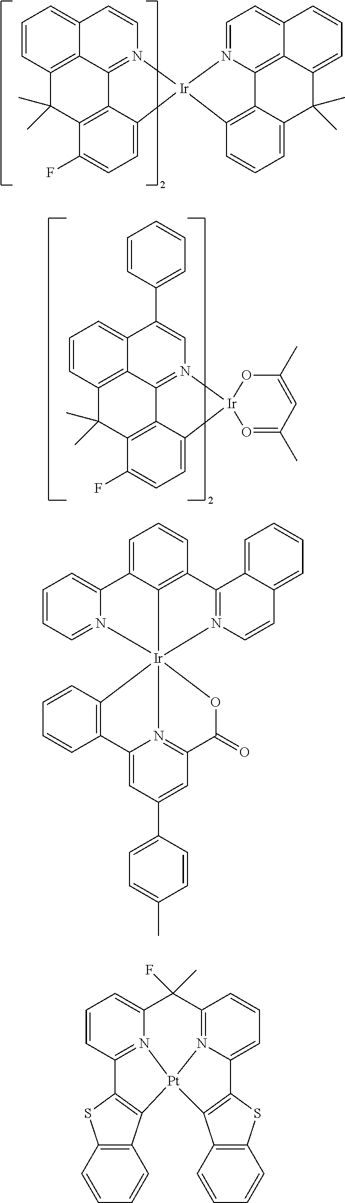

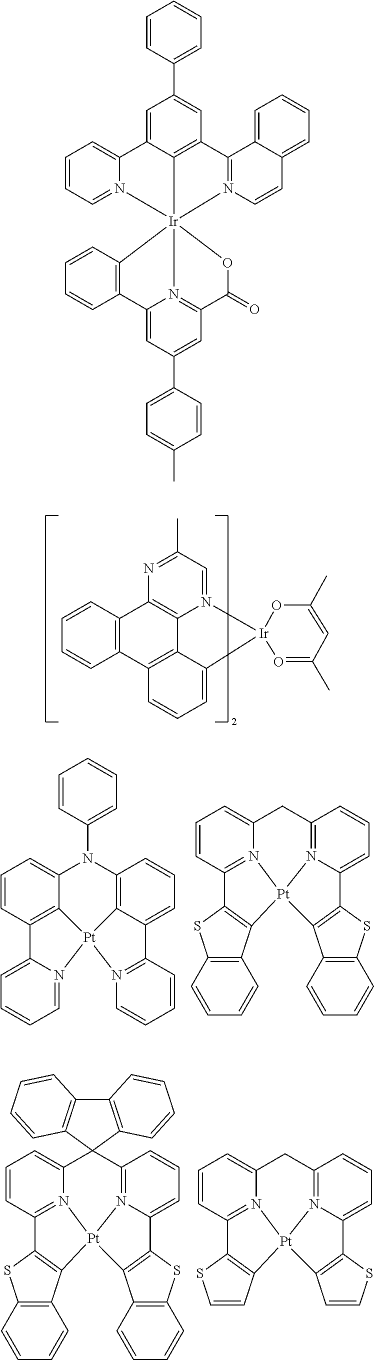

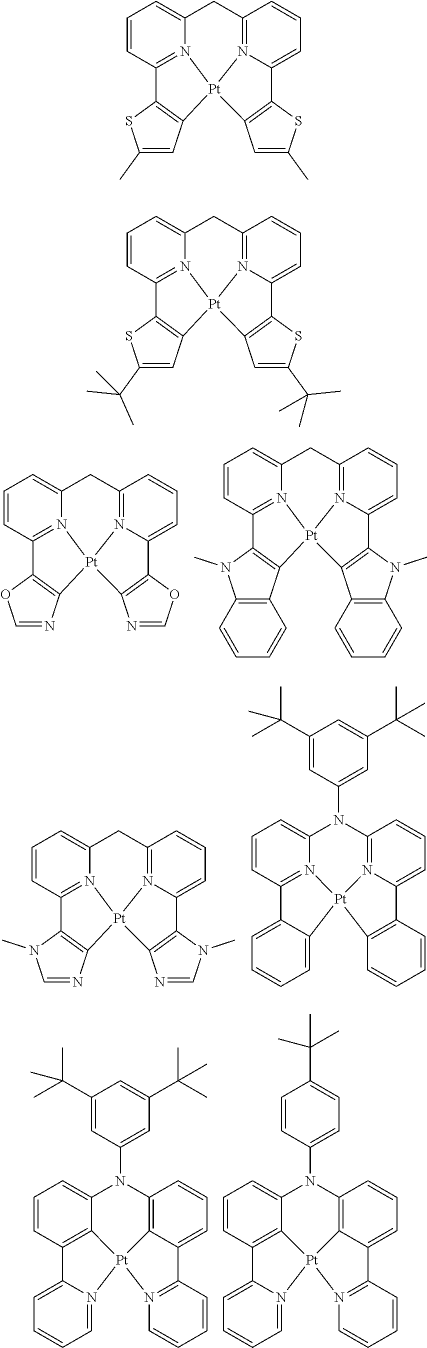

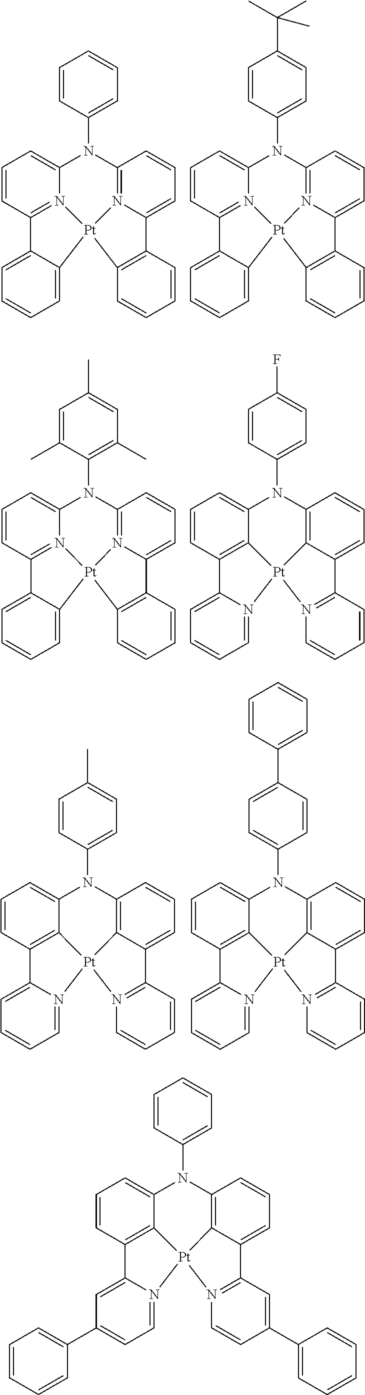

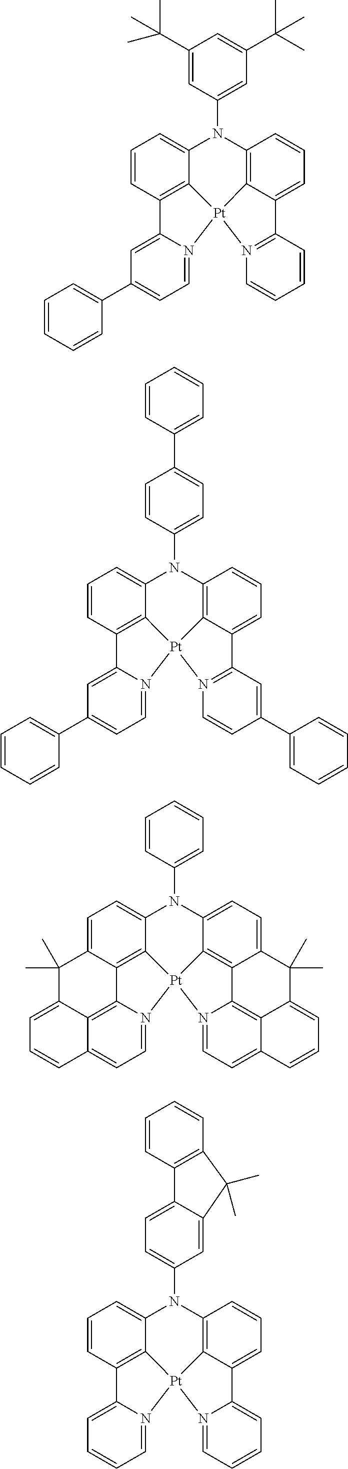

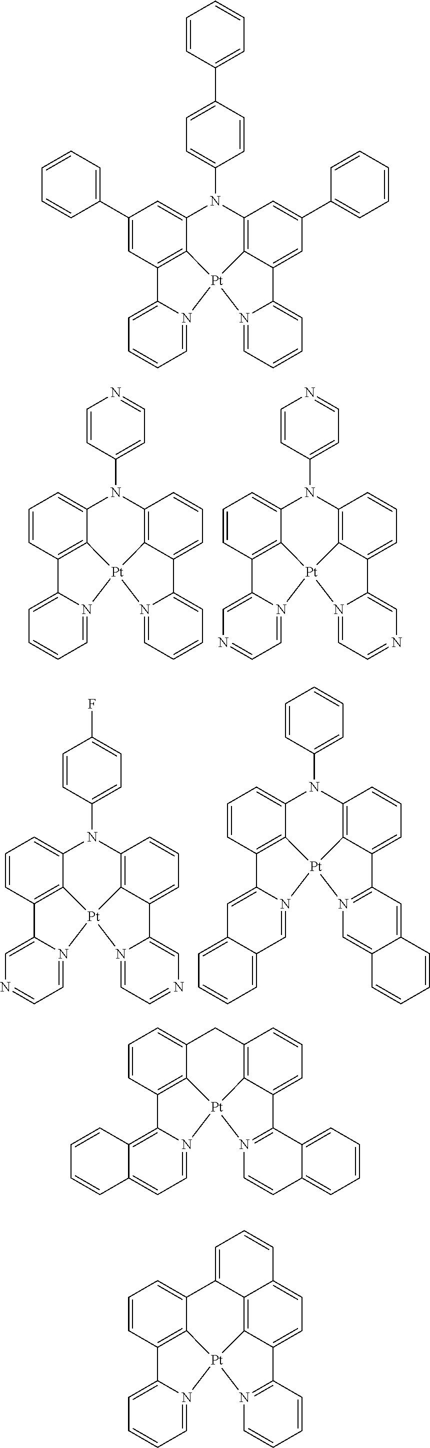

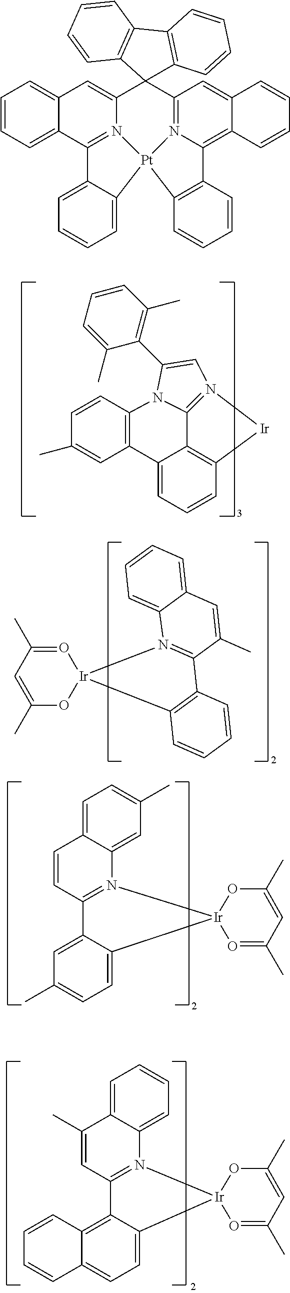

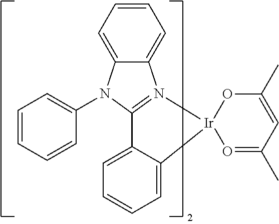

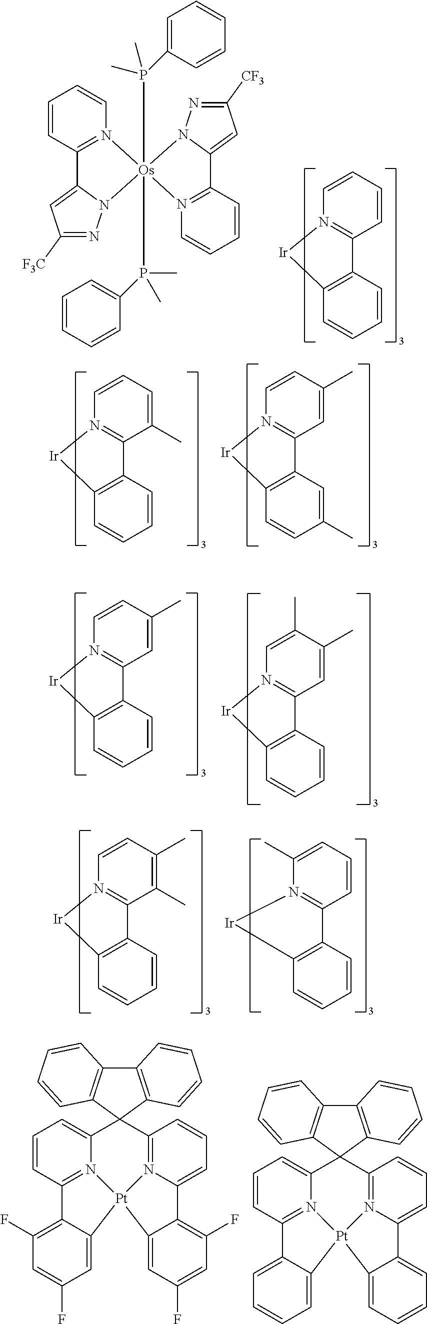

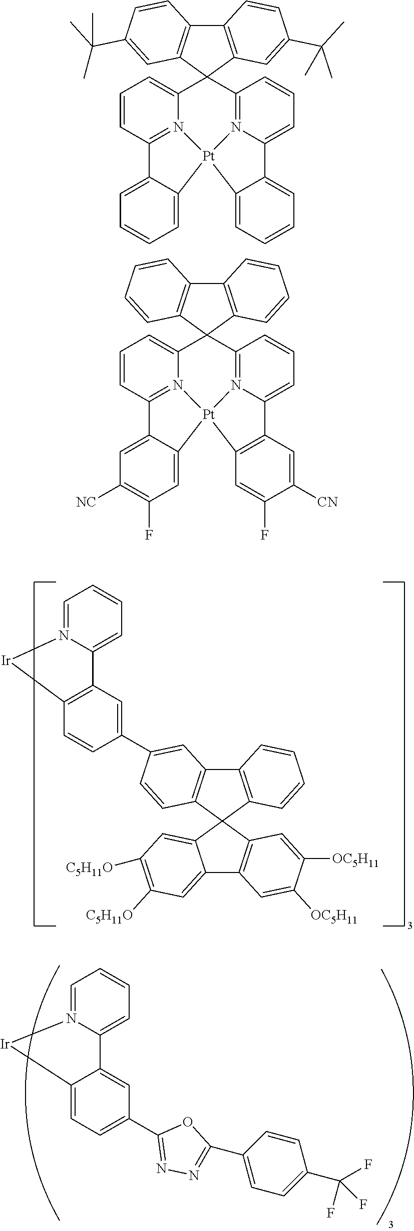

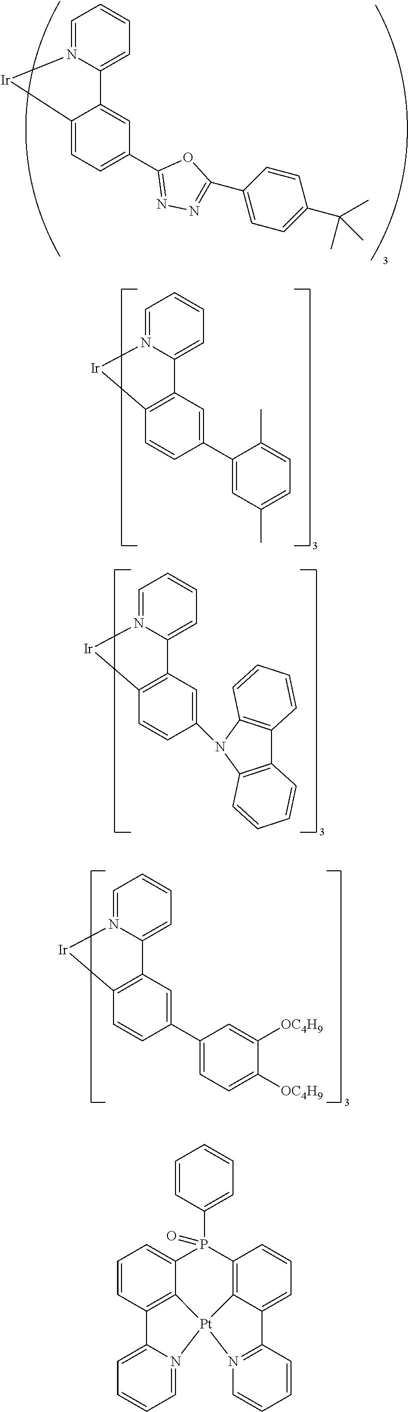

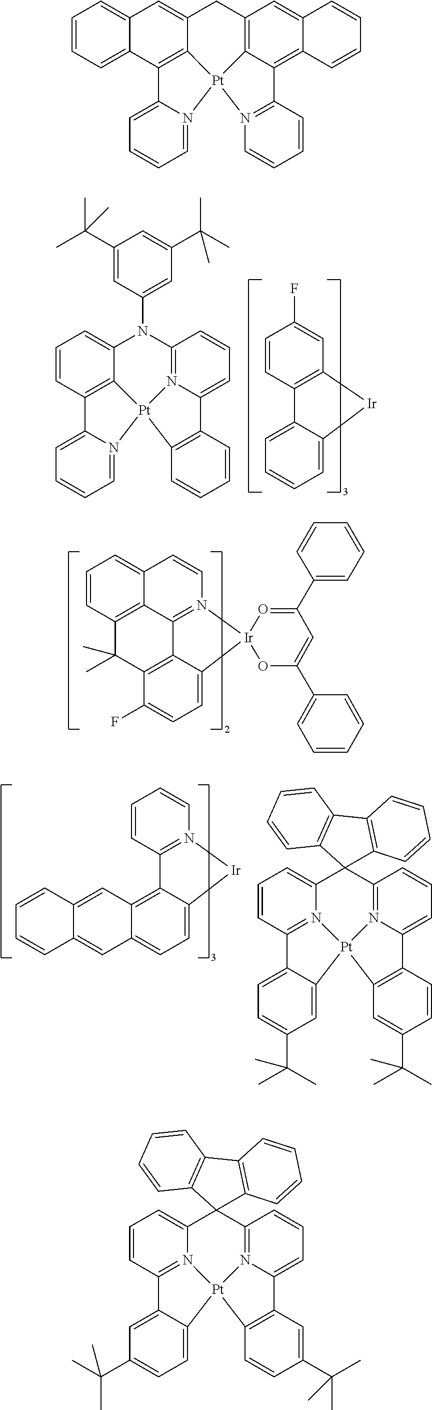

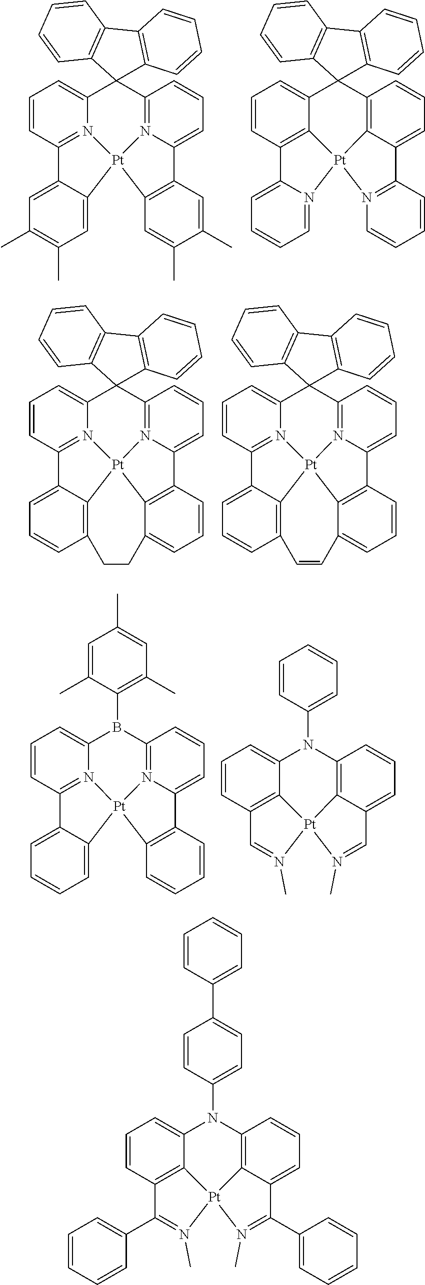

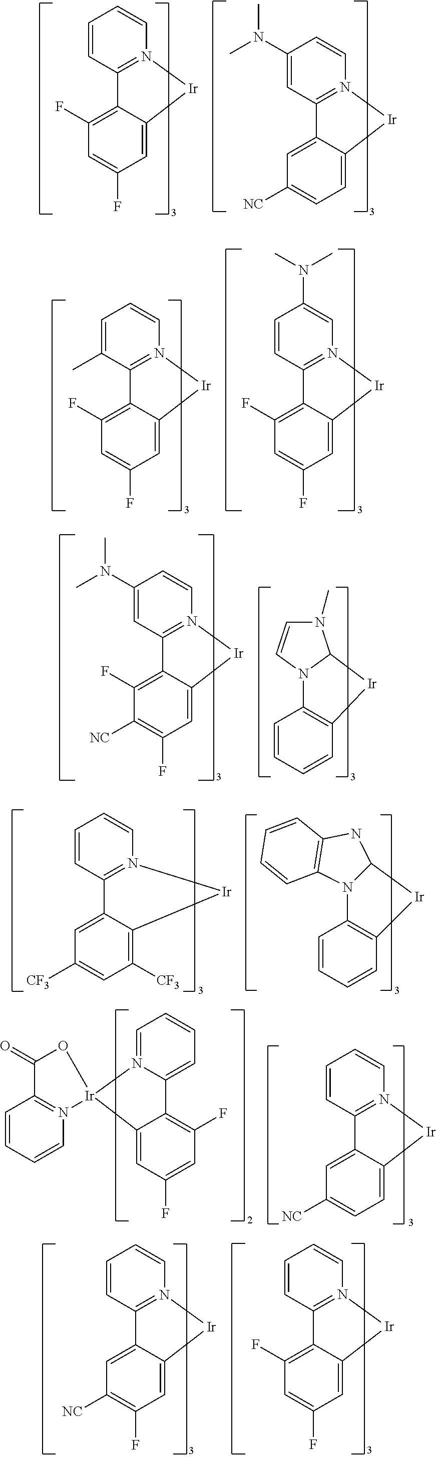

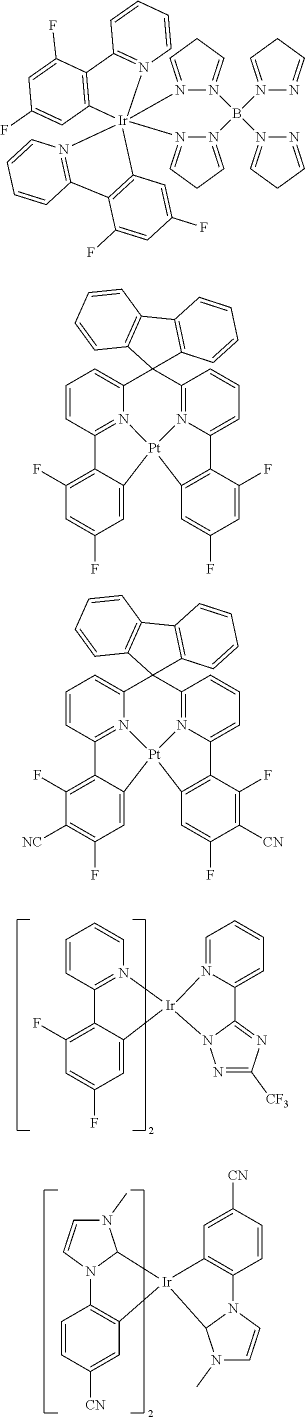

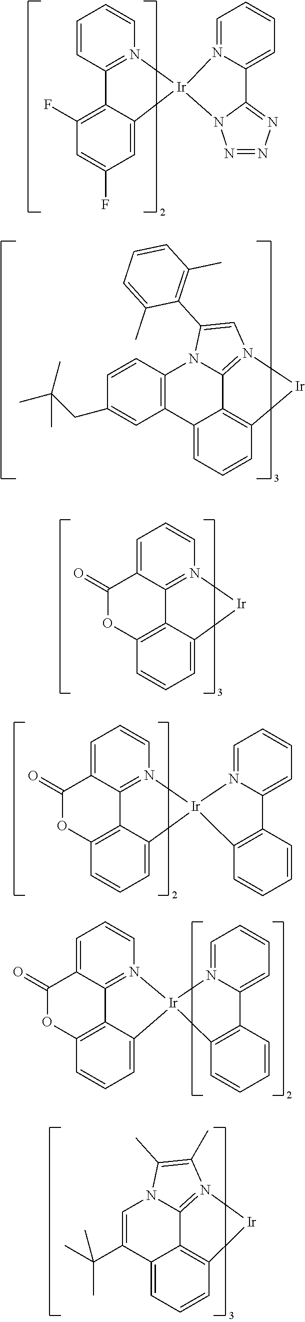

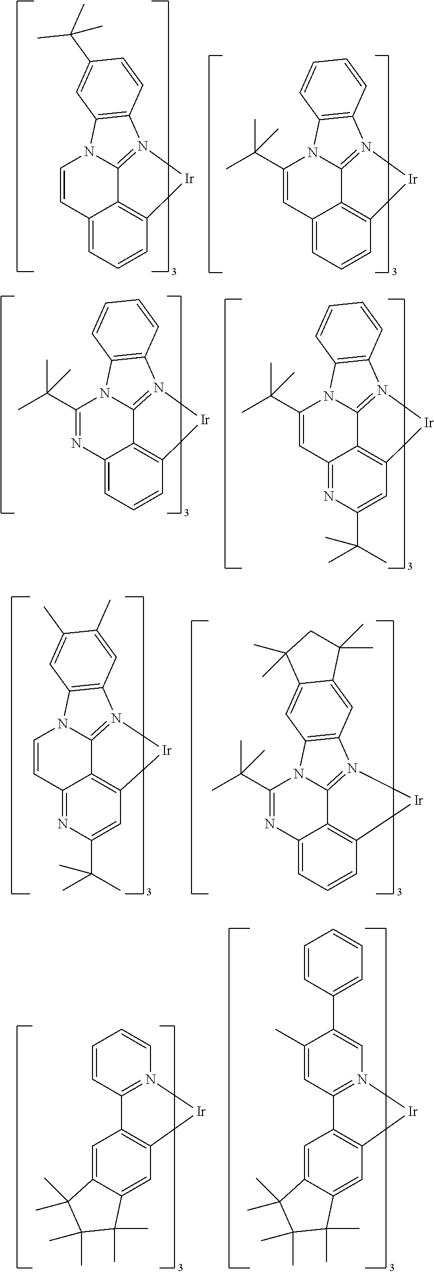

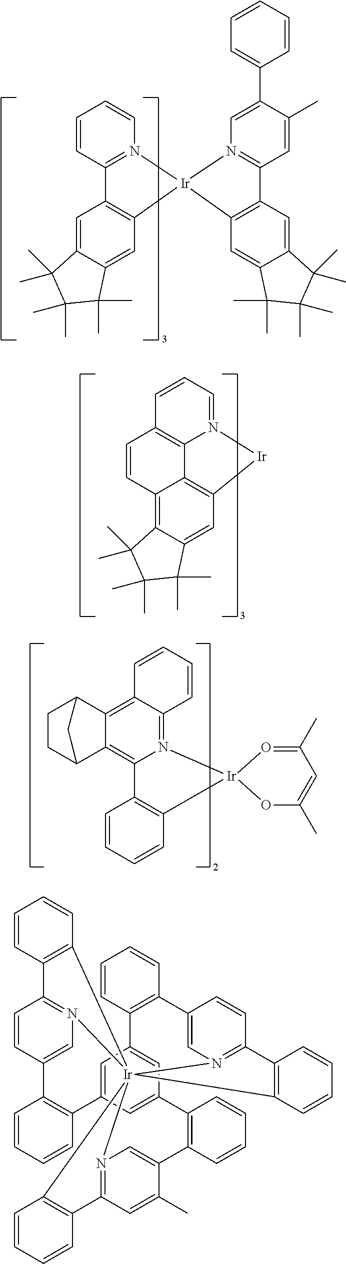

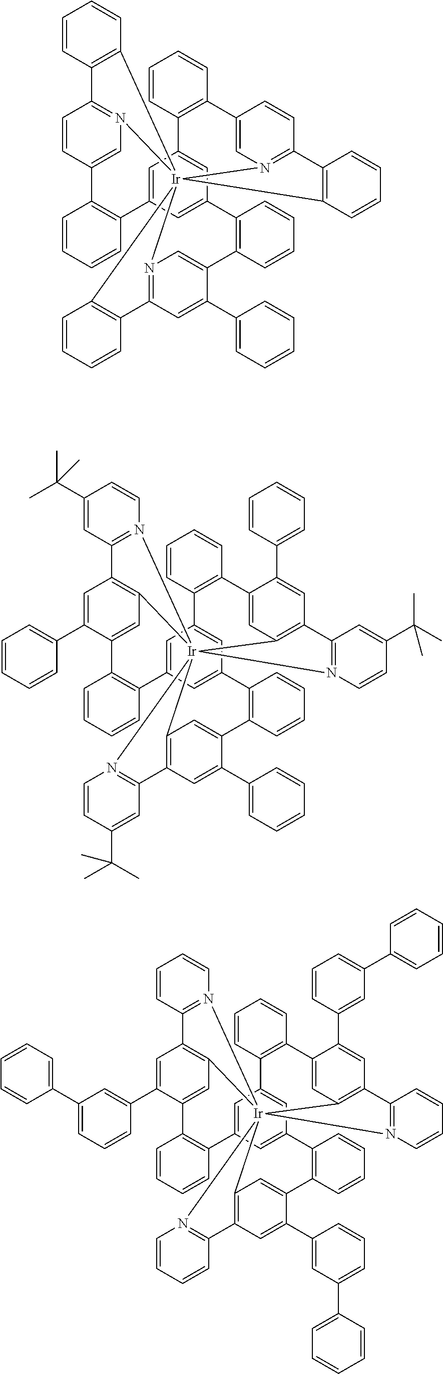

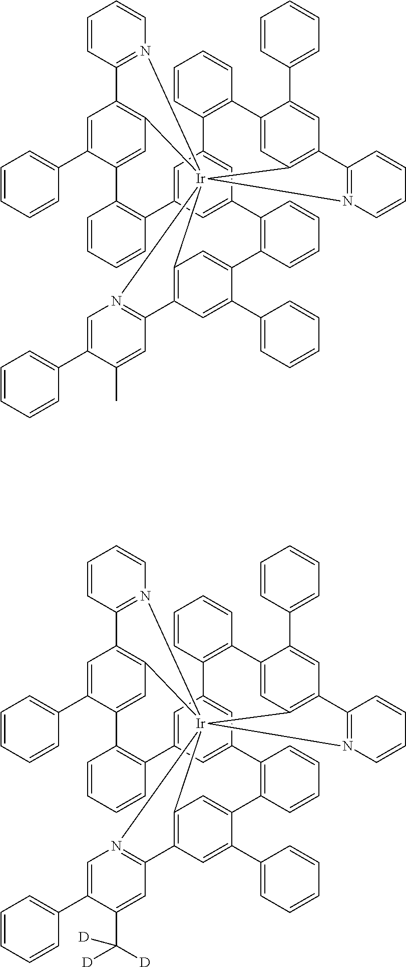

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms.

Preferred examples of these are aromatic anthracenamines, aromatic anthracenediamines, aromatic pyrenamines, aromatic pyrenediamines, aromatic chrysenamines or aromatic chrysenediamines. An aromatic anthracenamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 positions. Aromatic pyrenamines, pyrenediamines, chrysenamines and chrysenediamines are defined analogously, where the diarylamino groups are bonded to the pyrene preferably in the 1 position or 1,6 positions. Further preferred emitting compounds are indenofluorenamines or -fluorenediamines, for example according to WO 2006/108497 or WO 2006/122630, benzoindenofluorenamines or -fluorenediamines, for example according to WO 2008/006449, and dibenzoindenofluoreneamines or -diamines, for example according to WO 2007/140847, and the indenofluorene derivatives having fused aryl groups disclosed in WO 2010/012328. Likewise, preferred are the pyrenearylamines disclosed in WO 2012/048780 and in WO 2013/185871. Likewise, preferred are the benzoindenofluorenamines disclosed in WO 2014/037077, the benzofluorenamines disclosed in WO 2014/106522, the extended benzoindenofluorenes disclosed in WO 2014/111269 and in WO 2017/036574, the phenoxazines disclosed in WO 2017/028940 and in WO 2017/028941, and the fluorene derivatives bonded to furan units or to thiophene units that are disclosed in WO 2016/150544.

Useful matrix materials, preferably for fluorescent emitting compounds, include materials of various substance classes. Preferred matrix materials are selected from the classes of the oligoarylenes (e.g. 2,2′,7,7-tetraphenylspirobifluorene according to EP 676461 or dinaphthylanthracene), especially of the oligoarylenes containing fused aromatic groups, the oligoarylenevinylenes (e.g. DPVBi or spiro-DPVBi according to EP 676461), the polypodal metal complexes (for example according to WO 2004/081017), the hole-conducting compounds (for example according to WO 2004/058911), the electron-conducting compounds, especially ketones, phosphine oxides, sulphoxides, etc. (for example according to WO 2005/084081 and WO 2005/084082), the atropisomers (for example according to WO 2006/048268), the boronic acid derivatives (for example according to WO 2006/117052) or the benzanthracenes (for example according to WO 2008/145239). Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulphoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another. Preference is further given to the anthracene derivatives disclosed in WO 2006/097208, WO 2006/131192, WO 2007/065550, WO 2007/110129, WO 2007/065678, WO 2008/145239, WO 2009/100925, WO 2011/054442 and EP 1553154, the pyrene compounds disclosed in EP 1749809, EP 1905754 and US 2012/0187826, the benzanthracenylanthracene compounds disclosed in WO 2015/158409, the indenobenzofurans disclosed in WO 2017/025165, and the phenanthrylanthracenes disclosed in WO 2017/036573.

Preferred matrix materials for phosphorescent emitting compounds are, as well as the compounds of the formula (I), aromatic ketones, aromatic phosphine oxides or aromatic sulphoxides or sulphones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527 or WO 2008/086851, indolocarbazole derivatives, for example according to WO 2007/063754 or WO 2008/056746, indenocarbazole derivatives, for example according to WO 2010/136109, WO 2011/000455 or WO 2013/041176, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2010/015306, WO 2007/063754 or WO 2008/056746, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to US 2009/0136779, WO 2010/050778, WO 2011/042107, WO 2011/088877 or WO 2012/143080, triphenylene derivatives, for example according to WO 2012/048781, or lactams, for example according to WO 2011/116865 or WO 2011/137951.

Suitable charge transport materials as usable in the hole injection or hole transport layer or electron blocking layer or in the electron transport layer of the electronic device of the invention are, as well as the compounds of the formula (I), for example, the compounds disclosed in Y. Shirota et al., Chem. Rev. 2007, 107(4), 953-1010, or other materials as used in these layers according to the prior art.

Preferably, the inventive OLED comprises two or more different hole-transporting layers. The compound of the formula (I) may be used here in one or more of or in all the hole-transporting layers. In a preferred embodiment, the compound of the formula (I) is used in exactly one or exactly two hole-transporting layers, and other compounds, preferably aromatic amine compounds, are used in the further hole-transporting layers present. Further compounds which are used alongside the compounds of the formula (I), preferably in hole-transporting layers of the OLEDs of the invention, are especially indenofluorenamine derivatives (for example according to WO 06/122630 or WO 06/100896), the amine derivatives disclosed in EP 1661888, hexaazatriphenylene derivatives (for example according to WO 01/049806), amine derivatives with fused aromatics (for example according to U.S. Pat. No. 5,061,569), the amine derivatives disclosed in WO 95/09147, monobenzoindenofluorenamines (for example according to WO 08/006449), dibenzoindenofluorenamines (for example according to WO 07/140847), spirobifluorenamines (for example according to WO 2012/034627 or WO 2013/120577), fluorenamines (for example according to WO 2014/015937, WO 2014/015938, WO 2014/015935 and WO 2015/082056), spirodibenzopyranamines (for example according to WO 2013/083216), dihydroacridine derivatives (for example according to WO 2012/150001), spirodibenzofurans and spirodibenzothiophenes, for example according to WO 2015/022051, WO 2016/102048 and WO 2016/131521, phenanthrenediarylamines, for example according to WO 2015/131976, spirotribenzotropolones, for example according to WO 2016/087017, spirobifluorenes with meta-phenyldiamine groups, for example according to WO 2016/078738, spirobisacridines, for example according to WO 2015/158411, xanthenediarylamines, for example according to WO 2014/072017, and 9,10-dihydroanthracene spiro compounds with diarylamino groups according to WO 2015/086108.

Very particular preference is given to the use of spirobifluorenes substituted by diarylamino groups in the 4 position as hole-transporting compounds, especially to the use of those compounds that are claimed and disclosed in WO 2013/120577, and to the use of spirobifluorenes substituted by diarylamino groups in the 2 position as hole-transporting compounds, especially to the use of those compounds that are claimed and disclosed in WO 2012/034627.

Materials used for the electron transport layer may be any materials as used according to the prior art as electron transport materials in the electron transport layer. Especially suitable are aluminum complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Further suitable materials are derivatives of the abovementioned compounds as disclosed in JP 2000/053957, WO 2003/060956, WO 2004/028217, WO 2004/080975 and WO 2010/072300.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally, suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable the irradiation of the organic material (organic solar cell) or the emission of light (OLED, O-laser). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

The device is structured appropriately (according to the application), contact-connected and finally sealed, in order to rule out damaging effects by water and air.

In a preferred embodiment, the electronic device is characterized in that one or more layers are coated by a sublimation process. In this case, the materials are applied by vapour deposition in vacuum sublimation systems at an initial pressure of less than 10⁻⁵ mbar, preferably less than 10⁻⁶ mbar. In this case, however, it is also possible that the initial pressure is even lower, for example less than 10⁻⁷ mbar.

Preference is likewise given to an electronic device, characterized in that one or more layers are coated by the OVPD (organic vapour phase deposition) method or with the aid of a carrier gas sublimation. In this case, the materials are applied at a pressure between 10⁻⁵ mbar and 1 bar. A special case of this method is the OVJP (organic vapour jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (I) are needed. High solubility can be achieved by suitable substitution of the compounds.

It is further preferable that an electronic device of the invention is produced by applying one or more layers from solution and one or more layers by a sublimation method.

According to the invention, the electronic devices comprising one or more compounds of formula (I) can be used in displays, as light sources in lighting applications and as light sources in medical and/or cosmetic applications (e.g. light therapy).

The compounds according to the present invention and the electronic devices according to the present invention, respectively, exhibit the following surprising and advantageous effects compared to the prior art:
1. The compounds according to the present invention are particularly suitable as hole-transporting materials in an electron blocking layer in electronic devices, such as electroluminescent devices, which is particularly due to their very good very good electron-blocking properties and hole-conducting properties
2. The compounds according to the present invention are characterized by low sublimation temperature, high thermal stability, high oxidation stability, high glass transition temperature and high solubility, which is advantageous in terms of their processability, for example from the liquid phase or from the gaseous phase and makes them particularly suitable for being used in electronic devices.
3. When used in electronic devices, in particular as hole-transporting materials, the compounds according to the present invention lead to excellent results in terms of lifetime, operating voltage and quantum efficiency of the devices.
4. The compounds containing deuterium are more thermally stable, the devices containing the compounds show a longer lifetime and an improved efficiency

The invention is described in more detail below with the help of examples which are not to be considered as limiting the scope of the invention.

### EXAMPLES

### A) Synthesis Examples

The following syntheses are carried out under a protective-gas atmosphere, unless indicated otherwise. The starting materials can be purchased from ALDRICH or ABCR. The numbers in square brackets in the case of the starting materials known from the literature are the corresponding CAS numbers.

### Example 1

### Synthesis of 2-{[1,1′-biphenyl]-2-yl}-5-bromobenzoate methyl ester 1a

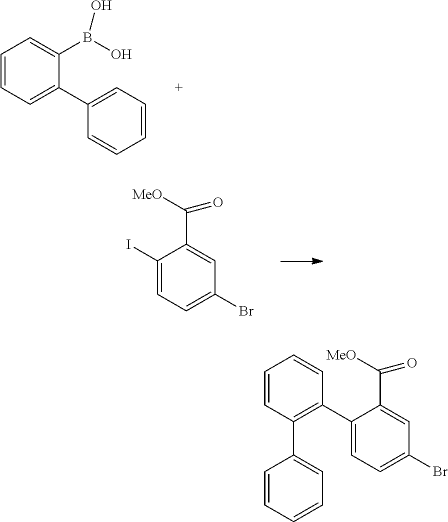 [SMILES: OB(O)c1ccccc1-c1ccccc1 ; COC(=O)c1cc(Br)ccc1I ; COC(=O)c1cc(Br)ccc1-c1ccccc1-c1ccccc1]

2.9 g (14.9 mmol) of (1,1′-biphenyl)-2-yl-boronic acid, 4.6 g (13.6 mmol) of methyl 5-bromo-2-iodobenzoate, 314 mg (0.3 mmol, 0.02 eq.) of Pd(P(Ph₃))₄, 5.6 g (40.7 mmol, 3 eq.) of Na₂CO₃ are dissolved in 7 mL of water and 30 mL of toluene. The reaction mixture is stirred at 85° C. and agitated under an argon atmosphere for 12 hours and after cooling to room temperature, the mixture is filtered through Celite. The filtrate is evaporated in vacuo, and the residue is purified by chromatography (mixture heptane/AcOEt). The product is isolated in the form of an off-white solid (4.5 g 91% of theory).

The synthesis of further derivatives is carried out analogously:

| | Boronic acid | | Product | Yield |
|---|---|---|---|---|
| 1b | 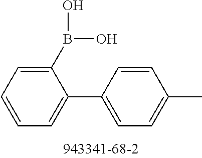 [SMILES: Cc1ccc(-c2ccccc2B(O)O)cc1] | 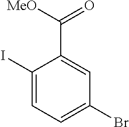 [SMILES: COC(=O)c1cc(Br)ccc1I] | 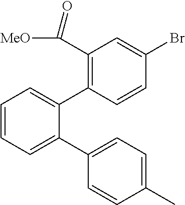 [SMILES: COC(=O)c1cc(Br)ccc1-c1ccccc1-c1ccc(C)cc1] | 86% |
| 1c | 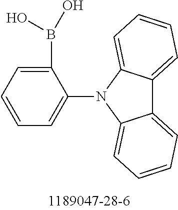 [SMILES: OB(O)c1ccccc1-n1c2ccccc2c2ccccc21] | 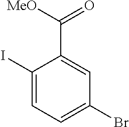 [SMILES: COC(=O)c1cc(Br)ccc1I] | 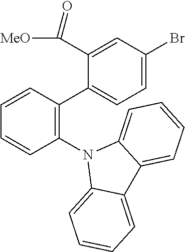 [SMILES: COC(=O)c1cc(Br)ccc1-c1ccccc1-n1c2ccccc2c2ccccc21] | 84% |
| 1d | 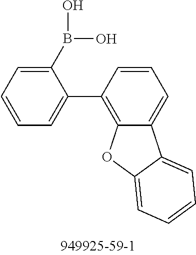 [SMILES: OB(O)c1ccccc1-c1cccc2c1oc1ccccc12] | 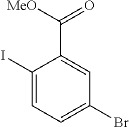 [SMILES: COC(=O)c1cc(Br)ccc1I] | 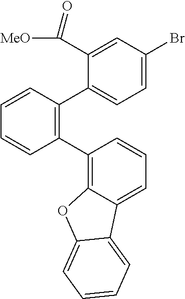 [SMILES: COC(=O)c1cc(Br)ccc1-c1ccccc1-c1cccc2c1oc1ccccc12] | 70% |
| 1f | 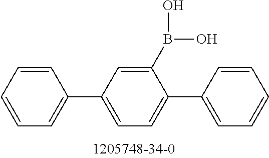 [SMILES: OB(O)c1cc(-c2ccccc2)ccc1-c1ccccc1] | 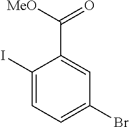 [SMILES: COC(=O)c1cc(Br)ccc1I] | 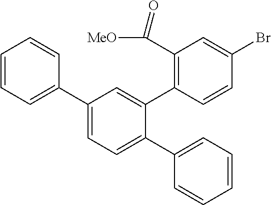 [SMILES: COC(=O)c1cc(Br)ccc1-c1cc(-c2ccccc2)ccc1-c1ccccc1] | 80% |
| 1g | 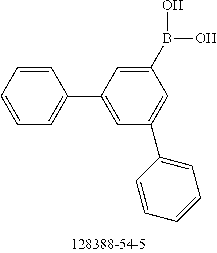 [SMILES: OB(O)c1cc(-c2ccccc2)cc(-c2ccccc2)c1] | 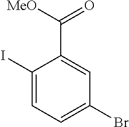 [SMILES: COC(=O)c1cc(Br)ccc1I] | 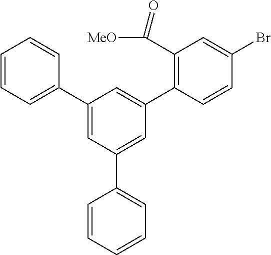 [SMILES: COC(=O)c1cc(Br)ccc1-c1cc(-c2ccccc2)cc(-c2ccccc2)c1] | 66% |
| 1h | 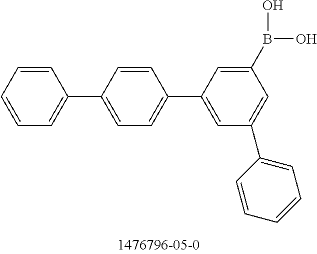 [SMILES: OB(O)c1cc(-c2ccccc2)cc(-c2ccc(-c3ccccc3)cc2)c1] | 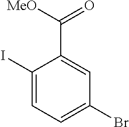 [SMILES: COC(=O)c1cc(Br)ccc1I] | 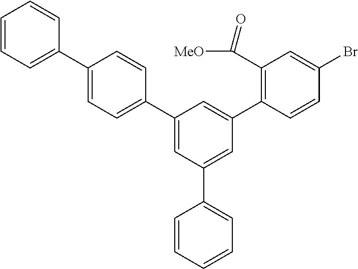 [SMILES: COC(=O)c1cc(Br)ccc1-c1cc(-c2ccccc2)cc(-c2ccc(-c3ccccc3)cc2)c1] | 81% |
| 1i | 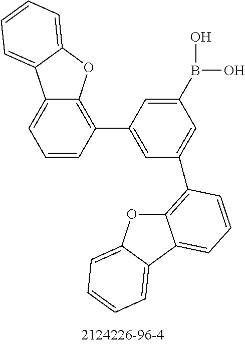 [SMILES: OB(O)c1cc(-c2cccc3c2oc2ccccc23)cc(-c2cccc3c2oc2ccccc23)c1] | 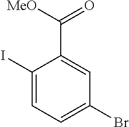 [SMILES: COC(=O)c1cc(Br)ccc1I] | 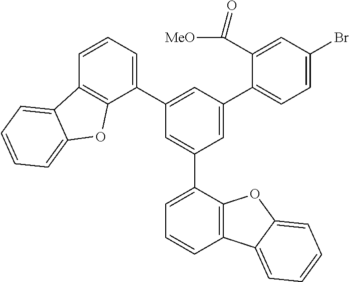 [SMILES: COC(=O)c1cc(Br)ccc1-c1cc(-c2cccc3c2oc2ccccc23)cc(-c2cccc3c2oc2ccccc23)c1] | 77% |
| 1j | 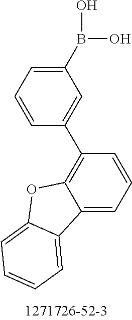 [SMILES: OB(O)c1cccc(-c2cccc3c2oc2ccccc23)c1] | 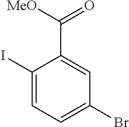 [SMILES: COC(=O)c1cc(Br)ccc1I] | 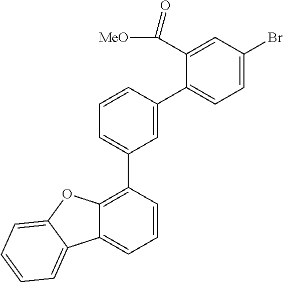 [SMILES: COC(=O)c1cc(Br)ccc1-c1cccc(-c2cccc3c2oc2ccccc23)c1] | 85% |
| 1k | 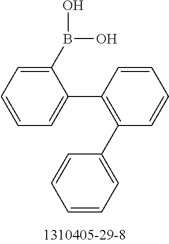 [SMILES: OB(O)c1ccccc1-c1ccccc1-c1ccccc1] | 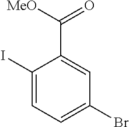 [SMILES: COC(=O)c1cc(Br)ccc1I] | 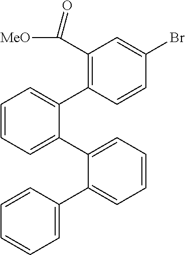 [SMILES: COC(=O)c1cc(Br)ccc1-c1ccccc1-c1ccccc1-c1ccccc1] | 81% |
| 1l | 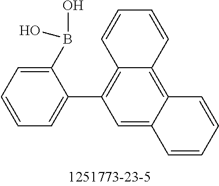 [SMILES: OB(O)c1ccccc1-c1cc2ccccc2c2ccccc12] | 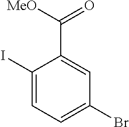 [SMILES: COC(=O)c1cc(Br)ccc1I] | 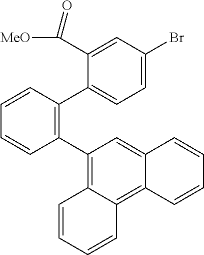 [SMILES: COC(=O)c1cc(Br)ccc1-c1ccccc1-c1cc2ccccc2c2ccccc12] | 80% |
| 1ll | 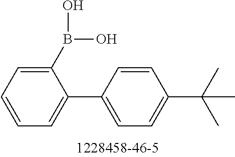 [SMILES: CC(C)(C)c1ccc(-c2ccccc2B(O)O)cc1] | 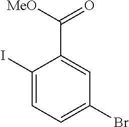 [SMILES: COC(=O)c1cc(Br)ccc1I] | 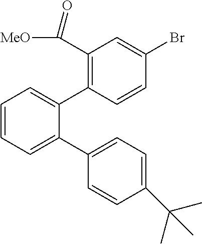 [SMILES: COC(=O)c1cc(Br)ccc1-c1ccccc1-c1ccc(C(C)(C)C)cc1] | 81% |
| 1m | 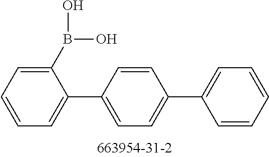 [SMILES: OB(O)c1ccccc1-c1ccc(-c2ccccc2)cc1] | 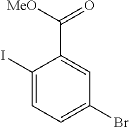 [SMILES: COC(=O)c1cc(Br)ccc1I] | 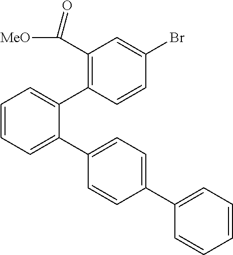 [SMILES: COC(=O)c1cc(Br)ccc1-c1ccccc1-c1ccc(-c2ccccc2)cc1] | 76% |
| 1n | 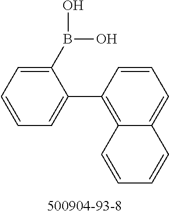 [SMILES: OB(O)c1ccccc1-c1cccc2ccccc12] | 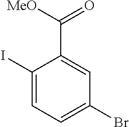 [SMILES: COC(=O)c1cc(Br)ccc1I] | 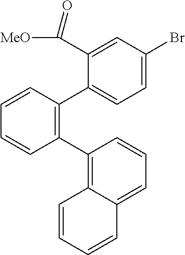 [SMILES: COC(=O)c1cc(Br)ccc1-c1ccccc1-c1cccc2ccccc12] | 81% |
| 1o | 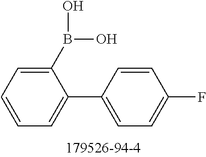 [SMILES: OB(O)c1ccccc1-c1ccc(F)cc1] | 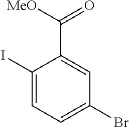 [SMILES: COC(=O)c1cc(Br)ccc1I] | 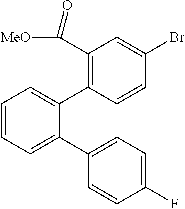 [SMILES: COC(=O)c1cc(Br)ccc1-c1ccccc1-c1ccc(F)cc1] | 78% |
| 1p | 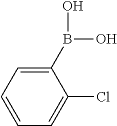 | 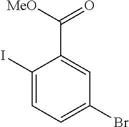 [SMILES: COC(=O)c1cc(Br)ccc1I] | 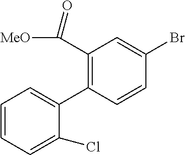 [SMILES: COC(=O)c1cc(Br)ccc1-c1ccccc1Cl] | 70% |
| 1q | 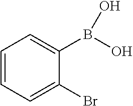 | 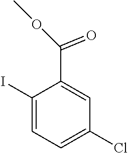 | 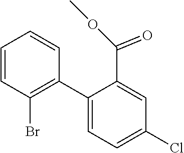 [SMILES: COC(=O)c1cc(Cl)ccc1-c1ccccc1Br] | 75% |
| 1r | 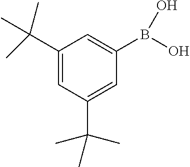 [SMILES: CC(C)(C)c1cc(B(O)O)cc(C(C)(C)C)c1] | 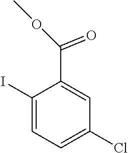 | 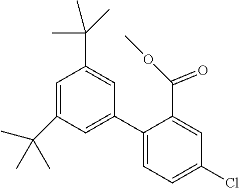 [SMILES: COC(=O)c1cc(Cl)ccc1-c1cc(C(C)(C)C)cc(C(C)(C)C)c1] | 65% |

### Synthesis of 2-(2-{[1,1′-biphenyl]-2-yl}-5-bromophenyl)propan-2-ol 2a

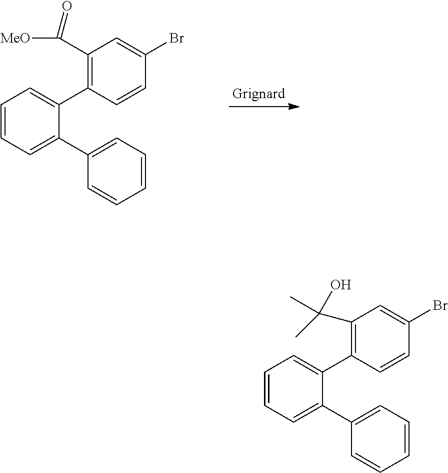 [SMILES: COC(=O)c1cc(Br)ccc1-c1ccccc1-c1ccccc1 ; CC(C)(O)c1cc(Br)ccc1-c1ccccc1-c1ccccc1]

A solution of 2-{[1,1′-biphenyl]-2-yl}-5-bromobenzoate methyl ester (3 g, 8.2 mmol) in THF (30 ml) is treated with 16 mL of MeMgCl (3 M in THF, 49 mmol, 6 eq.) under argon at −10° C. The reaction proceeds at −10° C. for 30 minutes and then is stirred at room temperature overnight. The reaction is quenched with a solution of saturated NH₄Cl and the mixture is extracted with EtOAc. The organic phase is dried with MgSO₄ and concentrated to dryness. The residue is purified by chromatography (mixture heptane/AcOEt) to isolate pure 2a (1.8 g, 61% of theory).

The following compounds are synthesized analogously:

| Ex. | | Grignard reagent | Product | Yield |
|---|---|---|---|---|
| 2b | 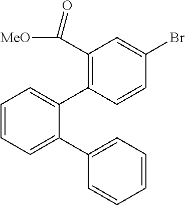 [SMILES: COC(=O)c1cc(Br)ccc1-c1ccccc1-c1ccccc1] | PhMgCl | 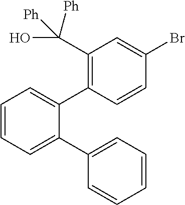 [SMILES: OC(c1ccccc1)(c1ccccc1)c1cc(Br)ccc1-c1ccccc1-c1ccccc1] | 86% |
| 2c | 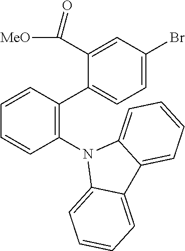 [SMILES: COC(=O)c1cc(Br)ccc1-c1ccccc1-n1c2ccccc2c2ccccc21] | MeMgCl | 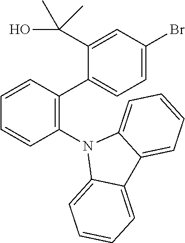 [SMILES: CC(C)(O)c1cc(Br)ccc1-c1ccccc1-n1c2ccccc2c2ccccc21] | 82% |
| 2d | 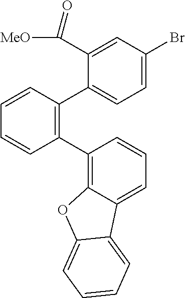 [SMILES: COC(=O)c1cc(Br)ccc1-c1ccccc1-c1cccc2c1oc1ccccc12] | PhMgCl | 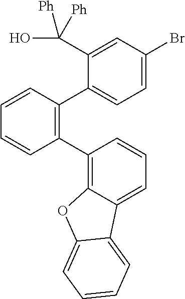 [SMILES: OC(c1ccccc1)(c1ccccc1)c1cc(Br)ccc1-c1ccccc1-c1cccc2c1oc1ccccc12] | 43% |
| 2e | 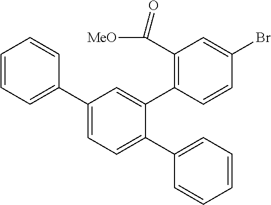 [SMILES: COC(=O)c1cc(Br)ccc1-c1cc(-c2ccccc2)ccc1-c1ccccc1] | MeMgCl | 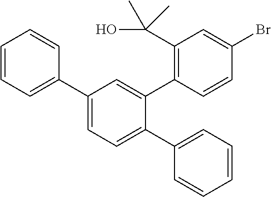 [SMILES: CC(C)(O)c1cc(Br)ccc1-c1cc(-c2ccccc2)ccc1-c1ccccc1] | 92% |
| 2f | 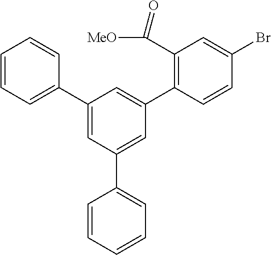 [SMILES: COC(=O)c1cc(Br)ccc1-c1cc(-c2ccccc2)cc(-c2ccccc2)c1] | MeMgCl | 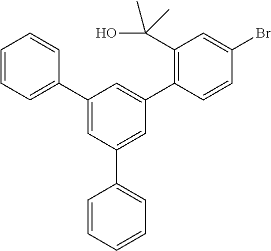 [SMILES: CC(C)(O)c1cc(Br)ccc1-c1cc(-c2ccccc2)cc(-c2ccccc2)c1] | 90% |
| 2g | 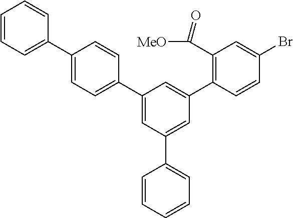 [SMILES: COC(=O)c1cc(Br)ccc1-c1cc(-c2ccccc2)cc(-c2ccc(-c3ccccc3)cc2)c1] | PhMgCl | 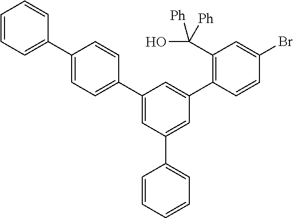 [SMILES: OC(c1ccccc1)(c1ccccc1)c1cc(Br)ccc1-c1cc(-c2ccccc2)cc(-c2ccc(-c3ccccc3)cc2)c1] | 85% |
| 2h | 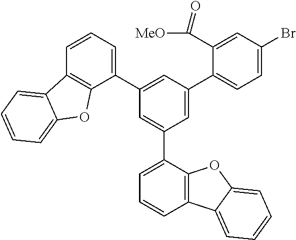 [SMILES: COC(=O)c1cc(Br)ccc1-c1cc(-c2cccc3c2oc2ccccc23)cc(-c2cccc3c2oc2ccccc23)c1] | MeMgCl | 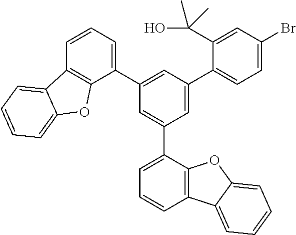 [SMILES: CC(C)(O)c1cc(Br)ccc1-c1cc(-c2cccc3c2oc2ccccc23)cc(-c2cccc3c2oc2ccccc23)c1] | 55% |
| 2i | 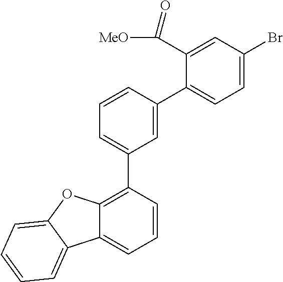 [SMILES: COC(=O)c1cc(Br)ccc1-c1cccc(-c2cccc3c2oc2ccccc23)c1] | PhMgCl | 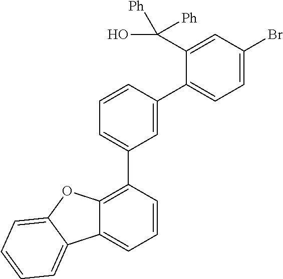 [SMILES: OC(c1ccccc1)(c1ccccc1)c1cc(Br)ccc1-c1cccc(-c2cccc3c2oc2ccccc23)c1] | 54% |
| 2j | 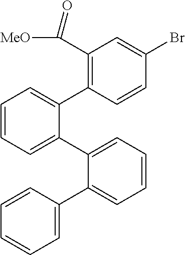 [SMILES: COC(=O)c1cc(Br)ccc1-c1ccccc1-c1ccccc1-c1ccccc1] | MeMgCl | 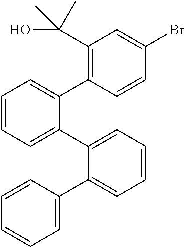 [SMILES: CC(C)(O)c1cc(Br)ccc1-c1ccccc1-c1ccccc1-c1ccccc1] | 40% |
| 2k | 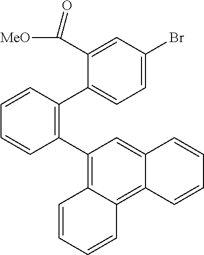 [SMILES: COC(=O)c1cc(Br)ccc1-c1ccccc1-c1cc2ccccc2c2ccccc12] | MeMgCl | 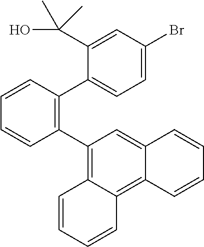 [SMILES: CC(C)(O)c1cc(Br)ccc1-c1ccccc1-c1cc2ccccc2c2ccccc12] | 50% |
| 2l | 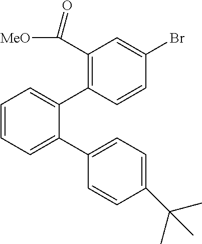 [SMILES: COC(=O)c1cc(Br)ccc1-c1ccccc1-c1ccc(C(C)(C)C)cc1] | PhMgCl | 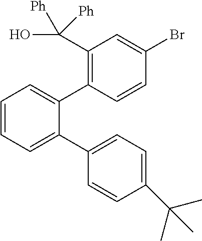 [SMILES: CC(C)(C)c1ccc(-c2ccccc2-c2ccc(Br)cc2C(O)(c2ccccc2)c2ccccc2)cc1] | 80% |
| 2m | 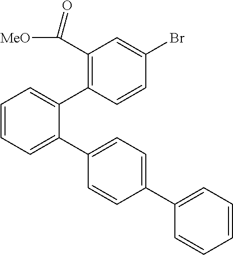 [SMILES: COC(=O)c1cc(Br)ccc1-c1ccccc1-c1ccc(-c2ccccc2)cc1] | MeMgCl | 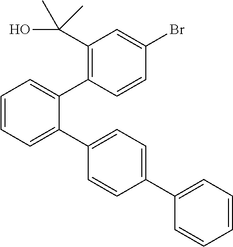 [SMILES: CC(C)(O)c1cc(Br)ccc1-c1ccccc1-c1ccc(-c2ccccc2)cc1] | 85% |
| 2n | 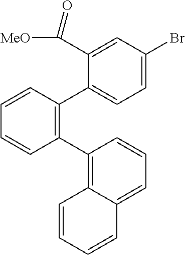 [SMILES: COC(=O)c1cc(Br)ccc1-c1ccccc1-c1cccc2ccccc12] | PhMgCl | 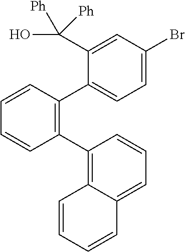 [SMILES: OC(c1ccccc1)(c1ccccc1)c1cc(Br)ccc1-c1ccccc1-c1cccc2ccccc12] | 60% |
| 2o | 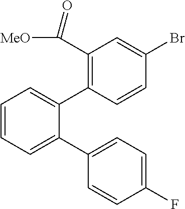 [SMILES: COC(=O)c1cc(Br)ccc1-c1ccccc1-c1ccc(F)cc1] | PhMgCl | 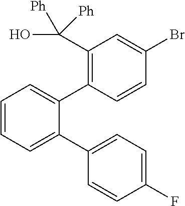 [SMILES: OC(c1ccccc1)(c1ccccc1)c1cc(Br)ccc1-c1ccccc1-c1ccc(F)cc1] | 67% |
| 2p | 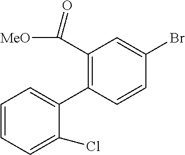 [SMILES: COC(=O)c1cc(Br)ccc1-c1ccccc1Cl] | PhMgCl | 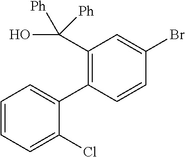 [SMILES: OC(c1ccccc1)(c1ccccc1)c1cc(Br)ccc1-c1ccccc1Cl] | 82% |
| 2q | 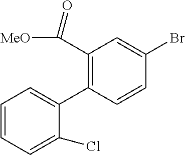 [SMILES: COC(=O)c1cc(Br)ccc1-c1ccccc1Cl] | MeMgCl | 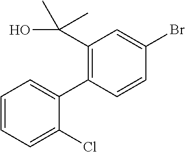 [SMILES: CC(C)(O)c1cc(Br)ccc1-c1ccccc1Cl] | 74% |
| 2r | 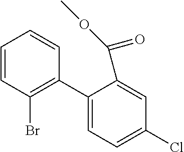 [SMILES: COC(=O)c1cc(Cl)ccc1-c1ccccc1Br] | PhMgCl | 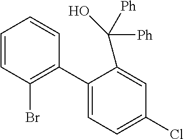 [SMILES: OC(c1ccccc1)(c1ccccc1)c1cc(Cl)ccc1-c1ccccc1Br] | 80% |
| 2s | 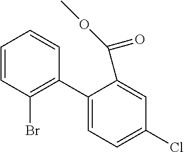 [SMILES: COC(=O)c1cc(Cl)ccc1-c1ccccc1Br] | MeMgCl | 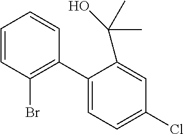 [SMILES: CC(C)(O)c1cc(Cl)ccc1-c1ccccc1Br] | 75% |
| 2t | 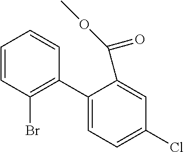 [SMILES: COC(=O)c1cc(Cl)ccc1-c1ccccc1Br] | 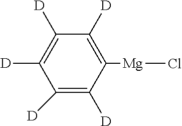 | 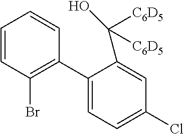 | 85% |
| 2u | 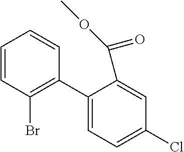 [SMILES: COC(=O)c1cc(Cl)ccc1-c1ccccc1Br] | CD₃MgCl | 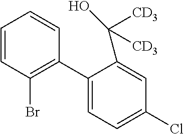 [SMILES: [2H]C([2H])([2H])C(O)(c1cc(Cl)ccc1-c1ccccc1Br)C([2H])([2H])[2H]] | 78% |
| 2v | 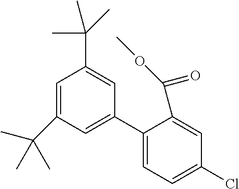 [SMILES: COC(=O)c1cc(Cl)ccc1-c1cc(C(C)(C)C)cc(C(C)(C)C)c1] | CH₃MgCl | 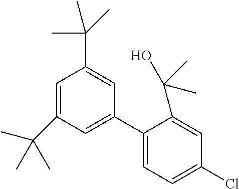 [SMILES: CC(C)(C)c1cc(-c2ccc(Cl)cc2C(C)(C)O)cc(C(C)(C)C)c1] | 73% |
| 2w | 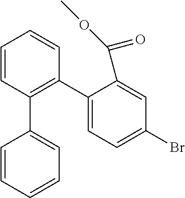 [SMILES: COC(=O)c1cc(Br)ccc1-c1ccccc1-c1ccccc1] | 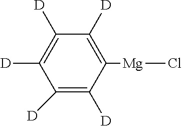 | 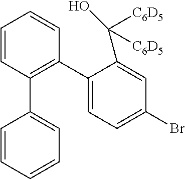 | 80% |
| 2x | 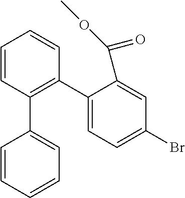 [SMILES: COC(=O)c1cc(Br)ccc1-c1ccccc1-c1ccccc1] | CD₃MgCl | 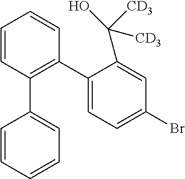 [SMILES: [2H]C([2H])([2H])C(O)(c1cc(Br)ccc1-c1ccccc1-c1ccccc1)C([2H])([2H])[2H]] | 79% |

### Synthesis of 2-bromo-9,9-dimethyl-5-phenyl-9H-fluorene 3a

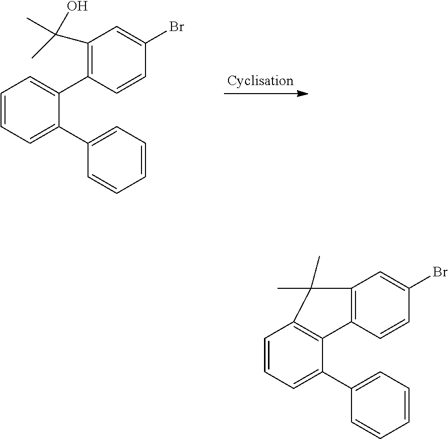 [SMILES: CC(C)(O)c1cc(Br)ccc1-c1ccccc1-c1ccccc1 ; CC1(C)c2cc(Br)ccc2-c2c(-c3ccccc3)cccc21]

A solution of 2-(2-{[1,1′-biphenyl]-2-yl}-5-bromophenyl)propan-2-ol (1.3 g, 3.5 mmol) in CH₂Cl₂ (26 mL) is treated with 0.54 mL of BF₃.Et₂O (4.6 mmol, 1.3 eq.) under argon at 0° C. The mixture is stirred for 30 minutes. The reaction is stirred at room temperature for 2 hours. The reaction is quenched with a solution of saturated NaHCO₃ and the mixture is extracted with CH₂Cl₂. The organic phase is dried with MgSO₄ and concentrated to dryness. The residue is purified by chromatography (mixture heptane/AcOEt) to isolate pure 3a (0.9 g, 72% of theory).

The following compounds are synthesized analogously:

| Ex. | | Product | Yield |
|---|---|---|---|
| 3b | 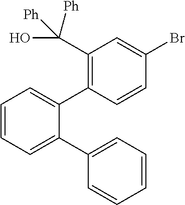 [SMILES: OC(c1ccccc1)(c1ccccc1)c1cc(Br)ccc1-c1ccccc1-c1ccccc1] | 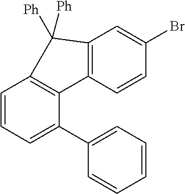 [SMILES: Brc1ccc2c(c1)C(c1ccccc1)(c1ccccc1)c1cccc(-c3ccccc3)c1-2] | 76% |
| 3c | 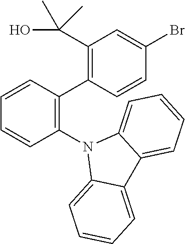 [SMILES: CC(C)(O)c1cc(Br)ccc1-c1ccccc1-n1c2ccccc2c2ccccc21] | 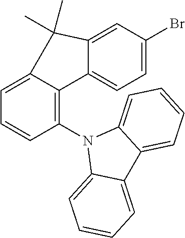 [SMILES: CC1(C)c2cc(Br)ccc2-c2c(-n3c4ccccc4c4ccccc43)cccc21] | 62% |
| 3d | 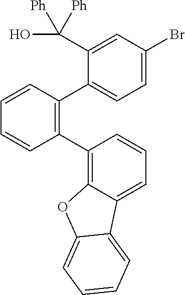 [SMILES: OC(c1ccccc1)(c1ccccc1)c1cc(Br)ccc1-c1ccccc1-c1cccc2c1oc1ccccc12] | 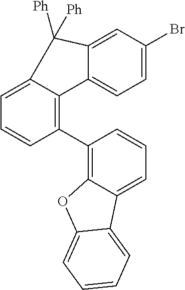 [SMILES: Brc1ccc2c(c1)C(c1ccccc1)(c1ccccc1)c1cccc(-c3cccc4c3oc3ccccc34)c1-2] | 83% |
| 3e | 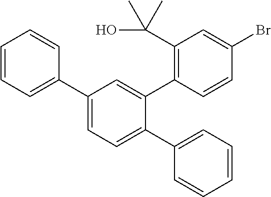 [SMILES: CC(C)(O)c1cc(Br)ccc1-c1cc(-c2ccccc2)ccc1-c1ccccc1] | 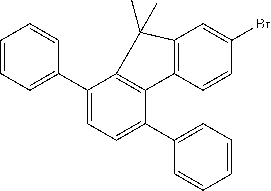 [SMILES: CC1(C)c2cc(Br)ccc2-c2c(-c3ccccc3)ccc(-c3ccccc3)c21] | 72% |
| 3f | 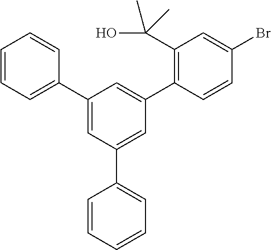 [SMILES: CC(C)(O)c1cc(Br)ccc1-c1cc(-c2ccccc2)cc(-c2ccccc2)c1] | 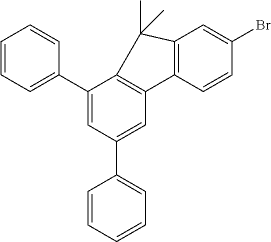 [SMILES: CC1(C)c2cc(Br)ccc2-c2cc(-c3ccccc3)cc(-c3ccccc3)c21] | 80% |
| 3g | 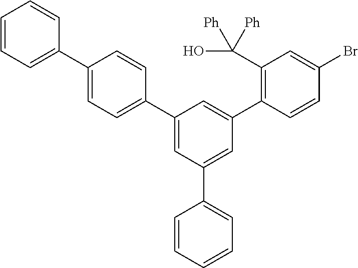 [SMILES: OC(c1ccccc1)(c1ccccc1)c1cc(Br)ccc1-c1cc(-c2ccccc2)cc(-c2ccc(-c3ccccc3)cc2)c1] | 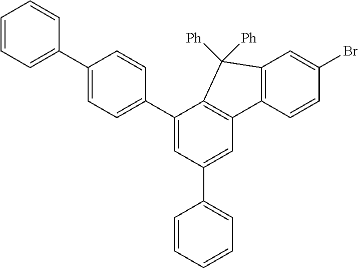 [SMILES: Brc1ccc2c(c1)C(c1ccccc1)(c1ccccc1)c1c(-c3ccc(-c4ccccc4)cc3)cc(-c3ccccc3)cc1-2] | 75% |
| 3h | 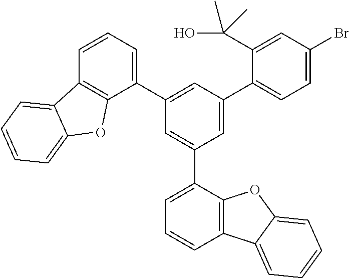 [SMILES: CC(C)(O)c1cc(Br)ccc1-c1cc(-c2cccc3c2oc2ccccc23)cc(-c2cccc3c2oc2ccccc23)c1] | 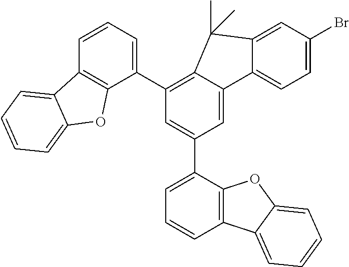 [SMILES: CC1(C)c2cc(Br)ccc2-c2cc(-c3cccc4c3oc3ccccc34)cc(-c3cccc4c3oc3ccccc34)c21] | 65% |
| 3i | 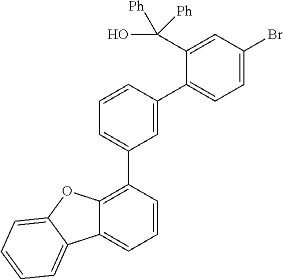 [SMILES: OC(c1ccccc1)(c1ccccc1)c1cc(Br)ccc1-c1cccc(-c2cccc3c2oc2ccccc23)c1] | 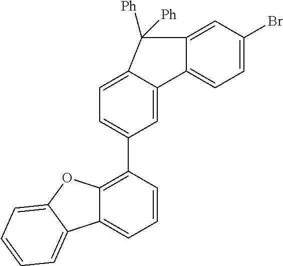 [SMILES: Brc1ccc2c(c1)C(c1ccccc1)(c1ccccc1)c1ccc(-c3cccc4c3oc3ccccc34)cc1-2] | 74% |
| 3j | 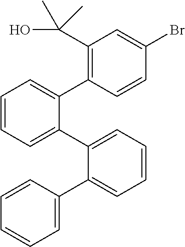 [SMILES: CC(C)(O)c1cc(Br)ccc1-c1ccccc1-c1ccccc1-c1ccccc1] | 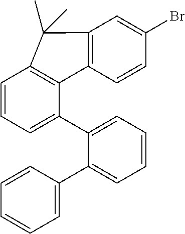 [SMILES: CC1(C)c2cc(Br)ccc2-c2c(-c3ccccc3-c3ccccc3)cccc21] | 60% |
| 3k | 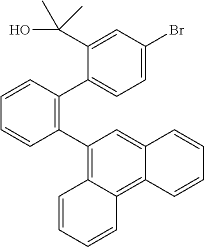 [SMILES: CC(C)(O)c1cc(Br)ccc1-c1ccccc1-c1cc2ccccc2c2ccccc12] | 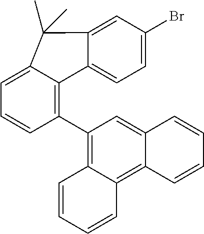 [SMILES: CC1(C)c2cc(Br)ccc2-c2c(-c3cc4ccccc4c4ccccc34)cccc21] | 70% |
| 3l | 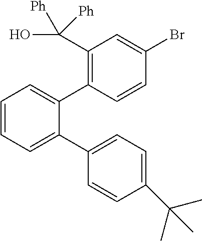 [SMILES: CC(C)(C)c1ccc(-c2ccccc2-c2ccc(Br)cc2C(O)(c2ccccc2)c2ccccc2)cc1] | 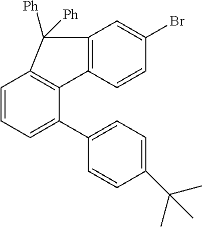 [SMILES: CC(C)(C)c1ccc(-c2cccc3c2-c2ccc(Br)cc2C3(c2ccccc2)c2ccccc2)cc1] | 60% |
| 3m | 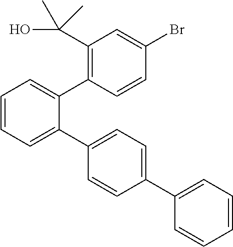 [SMILES: CC(C)(O)c1cc(Br)ccc1-c1ccccc1-c1ccc(-c2ccccc2)cc1] | 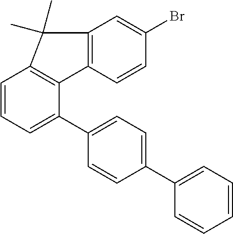 [SMILES: CC1(C)c2cc(Br)ccc2-c2c(-c3ccc(-c4ccccc4)cc3)cccc21] | 75% |
| 3n | 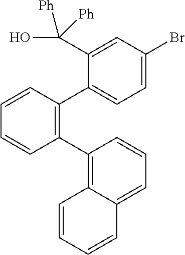 [SMILES: OC(c1ccccc1)(c1ccccc1)c1cc(Br)ccc1-c1ccccc1-c1cccc2ccccc12] | 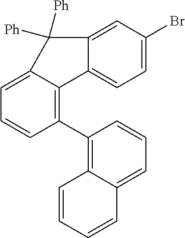 [SMILES: Brc1ccc2c(c1)C(c1ccccc1)(c1ccccc1)c1cccc(-c3cccc4ccccc34)c1-2] | 80% |
| 3o | 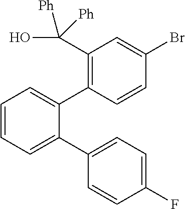 [SMILES: OC(c1ccccc1)(c1ccccc1)c1cc(Br)ccc1-c1ccccc1-c1ccc(F)cc1] | 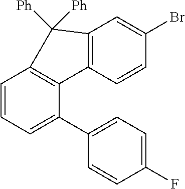 [SMILES: Fc1ccc(-c2cccc3c2-c2ccc(Br)cc2C3(c2ccccc2)c2ccccc2)cc1] | 82% |
| 3p | 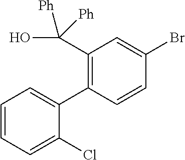 [SMILES: OC(c1ccccc1)(c1ccccc1)c1cc(Br)ccc1-c1ccccc1Cl] | 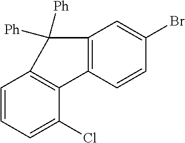 [SMILES: Clc1cccc2c1-c1ccc(Br)cc1C2(c1ccccc1)c1ccccc1] | 78% |
| 3q | 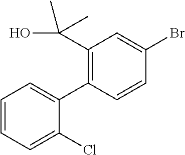 [SMILES: CC(C)(O)c1cc(Br)ccc1-c1ccccc1Cl] | 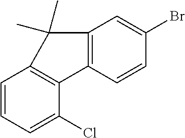 [SMILES: CC1(C)c2cc(Br)ccc2-c2c(Cl)cccc21] | 68% |
| 3r | 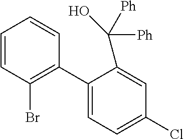 [SMILES: OC(c1ccccc1)(c1ccccc1)c1cc(Cl)ccc1-c1ccccc1Br] | 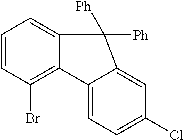 [SMILES: Clc1ccc2c(c1)C(c1ccccc1)(c1ccccc1)c1cccc(Br)c1-2] | 75% |
| 3s | 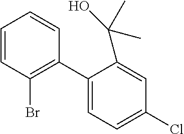 [SMILES: CC(C)(O)c1cc(Cl)ccc1-c1ccccc1Br] | 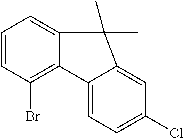 [SMILES: CC1(C)c2cc(Cl)ccc2-c2c(Br)cccc21] | 65% |
| 3t | 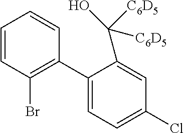 | 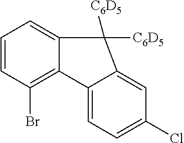 | 68% |
| 3u | 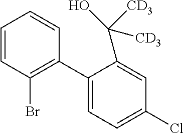 [SMILES: [2H]C([2H])([2H])C(O)(c1cc(Cl)ccc1-c1ccccc1Br)C([2H])([2H])[2H]] | 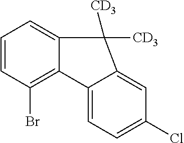 [SMILES: [2H]C([2H])([2H])C1(C([2H])([2H])[2H])c2cc(Cl)ccc2-c2c(Br)cccc21] | 78% |
| 3v | 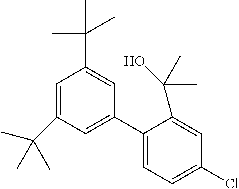 [SMILES: CC(C)(C)c1cc(-c2ccc(Cl)cc2C(C)(C)O)cc(C(C)(C)C)c1] | 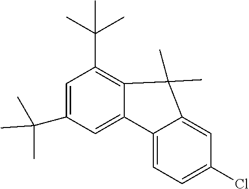 [SMILES: CC(C)(C)c1cc2c(c(C(C)(C)C)c1)C(C)(C)c1cc(Cl)ccc1-2] | 60% |
| 3w | 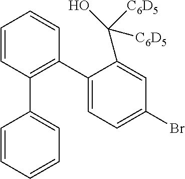 | 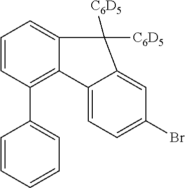 | 73% |
| 3x | 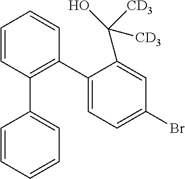 [SMILES: [2H]C([2H])([2H])C(O)(c1cc(Br)ccc1-c1ccccc1-c1ccccc1)C([2H])([2H])[2H]] | 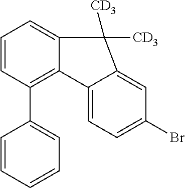 [SMILES: [2H]C([2H])([2H])C1(C([2H])([2H])[2H])c2cc(Br)ccc2-c2c(-c3ccccc3)cccc21] | 76% |

### Synthesis of N-{[1,1′-biphenyl]-4-yl}-N-(9,9-dimethyl-9H-fluoren-2-yl)-9,9-dimethyl-5-phenyl-9H-fluoren-2-amine 4a

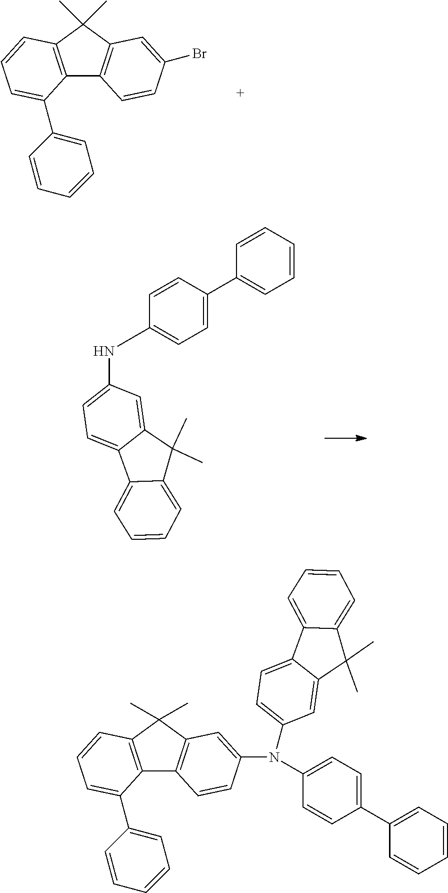 [SMILES: CC1(C)c2cc(Br)ccc2-c2c(-c3ccccc3)cccc21 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc4c(c3)C(C)(C)c3cccc(-c5ccccc5)c3-4)cc21]

S-Phos (1.06 g, 2.6 mmol), Pd₂(dba)₃ (1.18 g, 1.29 mmol) and sodium tert-butoxide (48.3 g, 85.9 mmol) are added to a solution of biphenyl-4-yl-(9,9-dimethyl-9H-fluoren-2-yl)-amine (15.5 g, 42.9 mmol) and 2-bromo-9,9-dimethyl-5-phenyl-9H-fluorene (15 g, 42.9 mmol) in degassed toluene (200 ml), and the mixture is heated under reflux for 10 h. The reaction mixture is cooled to room temperature, extended with toluene and filtered through Celite. The filtrate is evaporated in vacuo, and the residue is crystallised from toluene/heptane. The crude product is extracted in a Soxhlet extractor (toluene) and purified by zone sublimation in vacuo twice. The product is isolated in the form of an off-white solid (12 g, 45% of theory).

The following compounds are obtained analogously:

| Ex. | Halogenated Fluorene | Amine |
|---|---|---|
| 4b | 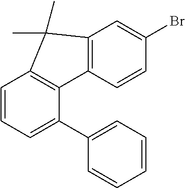 [SMILES: CC1(C)c2cc(Br)ccc2-c2c(-c3ccccc3)cccc21] | 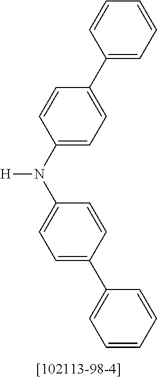 [SMILES: c1ccc(-c2ccc(Nc3ccc(-c4ccccc4)cc3)cc2)cc1] |
| 4c | 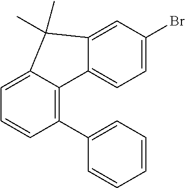 [SMILES: CC1(C)c2cc(Br)ccc2-c2c(-c3ccccc3)cccc21] | 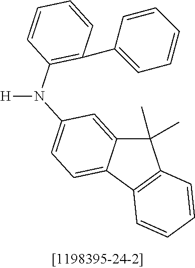 [SMILES: CC1(C)c2ccccc2-c2ccc(Nc3ccccc3-c3ccccc3)cc21] |
| 4d | 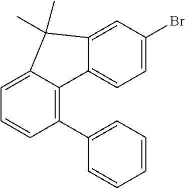 [SMILES: CC1(C)c2cc(Br)ccc2-c2c(-c3ccccc3)cccc21] | 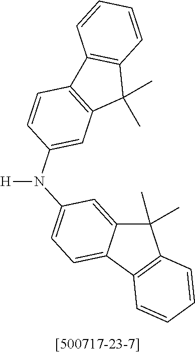 [SMILES: CC1(C)c2ccccc2-c2ccc(Nc3ccc4c(c3)C(C)(C)c3ccccc3-4)cc21] |
| 4e | 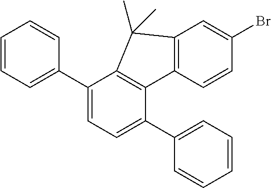 [SMILES: CC1(C)c2cc(Br)ccc2-c2c(-c3ccccc3)ccc(-c3ccccc3)c21] | 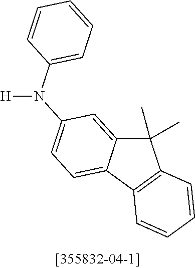 [SMILES: CC1(C)c2ccccc2-c2ccc(Nc3ccccc3)cc21] |
| 4f | 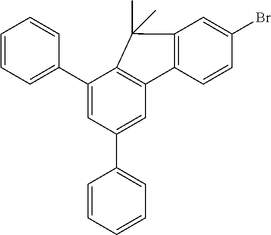 [SMILES: CC1(C)c2cc(Br)ccc2-c2cc(-c3ccccc3)cc(-c3ccccc3)c21] | 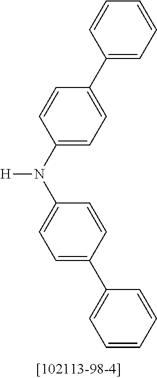 [SMILES: c1ccc(-c2ccc(Nc3ccc(-c4ccccc4)cc3)cc2)cc1] |
| 4g | 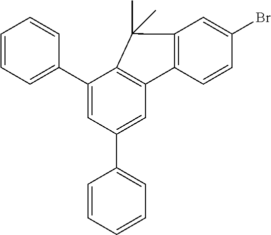 [SMILES: CC1(C)c2cc(Br)ccc2-c2cc(-c3ccccc3)cc(-c3ccccc3)c21] | 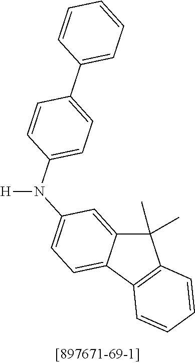 [SMILES: CC1(C)c2ccccc2-c2ccc(Nc3ccc(-c4ccccc4)cc3)cc21] |
| 4h | 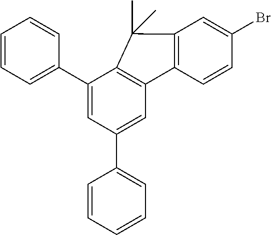 [SMILES: CC1(C)c2cc(Br)ccc2-c2cc(-c3ccccc3)cc(-c3ccccc3)c21] | 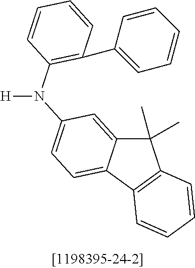 [SMILES: CC1(C)c2ccccc2-c2ccc(Nc3ccccc3-c3ccccc3)cc21] |
| 4i | 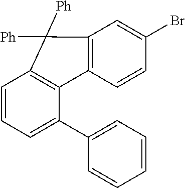 [SMILES: Brc1ccc2c(c1)C(c1ccccc1)(c1ccccc1)c1cccc(-c3ccccc3)c1-2] | 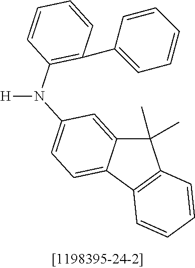 [SMILES: CC1(C)c2ccccc2-c2ccc(Nc3ccccc3-c3ccccc3)cc21] |
| 4j | 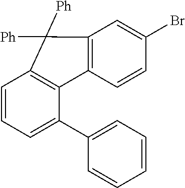 [SMILES: Brc1ccc2c(c1)C(c1ccccc1)(c1ccccc1)c1cccc(-c3ccccc3)c1-2] | 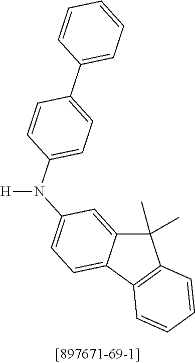 [SMILES: CC1(C)c2ccccc2-c2ccc(Nc3ccc(-c4ccccc4)cc3)cc21] |
| 4k | 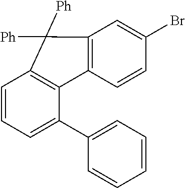 [SMILES: Brc1ccc2c(c1)C(c1ccccc1)(c1ccccc1)c1cccc(-c3ccccc3)c1-2] | 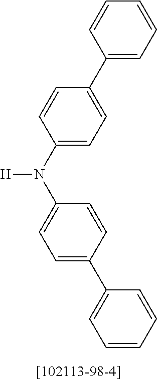 [SMILES: c1ccc(-c2ccc(Nc3ccc(-c4ccccc4)cc3)cc2)cc1] |
| 4l | 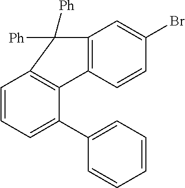 [SMILES: Brc1ccc2c(c1)C(c1ccccc1)(c1ccccc1)c1cccc(-c3ccccc3)c1-2] | 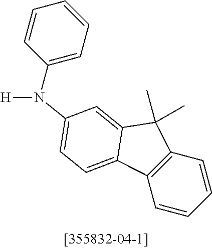 [SMILES: CC1(C)c2ccccc2-c2ccc(Nc3ccccc3)cc21] |
| 4m | 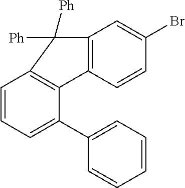 [SMILES: Brc1ccc2c(c1)C(c1ccccc1)(c1ccccc1)c1cccc(-c3ccccc3)c1-2] | 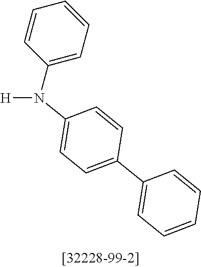 [SMILES: c1ccc(Nc2ccc(-c3ccccc3)cc2)cc1] |
| 4n | 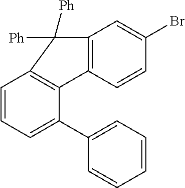 [SMILES: Brc1ccc2c(c1)C(c1ccccc1)(c1ccccc1)c1cccc(-c3ccccc3)c1-2] | 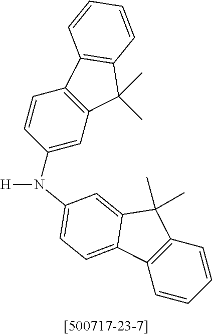 [SMILES: CC1(C)c2ccccc2-c2ccc(Nc3ccc4c(c3)C(C)(C)c3ccccc3-4)cc21] |
| 4o | 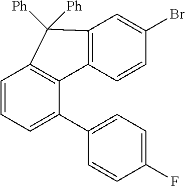 [SMILES: Fc1ccc(-c2cccc3c2-c2ccc(Br)cc2C3(c2ccccc2)c2ccccc2)cc1] | 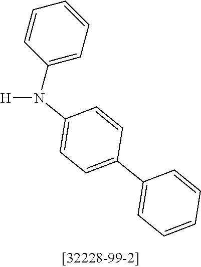 [SMILES: c1ccc(Nc2ccc(-c3ccccc3)cc2)cc1] |
| 4p | 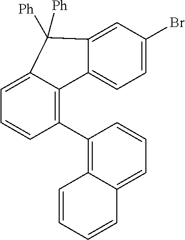 [SMILES: Brc1ccc2c(c1)C(c1ccccc1)(c1ccccc1)c1cccc(-c3cccc4ccccc34)c1-2] | 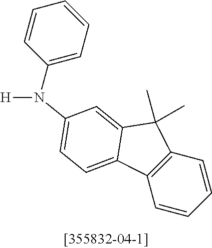 [SMILES: CC1(C)c2ccccc2-c2ccc(Nc3ccccc3)cc21] |
| 4q | 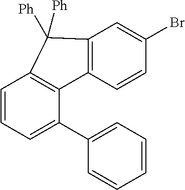 [SMILES: Brc1ccc2c(c1)C(c1ccccc1)(c1ccccc1)c1cccc(-c3ccccc3)c1-2] | 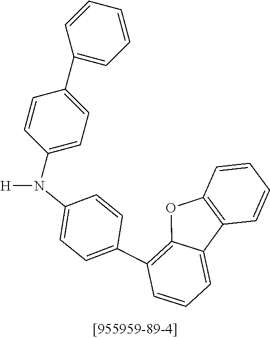 [SMILES: c1ccc(-c2ccc(Nc3ccc(-c4cccc5c4oc4ccccc45)cc3)cc2)cc1] |
| 4r | 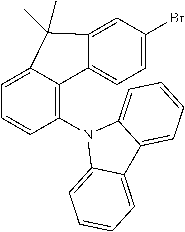 [SMILES: CC1(C)c2cc(Br)ccc2-c2c(-n3c4ccccc4c4ccccc43)cccc21] | 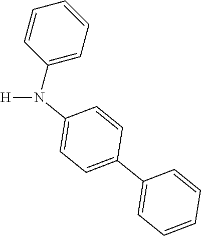 [SMILES: c1ccc(Nc2ccc(-c3ccccc3)cc2)cc1] |
| 4s | 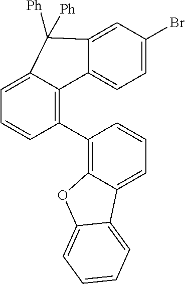 [SMILES: Brc1ccc2c(c1)C(c1ccccc1)(c1ccccc1)c1cccc(-c3cccc4c3oc3ccccc34)c1-2] | 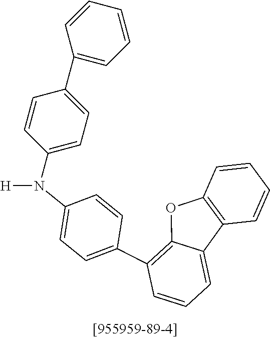 [SMILES: c1ccc(-c2ccc(Nc3ccc(-c4cccc5c4oc4ccccc45)cc3)cc2)cc1] |
| 4t | 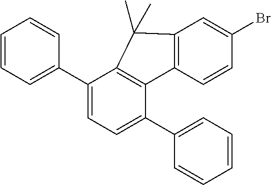 [SMILES: CC1(C)c2cc(Br)ccc2-c2c(-c3ccccc3)ccc(-c3ccccc3)c21] | 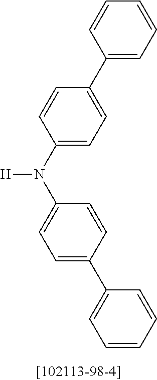 [SMILES: c1ccc(-c2ccc(Nc3ccc(-c4ccccc4)cc3)cc2)cc1] |
| 4u | 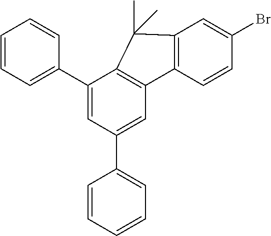 [SMILES: CC1(C)c2cc(Br)ccc2-c2cc(-c3ccccc3)cc(-c3ccccc3)c21] | 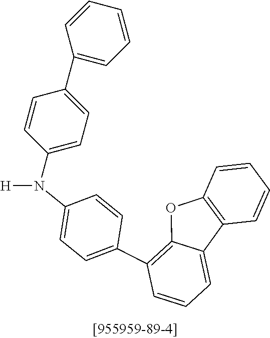 [SMILES: c1ccc(-c2ccc(Nc3ccc(-c4cccc5c4oc4ccccc45)cc3)cc2)cc1] |
| 4v | 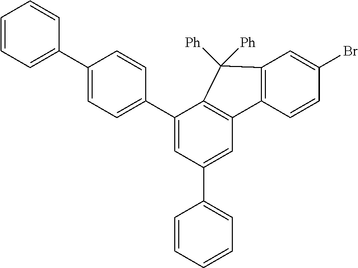 [SMILES: Brc1ccc2c(c1)C(c1ccccc1)(c1ccccc1)c1c(-c3ccc(-c4ccccc4)cc3)cc(-c3ccccc3)cc1-2] | 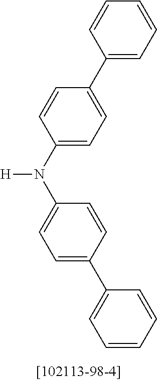 [SMILES: c1ccc(-c2ccc(Nc3ccc(-c4ccccc4)cc3)cc2)cc1] |
| 4w | 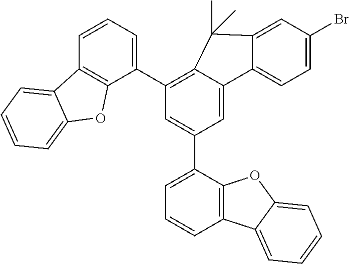 [SMILES: CC1(C)c2cc(Br)ccc2-c2cc(-c3cccc4c3oc3ccccc34)cc(-c3cccc4c3oc3ccccc34)c21] | 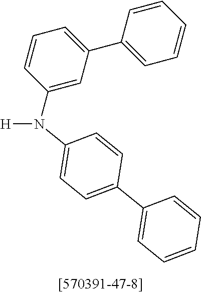 [SMILES: c1ccc(-c2ccc(Nc3cccc(-c4ccccc4)c3)cc2)cc1] |
| 4x | 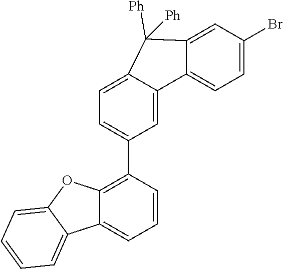 [SMILES: Brc1ccc2c(c1)C(c1ccccc1)(c1ccccc1)c1ccc(-c3cccc4c3oc3ccccc34)cc1-2] | 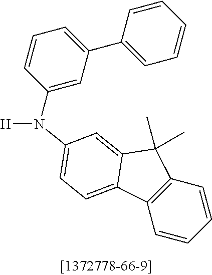 [SMILES: CC1(C)c2ccccc2-c2ccc(Nc3cccc(-c4ccccc4)c3)cc21] |
| 4y | 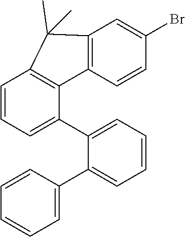 [SMILES: CC1(C)c2cc(Br)ccc2-c2c(-c3ccccc3-c3ccccc3)cccc21] | 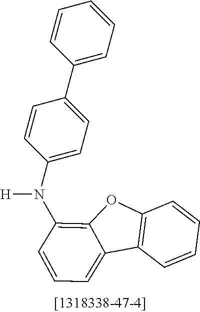 [SMILES: c1ccc(-c2ccc(Nc3cccc4c3oc3ccccc34)cc2)cc1] |
| 4z | 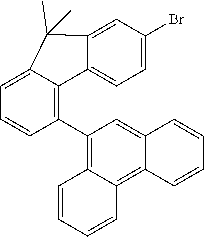 [SMILES: CC1(C)c2cc(Br)ccc2-c2c(-c3cc4ccccc4c4ccccc34)cccc21] | 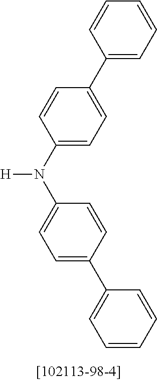 [SMILES: c1ccc(-c2ccc(Nc3ccc(-c4ccccc4)cc3)cc2)cc1] |
| 4aa | 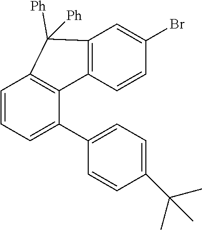 [SMILES: CC(C)(C)c1ccc(-c2cccc3c2-c2ccc(Br)cc2C3(c2ccccc2)c2ccccc2)cc1] | 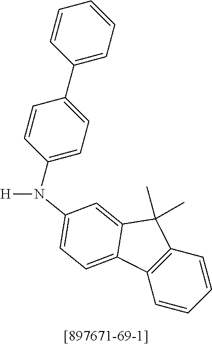 [SMILES: CC1(C)c2ccccc2-c2ccc(Nc3ccc(-c4ccccc4)cc3)cc21] |
| 4ab | 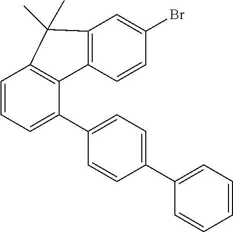 [SMILES: CC1(C)c2cc(Br)ccc2-c2c(-c3ccc(-c4ccccc4)cc3)cccc21] | 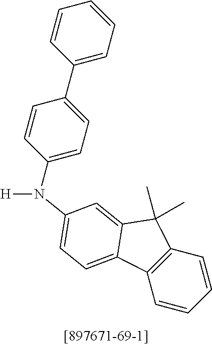 [SMILES: CC1(C)c2ccccc2-c2ccc(Nc3ccc(-c4ccccc4)cc3)cc21] |
| 4ac | 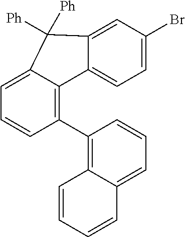 [SMILES: Brc1ccc2c(c1)C(c1ccccc1)(c1ccccc1)c1cccc(-c3cccc4ccccc34)c1-2] | 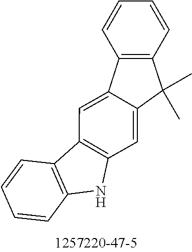 [SMILES: CC1(C)c2ccccc2-c2cc3c(cc21)[nH]c1ccccc13] |
| 4ad | 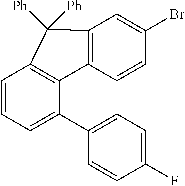 [SMILES: Fc1ccc(-c2cccc3c2-c2ccc(Br)cc2C3(c2ccccc2)c2ccccc2)cc1] | 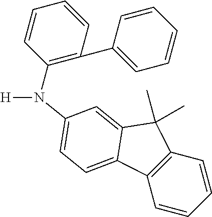 [SMILES: CC1(C)c2ccccc2-c2ccc(Nc3ccccc3-c3ccccc3)cc21] |
| 4ae | 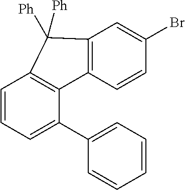 [SMILES: Brc1ccc2c(c1)C(c1ccccc1)(c1ccccc1)c1cccc(-c3ccccc3)c1-2] | 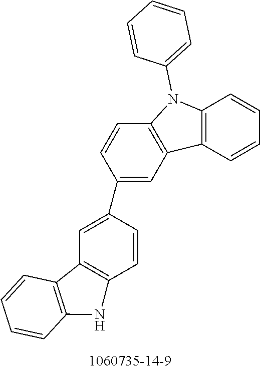 [SMILES: c1ccc(-n2c3ccccc3c3cc(-c4ccc5[nH]c6ccccc6c5c4)ccc32)cc1] |
| 4af | 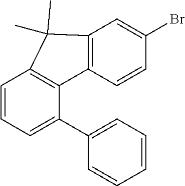 [SMILES: CC1(C)c2cc(Br)ccc2-c2c(-c3ccccc3)cccc21] | 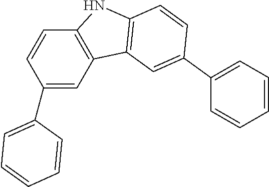 [SMILES: c1ccc(-c2ccc3[nH]c4ccc(-c5ccccc5)cc4c3c2)cc1] |
| 4ag | 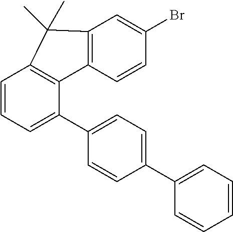 [SMILES: CC1(C)c2cc(Br)ccc2-c2c(-c3ccc(-c4ccccc4)cc3)cccc21] | 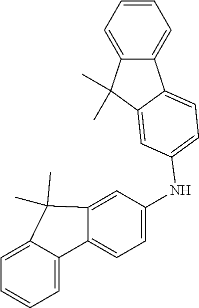 [SMILES: CC1(C)c2ccccc2-c2ccc(Nc3ccc4c(c3)C(C)(C)c3ccccc3-4)cc21] |
| 4ah | 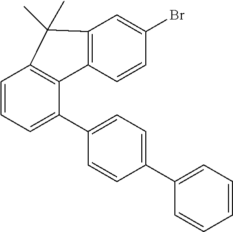 [SMILES: CC1(C)c2cc(Br)ccc2-c2c(-c3ccc(-c4ccccc4)cc3)cccc21] | 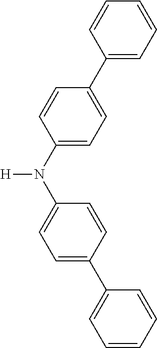 [SMILES: c1ccc(-c2ccc(Nc3ccc(-c4ccccc4)cc3)cc2)cc1] |
| 4ai | 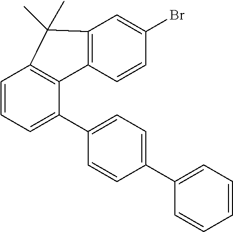 [SMILES: CC1(C)c2cc(Br)ccc2-c2c(-c3ccc(-c4ccccc4)cc3)cccc21] | 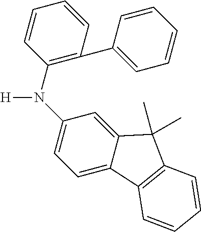 [SMILES: CC1(C)c2ccccc2-c2ccc(Nc3ccccc3-c3ccccc3)cc21] |
| 4aj | 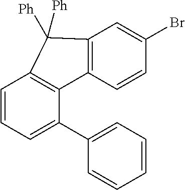 [SMILES: Brc1ccc2c(c1)C(c1ccccc1)(c1ccccc1)c1cccc(-c3ccccc3)c1-2] | 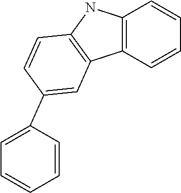 [SMILES: c1ccc(-c2ccc3[nH]c4ccccc4c3c2)cc1] |
| 4ak | 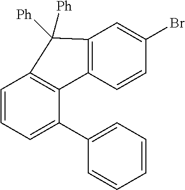 [SMILES: Brc1ccc2c(c1)C(c1ccccc1)(c1ccccc1)c1cccc(-c3ccccc3)c1-2] | 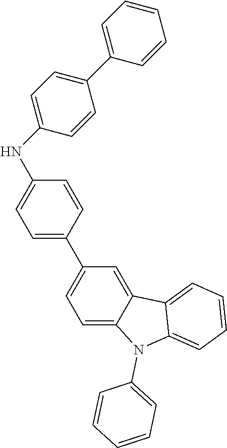 [SMILES: c1ccc(-c2ccc(Nc3ccc(-c4ccc5c(c4)c4ccccc4n5-c4ccccc4)cc3)cc2)cc1] |
| 4al | 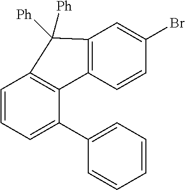 [SMILES: Brc1ccc2c(c1)C(c1ccccc1)(c1ccccc1)c1cccc(-c3ccccc3)c1-2] | 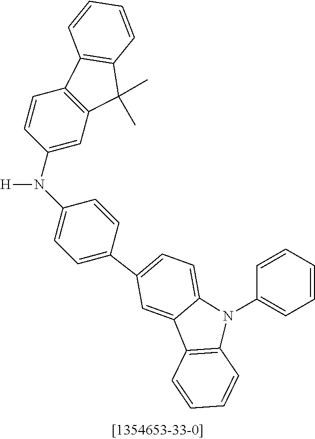 [SMILES: CC1(C)c2ccccc2-c2ccc(Nc3ccc(-c4ccc5c(c4)c4ccccc4n5-c4ccccc4)cc3)cc21] |
| 4am | 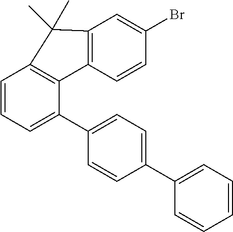 [SMILES: CC1(C)c2cc(Br)ccc2-c2c(-c3ccc(-c4ccccc4)cc3)cccc21] | 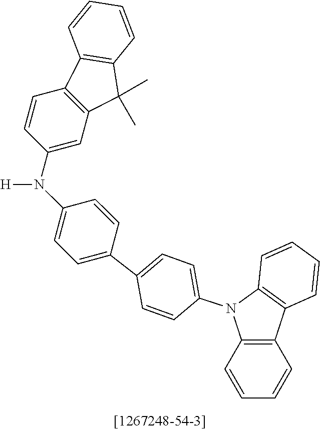 [SMILES: CC1(C)c2ccccc2-c2ccc(Nc3ccc(-c4ccc(-n5c6ccccc6c6ccccc65)cc4)cc3)cc21] |
| 4an | 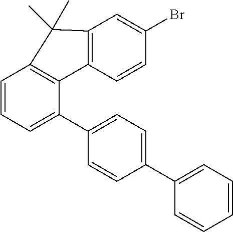 [SMILES: CC1(C)c2cc(Br)ccc2-c2c(-c3ccc(-c4ccccc4)cc3)cccc21] | 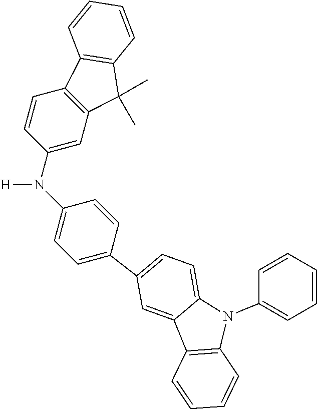 [SMILES: CC1(C)c2ccccc2-c2ccc(Nc3ccc(-c4ccc5c(c4)c4ccccc4n5-c4ccccc4)cc3)cc21] |
| 4ao | 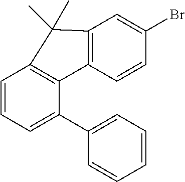 [SMILES: CC1(C)c2cc(Br)ccc2-c2c(-c3ccccc3)cccc21] | 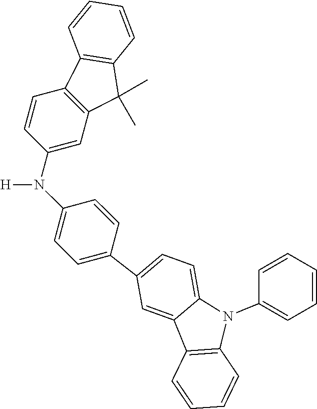 [SMILES: CC1(C)c2ccccc2-c2ccc(Nc3ccc(-c4ccc5c(c4)c4ccccc4n5-c4ccccc4)cc3)cc21] |
| 4ap | 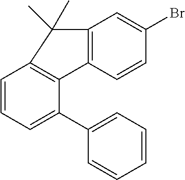 [SMILES: CC1(C)c2cc(Br)ccc2-c2c(-c3ccccc3)cccc21] | 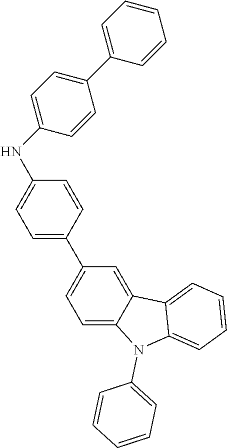 [SMILES: c1ccc(-c2ccc(Nc3ccc(-c4ccc5c(c4)c4ccccc4n5-c4ccccc4)cc3)cc2)cc1] |
| 4ao | 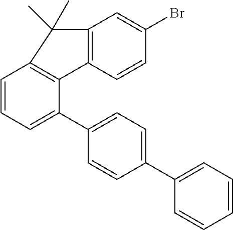 [SMILES: CC1(C)c2cc(Br)ccc2-c2c(-c3ccc(-c4ccccc4)cc3)cccc21] | 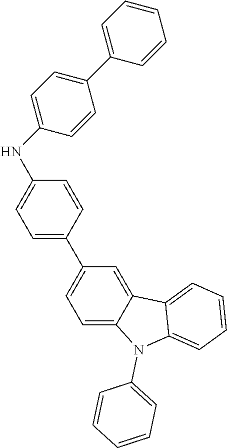 [SMILES: c1ccc(-c2ccc(Nc3ccc(-c4ccc5c(c4)c4ccccc4n5-c4ccccc4)cc3)cc2)cc1] |
| 4ap | 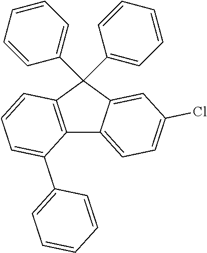 [SMILES: Clc1ccc2c(c1)C(c1ccccc1)(c1ccccc1)c1cccc(-c3ccccc3)c1-2] | 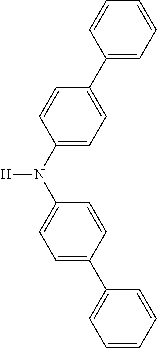 [SMILES: c1ccc(-c2ccc(Nc3ccc(-c4ccccc4)cc3)cc2)cc1] |
| 4aq | 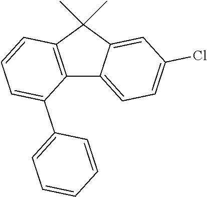 [SMILES: CC1(C)c2cc(Cl)ccc2-c2c(-c3ccccc3)cccc21] | 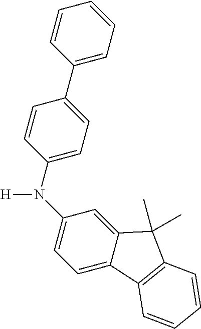 [SMILES: CC1(C)c2ccccc2-c2ccc(Nc3ccc(-c4ccccc4)cc3)cc21] |
| 4ar | 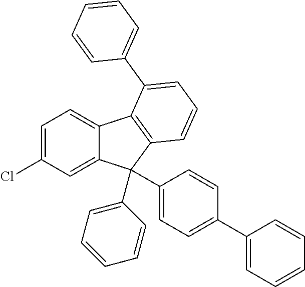 [SMILES: Clc1ccc2c(c1)C(c1ccccc1)(c1ccc(-c3ccccc3)cc1)c1cccc(-c3ccccc3)c1-2] | 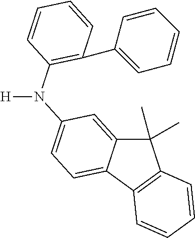 [SMILES: CC1(C)c2ccccc2-c2ccc(Nc3ccccc3-c3ccccc3)cc21] |
| 4as | 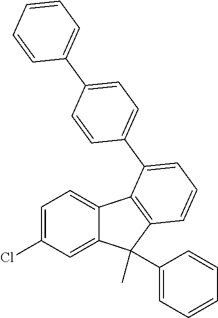 [SMILES: CC1(c2ccccc2)c2cc(Cl)ccc2-c2c(-c3ccc(-c4ccccc4)cc3)cccc21] | 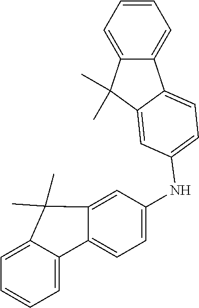 [SMILES: CC1(C)c2ccccc2-c2ccc(Nc3ccc4c(c3)C(C)(C)c3ccccc3-4)cc21] |
| 4at | 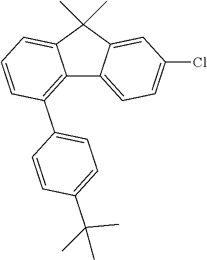 [SMILES: CC(C)(C)c1ccc(-c2cccc3c2-c2ccc(Cl)cc2C3(C)C)cc1] | 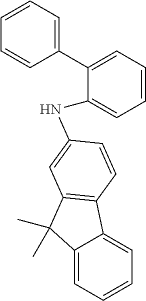 [SMILES: CC1(C)c2ccccc2-c2ccc(Nc3ccccc3-c3ccccc3)cc21] |
| 4au | 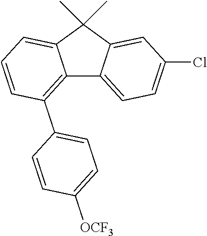 [SMILES: CC1(C)c2cc(Cl)ccc2-c2c(-c3ccc(OC(F)(F)F)cc3)cccc21] | 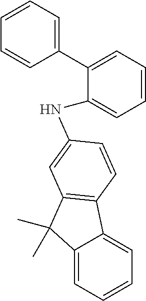 [SMILES: CC1(C)c2ccccc2-c2ccc(Nc3ccccc3-c3ccccc3)cc21] |
| 4av | 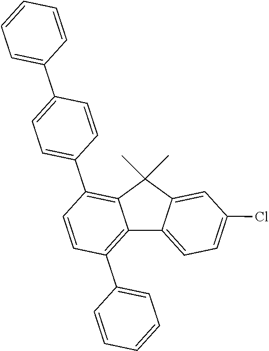 [SMILES: CC1(C)c2cc(Cl)ccc2-c2c(-c3ccccc3)ccc(-c3ccc(-c4ccccc4)cc3)c21] | 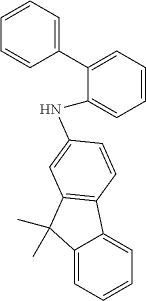 [SMILES: CC1(C)c2ccccc2-c2ccc(Nc3ccccc3-c3ccccc3)cc21] |
| 4aw | 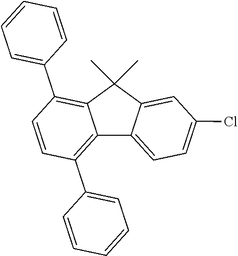 [SMILES: CC1(C)c2cc(Cl)ccc2-c2c(-c3ccccc3)ccc(-c3ccccc3)c21] | 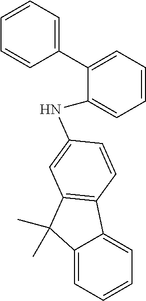 [SMILES: CC1(C)c2ccccc2-c2ccc(Nc3ccccc3-c3ccccc3)cc21] |
| 4ax | 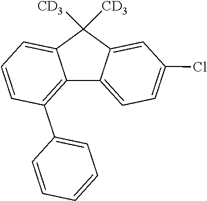 [SMILES: [2H]C([2H])([2H])C1(C([2H])([2H])[2H])c2cc(Cl)ccc2-c2c(-c3ccccc3)cccc21] | 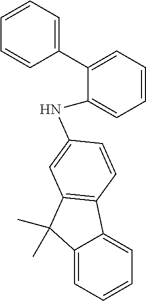 [SMILES: CC1(C)c2ccccc2-c2ccc(Nc3ccccc3-c3ccccc3)cc21] |
| 4ay | 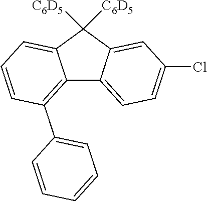 | 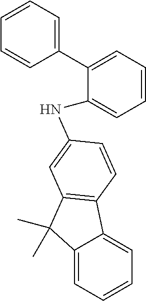 [SMILES: CC1(C)c2ccccc2-c2ccc(Nc3ccccc3-c3ccccc3)cc21] |
| 4az | 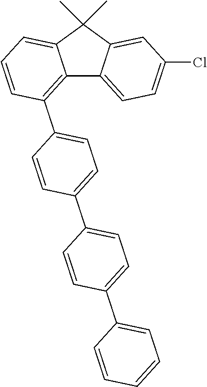 [SMILES: CC1(C)c2cc(Cl)ccc2-c2c(-c3ccc(-c4ccc(-c5ccccc5)cc4)cc3)cccc21] | 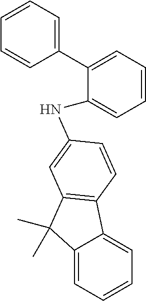 [SMILES: CC1(C)c2ccccc2-c2ccc(Nc3ccccc3-c3ccccc3)cc21] |
| 4ba | 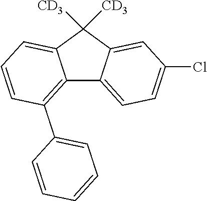 [SMILES: [2H]C([2H])([2H])C1(C([2H])([2H])[2H])c2cc(Cl)ccc2-c2c(-c3ccccc3)cccc21] | 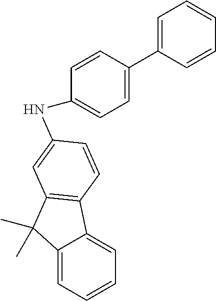 [SMILES: CC1(C)c2ccccc2-c2ccc(Nc3ccc(-c4ccccc4)cc3)cc21] |
| 4bb | 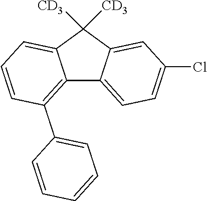 [SMILES: [2H]C([2H])([2H])C1(C([2H])([2H])[2H])c2cc(Cl)ccc2-c2c(-c3ccccc3)cccc21] | 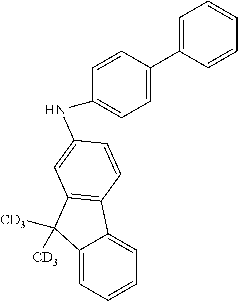 [SMILES: [2H]C([2H])([2H])C1(C([2H])([2H])[2H])c2ccccc2-c2ccc(Nc3ccc(-c4ccccc4)cc3)cc21] |

| Ex. | Product | Yield |
|---|---|---|
| 4b | 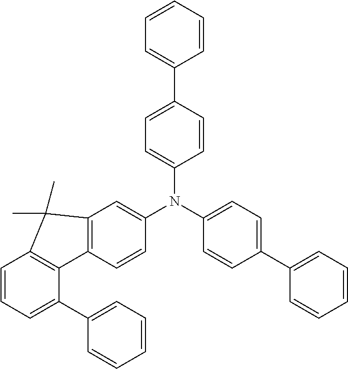 [SMILES: CC1(C)c2cc(N(c3ccc(-c4ccccc4)cc3)c3ccc(-c4ccccc4)cc3)ccc2-c2c(-c3ccccc3)cccc21] | 43% |
| 4c | 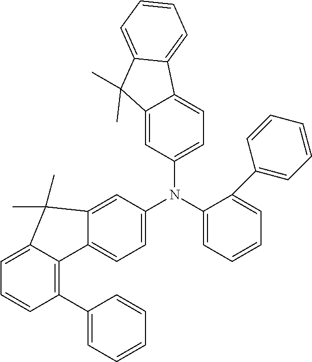 [SMILES: CC1(C)c2ccccc2-c2ccc(N(c3ccc4c(c3)C(C)(C)c3cccc(-c5ccccc5)c3-4)c3ccccc3-c3ccccc3)cc21] | 85% |
| 4d | 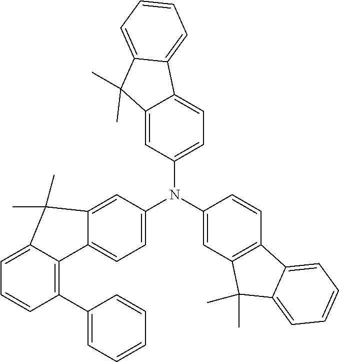 [SMILES: CC1(C)c2ccccc2-c2ccc(N(c3ccc4c(c3)C(C)(C)c3ccccc3-4)c3ccc4c(c3)C(C)(C)c3cccc(-c5ccccc5)c3-4)cc21] | 55% |
| 4e | 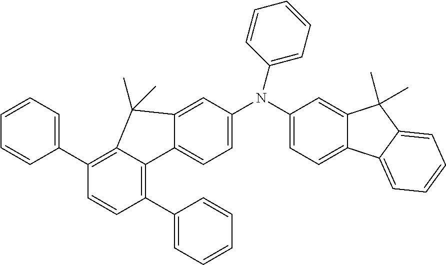 [SMILES: CC1(C)c2ccccc2-c2ccc(N(c3ccccc3)c3ccc4c(c3)C(C)(C)c3c(-c5ccccc5)ccc(-c5ccccc5)c3-4)cc21] | 64% |
| 4f | 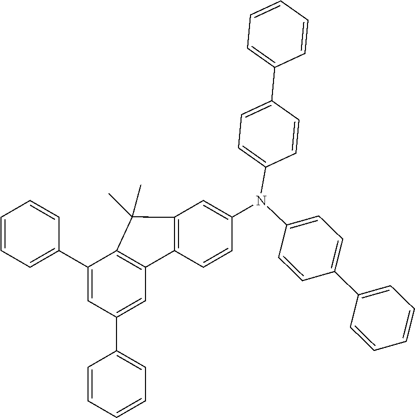 [SMILES: CC1(C)c2cc(N(c3ccc(-c4ccccc4)cc3)c3ccc(-c4ccccc4)cc3)ccc2-c2cc(-c3ccccc3)cc(-c3ccccc3)c21] | 56% |
| 4g | 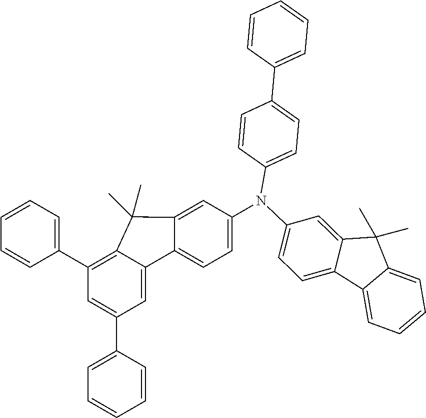 [SMILES: CC1(C)c2ccccc2-c2ccc(N(c3ccc(-c4ccccc4)cc3)c3ccc4c(c3)C(C)(C)c3c(-c5ccccc5)cc(-c5ccccc5)cc3-4)cc21] | 73% |
| 4h | 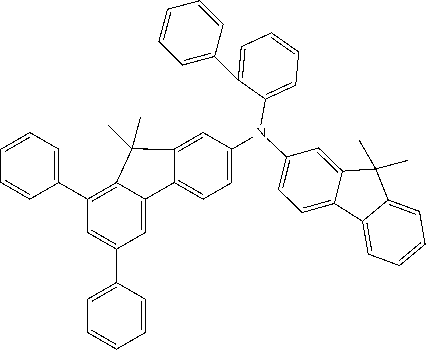 [SMILES: CC1(C)c2ccccc2-c2ccc(N(c3ccc4c(c3)C(C)(C)c3c(-c5ccccc5)cc(-c5ccccc5)cc3-4)c3ccccc3-c3ccccc3)cc21] | 65% |
| 4i | 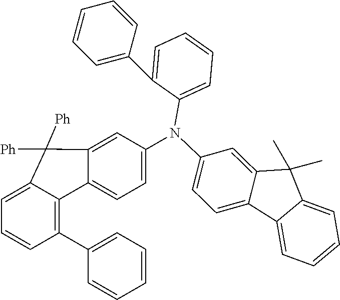 [SMILES: CC1(C)c2ccccc2-c2ccc(N(c3ccc4c(c3)C(c3ccccc3)(c3ccccc3)c3cccc(-c5ccccc5)c3-4)c3ccccc3-c3ccccc3)cc21] | 67% |
| 4j | 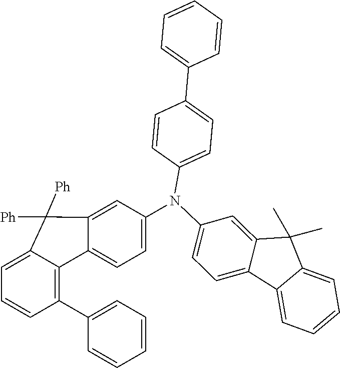 [SMILES: CC1(C)c2ccccc2-c2ccc(N(c3ccc(-c4ccccc4)cc3)c3ccc4c(c3)C(c3ccccc3)(c3ccccc3)c3cccc(-c5ccccc5)c3-4)cc21] | 66% |
| 4k | 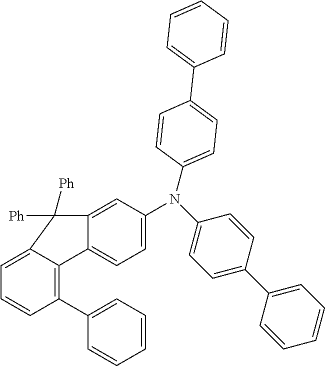 [SMILES: c1ccc(-c2ccc(N(c3ccc(-c4ccccc4)cc3)c3ccc4c(c3)C(c3ccccc3)(c3ccccc3)c3cccc(-c5ccccc5)c3-4)cc2)cc1] | 50% |
| 4l | 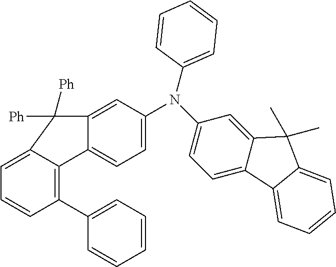 [SMILES: CC1(C)c2ccccc2-c2ccc(N(c3ccccc3)c3ccc4c(c3)C(c3ccccc3)(c3ccccc3)c3cccc(-c5ccccc5)c3-4)cc21] | 62% |
| 4m | 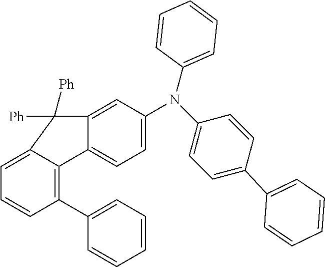 [SMILES: c1ccc(-c2ccc(N(c3ccccc3)c3ccc4c(c3)C(c3ccccc3)(c3ccccc3)c3cccc(-c5ccccc5)c3-4)cc2)cc1] | 73% |
| 4n | 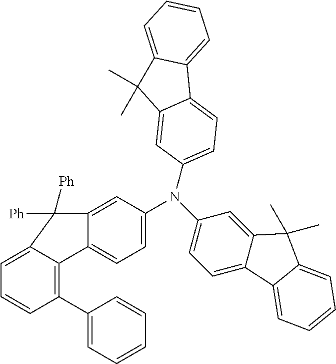 [SMILES: CC1(C)c2ccccc2-c2ccc(N(c3ccc4c(c3)C(C)(C)c3ccccc3-4)c3ccc4c(c3)C(c3ccccc3)(c3ccccc3)c3cccc(-c5ccccc5)c3-4)cc21] | 69% |
| 4o | 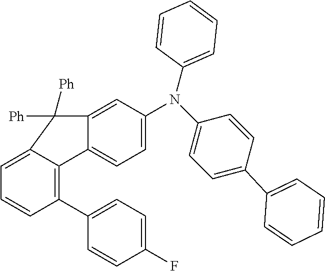 [SMILES: Fc1ccc(-c2cccc3c2-c2ccc(N(c4ccccc4)c4ccc(-c5ccccc5)cc4)cc2C3(c2ccccc2)c2ccccc2)cc1] | 57% |
| 4p | 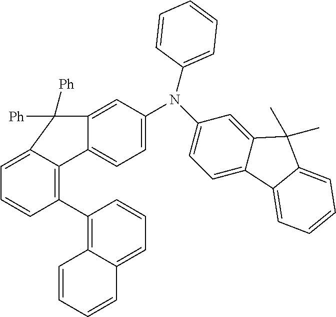 [SMILES: CC1(C)c2ccccc2-c2ccc(N(c3ccccc3)c3ccc4c(c3)C(c3ccccc3)(c3ccccc3)c3cccc(-c5cccc6ccccc56)c3-4)cc21] | 65% |
| 4q | 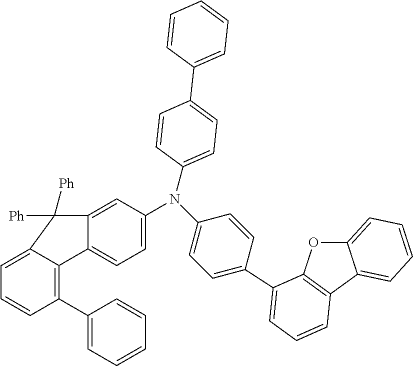 [SMILES: c1ccc(-c2ccc(N(c3ccc(-c4cccc5c4oc4ccccc45)cc3)c3ccc4c(c3)C(c3ccccc3)(c3ccccc3)c3cccc(-c5ccccc5)c3-4)cc2)cc1] | 71% |
| 4r | 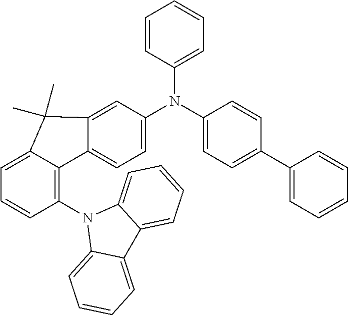 [SMILES: CC1(C)c2cc(N(c3ccccc3)c3ccc(-c4ccccc4)cc3)ccc2-c2c(-n3c4ccccc4c4ccccc43)cccc21] | 71% |
| 4s | 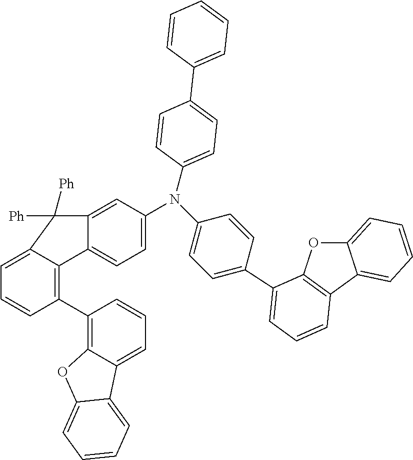 [SMILES: c1ccc(-c2ccc(N(c3ccc(-c4cccc5c4oc4ccccc45)cc3)c3ccc4c(c3)C(c3ccccc3)(c3ccccc3)c3cccc(-c5cccc6c5oc5ccccc56)c3-4)cc2)cc1] | 72% |
| 4t | 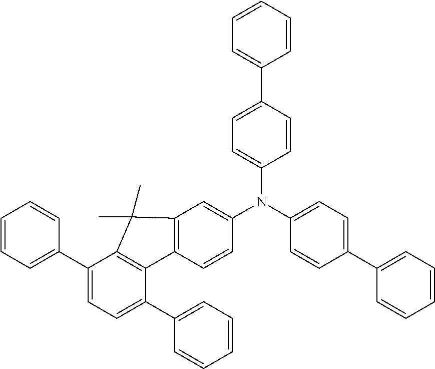 [SMILES: CC1(C)c2cc(N(c3ccc(-c4ccccc4)cc3)c3ccc(-c4ccccc4)cc3)ccc2-c2c(-c3ccccc3)ccc(-c3ccccc3)c21] | 62% |
| 4u | 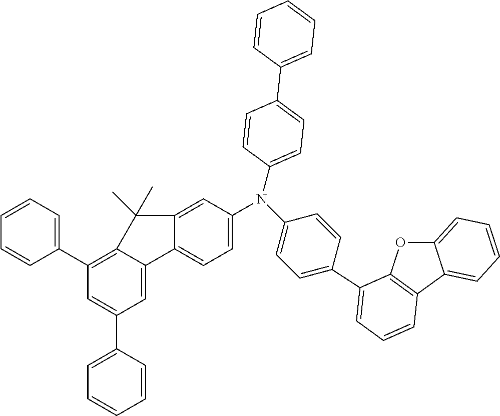 [SMILES: CC1(C)c2cc(N(c3ccc(-c4ccccc4)cc3)c3ccc(-c4cccc5c4oc4ccccc45)cc3)ccc2-c2cc(-c3ccccc3)cc(-c3ccccc3)c21] | 45% |
| 4v | 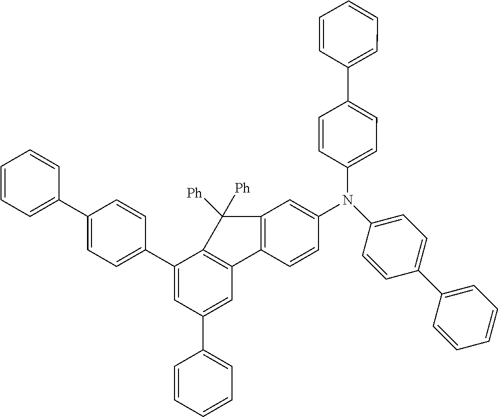 [SMILES: c1ccc(-c2ccc(-c3cc(-c4ccccc4)cc4c3C(c3ccccc3)(c3ccccc3)c3cc(N(c5ccc(-c6ccccc6)cc5)c5ccc(-c6ccccc6)cc5)ccc3-4)cc2)cc1] | 66% |
| 4w | 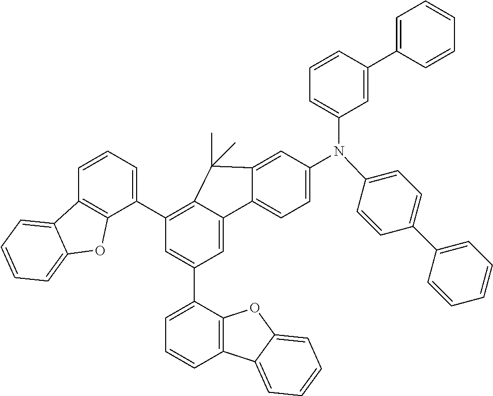 [SMILES: CC1(C)c2cc(N(c3ccc(-c4ccccc4)cc3)c3cccc(-c4ccccc4)c3)ccc2-c2cc(-c3cccc4c3oc3ccccc34)cc(-c3cccc4c3oc3ccccc34)c21] | 66% |
| 4x | 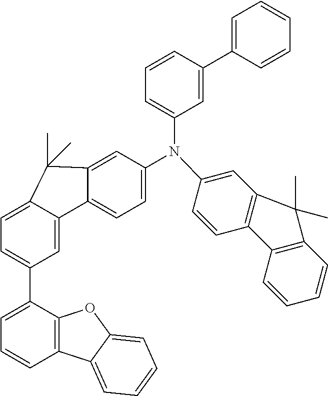 [SMILES: CC1(C)c2ccccc2-c2ccc(N(c3cccc(-c4ccccc4)c3)c3ccc4c(c3)C(C)(C)c3ccc(-c5cccc6c5oc5ccccc56)cc3-4)cc21] | 71% |
| 4y | 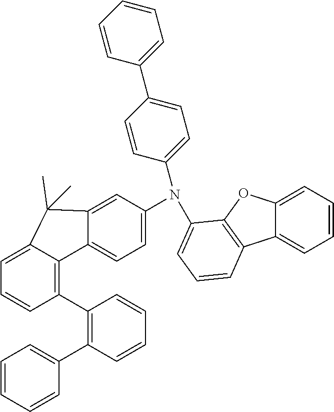 [SMILES: CC1(C)c2cc(N(c3ccc(-c4ccccc4)cc3)c3cccc4c3oc3ccccc34)ccc2-c2c(-c3ccccc3-c3ccccc3)cccc21] | 49% |
| 4z | 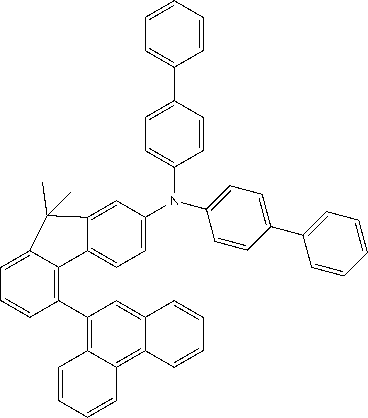 [SMILES: CC1(C)c2cc(N(c3ccc(-c4ccccc4)cc3)c3ccc(-c4ccccc4)cc3)ccc2-c2c(-c3cc4ccccc4c4ccccc34)cccc21] | 65% |
| 4aa | 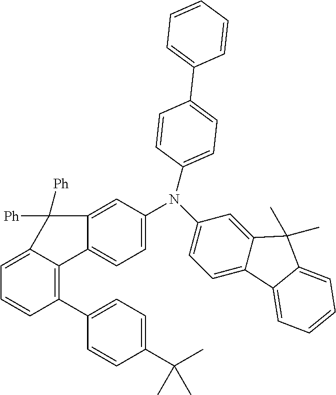 [SMILES: CC(C)(C)c1ccc(-c2cccc3c2-c2ccc(N(c4ccc(-c5ccccc5)cc4)c4ccc5c(c4)C(C)(C)c4ccccc4-5)cc2C3(c2ccccc2)c2ccccc2)cc1] | 73% |
| 4ab | 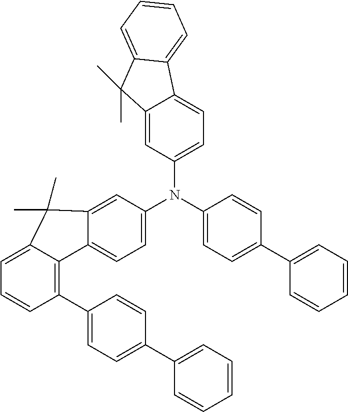 [SMILES: CC1(C)c2ccccc2-c2ccc(N(c3ccc(-c4ccccc4)cc3)c3ccc4c(c3)C(C)(C)c3cccc(-c5ccc(-c6ccccc6)cc5)c3-4)cc21] | 70% |
| 4ac | 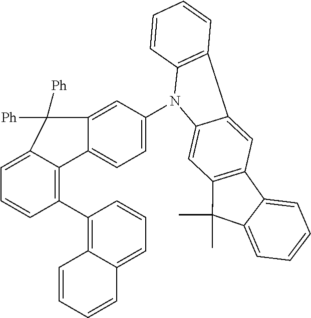 [SMILES: CC1(C)c2ccccc2-c2cc3c4ccccc4n(-c4ccc5c(c4)C(c4ccccc4)(c4ccccc4)c4cccc(-c6cccc7ccccc67)c4-5)c3cc21] | 80% |
| 4ad | 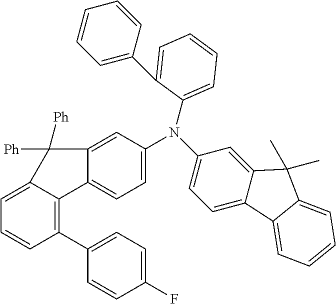 [SMILES: CC1(C)c2ccccc2-c2ccc(N(c3ccc4c(c3)C(c3ccccc3)(c3ccccc3)c3cccc(-c5ccc(F)cc5)c3-4)c3ccccc3-c3ccccc3)cc21] | 75% |
| 4ae | 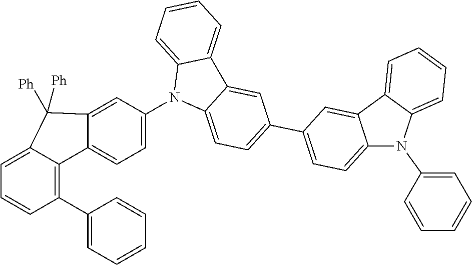 [SMILES: c1ccc(-c2cccc3c2-c2ccc(-n4c5ccccc5c5cc(-c6ccc7c(c6)c6ccccc6n7-c6ccccc6)ccc54)cc2C3(c2ccccc2)c2ccccc2)cc1] | 60% |
| 4af | 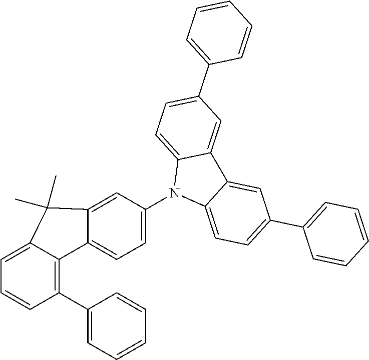 [SMILES: CC1(C)c2cc(-n3c4ccc(-c5ccccc5)cc4c4cc(-c5ccccc5)ccc43)ccc2-c2c(-c3ccccc3)cccc21] | 80% |
| 4ag | 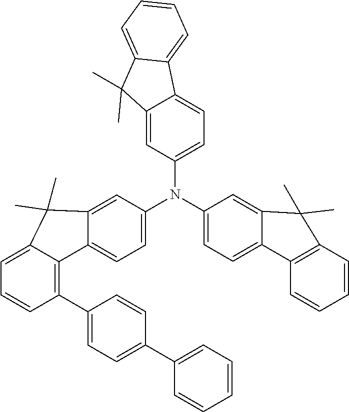 [SMILES: CC1(C)c2ccccc2-c2ccc(N(c3ccc4c(c3)C(C)(C)c3ccccc3-4)c3ccc4c(c3)C(C)(C)c3cccc(-c5ccc(-c6ccccc6)cc5)c3-4)cc21] | 76% |
| 4ah | 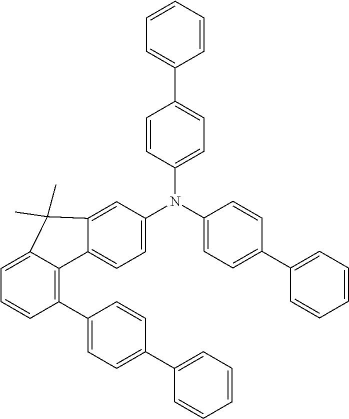 [SMILES: CC1(C)c2cc(N(c3ccc(-c4ccccc4)cc3)c3ccc(-c4ccccc4)cc3)ccc2-c2c(-c3ccc(-c4ccccc4)cc3)cccc21] | 54% |
| 4ai | 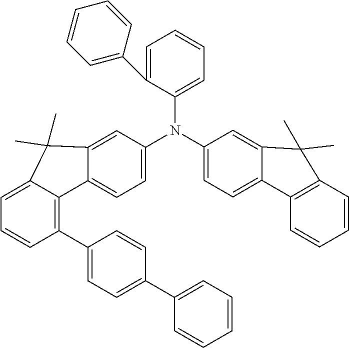 [SMILES: CC1(C)c2ccccc2-c2ccc(N(c3ccc4c(c3)C(C)(C)c3cccc(-c5ccc(-c6ccccc6)cc5)c3-4)c3ccccc3-c3ccccc3)cc21] | 47% |
| 4aj | 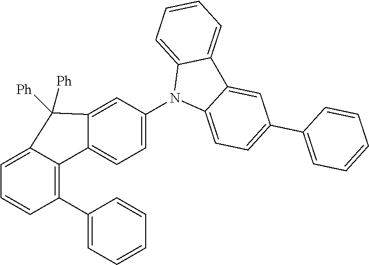 [SMILES: c1ccc(-c2ccc3c(c2)c2ccccc2n3-c2ccc3c(c2)C(c2ccccc2)(c2ccccc2)c2cccc(-c4ccccc4)c2-3)cc1] | 40% |
| 4ak | 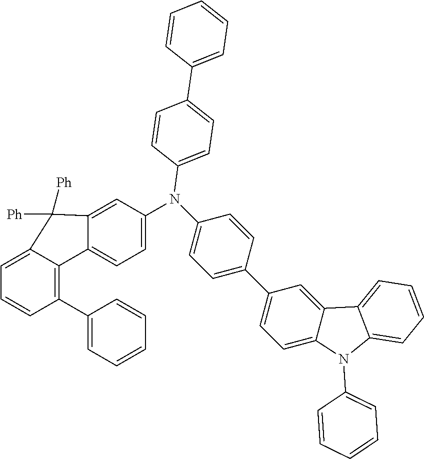 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3cccc(-c5ccccc5)c3-4)cc2)cc1] | 37% |
| 4al | 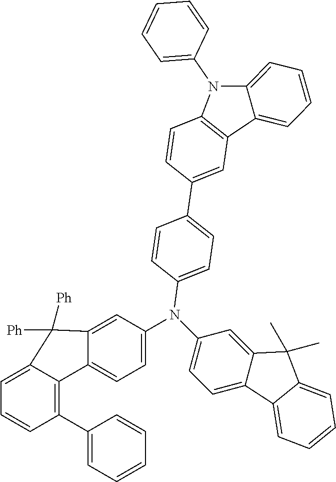 [SMILES: CC1(C)c2ccccc2-c2ccc(N(c3ccc(-c4ccc5c(c4)c4ccccc4n5-c4ccccc4)cc3)c3ccc4c(c3)C(c3ccccc3)(c3ccccc3)c3cccc(-c5ccccc5)c3-4)cc21] | 35% |
| 4am | 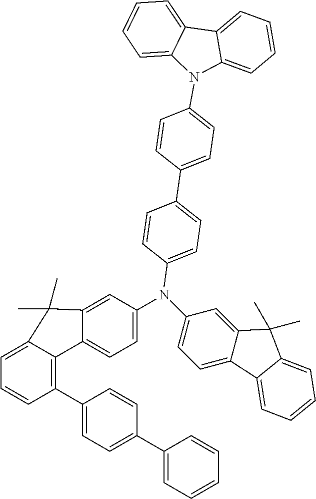 [SMILES: CC1(C)c2ccccc2-c2ccc(N(c3ccc(-c4ccc(-n5c6ccccc6c6ccccc65)cc4)cc3)c3ccc4c(c3)C(C)(C)c3cccc(-c5ccc(-c6ccccc6)cc5)c3-4)cc21] | 45% |
| 4an | 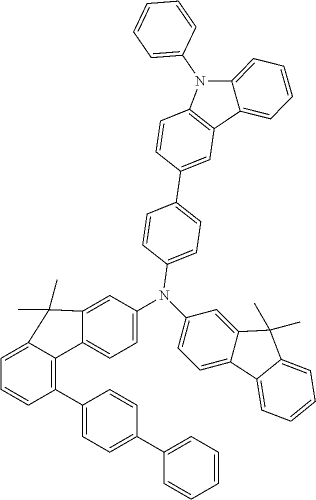 [SMILES: CC1(C)c2ccccc2-c2ccc(N(c3ccc(-c4ccc5c(c4)c4ccccc4n5-c4ccccc4)cc3)c3ccc4c(c3)C(C)(C)c3cccc(-c5ccc(-c6ccccc6)cc5)c3-4)cc21] | 47% |
| 4ao | 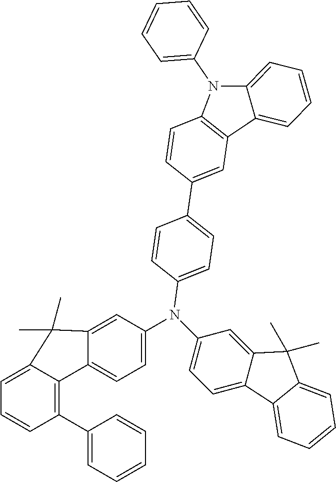 [SMILES: CC1(C)c2ccccc2-c2ccc(N(c3ccc(-c4ccc5c(c4)c4ccccc4n5-c4ccccc4)cc3)c3ccc4c(c3)C(C)(C)c3cccc(-c5ccccc5)c3-4)cc21] | 53% |
| 4ap | 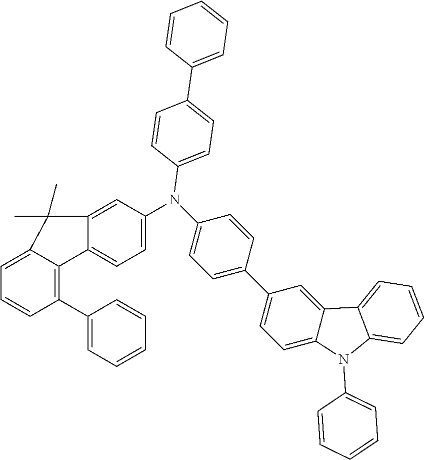 [SMILES: CC1(C)c2cc(N(c3ccc(-c4ccccc4)cc3)c3ccc(-c4ccc5c(c4)c4ccccc4n5-c4ccccc4)cc3)ccc2-c2c(-c3ccccc3)cccc21] | 57% |
| 4ao | 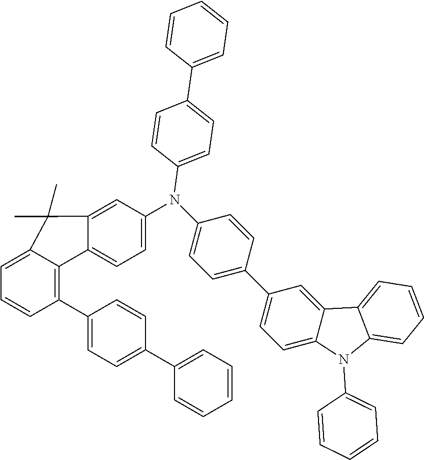 [SMILES: CC1(C)c2cc(N(c3ccc(-c4ccccc4)cc3)c3ccc(-c4ccc5c(c4)c4ccccc4n5-c4ccccc4)cc3)ccc2-c2c(-c3ccc(-c4ccccc4)cc3)cccc21] | 43% |
| 4ap | 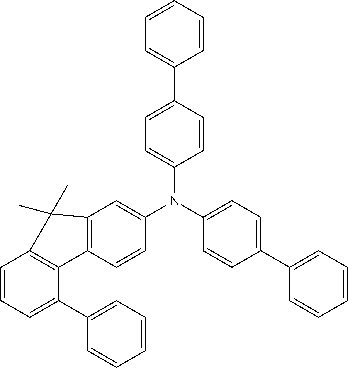 [SMILES: CC1(C)c2cc(N(c3ccc(-c4ccccc4)cc3)c3ccc(-c4ccccc4)cc3)ccc2-c2c(-c3ccccc3)cccc21] | 56% |
| 4aq | 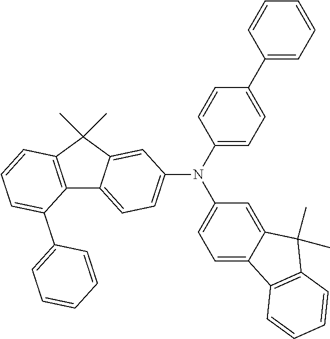 [SMILES: CC1(C)c2ccccc2-c2ccc(N(c3ccc(-c4ccccc4)cc3)c3ccc4c(c3)C(C)(C)c3cccc(-c5ccccc5)c3-4)cc21] | 48% |
| 4ar | 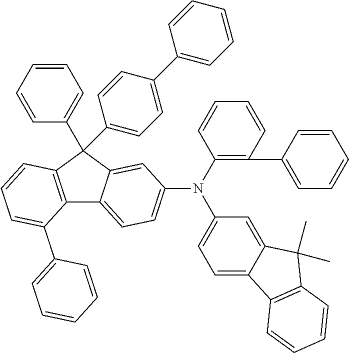 [SMILES: CC1(C)c2ccccc2-c2ccc(N(c3ccc4c(c3)C(c3ccccc3)(c3ccc(-c5ccccc5)cc3)c3cccc(-c5ccccc5)c3-4)c3ccccc3-c3ccccc3)cc21] | 61% |
| 4as | 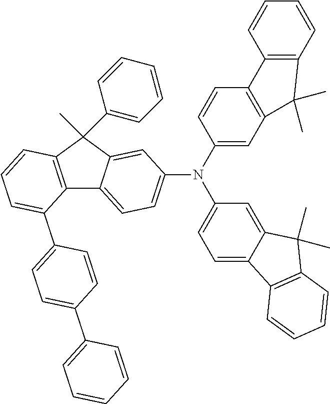 [SMILES: CC1(C)c2ccccc2-c2ccc(N(c3ccc4c(c3)C(C)(C)c3ccccc3-4)c3ccc4c(c3)C(C)(c3ccccc3)c3cccc(-c5ccc(-c6ccccc6)cc5)c3-4)cc21] | 49% |
| 4at | 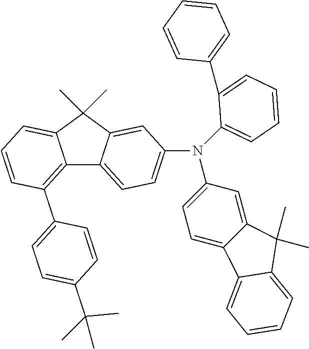 [SMILES: CC(C)(C)c1ccc(-c2cccc3c2-c2ccc(N(c4ccc5c(c4)C(C)(C)c4ccccc4-5)c4ccccc4-c4ccccc4)cc2C3(C)C)cc1] | 50% |
| 4au | 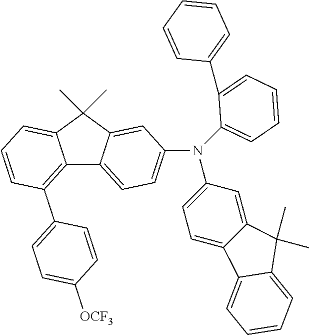 [SMILES: CC1(C)c2ccccc2-c2ccc(N(c3ccc4c(c3)C(C)(C)c3cccc(-c5ccc(OC(F)(F)F)cc5)c3-4)c3ccccc3-c3ccccc3)cc21] | 55% |
| 4av | 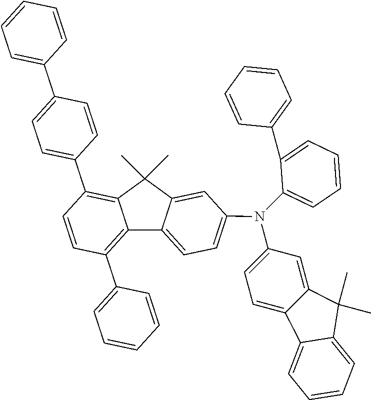 [SMILES: CC1(C)c2ccccc2-c2ccc(N(c3ccc4c(c3)C(C)(C)c3c(-c5ccc(-c6ccccc6)cc5)ccc(-c5ccccc5)c3-4)c3ccccc3-c3ccccc3)cc21] | 60% |
| 4aw | 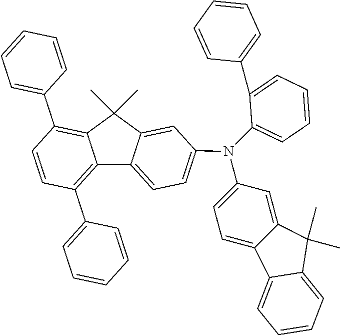 [SMILES: CC1(C)c2ccccc2-c2ccc(N(c3ccc4c(c3)C(C)(C)c3c(-c5ccccc5)ccc(-c5ccccc5)c3-4)c3ccccc3-c3ccccc3)cc21] | 64% |
| 4ax | 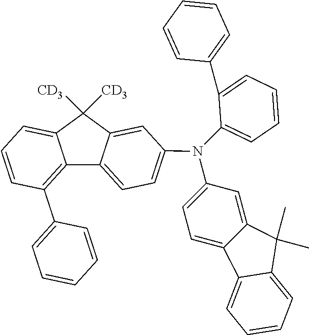 [SMILES: [2H]C([2H])([2H])C1(C([2H])([2H])[2H])c2cc(N(c3ccc4c(c3)C(C)(C)c3ccccc3-4)c3ccccc3-c3ccccc3)ccc2-c2c(-c3ccccc3)cccc21] | 52% |
| 4ay | 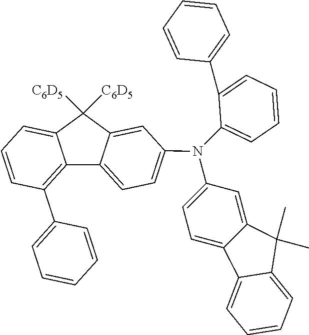 | 68% |
| 4az | 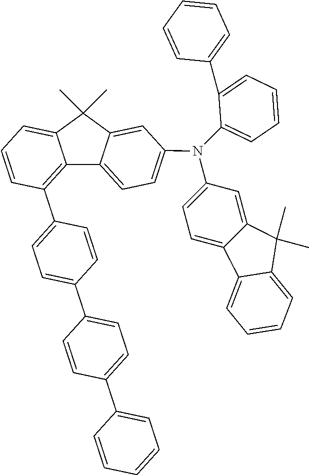 [SMILES: CC1(C)c2ccccc2-c2ccc(N(c3ccc4c(c3)C(C)(C)c3cccc(-c5ccc(-c6ccc(-c7ccccc7)cc6)cc5)c3-4)c3ccccc3-c3ccccc3)cc21] | 70% |
| 4ba | 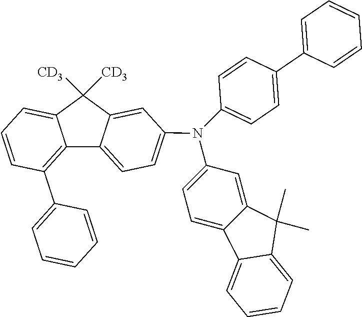 [SMILES: [2H]C([2H])([2H])C1(C([2H])([2H])[2H])c2cc(N(c3ccc(-c4ccccc4)cc3)c3ccc4c(c3)C(C)(C)c3ccccc3-4)ccc2-c2c(-c3ccccc3)cccc21] | 61% |
| 4bb | 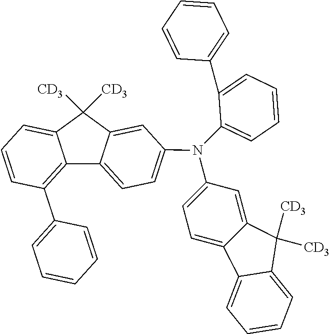 [SMILES: [2H]C([2H])([2H])C1(C([2H])([2H])[2H])c2ccccc2-c2ccc(N(c3ccc4c(c3)C(C([2H])([2H])[2H])(C([2H])([2H])[2H])c3cccc(-c5ccccc5)c3-4)c3ccccc3-c3ccccc3)cc21] | 54% |

### Synthesis of N-{[1,1′-biphenyl]-4-yl}-N-[4-(9,9-dimethyl-5-phenyl-9H-fluoren-2-yl)phenyl]-9,9-dimethyl-9H-fluoren-2-amine 5a

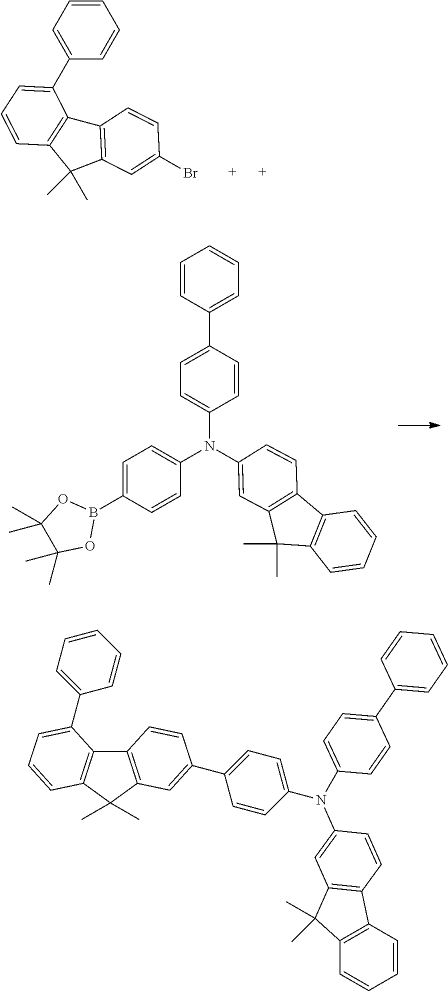 [SMILES: CC1(C)c2cc(Br)ccc2-c2c(-c3ccccc3)cccc21 ; CC1(C)c2ccccc2-c2ccc(N(c3ccc(B4OC(C)(C)C(C)(C)O4)cc3)c3ccc(-c4ccccc4)cc3)cc21 ; CC1(C)c2ccccc2-c2ccc(N(c3ccc(-c4ccccc4)cc3)c3ccc(-c4ccc5c(c4)C(C)(C)c4cccc(-c6ccccc6)c4-5)cc3)cc21]

59.1 g (101.8 mmol) of biphenyl-4-yl-(9,9-dimethyl-9H-fluoren-2-yl (4,4,5,5-tetramethyl-[1,3,2]dioxaborolan-2-yl)-phenyl]-amine, 35.5 g (101.8 mmol) of 2-bromo-9,9-dimethyl-5-phenyl-9H-fluorene, 3.88 g (5.14 mmol) of PdCl₂(Cy)₃, 31.2 g (205.6 mmol) of cesium fluoride are dissolved in 800 mL of toluene. The reaction mixture is refluxed and agitated under an argon atmosphere for 12 hours and after cooling to room temperature, the mixture is filtered through Celite. The filtrate is evaporated in vacuo, and the residue is crystallised from heptane. The crude product is extracted in a Soxhlet extractor (toluene) and purified by zone sublimation in vacuo twice. The product is isolated in the form of a white solid (42 g, 59% of theory).

The following compounds are synthesized analogously:

| Ex. | Halogenated Fluorene | Amine |
|---|---|---|
| 5b | 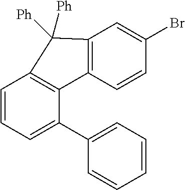 [SMILES: Brc1ccc2c(c1)C(c1ccccc1)(c1ccccc1)c1cccc(-c3ccccc3)c1-2] | 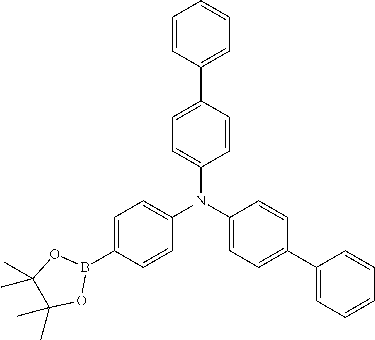 [SMILES: CC1(C)OB(c2ccc(N(c3ccc(-c4ccccc4)cc3)c3ccc(-c4ccccc4)cc3)cc2)OC1(C)C] |
| 5c | 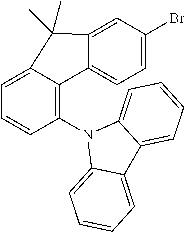 [SMILES: CC1(C)c2cc(Br)ccc2-c2c(-n3c4ccccc4c4ccccc43)cccc21] | 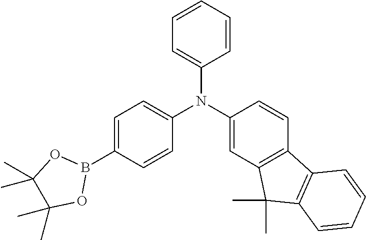 [SMILES: CC1(C)c2ccccc2-c2ccc(N(c3ccccc3)c3ccc(B4OC(C)(C)C(C)(C)O4)cc3)cc21] |
| 5d | 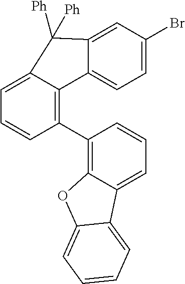 [SMILES: Brc1ccc2c(c1)C(c1ccccc1)(c1ccccc1)c1cccc(-c3cccc4c3oc3ccccc34)c1-2] | 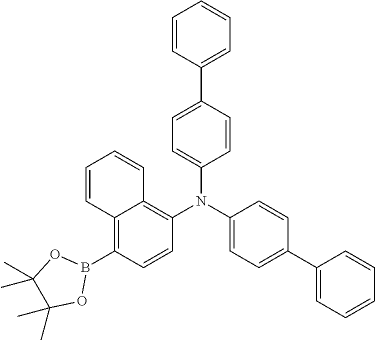 [SMILES: CC1(C)OB(c2ccc(N(c3ccc(-c4ccccc4)cc3)c3ccc(-c4ccccc4)cc3)c3ccccc23)OC1(C)C] |
| 5e | 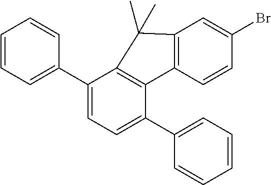 [SMILES: CC1(C)c2cc(Br)ccc2-c2c(-c3ccccc3)ccc(-c3ccccc3)c21] | 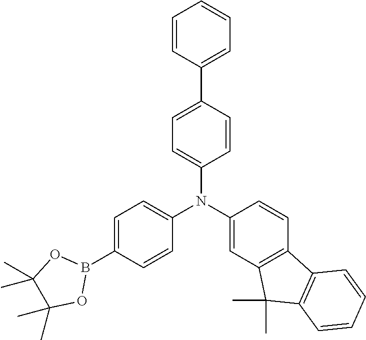 [SMILES: CC1(C)c2ccccc2-c2ccc(N(c3ccc(B4OC(C)(C)C(C)(C)O4)cc3)c3ccc(-c4ccccc4)cc3)cc21] |
| 5f | 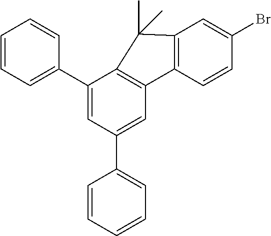 [SMILES: CC1(C)c2cc(Br)ccc2-c2cc(-c3ccccc3)cc(-c3ccccc3)c21] | 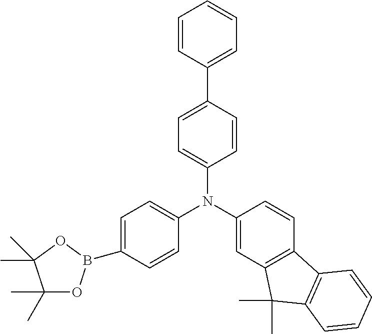 [SMILES: CC1(C)c2ccccc2-c2ccc(N(c3ccc(B4OC(C)(C)C(C)(C)O4)cc3)c3ccc(-c4ccccc4)cc3)cc21] |
| 5g | 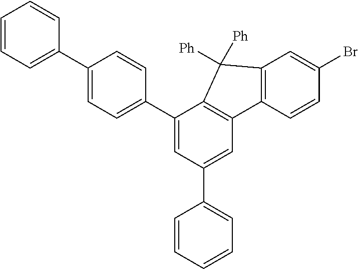 [SMILES: Brc1ccc2c(c1)C(c1ccccc1)(c1ccccc1)c1c(-c3ccc(-c4ccccc4)cc3)cc(-c3ccccc3)cc1-2] | 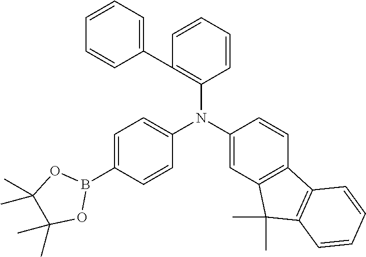 [SMILES: CC1(C)c2ccccc2-c2ccc(N(c3ccc(B4OC(C)(C)C(C)(C)O4)cc3)c3ccccc3-c3ccccc3)cc21] |
| 5h | 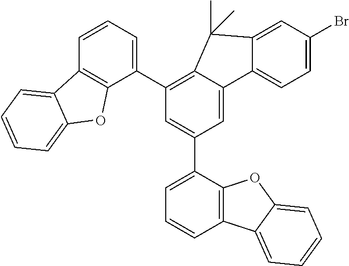 [SMILES: CC1(C)c2cc(Br)ccc2-c2cc(-c3cccc4c3oc3ccccc34)cc(-c3cccc4c3oc3ccccc34)c21] | 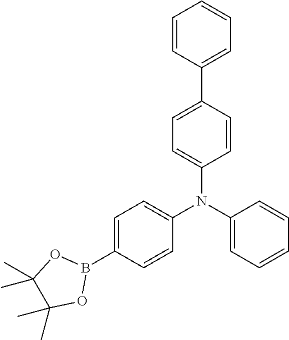 [SMILES: CC1(C)OB(c2ccc(N(c3ccccc3)c3ccc(-c4ccccc4)cc3)cc2)OC1(C)C] |
| 5i | 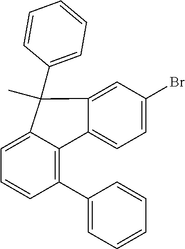 [SMILES: CC1(c2ccccc2)c2cc(Br)ccc2-c2c(-c3ccccc3)cccc21] | 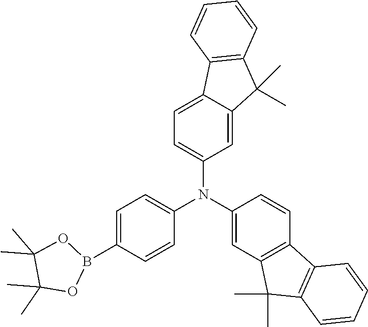 [SMILES: CC1(C)c2ccccc2-c2ccc(N(c3ccc(B4OC(C)(C)C(C)(C)O4)cc3)c3ccc4c(c3)C(C)(C)c3ccccc3-4)cc21] |
| 5j | 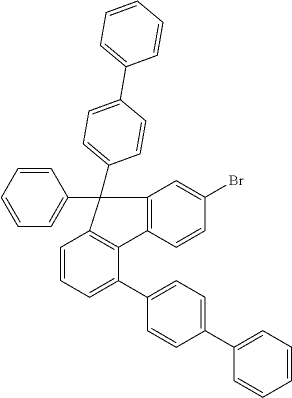 [SMILES: Brc1ccc2c(c1)C(c1ccccc1)(c1ccc(-c3ccccc3)cc1)c1cccc(-c3ccc(-c4ccccc4)cc3)c1-2] | 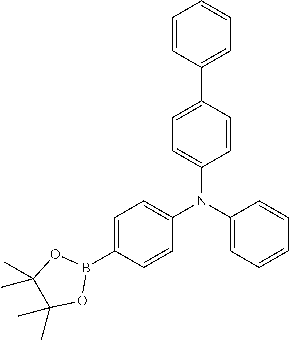 [SMILES: CC1(C)OB(c2ccc(N(c3ccccc3)c3ccc(-c4ccccc4)cc3)cc2)OC1(C)C] |
| 5k | 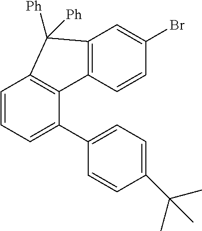 [SMILES: CC(C)(C)c1ccc(-c2cccc3c2-c2ccc(Br)cc2C3(c2ccccc2)c2ccccc2)cc1] | 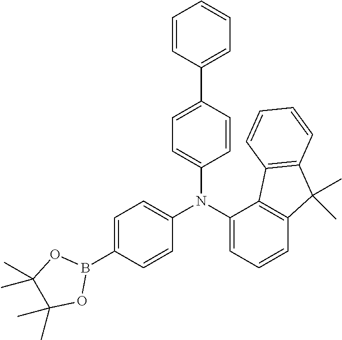 |
| 5l | 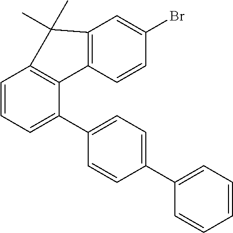 [SMILES: CC1(C)c2cc(Br)ccc2-c2c(-c3ccc(-c4ccccc4)cc3)cccc21] | 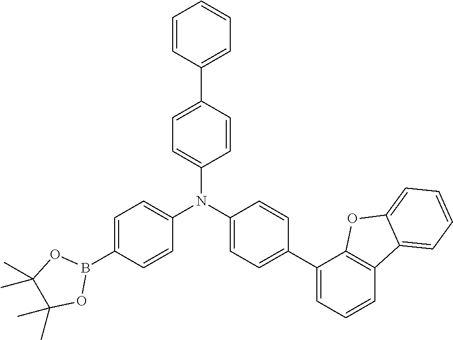 [SMILES: CC1(C)OB(c2ccc(N(c3ccc(-c4ccccc4)cc3)c3ccc(-c4cccc5c4oc4ccccc45)cc3)cc2)OC1(C)C] |
| 5m | 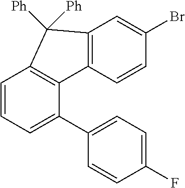 [SMILES: Fc1ccc(-c2cccc3c2-c2ccc(Br)cc2C3(c2ccccc2)c2ccccc2)cc1] | 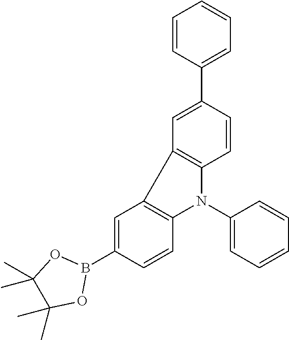 [SMILES: CC1(C)OB(c2ccc3c(c2)c2cc(-c4ccccc4)ccc2n3-c2ccccc2)OC1(C)C] |
| 5n | 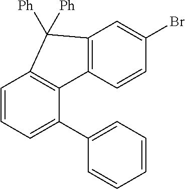 [SMILES: Brc1ccc2c(c1)C(c1ccccc1)(c1ccccc1)c1cccc(-c3ccccc3)c1-2] | 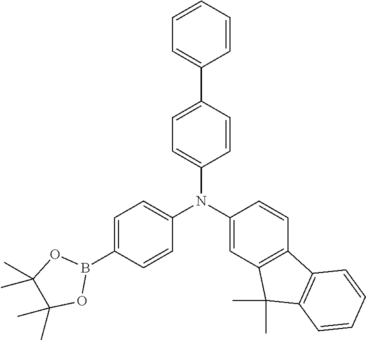 [SMILES: CC1(C)c2ccccc2-c2ccc(N(c3ccc(B4OC(C)(C)C(C)(C)O4)cc3)c3ccc(-c4ccccc4)cc3)cc21] |
| 5o | 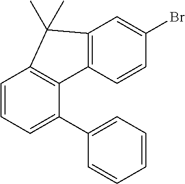 [SMILES: CC1(C)c2cc(Br)ccc2-c2c(-c3ccccc3)cccc21] | 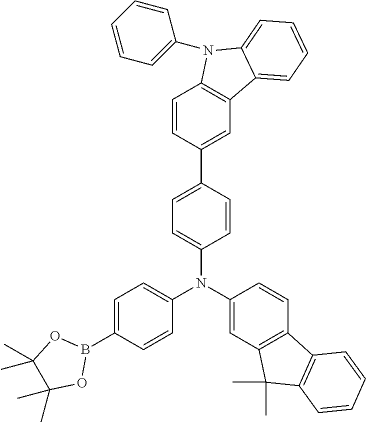 [SMILES: CC1(C)c2ccccc2-c2ccc(N(c3ccc(B4OC(C)(C)C(C)(C)O4)cc3)c3ccc(-c4ccc5c(c4)c4ccccc4n5-c4ccccc4)cc3)cc21] |
| 5p | 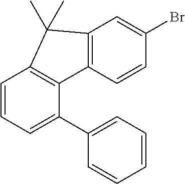 [SMILES: CC1(C)c2cc(Br)ccc2-c2c(-c3ccccc3)cccc21] | 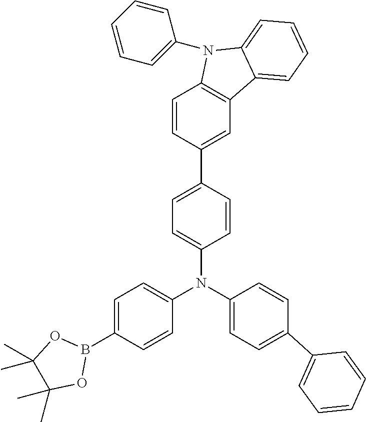 [SMILES: CC1(C)OB(c2ccc(N(c3ccc(-c4ccccc4)cc3)c3ccc(-c4ccc5c(c4)c4ccccc4n5-c4ccccc4)cc3)cc2)OC1(C)C] |
| 5q | 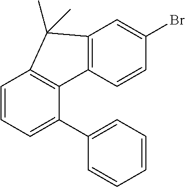 [SMILES: CC1(C)c2cc(Br)ccc2-c2c(-c3ccccc3)cccc21] | 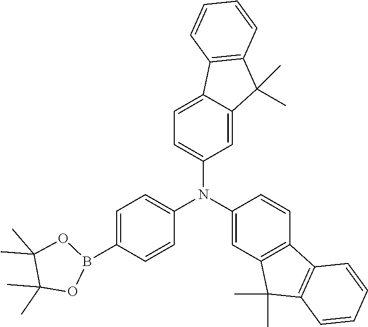 [SMILES: CC1(C)c2ccccc2-c2ccc(N(c3ccc(B4OC(C)(C)C(C)(C)O4)cc3)c3ccc4c(c3)C(C)(C)c3ccccc3-4)cc21] |
| 5r | 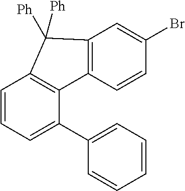 [SMILES: Brc1ccc2c(c1)C(c1ccccc1)(c1ccccc1)c1cccc(-c3ccccc3)c1-2] | 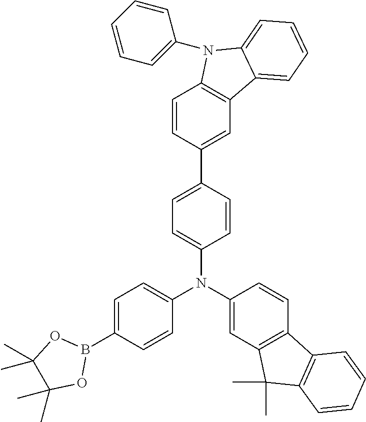 [SMILES: CC1(C)c2ccccc2-c2ccc(N(c3ccc(B4OC(C)(C)C(C)(C)O4)cc3)c3ccc(-c4ccc5c(c4)c4ccccc4n5-c4ccccc4)cc3)cc21] |
| 5s | 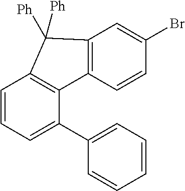 [SMILES: Brc1ccc2c(c1)C(c1ccccc1)(c1ccccc1)c1cccc(-c3ccccc3)c1-2] | 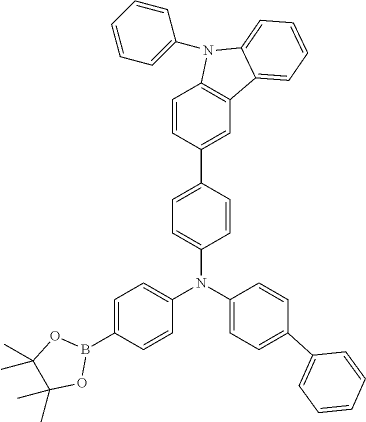 [SMILES: CC1(C)OB(c2ccc(N(c3ccc(-c4ccccc4)cc3)c3ccc(-c4ccc5c(c4)c4ccccc4n5-c4ccccc4)cc3)cc2)OC1(C)C] |
| 5t | 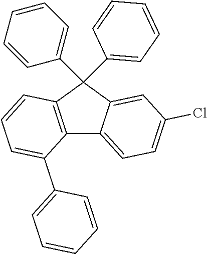 [SMILES: Clc1ccc2c(c1)C(c1ccccc1)(c1ccccc1)c1cccc(-c3ccccc3)c1-2] | 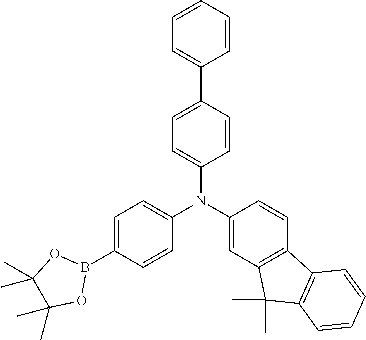 [SMILES: CC1(C)c2ccccc2-c2ccc(N(c3ccc(B4OC(C)(C)C(C)(C)O4)cc3)c3ccc(-c4ccccc4)cc3)cc21] |
| 5u | 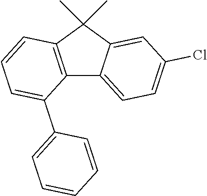 [SMILES: CC1(C)c2cc(Cl)ccc2-c2c(-c3ccccc3)cccc21] | 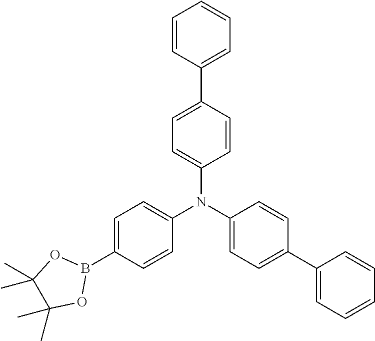 [SMILES: CC1(C)OB(c2ccc(N(c3ccc(-c4ccccc4)cc3)c3ccc(-c4ccccc4)cc3)cc2)OC1(C)C] |

| Ex. | Halogenated Fluorene | Yield |
|---|---|---|
| 5b | 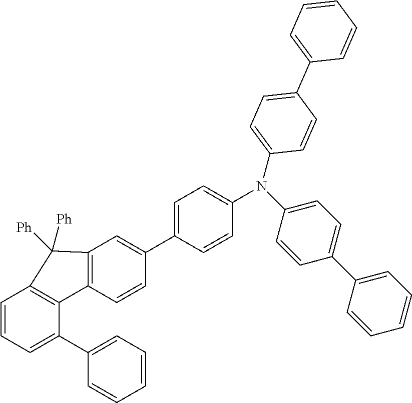 [SMILES: c1ccc(-c2ccc(N(c3ccc(-c4ccccc4)cc3)c3ccc(-c4ccc5c(c4)C(c4ccccc4)(c4ccccc4)c4cccc(-c6ccccc6)c4-5)cc3)cc2)cc1] | 50% |
| 5c | 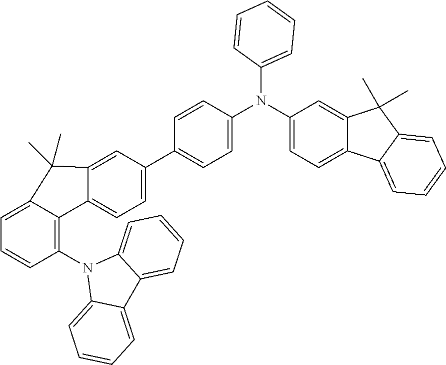 [SMILES: CC1(C)c2ccccc2-c2ccc(N(c3ccccc3)c3ccc(-c4ccc5c(c4)C(C)(C)c4cccc(-n6c7ccccc7c7ccccc76)c4-5)cc3)cc21] | 55% |
| 5d | 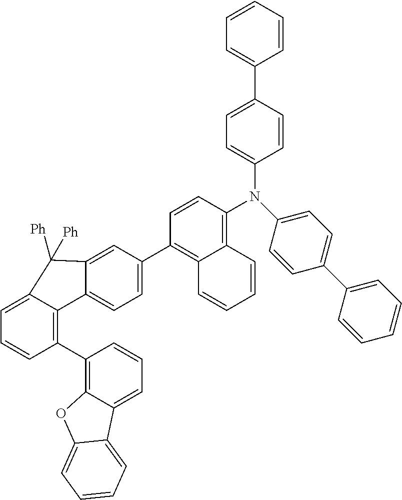 [SMILES: c1ccc(-c2ccc(N(c3ccc(-c4ccccc4)cc3)c3ccc(-c4ccc5c(c4)C(c4ccccc4)(c4ccccc4)c4cccc(-c6cccc7c6oc6ccccc67)c4-5)c4ccccc34)cc2)cc1] | 60% |
| 5e | 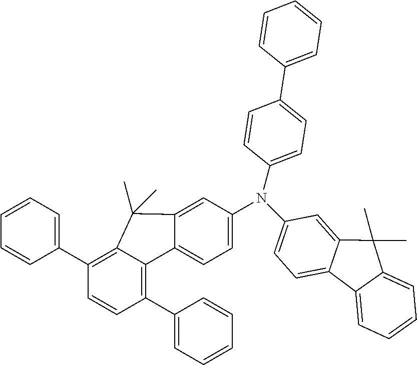 [SMILES: CC1(C)c2ccccc2-c2ccc(N(c3ccc(-c4ccccc4)cc3)c3ccc4c(c3)C(C)(C)c3c(-c5ccccc5)ccc(-c5ccccc5)c3-4)cc21] | 63% |
| 5f | 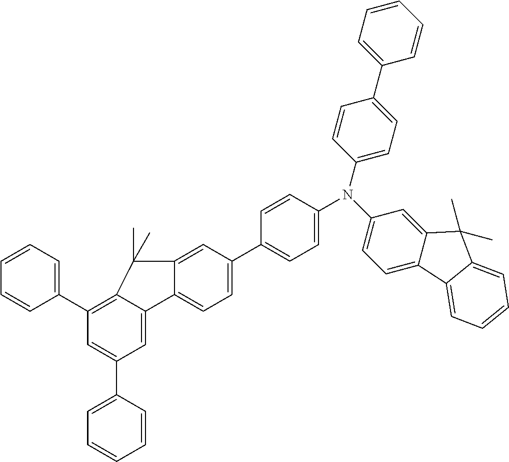 [SMILES: CC1(C)c2ccccc2-c2ccc(N(c3ccc(-c4ccccc4)cc3)c3ccc(-c4ccc5c(c4)C(C)(C)c4c(-c6ccccc6)cc(-c6ccccc6)cc4-5)cc3)cc21] | 70% |
| 5g | 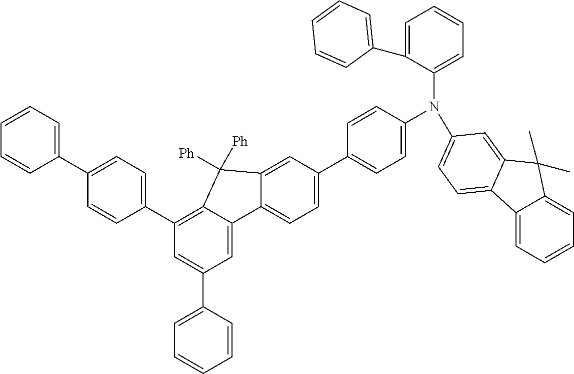 [SMILES: CC1(C)c2ccccc2-c2ccc(N(c3ccc(-c4ccc5c(c4)C(c4ccccc4)(c4ccccc4)c4c(-c6ccc(-c7ccccc7)cc6)cc(-c6ccccc6)cc4-5)cc3)c3ccccc3-c3ccccc3)cc21] | 75% |
| 5h | 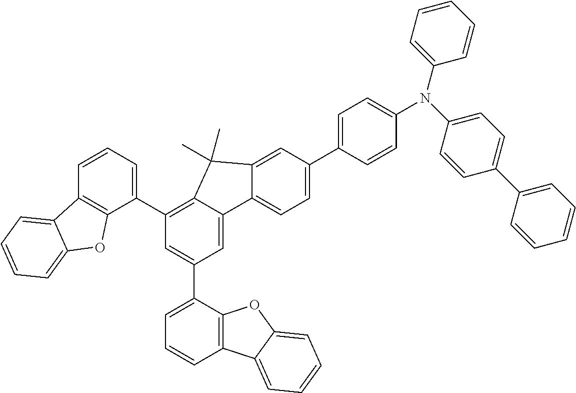 [SMILES: CC1(C)c2cc(-c3ccc(N(c4ccccc4)c4ccc(-c5ccccc5)cc4)cc3)ccc2-c2cc(-c3cccc4c3oc3ccccc34)cc(-c3cccc4c3oc3ccccc34)c21] | 55% |
| 5i | 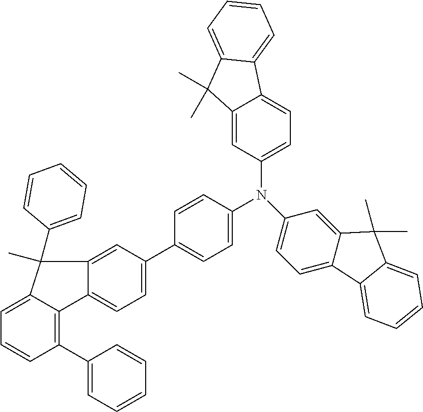 [SMILES: CC1(C)c2ccccc2-c2ccc(N(c3ccc(-c4ccc5c(c4)C(C)(c4ccccc4)c4cccc(-c6ccccc6)c4-5)cc3)c3ccc4c(c3)C(C)(C)c3ccccc3-4)cc21] | 64% |
| 5j | 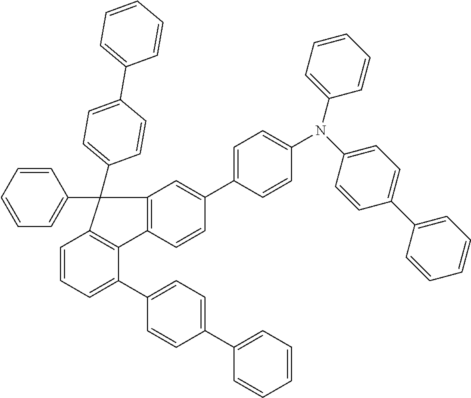 [SMILES: c1ccc(-c2ccc(-c3cccc4c3-c3ccc(-c5ccc(N(c6ccccc6)c6ccc(-c7ccccc7)cc6)cc5)cc3C4(c3ccccc3)c3ccc(-c4ccccc4)cc3)cc2)cc1] | 60% |
| 5k | 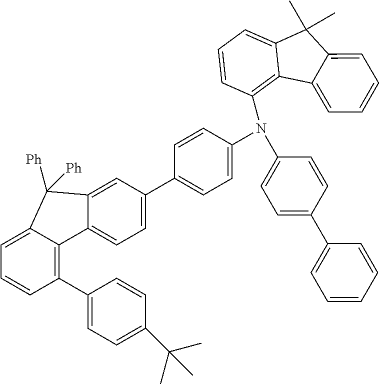 [SMILES: CC(C)(C)c1ccc(-c2cccc3c2-c2ccc(-c4ccc(N(c5ccc(-c6ccccc6)cc5)c5cccc6c5-c5ccccc5C6(C)C)cc4)cc2C3(c2ccccc2)c2ccccc2)cc1] | 75% |
| 5l | 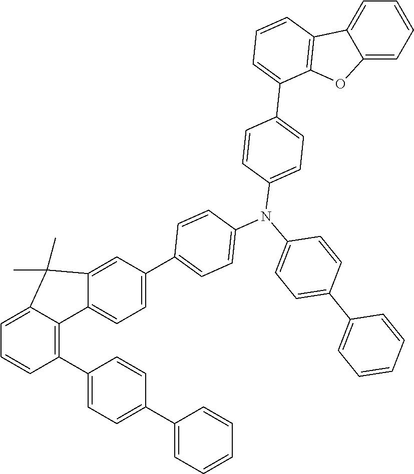 [SMILES: CC1(C)c2cc(-c3ccc(N(c4ccc(-c5ccccc5)cc4)c4ccc(-c5cccc6c5oc5ccccc56)cc4)cc3)ccc2-c2c(-c3ccc(-c4ccccc4)cc3)cccc21] | 81% |
| 5m | 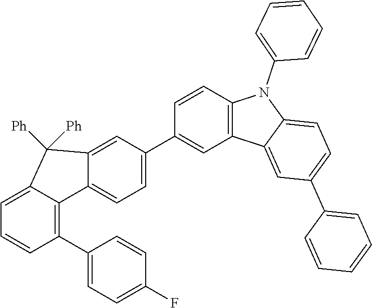 [SMILES: Fc1ccc(-c2cccc3c2-c2ccc(-c4ccc5c(c4)c4cc(-c6ccccc6)ccc4n5-c4ccccc4)cc2C3(c2ccccc2)c2ccccc2)cc1] | 77% |
| 5n | 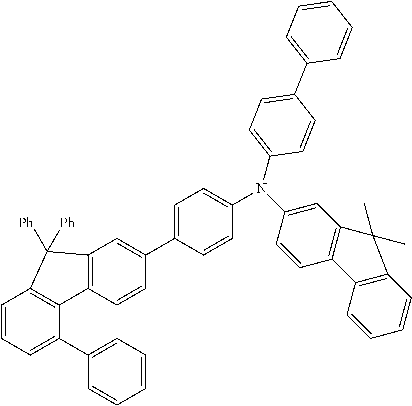 [SMILES: CC1(C)c2ccccc2-c2ccc(N(c3ccc(-c4ccccc4)cc3)c3ccc(-c4ccc5c(c4)C(c4ccccc4)(c4ccccc4)c4cccc(-c6ccccc6)c4-5)cc3)cc21] | 62% |
| 5o | 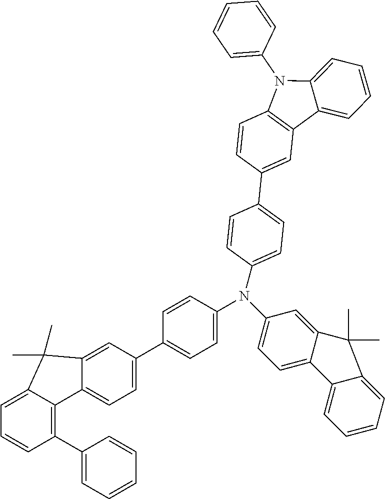 [SMILES: CC1(C)c2ccccc2-c2ccc(N(c3ccc(-c4ccc5c(c4)C(C)(C)c4cccc(-c6ccccc6)c4-5)cc3)c3ccc(-c4ccc5c(c4)c4ccccc4n5-c4ccccc4)cc3)cc21] | 60% |
| 5p | 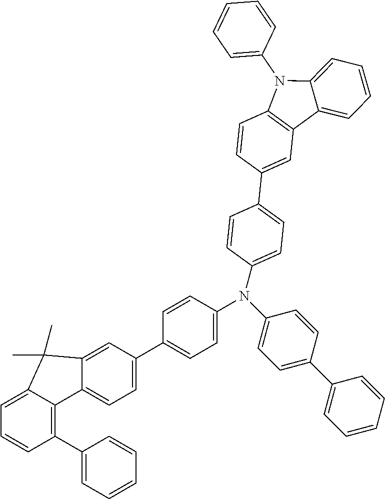 [SMILES: CC1(C)c2cc(-c3ccc(N(c4ccc(-c5ccccc5)cc4)c4ccc(-c5ccc6c(c5)c5ccccc5n6-c5ccccc5)cc4)cc3)ccc2-c2c(-c3ccccc3)cccc21] | 72% |
| 5q | 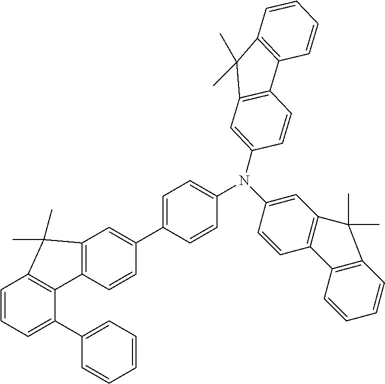 [SMILES: CC1(C)c2ccccc2-c2ccc(N(c3ccc(-c4ccc5c(c4)C(C)(C)c4cccc(-c6ccccc6)c4-5)cc3)c3ccc4c(c3)C(C)(C)c3ccccc3-4)cc21] | 45% |
| 5r | 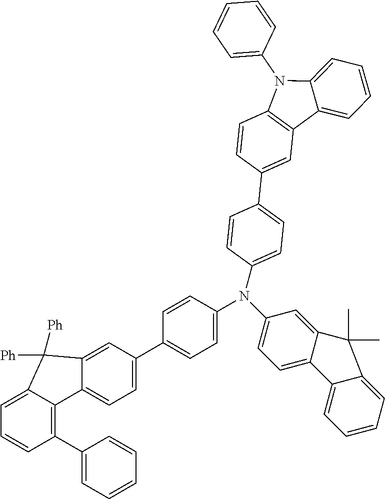 [SMILES: CC1(C)c2ccccc2-c2ccc(N(c3ccc(-c4ccc5c(c4)C(c4ccccc4)(c4ccccc4)c4cccc(-c6ccccc6)c4-5)cc3)c3ccc(-c4ccc5c(c4)c4ccccc4n5-c4ccccc4)cc3)cc21] | 56% |
| 5s | 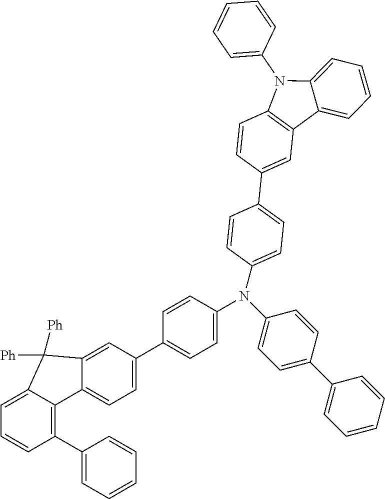 [SMILES: c1ccc(-c2ccc(N(c3ccc(-c4ccc5c(c4)C(c4ccccc4)(c4ccccc4)c4cccc(-c6ccccc6)c4-5)cc3)c3ccc(-c4ccc5c(c4)c4ccccc4n5-c4ccccc4)cc3)cc2)cc1] | 62% |
| 5t | 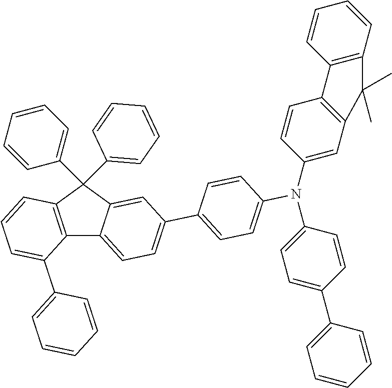 [SMILES: CC1(C)c2ccccc2-c2ccc(N(c3ccc(-c4ccccc4)cc3)c3ccc(-c4ccc5c(c4)C(c4ccccc4)(c4ccccc4)c4cccc(-c6ccccc6)c4-5)cc3)cc21] | 57% |
| 5u | 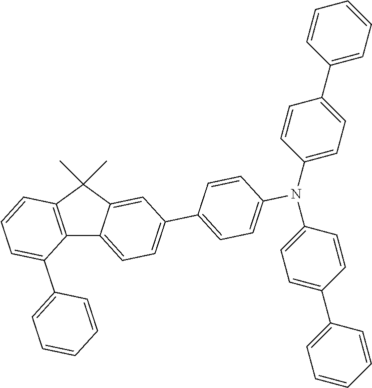 [SMILES: CC1(C)c2cc(-c3ccc(N(c4ccc(-c5ccccc5)cc4)c4ccc(-c5ccccc5)cc4)cc3)ccc2-c2c(-c3ccccc3)cccc21] | 54% |

### Synthesis of 5-bromo-2-chloro-9,9-diphenyl-9H-fluorene 6a

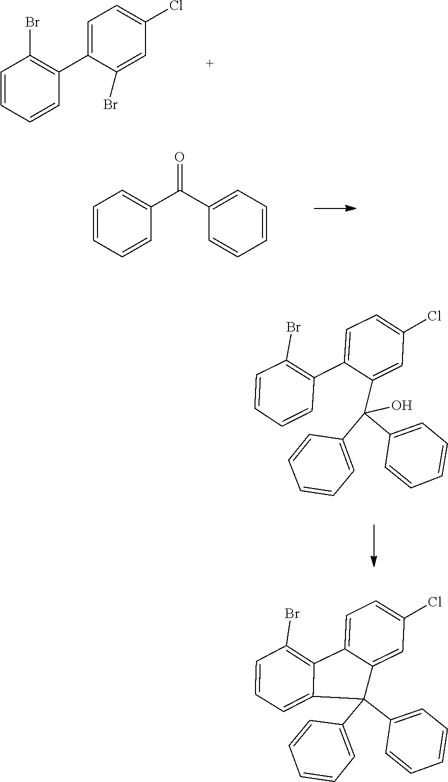 [SMILES: Clc1ccc(-c2ccccc2Br)c(Br)c1 ; O=C(c1ccccc1)c1ccccc1 ; OC(c1ccccc1)(c1ccccc1)c1cc(Cl)ccc1-c1ccccc1Br ; Clc1ccc2c(c1)C(c1ccccc1)(c1ccccc1)c1cccc(Br)c1-2]

A solution of 2,2′-dibromo-4-chloro-biphenyl (84 g, 239 mmol) in THF (200 ml) is treated with 109 mL of n-BuLi (2.2 M in hexane, 239 mmol) under argon at −78° C. The mixture is stirred for 30 minutes. A solution of benzophenone (43.5 g, 239 mmol) in 150 mL THF is added dropwise. The reaction proceeds at −78° C. for 30 minutes and then is stirred at room temperature overnight. The reaction is quenched with water and the solid is filtered. Without further purification, a solution of the alcohol in 966 mL toluene and 2.9 g p-toluenesulfonic acid is refluxed overnight. After cooling, the organic phase is washed with water and the solvent is removed under vacuum. The product is isolated in the form of a white solid (60 g, 90% of theory).

The synthesis of further halogenated fluorene derivatives is carried out analogously:

| | Bromo-biphenyl | Aryl-fluoenone | Product | Yield |
|---|---|---|---|---|
| 6b | 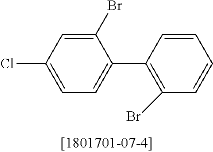 [SMILES: Clc1ccc(-c2ccccc2Br)c(Br)c1] | 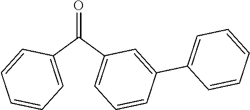 [SMILES: O=C(c1ccccc1)c1cccc(-c2ccccc2)c1] | 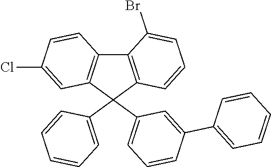 [SMILES: Clc1ccc2c(c1)C(c1ccccc1)(c1cccc(-c3ccccc3)c1)c1cccc(Br)c1-2] | 86% |
| 6c | 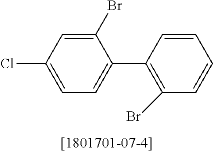 [SMILES: Clc1ccc(-c2ccccc2Br)c(Br)c1] | 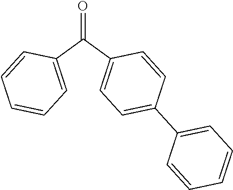 [SMILES: O=C(c1ccccc1)c1ccc(-c2ccccc2)cc1] | 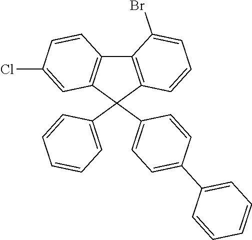 [SMILES: Clc1ccc2c(c1)C(c1ccccc1)(c1ccc(-c3ccccc3)cc1)c1cccc(Br)c1-2] | 84% |
| 6d | 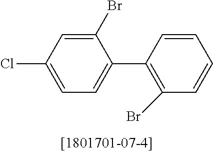 [SMILES: Clc1ccc(-c2ccccc2Br)c(Br)c1] | 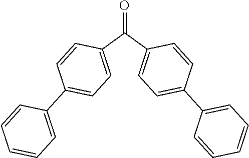 [SMILES: O=C(c1ccc(-c2ccccc2)cc1)c1ccc(-c2ccccc2)cc1] | 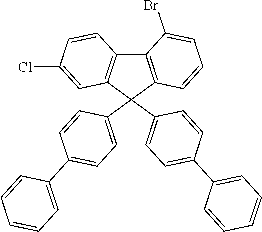 [SMILES: Clc1ccc2c(c1)C(c1ccc(-c3ccccc3)cc1)(c1ccc(-c3ccccc3)cc1)c1cccc(Br)c1-2] | 90% |
| 6f | 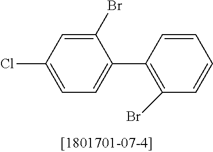 [SMILES: Clc1ccc(-c2ccccc2Br)c(Br)c1] | 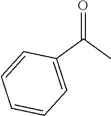 | 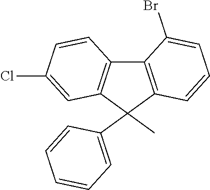 [SMILES: CC1(c2ccccc2)c2cc(Cl)ccc2-c2c(Br)cccc21] | 90% |
| 6g | 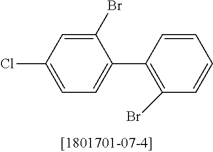 [SMILES: Clc1ccc(-c2ccccc2Br)c(Br)c1] | 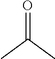 | 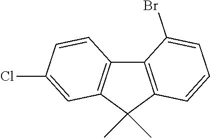 [SMILES: CC1(C)c2cc(Cl)ccc2-c2c(Br)cccc21] | 66% |
| 6h | 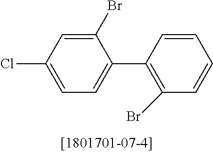 [SMILES: Clc1ccc(-c2ccccc2Br)c(Br)c1] | 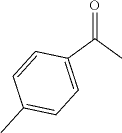 | 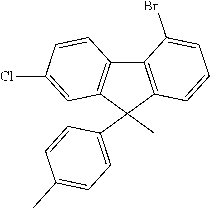 [SMILES: Cc1ccc(C2(C)c3cc(Cl)ccc3-c3c(Br)cccc32)cc1] | 81% |
| 6i | 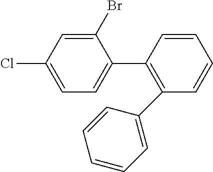 [SMILES: Clc1ccc(-c2ccccc2-c2ccccc2)c(Br)c1] | 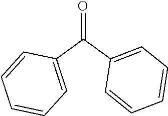 [SMILES: O=C(c1ccccc1)c1ccccc1] | 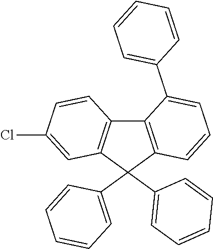 [SMILES: Clc1ccc2c(c1)C(c1ccccc1)(c1ccccc1)c1cccc(-c3ccccc3)c1-2] | 77% |
| 6j | 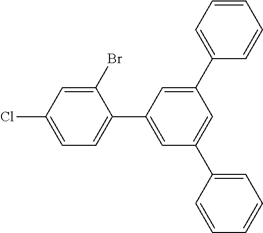 [SMILES: Clc1ccc(-c2cc(-c3ccccc3)cc(-c3ccccc3)c2)c(Br)c1] | 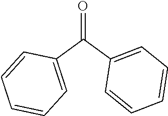 [SMILES: O=C(c1ccccc1)c1ccccc1] | 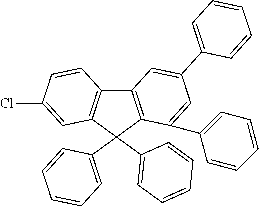 [SMILES: Clc1ccc2c(c1)C(c1ccccc1)(c1ccccc1)c1c(-c3ccccc3)cc(-c3ccccc3)cc1-2] | 85% |
| 6k | 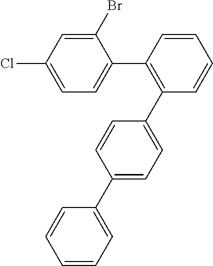 [SMILES: Clc1ccc(-c2ccccc2-c2ccc(-c3ccccc3)cc2)c(Br)c1] | 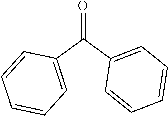 [SMILES: O=C(c1ccccc1)c1ccccc1] | 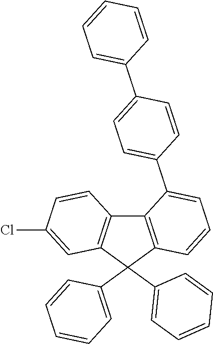 [SMILES: Clc1ccc2c(c1)C(c1ccccc1)(c1ccccc1)c1cccc(-c3ccc(-c4ccccc4)cc3)c1-2] | 81% |
| 6l | 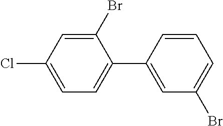 [SMILES: Clc1ccc(-c2cccc(Br)c2)c(Br)c1] | 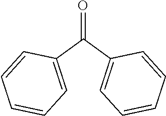 [SMILES: O=C(c1ccccc1)c1ccccc1] | 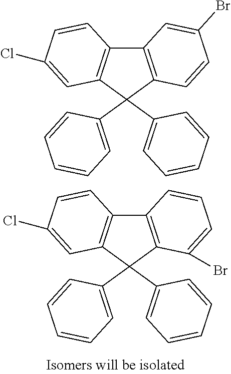 | 80% |
| 6m | 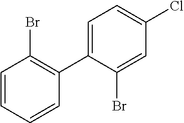 [SMILES: Clc1ccc(-c2ccccc2Br)c(Br)c1] | 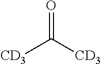 | 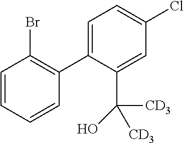 [SMILES: [2H]C([2H])([2H])C(O)(c1cc(Cl)ccc1-c1ccccc1Br)C([2H])([2H])[2H]] | 87% |
| 6n | 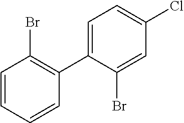 [SMILES: Clc1ccc(-c2ccccc2Br)c(Br)c1] | 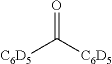 | 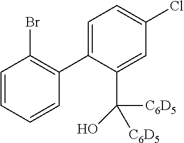 | 89% |
| 6o | 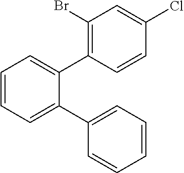 [SMILES: Clc1ccc(-c2ccccc2-c2ccccc2)c(Br)c1] | 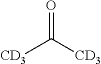 | 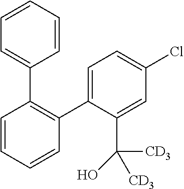 [SMILES: [2H]C([2H])([2H])C(O)(c1cc(Cl)ccc1-c1ccccc1-c1ccccc1)C([2H])([2H])[2H]] | 78% |
| 6p | 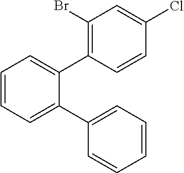 [SMILES: Clc1ccc(-c2ccccc2-c2ccccc2)c(Br)c1] | 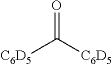 | 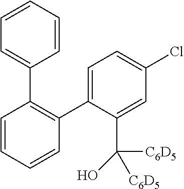 | 82% |

### Synthesis of 2-chloro-5,9,9-triphenyl-9H-fluorene 7a

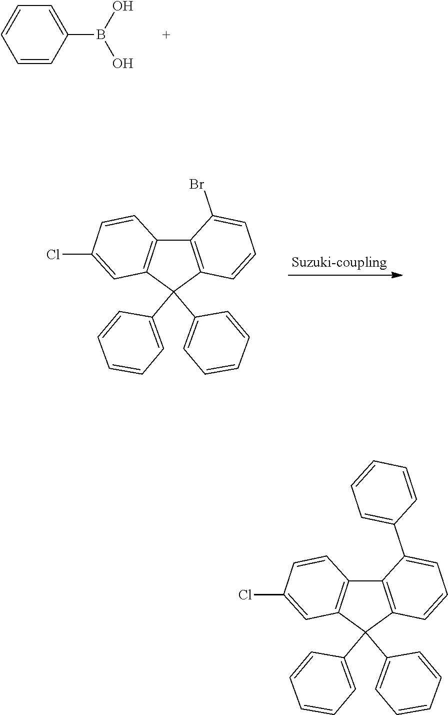 [SMILES: OB(O)c1ccccc1 ; Clc1ccc2c(c1)C(c1ccccc1)(c1ccccc1)c1cccc(Br)c1-2 ; Clc1ccc2c(c1)C(c1ccccc1)(c1ccccc1)c1cccc(-c3ccccc3)c1-2]

31.5 g (251 mmol) of phenyl-boronic acid, 108.4 g (251 mmol) of 5-bromo-2-chloro-9,9-diphenyl-9H-fluorene, 9.9 g (8.5 mmol) of Pd(P(Ph₃))₄, 66.8 g (627 mmol) of Na₂CO₃ are dissolved in 903 mL of water, 278 mL of ethanol and 1.9 L of toluene. The reaction mixture is refluxed and agitated under an argon atmosphere for 12 hours and after cooling to room temperature, the mixture is filtered through Celite. The filtrate is evaporated in vacuo, and the residue is crystallised from heptane. The product is isolated in the form of an off-white solid (100 g, 93% of theory).

The following compounds are synthesized analogously:

| Ex. | Product | Boron acid | Product | Yield |
|---|---|---|---|---|
| 7b | 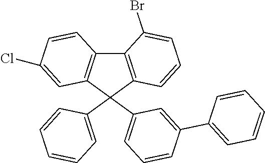 [SMILES: Clc1ccc2c(c1)C(c1ccccc1)(c1cccc(-c3ccccc3)c1)c1cccc(Br)c1-2] | 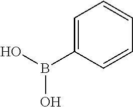 | 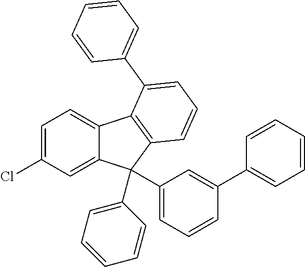 [SMILES: Clc1ccc2c(c1)C(c1ccccc1)(c1cccc(-c3ccccc3)c1)c1cccc(-c3ccccc3)c1-2] | 76% |
| 7c | 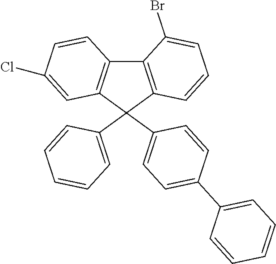 [SMILES: Clc1ccc2c(c1)C(c1ccccc1)(c1ccc(-c3ccccc3)cc1)c1cccc(Br)c1-2] | 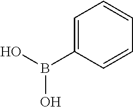 | 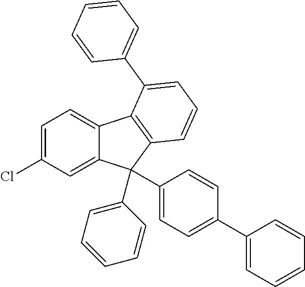 [SMILES: Clc1ccc2c(c1)C(c1ccccc1)(c1ccc(-c3ccccc3)cc1)c1cccc(-c3ccccc3)c1-2] | 82% |
| 7d | 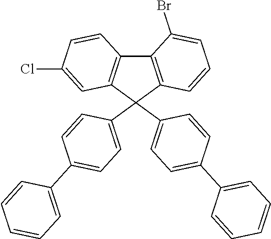 [SMILES: Clc1ccc2c(c1)C(c1ccc(-c3ccccc3)cc1)(c1ccc(-c3ccccc3)cc1)c1cccc(Br)c1-2] | 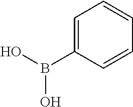 | 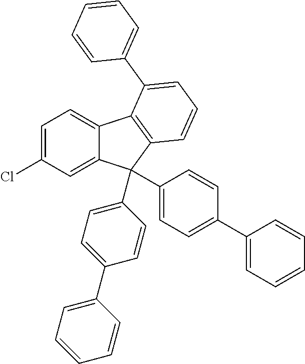 [SMILES: Clc1ccc2c(c1)C(c1ccc(-c3ccccc3)cc1)(c1ccc(-c3ccccc3)cc1)c1cccc(-c3ccccc3)c1-2] | 43% |
| 7e | 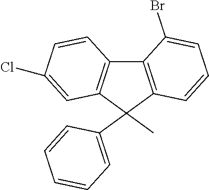 [SMILES: CC1(c2ccccc2)c2cc(Cl)ccc2-c2c(Br)cccc21] | 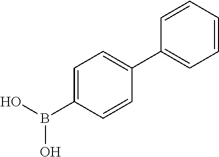 [SMILES: OB(O)c1ccc(-c2ccccc2)cc1] | 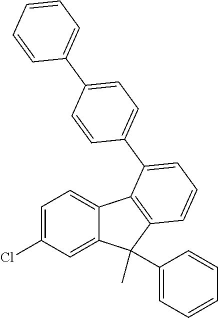 [SMILES: CC1(c2ccccc2)c2cc(Cl)ccc2-c2c(-c3ccc(-c4ccccc4)cc3)cccc21] | 72% |
| 7f | 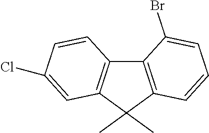 [SMILES: CC1(C)c2cc(Cl)ccc2-c2c(Br)cccc21] | 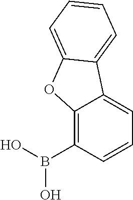 [SMILES: OB(O)c1cccc2c1oc1ccccc12] | 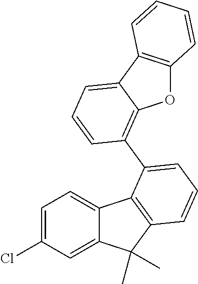 [SMILES: CC1(C)c2cc(Cl)ccc2-c2c(-c3cccc4c3oc3ccccc34)cccc21] | 60% |
| 7g | 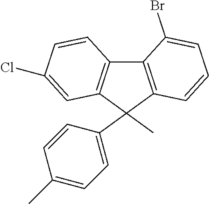 [SMILES: Cc1ccc(C2(C)c3cc(Cl)ccc3-c3c(Br)cccc32)cc1] | 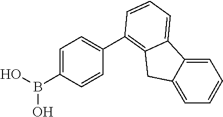 [SMILES: OB(O)c1ccc(-c2cccc3c2Cc2ccccc2-3)cc1] | 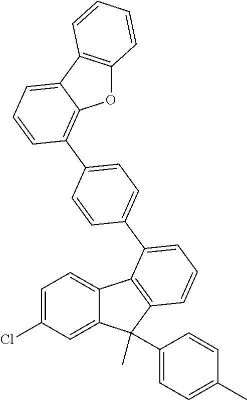 [SMILES: Cc1ccc(C2(C)c3cc(Cl)ccc3-c3c(-c4ccc(-c5cccc6c5oc5ccccc56)cc4)cccc32)cc1] | 85% |
| 7h | 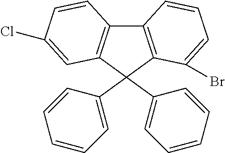 [SMILES: Clc1ccc2c(c1)C(c1ccccc1)(c1ccccc1)c1c(Br)cccc1-2] | 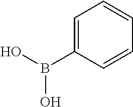 | 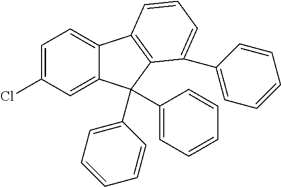 [SMILES: Clc1ccc2c(c1)C(c1ccccc1)(c1ccccc1)c1c(-c3ccccc3)cccc1-2] | 72% |
| 7i | 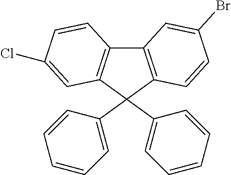 [SMILES: Clc1ccc2c(c1)C(c1ccccc1)(c1ccccc1)c1ccc(Br)cc1-2] | 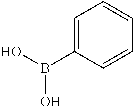 | 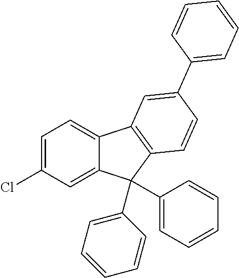 [SMILES: Clc1ccc2c(c1)C(c1ccccc1)(c1ccccc1)c1ccc(-c3ccccc3)cc1-2] | 50% |
| 7j | 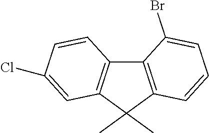 [SMILES: CC1(C)c2cc(Cl)ccc2-c2c(Br)cccc21] | 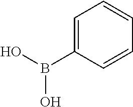 | 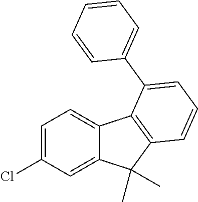 [SMILES: CC1(C)c2cc(Cl)ccc2-c2c(-c3ccccc3)cccc21] | 61% |
| 7k | 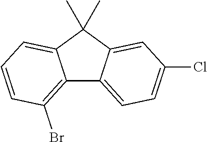 [SMILES: CC1(C)c2cc(Cl)ccc2-c2c(Br)cccc21] | 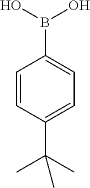 | 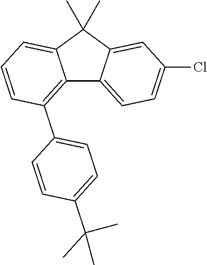 [SMILES: CC(C)(C)c1ccc(-c2cccc3c2-c2ccc(Cl)cc2C3(C)C)cc1] | 55% |
| 7l | 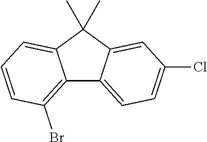 [SMILES: CC1(C)c2cc(Cl)ccc2-c2c(Br)cccc21] | 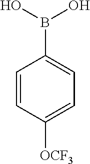 [SMILES: OB(O)c1ccc(OC(F)(F)F)cc1] | 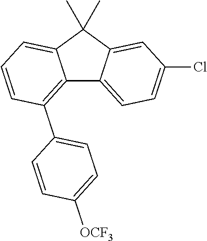 [SMILES: CC1(C)c2cc(Cl)ccc2-c2c(-c3ccc(OC(F)(F)F)cc3)cccc21] | 50% |
| 7m | 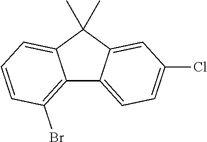 [SMILES: CC1(C)c2cc(Cl)ccc2-c2c(Br)cccc21] | 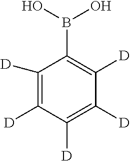 [SMILES: [2H]c1c([2H])c([2H])c(B(O)O)c([2H])c1[2H]] | 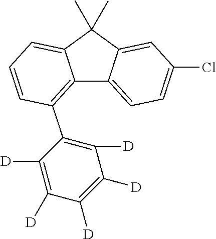 [SMILES: [2H]c1c([2H])c([2H])c(-c2cccc3c2-c2ccc(Cl)cc2C3(C)C)c([2H])c1[2H]] | 65% |

### B) Production of OLEDs

OLEDs according to the invention and OLEDs in accordance with the prior art are produced by a general process in accordance with WO 2004/058911, which is adapted to the circumstances described herein (e. g. materials).

The data for various OLEDs are presented in examples below (see Tables 1 to 7). The substrates used are glass plates coated with structured ITO (indium tin oxide) in a thickness of 50 nm. The OLEDs basically have the following layer structure: substrate/hole-injection layer (HIL)/hole-transport layer (HTL)/electron-blocking layer (EBL)/emission layer (EML)/electron-transport layer (ETL)/electron-injection layer (EIL) and finally a cathode. The cathode is formed by an aluminium layer with a thickness of 100 nm. The materials required for the production of the OLEDs are shown in Table 7.

All materials are applied by thermal vapour deposition in a vacuum chamber. The emission layer here always consists of at least one matrix material (host material) and an emitting dopant (emitter), which is admixed with the matrix material or matrix materials in a certain proportion by volume by co-evaporation. An expression such as H1:SEB (5%) here means that material H1 is present in the layer in a proportion by volume of 95% and SEB is present in the layer in a proportion of 5%. Analogously, other layers may also consist of a mixture of two or more materials.

The OLEDs are characterised by standard methods. For this purpose, the electroluminescence spectra and the external quantum efficiency (EQE, measured in percent) as a function of the luminous density, calculated from current/voltage/luminous density characteristic lines (IUL characteristic lines) assuming Lambert emission characteristics and the lifetime are determined. The expression EQE @ 10 mA/cm² denotes the external quantum efficiency at an operating current density of 10 mA/cm². LT80@60 mA/cm² is the lifetime until the OLED has dropped from its initial luminance of i.e. 5000 cd/m² to 80% of the initial intensity, i.e. to 4000 cd/m² without using any acceleration factor. The data for the various OLEDs containing inventive material are summarised in Tables 2 to 6.

Use of Compounds According to the Invention in Fluorescent and Phosphorescent OLEDs

In particular, compounds according to the invention are suitable as HIL, HTL, EBL or matrix material in the EML in OLEDs. They are suitable for use as a single layer, but also for use as mixed component as HIL, HTL, EBL or within the EML.

OLED devices with the structures are shown in the following Tables 1, 3, 4 and 5. Tables 2 and 6 provide the device data.

OLEDs E1 to E27 are OLEDs according to the present application, which comprise the inventive compounds HTM-1 to HTM-14 as HTL and EBL, respectively. COMP-1 and COMP-2 are comparative examples. OLEDs E1 to E27 according to the present application all show high lifetimes, low voltage and good efficiency in singlet blue and also in triplet green devices. Particularly, as compared to the comparative examples the examples according to the invention clearly show statistically and physically significant improvements regarding efficiencies.

TABLE 1
Device Setup

| | HIL | HTL | EBL | EML | ETL | EIL |
|---|---|---|---|---|---|---|
| Ex. | Thickness/nm | Thickness/nm | Thickness/nm | Thickness/nm | Thickness/nm | Thickness/nm |
| E1 | HTM-1: p- | HTM-1 | EBL | H:SEB(5%) | ETM:LiQ(50%) | LiQ |
| | doped(5%) | 180 nm | 10 nm | 20 nm | 30 nm | 1 nm |
| | 20 nm | | | | | |
| E2 | HTM: p- | HTM | HTM-1 | TMM-1: TMM- | ETM:LiQ(50%) | LiQ |
| | doped(5%) | 220 nm | 10 nm | 2(28%):TEG(12%) | 30 nm | 1 nm |
| | 20 nm | | | 30 nm | | |
| E3 | HTM-2: p- | HTM-2 | EBL | H:SEB(5%) | ETM:LiQ(50%) | LiQ |
| | doped (5%) | 180 nm | 10 nm | 20 nm | 30 nm | 1 nm |
| | 20 nm | | | | | |
| E4 | HTM: p- | HTM | HTM-2 | TMM-1: TMM- | ETM:LiQ(50%) | LiQ |
| | doped(5%) | 220 nm | 10 nm | 2(28%):TEG(12%) | 30 nm | 1 nm |
| | 20 nm | | | 30 nm | | |
| E5 | HTM-3: p- | HTM-3 | EBL | H:SEB(5%) | ETM:LiQ(50%) | LiQ |
| | doped(5%) | 180 nm | 10 nm | 20 nm | 30 nm | 1 nm |
| | 20 nm | | | | | |
| E6 | HTM: p- | HTM | HTM-3 | TMM-1: TMM- | ETM:LiQ(50%) | LiQ |
| | doped(5%) | 220 nm | 10 nm | 2(28%):TEG(12%) | 30 nm | 1 nm |
| | 20 nm | | | 30 nm | | |
| E7 | HTM: p- | HTM | HTM-4 | TMM-1: TMM- | ETM:LiQ(50%) | LiQ |
| | doped(5%) | 220 nm | 10 nm | 2(28%):TEG(12%) | 30 nm | 1 nm |
| | 20 nm | | | 30 nm | | |
| E8 | HTM-5: p- | HTM-5 | EBL | H:SEB(5%) | ETM:LiQ(50%) | LiQ |
| | doped(5%) | 180 nm | 10 nm | 20 nm | 30 nm | 1 nm |
| | 20 nm | | | | | |
| E9 | HTM: p- | HTM | HTM-5 | TMM-1: TMM- | ETM:LiQ(50%) | LiQ |
| | doped(5%) | 220 nm | 10 nm | 2(28%):TEG(12%) | 30 nm | 1 nm |
| | 20 nm | | | 30 nm | | |
| C1 | HTM: p- | HTM | COMP-1 | TMM-1: TMM- | ETM:LiQ(50%) | LiQ |
| | doped(5%) | 220 nm | 10 nm | 2(28%):TEG(12%) | 30 nm | 1 nm |
| | 20 nm | | | 30 nm | | |
| E10 | HTM: p- | HTM | HTM-7 | TMM-1: TMM- | ETM:LiQ(50%) | LiQ |
| | doped(5%) | 220 nm | 10 nm | 2(28%):TEG(12%) | 30 nm | 1 nm |
| | 20 nm | | | 30 nm | | |
| C2 | HTM: p- | HTM | COMP-2 | TMM-1: TMM- | EMT:LiQ(50%) | LiQ |
| | doped(5%) | 220 nm | 10 nm | 2(28%):TEG(12%) | 30 nm | 1 nm |
| | 20 nm | | | 30 nm | | |
| E11 | HTM: p- | HTM | HTM-9 | TMM-1: TMM- | ETM:LiQ(50%) | LiQ |
| | doped(5%) | 220 nm | 10 nm | 2(28%):TEG(12%) | 30 nm | 1 nm |
| | 20 nm | | | 30 nm | | |

TABLE 2
Data for the OLEDs

| | | | LT80 @ 60/40* |
|---|---|---|---|
| | U | EQE | mA/cm² |
| | [V] | [%] | [h] |
| E1 | 4.1 | 9.1 | 220 |
| E2 | 3.9 | 16.0 | 340* |
| E3 | 4.0 | 9.4 | 190 |
| E4 | 3.8 | 15.8 | 360* |
| E5 | 3.9 | 9.0 | 300 |
| E6 | 3.9 | 15.4 | 340* |
| E7 | 3.9 | 14.8 | 390* |
| E8 | 4.1 | 9.0 | 280 |
| E9 | 3.9 | 15.9 | 320* |
| C1 | 3.8 | 15.1 | 380* |
| E10 | 4.0 | 16.9 | 370* |
| C2 | 3.8 | 16.0 | 350* |
| E11 | 3.8 | 17.2 | 380* |

Tables 3 to 6 summarize further device data of OLEDs comprising the inventive compounds HTM-10 to HTM-14.

TABLE 3
Device Setup—green EBL devices

| | HIL | HTL | EBL | EML | ETL | EIL |
|---|---|---|---|---|---|---|
| Ex. | Thickness/nm | Thickness/nm | Thickness/nm | Thickness/nm | Thickness/nm | Thickness/nm |
| E14 | HTM: p-doped(5%) | HTM | HTM-10 | TMM-1: TMM-2(28%):TEG(12%) | ETM:LiQ(50%) | LiQ |
| | 20 nm | 220 nm | 10 nm | 30 nm | 30 nm | 1 nm |
| E15 | HTM: p-doped(5%) | HTM | HTM-11 | TMM-1: TMM-2(28%):TEG(12%) | ETM:LiQ(50%) | LiQ |
| | 20 nm | 220 nm | 10 nm | 30 nm | 30 nm | 1 nm |
| E16 | HTM: p-doped(5%) | HTM | HTM-12 | TMM-1: TMM-2(28%):TEG(12%) | ETM:LiQ(50%) | LiQ |
| | 20 nm | 220 nm | 10 nm | 30 nm | 30 nm | 1 nm |
| E17 | HTM: p-doped(5%) | HTM | HTM-13 | TMM-1: TMM-2(28%):TEG(12%) | ETM:LiQ(50%) | LiQ |
| | 20 nm | 220 nm | 10 nm | 30 nm | ×30 nm | 1 nm |
| E18 | HTM: p-doped(5%) | HTM | HTM-14 | TMM-1: TMM-2(28%):TEG(12%) | ETM:LiQ(50%) | LiQ |
| | 20 nm | 220 nm | 10 nm | 30 nm | 30 nm | 1 nm |

TABLE 4
Device Setup—blue EBL devices

| | HIL | HTL | EBL | EML | ETL | EIL |
|---|---|---|---|---|---|---|
| Ex. | Thickness/nm | Thickness/nm | Thickness/nm | Thickness/nm | Thickness/nm | Thickness/nm |
| E19 | HTM: p-doped(5%) | HTM | HTM-10 | H:SEB(5%) | ETM:LiQ(50%) | LiQ |
| | 20 nm | 180 nm | 10 nm | 20 nm | 30 nm | 1 nm |
| E20 | HTM: p-doped(5%) | HTM | HTM-11 | H:SEB(5%) | ETM:LiQ(50%) | LiQ |
| | 20 nm | 180 nm | 10 nm | 20 nm | 30 nm | 1 nm |
| E21 | HTM: p-doped(5%) | HTM | HTM-13 | H:SEB(5%) | ETM:LiQ(50%) | LiQ |
| | 20 nm | 180 nm | 10 nm | 20 nm | 30 nm | 1 nm |
| E22 | HTM: p-doped(5%) | HTM | HTM-14 | H:SEB(5%) | ETM:LiQ(50%) | LiQ |
| | 20 nm | 180 nm | 10 nm | 20 nm | 30 nm | 1 nm |

TABLE 5
Device Setup—blue HTL devices

| | HIL | HTL | EBL | EML | ETL | EIL |
|---|---|---|---|---|---|---|
| Ex. | Thickness/nm | Thickness/nm | Thickness/nm | Thickness/nm | Thickness/nm | Thickness/nm |
| E23 | HTM-10: p-doped(5%) | HTM-10 | EBM | H:SEB(5%) | ETM:LiQ(50%) | LiQ |
| | 20 nm | 180 nm | 10 nm | 20 nm | 30 nm | 1 nm |
| E24 | HTM-11: p-doped(5%) | HTM-11 | EBM | H:SEB(5%) | ETM:LiQ(50%) | LiQ |
| | 20 nm | 180 nm | 10 nm | 20 nm | 30 nm | 1 nm |
| E25 | HTM-12: p-doped(5%) | HTM-12 | EBM | H:SEB(5%) | ETM:LiQ(50%) | LiQ |
| | 20 nm | 180 nm | 10 nm | 20 nm | 30 nm | 1 nm |
| E26 | HTM-13: p-doped(5%) | HTM-13 | EBM | H:SEB(5%) | ETM:LiQ(50%) | LiQ |
| | 20 nm | 180 nm | 10 nm | 20 nm | 30 nm | 1 nm |
| E27 | HTM-14: p-doped(5%) | HTM-14 | EBM | H:SEB(5%) | ETM:LiQ(50%) | LiQ |
| | 20 nm | 180 nm | 10 nm | 20 nm | 30 nm | 1 nm |

TABLE 6
Data of the OLEDs

| | U @ 10 | EQE @ 10 | LT80 @ 60/40* |
|---|---|---|---|
| | mA/cm² (V) | mA/cm² (%) | mA/cm² (h) |
| E14 | 4.0 | 15.6 | 360* |
| E15 | 3.7 | 17.2 | 320* |
| E16 | 3.7 | 16.7 | 310* |
| E17 | 3.7 | 16.6 | 430* |
| E18 | 3.7 | 16.8 | 380* |
| E19 | 4.0 | 7.8 | 300 |
| E20 | 3.6 | 10.2 | 300 |
| E21 | 3.6 | 10.0 | 370 |
| E22 | 3.6 | 9.6 | 400 |
| E23 | 4.0 | 7.4 | 350 |
| E24 | 4.4 | 9.1 | 230 |
| E25 | 4.1 | 10.0 | 280 |
| E26 | 4.2 | 9.6 | 270 |
| E27 | 4.0 | 9.8 | 340 |

TABLE 7
Materials

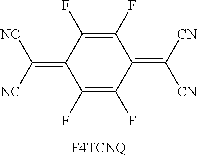 [SMILES: N#CC(C#N)=c1c(F)c(F)c(=C(C#N)C#N)c(F)c1F]

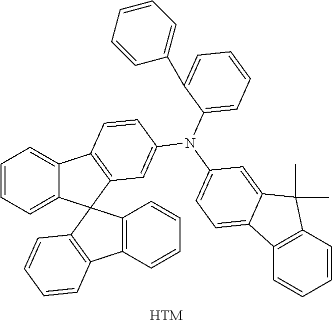

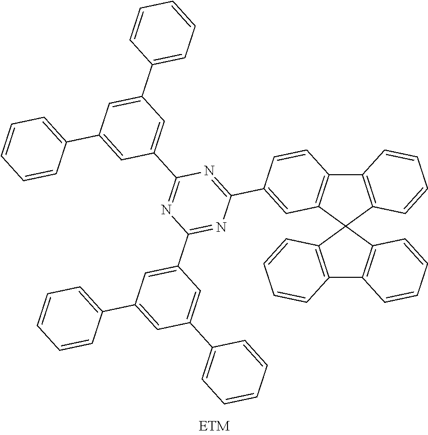 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

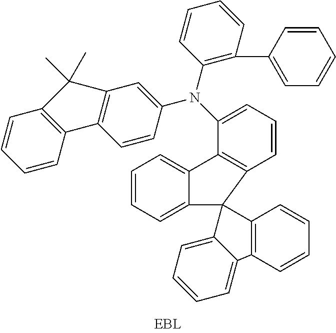 [SMILES: CC1(C)c2ccccc2-c2ccc(N(c3ccccc3-c3ccccc3)c3cccc4c3-c3ccccc3C43c4ccccc4-c4ccccc43)cc21]

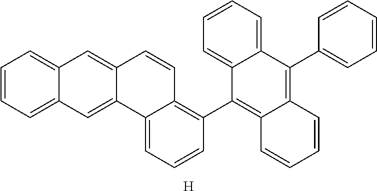

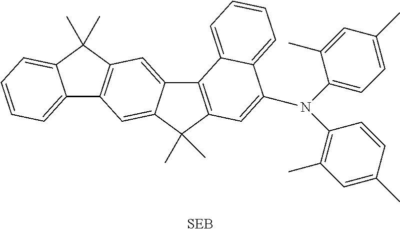 [SMILES: Cc1ccc(N(c2ccc(C)cc2C)c2cc3c(c4ccccc24)-c2cc4c(cc2C3(C)C)-c2ccccc2C4(C)C)c(C)c1]

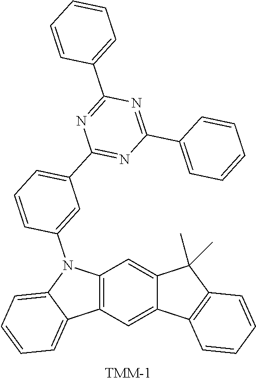

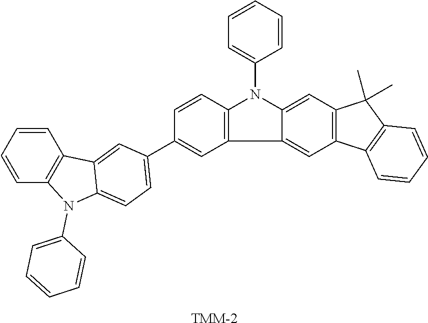

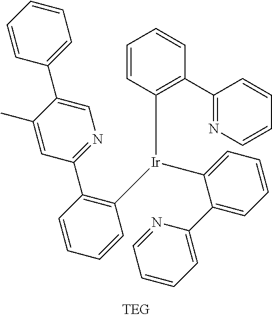

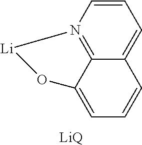

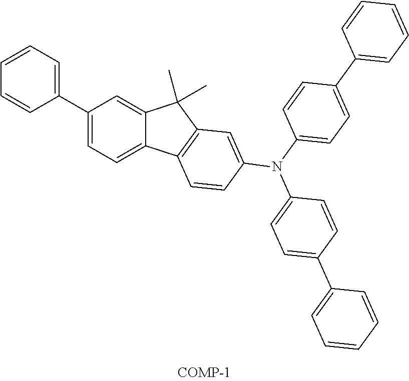 [SMILES: CC1(C)c2cc(-c3ccccc3)ccc2-c2ccc(N(c3ccc(-c4ccccc4)cc3)c3ccc(-c4ccccc4)cc3)cc21]

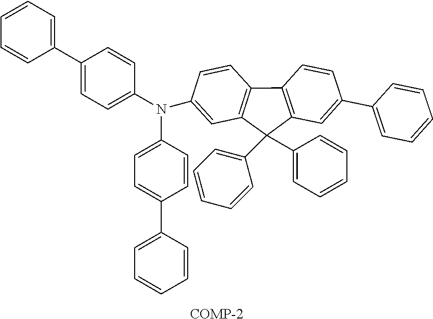

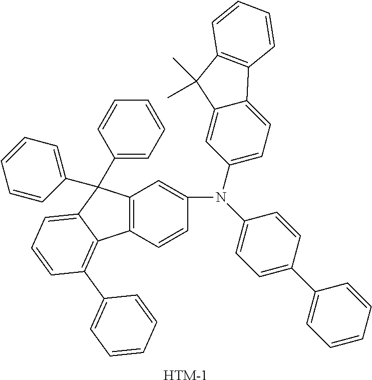

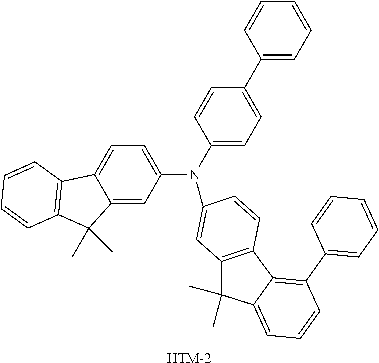

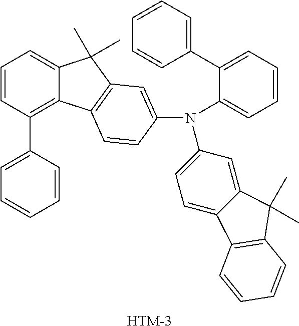

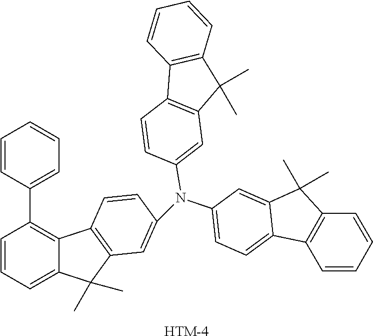

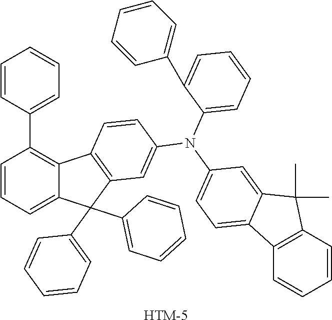

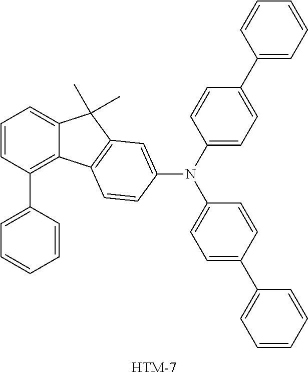

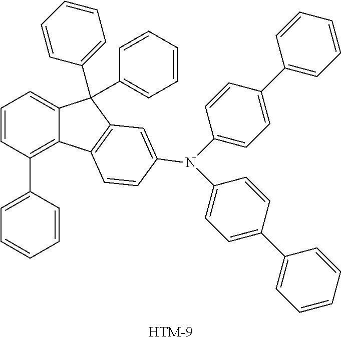

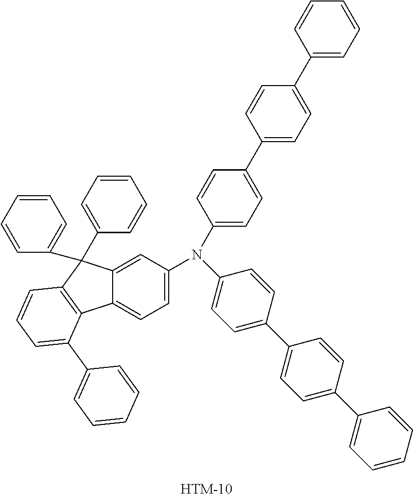

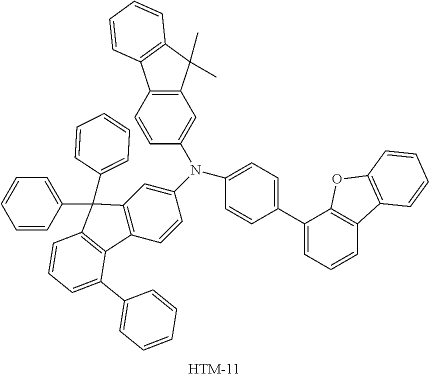

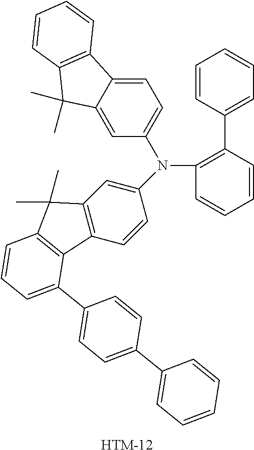

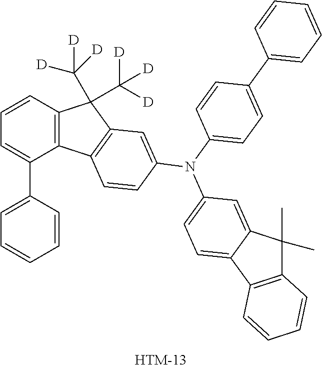

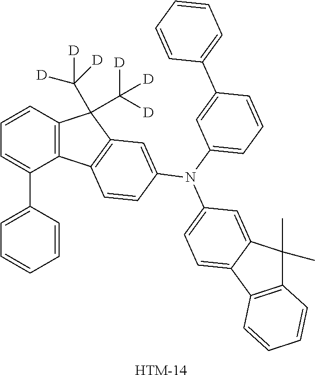

1.-30. (canceled)

31. A compound of formula (I) 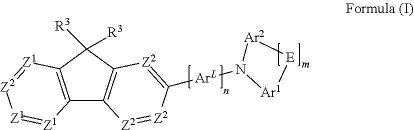
in which the variables are defined as follows:
Z¹ is, identically or differently on each occurrence, selected from CR¹, CR² and N;
Z² is, identically or differently on each occurrence, selected from CR² and N;
Ar^{L} is selected from aromatic ring systems having 6 to 40 aromatic ring atoms, which may be substituted by one or more radicals R⁴, and heteroaromatic ring systems having 5 to 40 aromatic ring atoms, which may be substituted by one or more radicals R⁴;
Ar¹, Ar² are, identically or differently, selected from aromatic ring systems having 6 to 40 aromatic ring atoms, which may be substituted by one or more radicals R⁴, and heteroaromatic ring systems having 5 to 40 aromatic ring atoms, which may be substituted by one or more radicals R⁴;
E is a single bond or is a divalent group selected from —C(R⁴)₂—, —N(R⁴)—, —O—, and —S—;
R¹ is selected, identically or differently on each occurrence, from Si(R⁵)₃, straight-chain alkyl, alkoxy or thioalkyl groups having 1 to 20 C atoms, branched or cyclic alkyl, alkoxy or thioalkyl groups having 3 to 20 C atoms, aromatic ring systems having 6 to 40 aromatic ring atoms, and heteroaromatic ring systems having 5 to 40 aromatic ring atoms, where the said alkyl, alkoxy and thioalkyl groups and the said aromatic and heteroaromatic ring systems may in each case be substituted by one or more radicals R⁵;
R² is selected, identically or differently on each occurrence, from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, SCN, SF₅, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R² may be connected to each other to form a ring; where the said alkyl, alkoxy, alkenyl and alkynyl groups and the said aromatic and heteroaromatic ring systems may in each case be substituted by one or more radicals R⁵, and where one or more CH₂ groups in the said alkyl, alkoxy, alkenyl and alkynyl groups may in each case be replaced by —R⁵C═CR⁵—, —C≡C—, Si(R⁵)₂, C═O, C═S, C═NR⁵, —C(═O)O—, —C(═O)NR⁵—, NR⁵, P(═O)(R⁵), —O—, —S—, SO or SO₂;
R³ is selected, identically or differently on each occurrence, from H, D, F, Cl, Br, I, C(═O)R⁴, CN, Si(R⁴)₃, NO₂, P(═O)(R⁴)₂, S(═O)R⁴, S(═O)₂R⁴, straight-chain alkyl, alkoxy or thioalkyl groups having 1 to 20 C atoms, or branched or cyclic alkyl, alkoxy or thioalkyl groups having 3 to 20 C atoms, alkenyl or alkynyl groups having 2 to 20 C atoms, where the said alkyl, alkoxy, thioalkyl, alkenyl and alkynyl groups may in each case be substituted by one or more radicals R⁵ and where one or more CH₂ groups in the said alkyl, alkoxy, thioalkyl, alkenyl and alkynyl groups may in each case be replaced by —R⁵C═CR⁵—, —C≡C—, Si(R⁵)₂, C═O, C═S, C═NR⁵, —C(═O)O—, —C(═O)NR⁵—, NR⁵, P(═O)(R⁵), —O—, —S—, SO or SO₂ and where in the said alkyl, alkoxy, thioalkyl, alkenyl and alkynyl groups one or more H atoms may be replaced by D, F, Cl, Br, I, CN or NO₂, or aromatic or heteroaromatic ring systems having 6 to 30 aromatic ring atoms, where the said aromatic and heteroaromatic ring systems may in each case be substituted by one or more radicals R⁵, or aryloxy groups having 5 to 60 aromatic ring atoms, or arylalkyl groups having 5 to 60 aromatic ring atoms, where the said aryloxy and arylalkyl groups may in each case be substituted by one or more radicals R⁵, where the two radicals R³ may be connected to each other to form a ring, so that a spiro compound is built at position 9 of the fluorene group, where spirobifluorenes are excluded;
R⁴ is, identically or differently on each occurrence, selected from H, D, F, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R⁴ may be connected to each other to form a ring; where the said alkyl, alkoxy, alkenyl and alkynyl groups and the said aromatic and heteroaromatic ring systems may in each case be substituted by one or more radicals R⁵, and where one or more CH₂ groups in the said alkyl, alkoxy, alkenyl and alkynyl groups may in each case be replaced by —R⁵C═CR⁵—, —C≡C—, Si(R⁵)₂, C═O, C═S, C═NR⁵, —C(═O)O—, —C(═O)NR⁵—, NR⁵, P(═O)(R⁵), —O—, —S—, SO or SO₂;
R⁵ is, identically or differently at each occurrence, selected from H, D, F, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 C atoms, branched or cyclic alkyl or alkoxy groups having 3 to 20 C atoms, alkenyl or alkynyl groups having 2 to 20 C atoms, aromatic ring systems having 6 to 40 aromatic ring atoms, and heteroaromatic ring systems having 5 to 40 aromatic ring atoms; where two or more radicals R⁵ may be connected to each other to form a ring; where the said alkyl, alkoxy, alkenyl and alkynyl groups and the said aromatic and heteroaromatic ring systems may in each case be substituted by one or more radicals R⁶, and where one or more CH₂ groups in the said alkyl, alkoxy, alkenyl and alkynyl groups may in each case be replaced by —R⁶C═CR⁶—, —C≡C—, Si(R⁶)₂, C═O, C═S, C═NR⁶, —C(═O)O—, —C(═O)NR⁶—, NR⁶, P(═O)(R⁶), —O—, —S—, SO or SO₂;
R⁶ is selected, identically or differently at each occurrence, from H, D, F, CN, alkyl groups having 1 to 20 C atoms, aromatic ring systems having 6 to 40 C atoms, and heteroaromatic ring systems having 5 to 40 aromatic ring atoms; where two or more radicals R⁶ may be connected to each other to form a ring; and where the said alkyl groups, aromatic ring systems and heteroaromatic ring systems may be substituted by F and CN;
m is 0 or 1, where in the case of m=0, the group E is not present and the groups Ar¹ and Ar² are not connected;
n is 0 or 1; where in the case of n=0, the group Ar^{L} is not present and the nitrogen atom and the fluorene group are directly connected;
characterized in that at least one of groups Z¹ is CR¹.

32. The compound according to claim 31, wherein group Ar^{L} is selected from divalent groups derived from benzene, biphenyl, terphenyl, naphthyl, fluorenyl, indenofluorenyl, spirobifluorenyl, dibenzofuranyl, dibenzothiophenyl, and carbazolyl, which may each be substituted by one or more radicals R⁴.

33. The compound according to claim 31, wherein at least one of groups Ar¹ and Ar² is selected from a radical comprising at least two rings selected from aromatic and heteroaromatic rings, which radical is optionally substituted by one or more R⁴.

34. The compound according to claim 33, characterized in that within said radical two aromatic or heteroaromatic rings are condensed or are connected to each other via a divalent group selected from —C(R⁴)₂—, —N(R⁴)—, —O—, and —S—.

35. The compound according to claim 33, wherein said radical comprises at least two aromatic rings.

36. The compound according to claim 31, wherein groups Ar¹ and Ar² are, identically or differently, selected from radicals each comprising at least two rings selected from aromatic and heteroaromatic rings, which radicals are each optionally substituted by one or more R⁴.

37. The compound according to claim 36, wherein within at least one of said radicals two aromatic or heteroaromatic rings are condensed or are connected to each other via a divalent group selected from —C(R⁴)₂—, —N(R⁴)—, —O—, and —S—.

38. The compound according to claim 36, wherein within both of said radicals two aromatic or heteroaromatic rings are condensed or are connected to each other via a divalent group selected from —C(R⁴)₂—, —N(R⁴)—, —O—, and —S—.

39. The compound according to claim 36, wherein said radicals comprise at least two aromatic rings.

40. The compound according to claim 31, wherein groups Ar¹ and Ar² are, identically or differently, selected from the following groups: phenyl, naphthyl-substituted phenyl, biphenyl, terphenyl, quarterphenyl, naphthyl, fluorenyl, benzofluorenyl, spirobifluorenyl, indenofluorenyl, dibenzofuranyl, dibenzothiophenyl, carbazolyl, benzofuranyl, benzothiophenyl, indolyl, quinolinyl, pyridyl, phenyl-substituted pyridyl, pyrimidyl, pyrazinyl, pyridazinyl and triazinyl, which are each optionally substituted by one or more radicals R⁴.

41. The compound according to claim 31, wherein m=0.

42. The compound according to claim 31, wherein it conforms to one of Formulae (I-A) to (I-G) 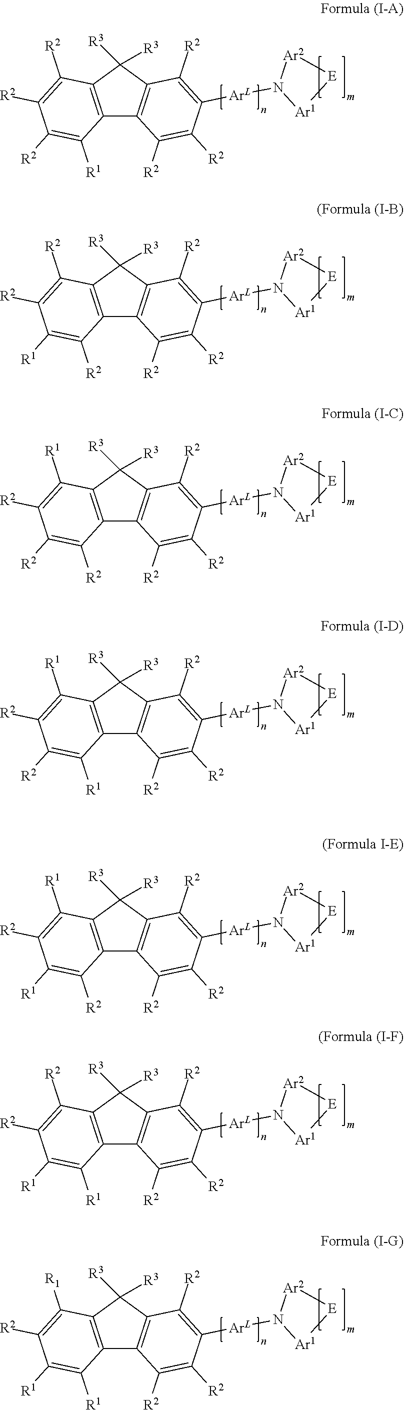
in which the variables occurring are as defined in claim 31.

43. The compound according to claim 31, wherein it conforms to one of formulae (I-A-1) to (I-G-1) 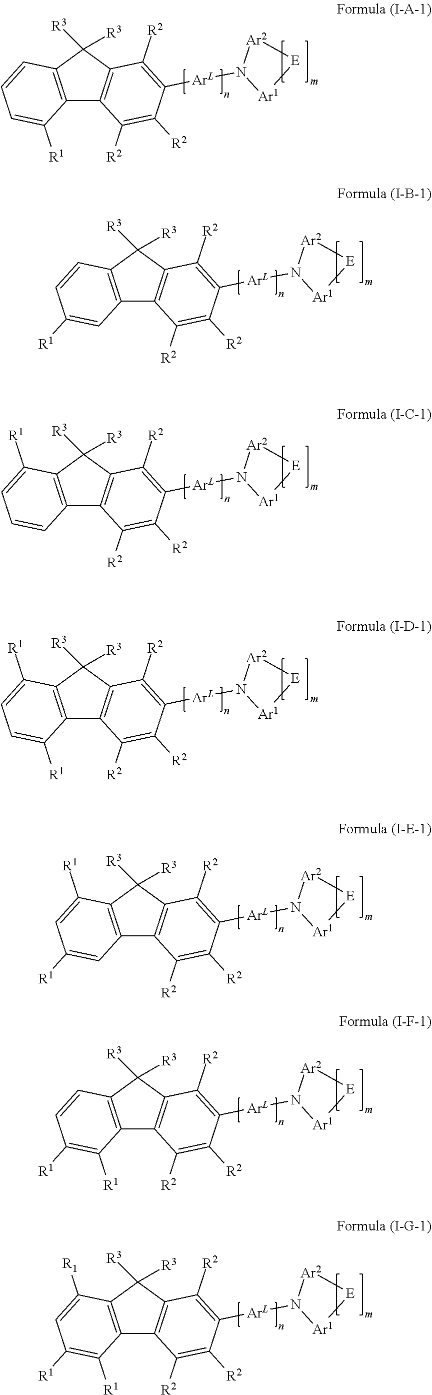
in which the variables occurring are as defined in claim 31.

44. The compound according to claim 31, wherein it conforms to one of formulae (I-A-2) to (I-K-2) 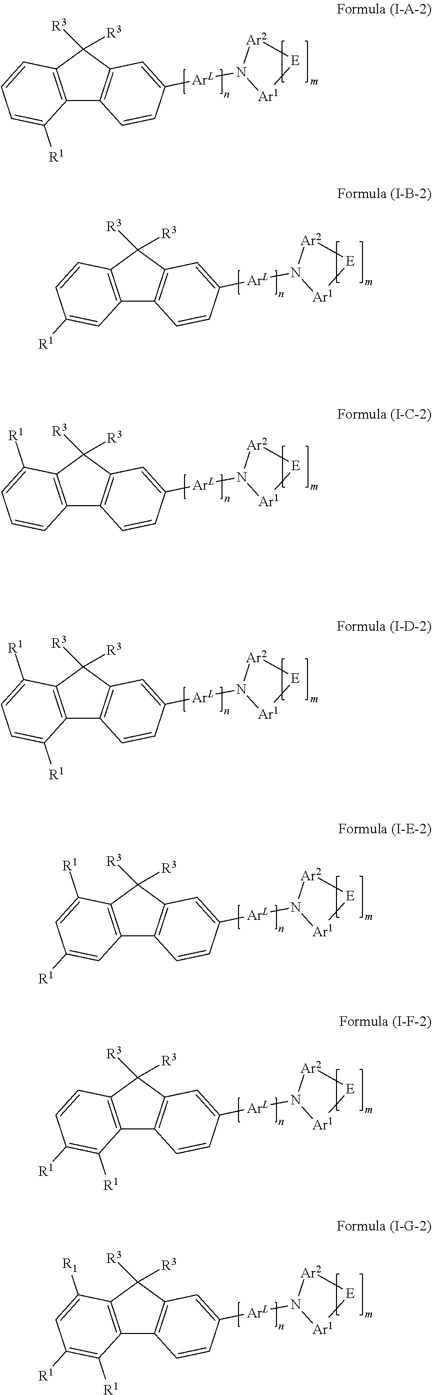 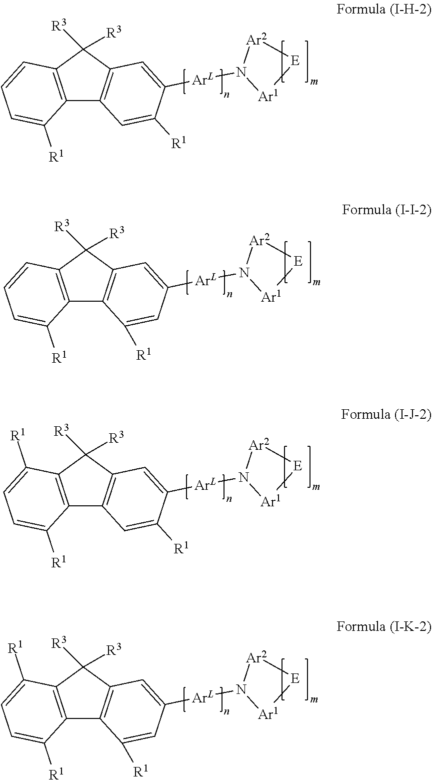
in which the variables occurring are as defined in claim 31.

45. The compound according to claim 31, wherein it conforms to one of the following formulae 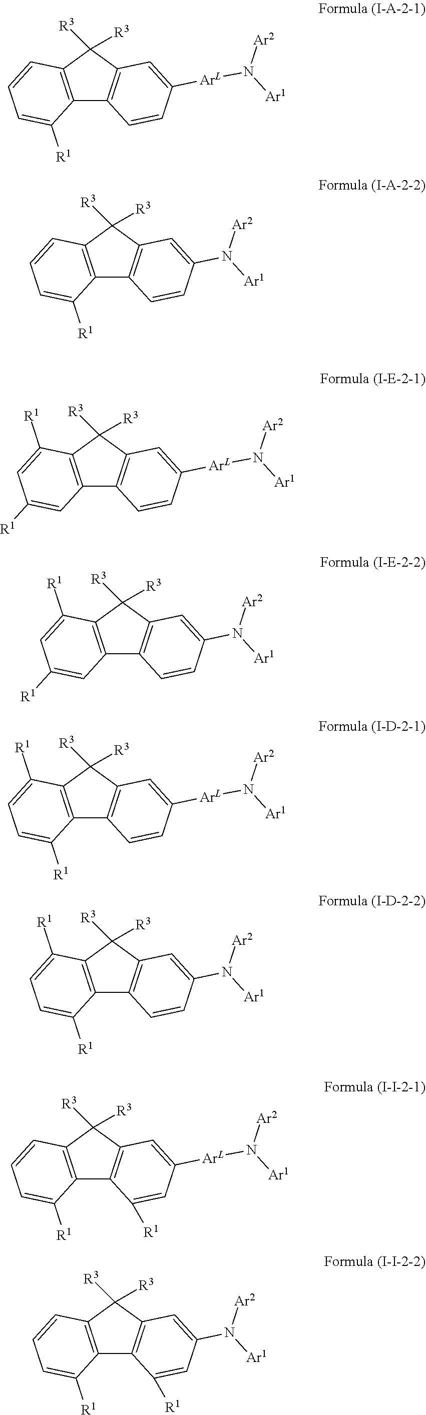 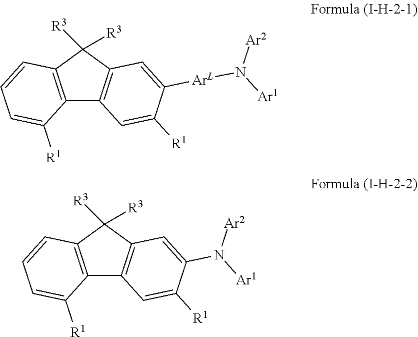
in which the variables occurring are as defined in claim 31, and wherein in Formulae (I-A-2-1) and (I-H-2-1) A^{L} is selected from divalent groups derived from benzene, biphenyl, terphenyl, naphthyl, dibenzofuranyl, dibenzothiophenyl, which may each be substituted by one or more radicals R⁴.

46. The compound according to claim 31, wherein R¹ is, identically or differently on each occurrence, selected from 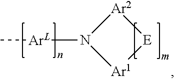
in which m is 1 and E is a single bond, aromatic ring systems having 6 to 30 aromatic ring atoms, and heteroaromatic ring systems having 5 to 30 aromatic ring atoms, where the said aromatic and heteroaromatic ring systems may in each case be substituted by one or more radicals R⁵.

47. The compound according to claim 31, wherein R¹ is, identically or differently on each occurrence, selected from 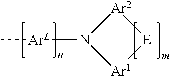
in which m is 1 and E is a single bond, phenyl, biphenyl, terphenyl, quarterphenyl, naphthyl, fluorenyl, especially 9,9′-dimethylfluorenyl and 9,9′-diphenylfluorenyl, benzofluorenyl, spirobifluorenyl, indenofluorenyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, each of which may optionally be substituted by one or more radicals R⁵.

48. The compound according to claim 31, wherein R³ is selected, identically or differently on each occurrence, from straight-chain alkyl groups having 1 to 20 C atoms, or cyclic alkyl groups having 3 to 20 C atoms, where the said alkyl groups or cyclic alkyl groups may be substituted by one or more radicals R⁵, or aromatic or heteroaromatic ring systems having 6 to 30 aromatic ring atoms, where the said aromatic and heteroaromatic ring systems may in each case be substituted by one or more radicals R⁵, where the two radicals R³ may be connected to each other to form a ring, so that a spiro compound is built at position 9 of the fluorene group, where spirobifluorenes are excluded.

49. The compound according to claim 31, wherein R³ is identically or different on each occurrence, selected from straight-chain alkyl groups having 1 to 10 C atoms, where the said alkyl groups may be substituted by one or more radicals R⁵, or aromatic ring systems having 6 to 24 aromatic ring atoms, where the said aromatic ring systems may in each case be substituted by one or more radicals R⁵, where the two radicals R³ may be connected to each other to form a ring, so that a spiro compound is built at position 9 of the fluorene group, where spirobifluorenes are excluded.

50. A process for the preparation of a compound of formula (I) according to claim 31, comprising introducing a diarylamino group by a C—N coupling reaction between a fluorene derivative, which is halogenated at 2-position, and a diarylamine derivative.

51. A process for the preparation of a compound of formula (I) according to claim 31, characterized in that the compound is prepared by reacting an alkyl 5-halo-2-iodobenzoate with an arylboronic acid.

52. Process according to claim 51, comprising the following reaction steps:
a) Reacting a methyl 5-halo-2-iodobenzoate of general formula (II) 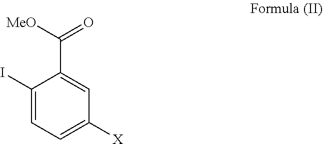
with an arylboronic acid derivative which conforms to one of formulae (III-1) to (III-8) 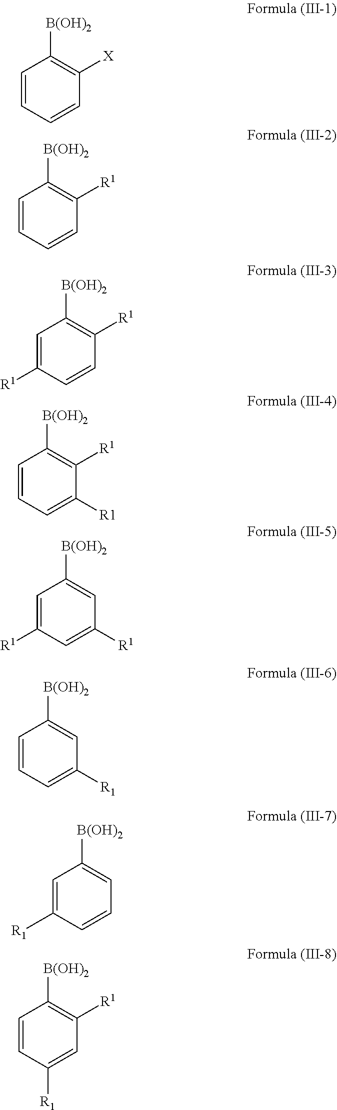
in which
R¹ is, identically or differently on each occurrence, and is selected from phenyl, biphenyl, terphenyl or quaterphenyl, each of which may optionally be substituted by one or more radicals R⁵ as defined above; and
X is Cl or Br,
to obtain a 5-halobenzoate methyl ester derivative, and subsequently
b) converting the ester derivative to a tertiary alcohol by using an alkyl- or aryl-magnesium halide, or phenyl-magnesium chloride, and subsequently
c) performing acid-catalyzed cyclisation to obtain a fluorene derivative, which is halogenated at 2-position, and subsequently
d) reacting the fluorene derivative with a diarylamine derivative to obtain a compound of formula (I).

53. A compound of formulae (IV-A) to (IV-Is) 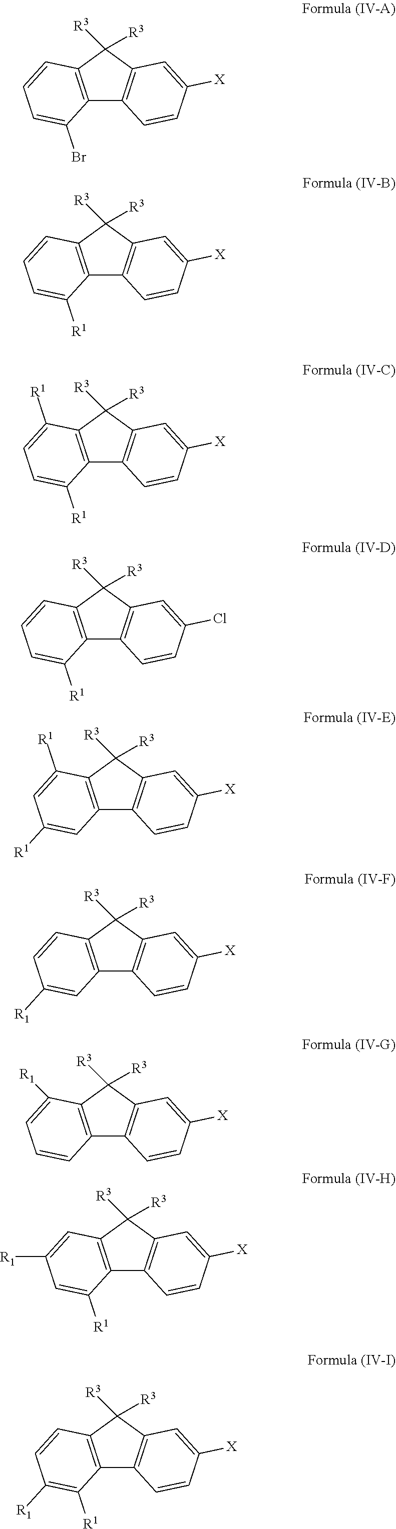 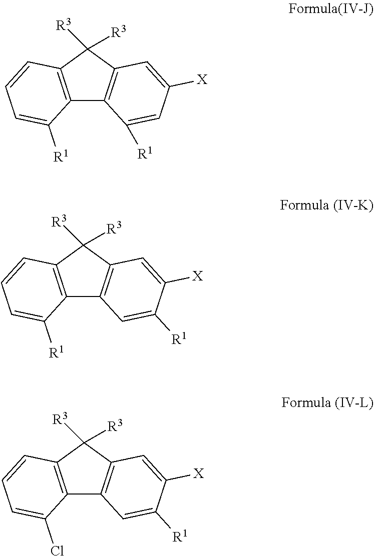
in which
R¹ is selected, identically or differently on each occurrence, from phenyl, biphenyl, terphenyl or quarterphenyl, each of which may optionally be substituted by one or more radicals R⁵ as defined above;
R³ is selected, identically or differently on each occurrence, from methyl and phenyl, each of which may optionally be substituted by one or more radicals R⁵ as defined above; and
X is Cl or Br.

54. A compound of formulae (IV-A) to (IV-Is) 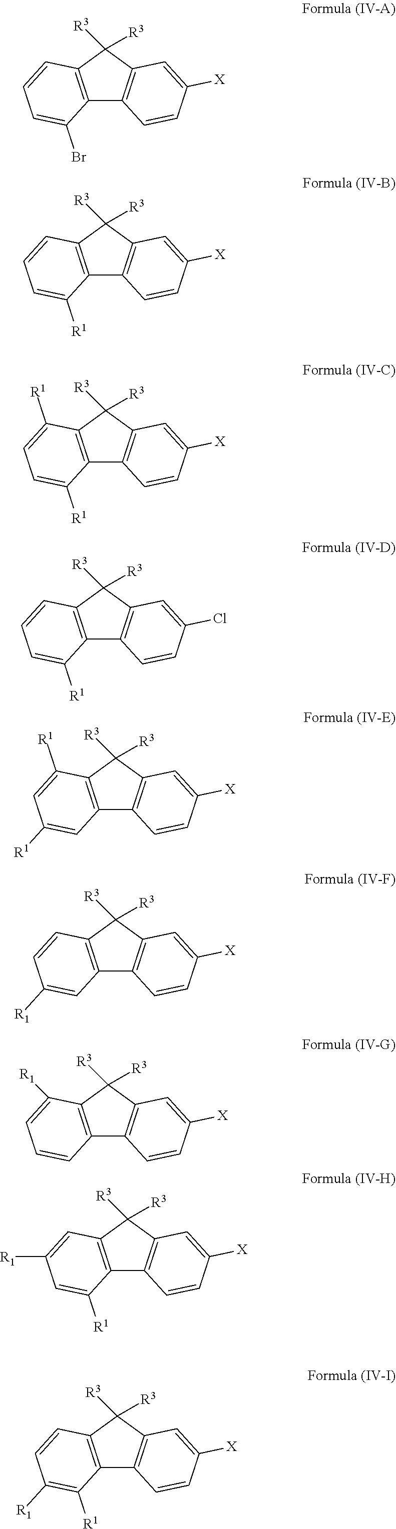 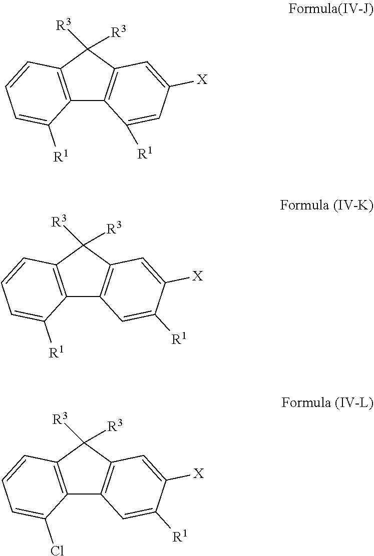
in which
R¹ is selected, identically or differently on each occurrence, from phenyl, biphenyl, terphenyl or quarterphenyl, each of which may optionally be substituted by one or more radicals R⁵ as defined above;
R³ is selected, identically or differently on each occurrence, from methyl and phenyl, each of which may optionally be substituted by one or more radicals R⁵ as defined above; and
X is Cl or Br and which is obtained or obtainable in reaction step c) of claim 52.

55. An oligomer, polymer or dendrimer, comprising one or more compounds of formula (I) according to claim 31, where the bond(s) to the polymer, oligomer or dendrimer may be localised at any desired positions in formula (I) substituted by R¹ to R⁶.

56. A composition, comprising one or more compounds of formula (I) according to claim 31 and at least one further organic functional material selected from the group consisting of fluorescent emitters, phosphorescent emitters, host materials, matrix materials, electron transporting materials, electron injecting materials, hole transporting materials, hole injecting materials, electron blocking materials, hole blocking materials, wide band gap materials, delayed fluorescent emitters and delayed fluorescent hosts.

57. A formulation comprising at least one compound of formula (I) according to claim 31, and at least one solvent.

58. An electronic device, comprising at least one compound according to claim 31.

59. An organic electroluminescent device, comprising anode, cathode and at least one emitting layer, where at least one organic layer of the device, which is an emitting layer, a hole transport layer, an electron blocking layer or a hole injection layer, comprises the at least one compound of formula (I) according to claim 31.
